(19) United States
(12) Patent Application Publication
(10) Pub. No.: US 2026/0069728 A1
(43) Pub. Date: Mar. 12, 2026

(54) MULTI-TARGETING COMPOUND AND USE THEREOF

(71) Applicants: Chengdu Shetai Medical Technology Co., Ltd., Chengdu (CN); Grand Pharma (China) Co., Ltd., Wuhan (CN)

(72) Inventors: Weikun Tang, Wuhan (CN); Shuheng Liang, Chengdu (CN); Yaxin Shi, Wuhan (CN); Feng Gao, Wuhan (CN); Wentao Huang, Wuhan (CN); Na Wu, Wuhan (CN); Yiran Zhang, Wuhan (CN); Xiaoyan Wu, Wuhan (CN); Xixi Hu, Wuhan (CN); Shimin Zhang, Wuhan (CN); Shuqun Wang, Wuhan (CN); Si Wen, Wuhan (CN); Xiaoke Huang, Wuhan (CN); Hongyu Yao, Wuhan (CN); Jianxia Chen, Wuhan (CN); Miao Yuan, Wuhan (CN); Lina Ye, Wuhan (CN); Dan Zhou, Wuhan (CN); Ju Yang, Wuhan (CN); Miao Wu, Wuhan (CN); Qiao Wang, Wuhan (CN)

(73) Assignees: Chengdu Shetai Medical Technology Co., Ltd., Chengdu (CN); Grand Pharma (China) Co., Ltd., Wuhan (CN)

(21) Appl. No.: 19/391,561

(22) Filed: Nov. 17, 2025

Related U.S. Application Data
(63) Continuation of application No. PCT/CN2024/093514, filed on May 15, 2024.

(30) Foreign Application Priority Data
May 15, 2023 (CN) 202310545693.1
May 15, 2023 (CN) 202310546451.4
May 15, 2023 (CN) 202310546461.8

(51) Int. Cl. A61K 51/08; A61K 51/04; A61P 35/00; C07K 1/107

(52) U.S. Cl. CPC A61K 51/088; A61K 51/0482; A61P 35/00; C07K 1/1077; A61K 2121/00; A61K 2123/00

(57) ABSTRACT

A multi-targeting compound, a pharmaceutical composition comprising or consisting of same, a kit comprising or consisting of the compound or pharmaceutical composition, and the use of the compound or pharmaceutical composition in the diagnosis or treatment of diseases characterized by abnormal expression of FAP, CXCR4, GRPR, and/or αᵥβ₃.

### CROSS-REFERENCE TO RELATED APPLICATIONS

This application claims priority to three Chinese patent applications with application numbers 2023105464618, 2023105464514, and 2023105456931 filed on May 15, 2023, the entire contents of which are incorporated herein by reference in their entirety.

### TECHNICAL FIELD

The present invention relates to a multi-targeting compound, a pharmaceutical composition comprising or consisting of the compound, a kit comprising or consisting of the compound or pharmaceutical composition, and the use of the compound or pharmaceutical composition in the diagnosis or treatment of diseases.

### BACKGROUND

Fibroblast activation protein (FAP) is a membrane-bound serine peptidase expressed on the surface of activated fibroblasts within the tumor stroma, and plays an important role in the occurrence and development of tumors. Previous studies have shown that FAP is typically not expressed in normal human tissues but is selectively highly expressed on the surface of stromal fibroblasts in over 90% of epithelial malignancies, including breast cancer, ovarian cancer, lung cancer, colorectal cancer, gastric cancer, and pancreatic cancer. Given its widespread expression and important role in tumors. FAP has become a crucial target for tumor imaging and therapy.

C-X-C chemokine receptor type 4 (CXCR4), also known as fusin or cluster of differentiation 184 (CD184), is a seven-transmembrane G-protein coupled receptor (GPCR) belonging to the class I GPCR or rhodopsin-like GPCR family. Under normal physiological conditions, CXCR4 plays multiple roles and is primarily expressed in the hematopoietic and immune systems. CXCR4 was initially identified as one of the co-receptors involved in human immunodeficiency virus (HIV) cell entry. Subsequent research has shown that it is expressed in many tissues, including the brain, thymus, lymphoid tissue, spleen, stomach, and small bowel, as well as in specific cell types such as hematopoietic stem cells (HSCs), mature lymphocytes, and fibroblasts. CXCL12, previously designated SDF-1α, is the only known ligand for CXCR4. CXCR4 mediates stem cell migration during embryonic development and in response to injury and inflammation. CXCR4 has been demonstrated to play multiple roles in human diseases such as cell proliferative disorders, Alzheimer's disease, HIV, rheumatoid arthritis, pulmonary fibrosis, etc.

Integrin αᵥβ₃ is a heterodimeric receptor located on the cell surface. It is rarely expressed in normal vascular endothelial and epithelial cells but is highly expressed on the surface of various solid tumor cells, such as lung cancer, osteosarcoma, neuroblastoma, breast cancer, prostate cancer, bladder cancer, glioblastoma, and invasive melanoma. Furthermore, it is highly expressed on the cell membranes of neovascular endothelial cells in all tumor tissues, suggesting that integrin αᵥβ₃ plays a key role in tumor growth, invasion, and metastasis. Polypeptides containing an arginine-glycine-aspartic acid (RGD) sequence can specifically bind to integrin αᵥβ₃. Various radionuclide-labeled RGD peptides have been successfully used for imaging investigations in multiple tumor-bearing animal models.

The action of gastrin-releasing peptide (GRP) is primarily mediated through binding to its receptor, the GRP receptor (GRPR). GRPR is a G-protein coupled receptor initially isolated from a small cell lung cancer cell line. Upregulation of the GRP/GRPR pathway has been reported in several cancers, including breast cancer, prostate cancer, uterine cancer, ovarian cancer, colon cancer, pancreatic cancer, gastric cancer, lung cancer (both small cell and non-small cell lung cancers), head and neck squamous cell carcinoma, and various brain tumors and neuromas.

In clinical practice, radionuclide-labeled targeting compounds have made significant progress in the field of precise tumor imaging. Radioactive ligands targeting a single target require a certain receptor to specifically highly express in the tumor tissue while not express or express at low levels in normal tissues. However, during tumor growth, receptors on the surface of tumor cells exhibit heterogeneity and non-uniformity. Even among patients with the same type of tumor, the receptor types or expression levels in the tumor tissue may vary. Probes currently reported have shortcomings such as low uptake values, and their sensitivity of diagnosis and therapeutic efficacy still need improvement.

### SUMMARY

To address at least one of the technical problems existing in the prior art, the present invention provides a multi-targeting compound, and the preparation method and use thereof. In addition to targeting the FAP receptor, this compound can also simultaneously target a CXCR4, an αᵥβ₃, or a GRPR receptor.

The present invention provides a compound having a structure of formula (I), or a stereoisomer, tautomer, or pharmaceutically acceptable salt thereof:

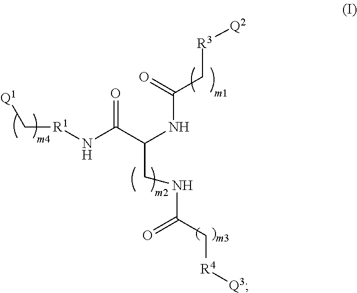

wherein,
R¹, R³ and R⁴ are each independently selected from the group consisting of a bond, —U¹-G¹-A¹-L-A²-G²-U²—, and a heteroalkylene, at least one of R¹, R³ and R⁴ is —U¹-G¹-A¹-L-A²-G²-U²—; or,

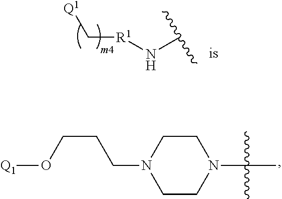

and at least one of R³ and R⁴ is —U¹-G¹-A¹-L-A²-G²-U²—;
L is

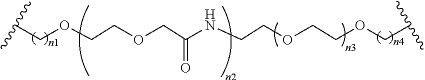

or a bond, n1, n2, n3 and n4 are each independently 0, 1, 2, or 3;
A¹ and A² are each independently selected from the group consisting of a bond, —C(═O)NH—, —NHC(═O)—, and —C(═O)—;
G¹ and G² are each independently selected from the group consisting of a bond, a heteroalkylene, and

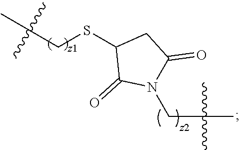

z1 and z2 are each independently selected from the group consisting of 0, 1, 2, and 3;
U¹ and U² are each independently selected from the group consisting of a bond,

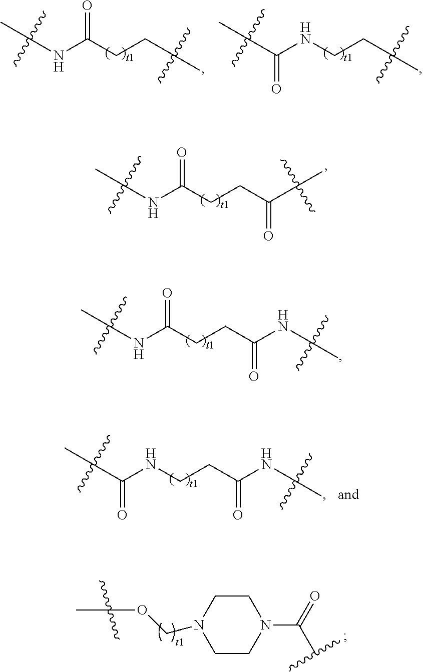

each t1 is independently selected from the group consisting of 1, 2, 3, and 4;
U¹, G¹, A¹, L, A², G² and U² are not a bond at the same time;
m1, m2, m3 and m4 are each independently selected from the group consisting of 0), 1, 2, 3, 4, and 5;
one of Q¹, Q², and Q³ is a chelating group, another is a FAP receptor-targeting moiety, and the third is an additional receptor-targeting moiety, wherein the additional receptor is selected from the group consisting of a CXCR4 receptor, an αᵥβ₃ receptor, and a GRPR receptor.

In some embodiments, Q¹ is a chelating group. In some embodiments, Q² is a FAP receptor-targeting moiety, Q³ is an additional receptor-targeting moiety, wherein the additional receptor is selected from the group consisting of a CXCR4 receptor, an αᵥβ₃ receptor, and a GRPR receptor. In some embodiments. Q² is an additional receptor-targeting moiety, wherein the additional receptor is selected from the group consisting of a CXCR4 receptor, an αᵥβ₃ receptor, and a GRPR receptor; and Q³ is a FAP receptor-targeting moiety.

In some embodiments, Q² is a chelating group. In some embodiments, Q¹ is a FAP receptor-targeting moiety, Q³ is an additional receptor-targeting moiety, wherein the additional receptor is selected from the group consisting of a CXCR4 receptor, an αᵥβ₃ receptor, and a GRPR receptor. In some embodiments, Q¹ is an additional receptor-targeting moiety, wherein the additional receptor is selected from the group consisting of a CXCR4 receptor, an αᵥβ₃ receptor, and a GRPR receptor; Q³ is a FAP receptor-targeting moiety.

In some embodiments, Q³ is a chelating group. In some embodiments, Q¹ is a FAP receptor-targeting moiety, Q² is an additional receptor-targeting moiety, wherein the additional receptor is selected from the group consisting of a CXCR4 receptor, an αᵥβ₃ receptor, and a GRPR receptor. In some embodiments, Q¹ is an additional receptor-targeting moiety, wherein the additional receptor is selected from the group consisting of a CXCR4 receptor, an αᵥβ₃ receptor, and a GRPR receptor; and Q² is a FAP receptor-targeting moiety.

In some embodiments, one of Q¹, Q² and Q³ is a chelating group, another is a FAP receptor-targeting moiety, and the third is a CXCR4 receptor-targeting moiety. In some embodiments, Q¹ is a FAP receptor-targeting moiety, Q² is a CXCR4 receptor-targeting moiety, and Q³ is a chelating group; or, Q² is a FAP receptor-targeting moiety, Q¹ is a CXCR4 receptor-targeting moiety, and Q³ is a chelating group.

In some embodiments, one of Q¹, Q² and Q³ is a chelating group, another is a FAP receptor-targeting moiety, and the third is an αᵥβ₃ receptor-targeting moiety. In some embodiments, Q¹ is a FAP receptor-targeting moiety, Q² is an αᵥβ₃ receptor-targeting moiety, Q³ is a chelating group; or, Q² is a FAP receptor-targeting moiety, Q¹ is an αᵥβ₃ receptor-targeting moiety. Q³ is a chelating group.

In some embodiments, one of Q¹. Q² and Q³ is a chelating group, another is a FAP receptor-targeting moiety, and the third is a GRPR receptor-targeting moiety. In some embodiments, Q¹ is a FAP receptor-targeting moiety, Q² is a GRPR receptor-targeting moiety, Q³ is a chelating group; or, Q² is a FAP receptor-targeting moiety, Q¹ is a GRPR receptor-targeting moiety, Q³ is a chelating group.

In some embodiments, Q¹ is a FAP receptor-targeting moiety. Q² is a CXCR4 receptor-targeting moiety, and Q³ is a chelating group.

In some embodiments, Q² is a FAP receptor-targeting moiety, Q¹ is a CXCR4 receptor-targeting moiety, and Q³ is a chelating group.

In some embodiments, Q¹ is a FAP receptor-targeting moiety, Q² is an αᵥβ₃ receptor-targeting moiety, and Q³ is a chelating group.

In some embodiments, Q² is a FAP receptor-targeting moiety, Q¹ is an αᵥβ₃ receptor-targeting moiety, and Q³ is a chelating group.

In some embodiments. Q¹ is a FAP receptor-targeting moiety, Q² is a GRPR receptor-targeting moiety, and Q³ is a chelating group.

In some embodiments, Q² is a FAP receptor-targeting moiety, Q¹ is a GRPR receptor-targeting moiety. Q³ is a chelating group.

In some embodiments, R³ comprises

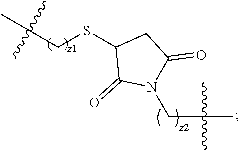

and the thioether bond in its main chain confers superior tumor-targeting property to the compound.

In some embodiments, the heteroalkylene is an alkylene comprising at least one sulfur atom. In some embodiments, the heteroalkylene is —(CH₂)_{q}—S—(CH₂)ₚ—, where p and q are each independently selected from the group consisting of 0, 1, 2, 3, 4, and 5, provide that p and q are not both 0. In some embodiments, p is 0 or 1; q is 1 or 2.

In some embodiments, the compound has a structure of formula (I′-A), or a stereoisomer, tautomer, or pharmaceutically acceptable salt thereof:

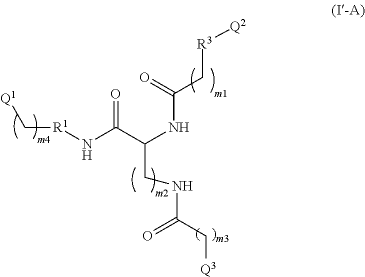

each symbol has the definition given in Formula (I),
preferably, Q¹ is a FAP receptor-targeting moiety, Q² is an additional receptor-targeting moiety such as a CXCR4 receptor, an αᵥβ₃ receptor, or a GRPR receptor, Q³ is a chelating group; or, Q² is a FAP receptor-targeting moiety, Q¹ is an additional receptor-targeting moiety such as a CXCR4 receptor, an αᵥβ₃ receptor, or a GRPR receptor, Q³ is a chelating group;
preferably, one of R¹ and R³ is —U¹-G¹-A¹-L-A²-G²-U²—, and the other is selected from the group consisting of a bond, U¹, and G¹;
L is

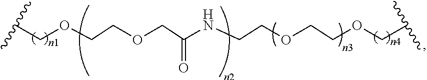

n1, n2, n3 and n4 are each independently 0, 1, 2, or 3;
A¹ and A² are each independently selected from the group consisting of a bond, —C(═O) NH—, —NHC(═O)—, and —C(═O)—;
G¹ and G² are each independently selected from the group consisting of a bond, —(CH₂)_{q}—S—(CH₂)ₚ—, and

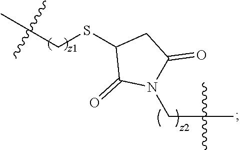

z1 and z2 are each independently selected from the group consisting of 0, 1, 2, and 3; p and q are each independently selected from the group consisting of 0, 1, 2, 3, 4, and 5, and p and q are not both 0;
U¹ and U² are each independently selected from the group consisting of a bond,

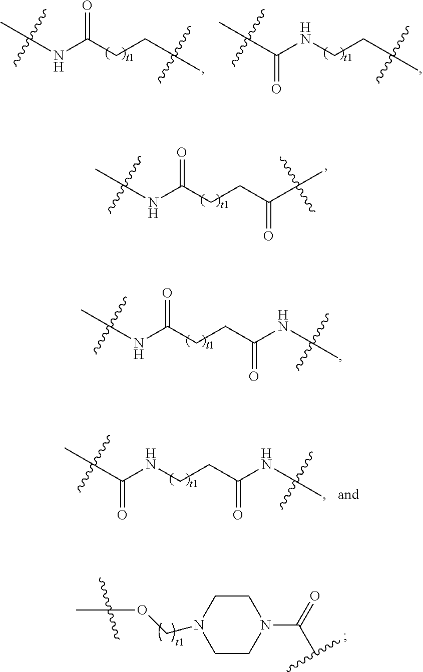

each t1 is independently selected from the group consisting of 1, 2, 3, and 4;
m1, m2, m3 and m4 are each independently selected from the group consisting of 0, 1, 2, 3, 4, and 5.

In some embodiments, the compound has a structure of formula I′-B, or a stereoisomer, tautomer, or pharmaceutically acceptable salt thereof:

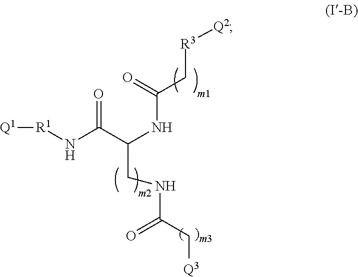

each symbol has the definition given in Formula (I),
preferably, Q¹ is a FAP receptor-targeting moiety, Q² is an additional receptor-targeting moiety such as a GRPR receptor. Q³ is a chelating group; or, Q² is a FAP receptor-targeting moiety, Q¹ is an additional receptor-targeting moiety such as a GRPR receptor, Q³ is a chelating group; one of R¹ and R³ is —U¹-G¹-A¹-L-A²-G²-U²—, and the other is selected from the group consisting of a bond, —(CH₂)_{q}—S—(CH₂)ₚ—, U¹, and G¹; p and q are each independently selected from the group consisting of 0, 1, 2, 3, 4, and 5, and, p and q are not both 0;
L is

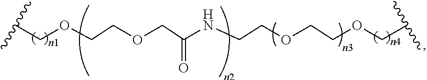

n1, n2, n3 and n4 are each independently 0, 1, 2, or 3;
A¹ and A² are each independently selected from the group consisting of a bond, —C(═O)NH—, —NHC(═O)—, and —C(═O)—;
G¹ and G² are each independently selected from the group consisting of a bond and

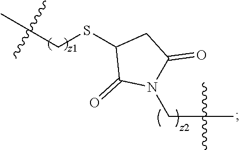

z1 and z2 are each independently selected from the group consisting of 0, 1, 2, and 3;
U¹ and U² are each independently selected from the group consisting of a bond,

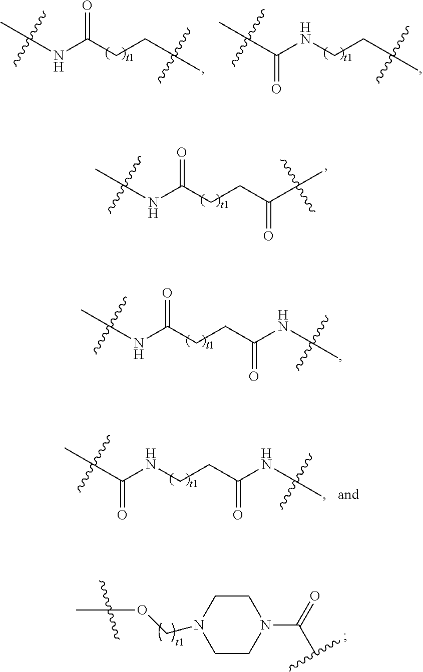

each t1 is independently selected from the group consisting of 1, 2, 3, and 4;
m1, m2, and m3 are each independently selected from the group consisting of 0, 1, 2, 3, 4, and 5.

In some embodiments, the compound has a structure of formula I′-C, or a stereoisomer, tautomer, or pharmaceutically acceptable salt thereof:

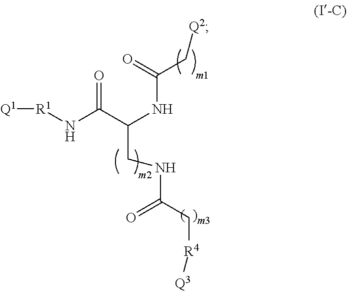

each symbol is as defined in Formula (I), preferably, Q¹ is a FAP receptor-targeting moiety, Q³ is an additional receptor-targeting moiety such as an αᵥβ₃ receptor, Q² is a chelating group; or Q³ is a FAP receptor-targeting moiety, Q¹ is an additional receptor-targeting moiety such as an αᵥβ₃ receptor, Q² is a chelating group;
preferably, one of R¹ and R⁴ is —U¹-G¹-A¹-L-A²-G²-U²—, and the other is selected from the group consisting of a bond, —(CH₂)_{q}—S—(CH₂)ₚ—, U¹, and G¹, p and q are each independently selected from the group consisting of 0, 1, 2, 3, 4, and 5, and, p and q are not both 0;
or,

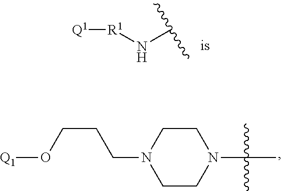

R⁴ is —U¹-G¹-A¹-L-A²-G²-U²—;
L is

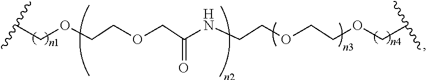

n1, n2, n3 and n4 are each independently 0, 1, 2, or 3;
A¹ and A² are each independently selected from the group consisting of a bond, —C(═O) NH—, —NHC(═O)—, and —C(═O)—;
G¹ and G² are each independently selected from the group consisting of a bond and

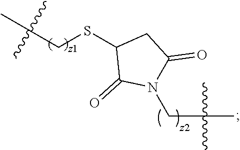

z1 and z2 are each independently selected from the group consisting of 0, 1, 2, and 3;
U¹ and U² are each independently selected from the group consisting of a bond,

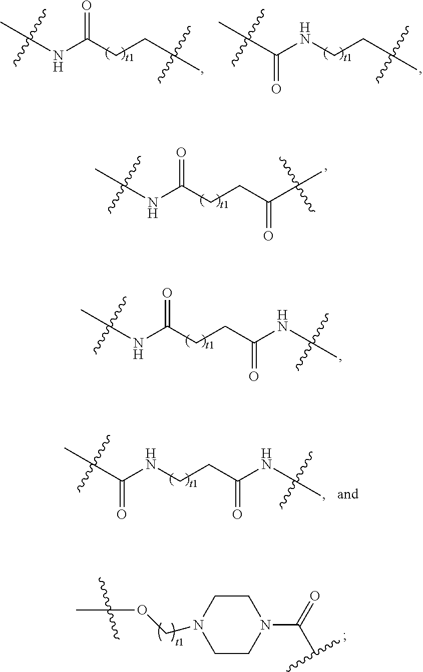

each t1 is independently selected from the group consisting of 1, 2, 3, and 4;
m1, m2 and m3 are each independently selected from the group consisting of 0, 1, 2, 3, 4, and 5.

In some embodiments, the compound has a structure of formula I-2, or a stereoisomer, tautomer, or pharmaceutically acceptable salt thereof:

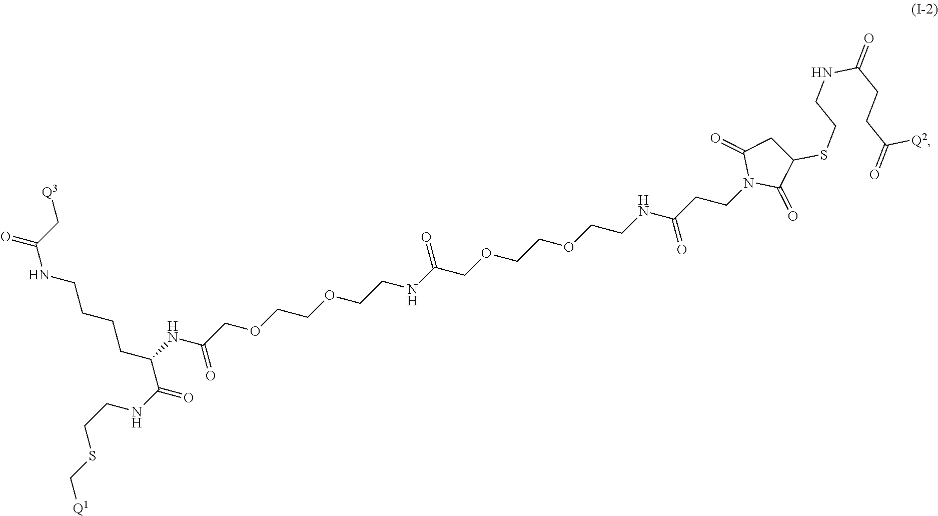

in formula I-2, Q¹, Q², Q³ are as defined in Formula (I); preferably, Q¹ is a FAP receptor-targeting moiety, Q² is an additional receptor-targeting moiety such as a CXCR4 receptor, Q³ is a chelating group; or Q¹ is an additional receptor-targeting moiety such as a CXCR4 receptor, Q² is a FAP receptor-targeting moiety, Q³ is a chelating group.

In some embodiments, the compound has a structure of formula I-1, I-3, or I-4, or a stereoisomer, tautomer, or pharmaceutically acceptable salt thereof:

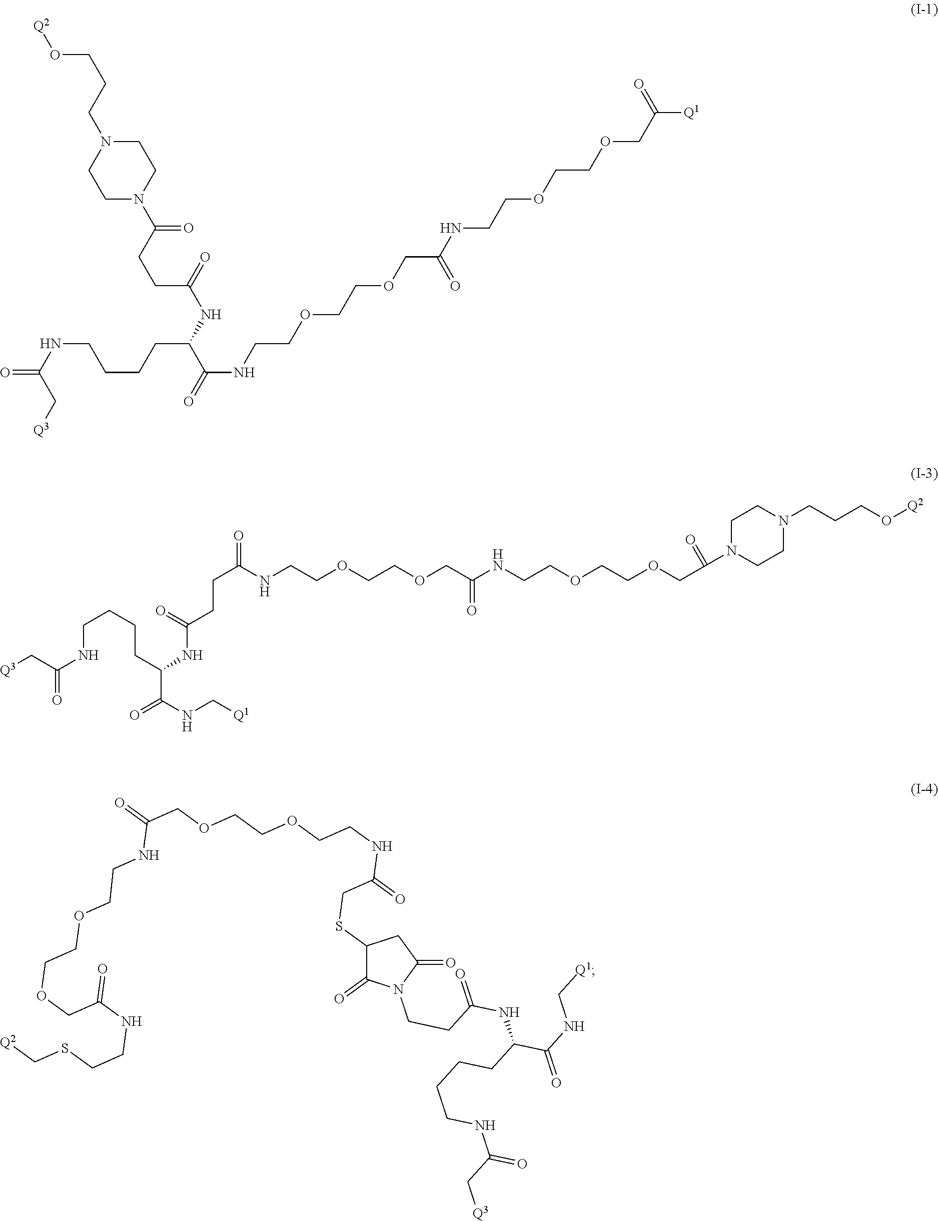

wherein, Q¹, Q², Q³ are as defined in Formula (I); preferably, Q² is a FAP receptor-targeting moiety, Q¹ is an additional receptor-targeting moiety such as a CXCR4 receptor, Q³ is a chelating group; or Q¹ is a FAP receptor-targeting moiety, Q² is an additional receptor-targeting moiety such as a CXCR4 receptor, Q³ is a chelating group.

In some embodiments, the compound has a structure of formula I-5, I-6, I-7, or I-8, or a stereoisomer, tautomer, or pharmaceutically acceptable salt thereof:

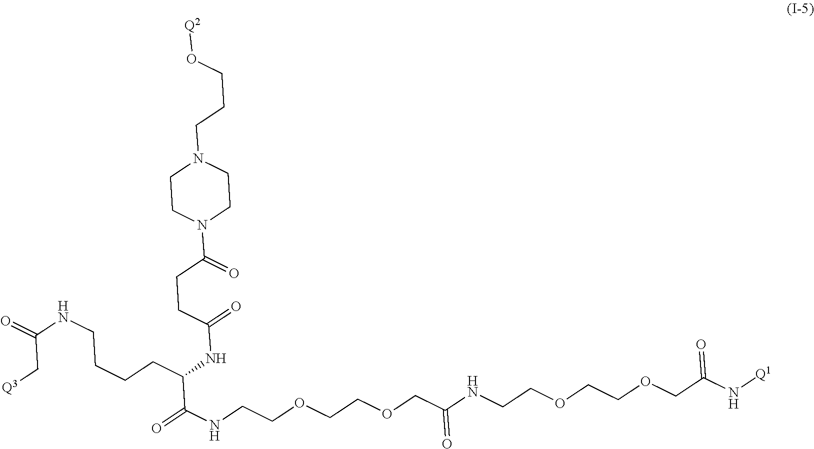

in formula I-5, Q¹, Q², Q³ are as defined in Formula (I); preferably, Q¹ is an additional receptor-targeting moiety such as a GRPR receptor, Q² is a FAP receptor-targeting moiety, Q³ is a chelating group; or Q² is an additional receptor-targeting moiety such as a GRPR receptor, Q¹ is a FAP receptor-targeting moiety, Q³ is a chelating group;

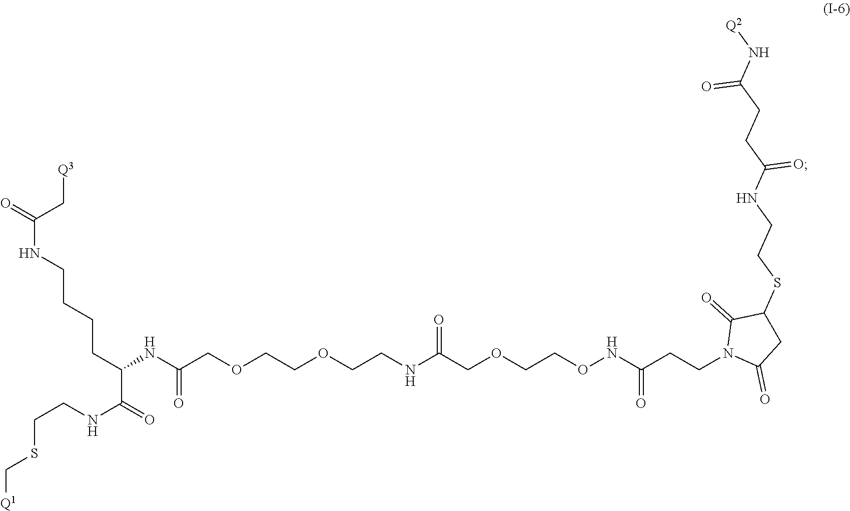

in formula I-6, Q¹, Q², Q³ are as defined in Formula (I); preferably, Q¹ is a FAP receptor-targeting moiety, Q² is an additional receptor-targeting moiety such as a GRPR receptor, Q³ is a chelating group; or Q² is a FAP receptor-targeting moiety, Q¹ is an additional receptor-targeting moiety such as a GRPR receptor, Q³ is a chelating group;

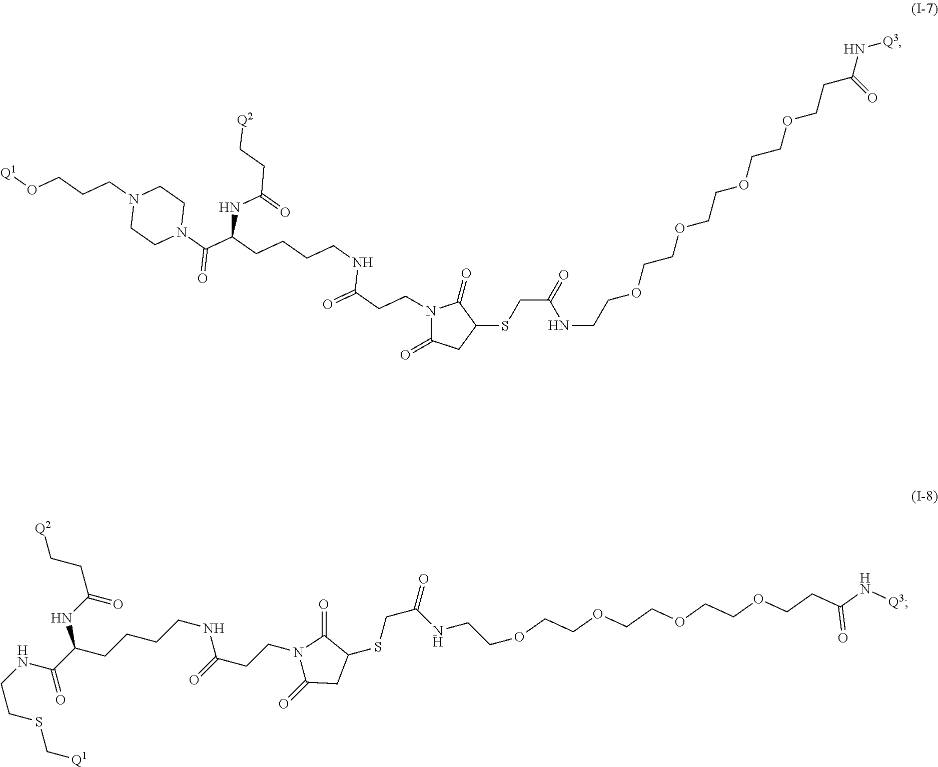

in formulas I-7 and I-8, Q¹, Q², Q³ are as defined in Formula (I); preferably, Q¹ is a FAP receptor-targeting moiety, Q² is a chelating group, Q³ is an additional receptor-targeting moiety such as an αᵥβ₃ receptor; or Q³ is a FAP receptor-targeting moiety, Q² is a chelating group, Q¹ is an additional receptor-targeting moiety such as an αᵥβ₃ receptor.

In some embodiments, the compound has a structure of formula II-A, II-B, II-C-1, II-C-2, or II-D, or a stereoisomer, tautomer, or pharmaceutically acceptable salt thereof:

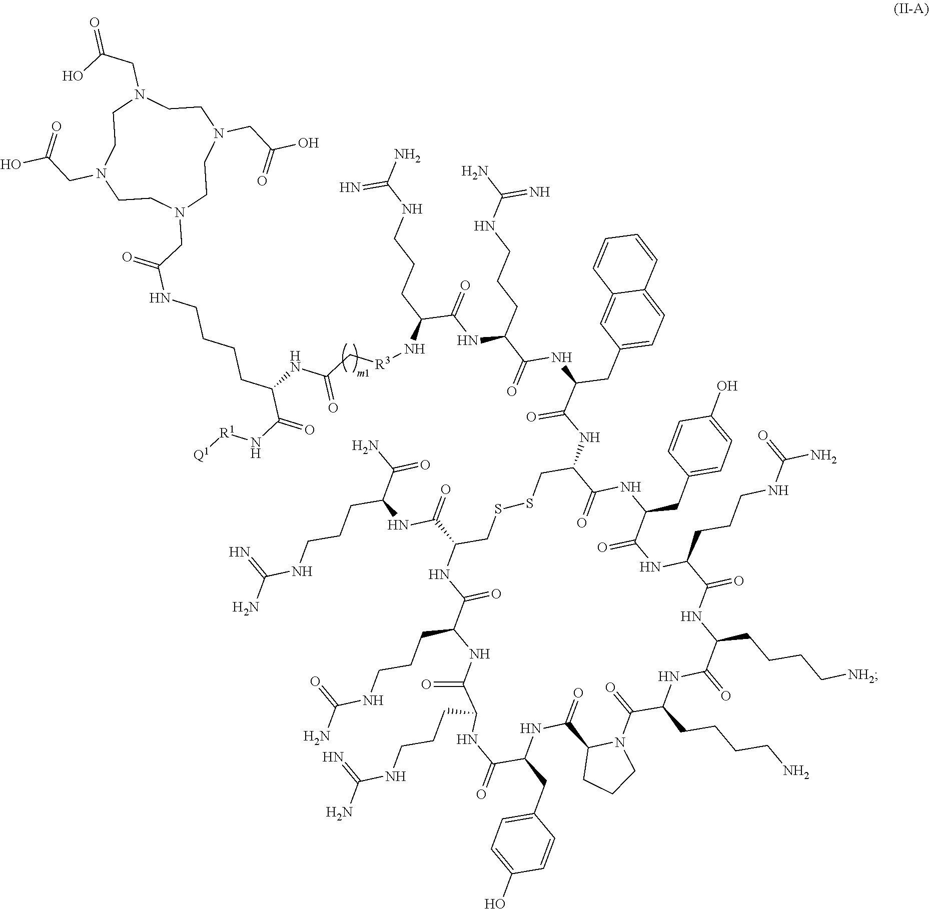

in formula II-A, each symbol is as defined in Formula (I), preferably, Q¹ is a FAP receptor-targeting moiety;

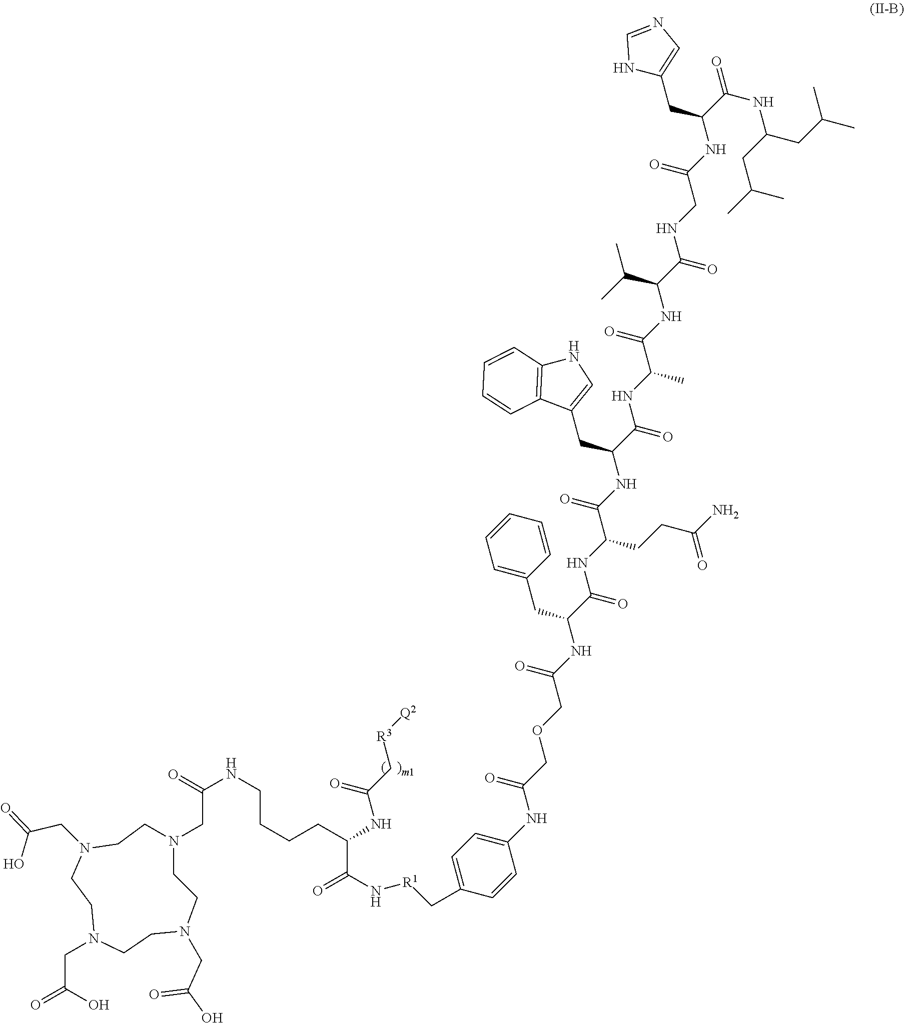

in formula II-B, each symbol is as defined in Formula (I), preferably, Q¹ is a FAP receptor-targeting moiety;

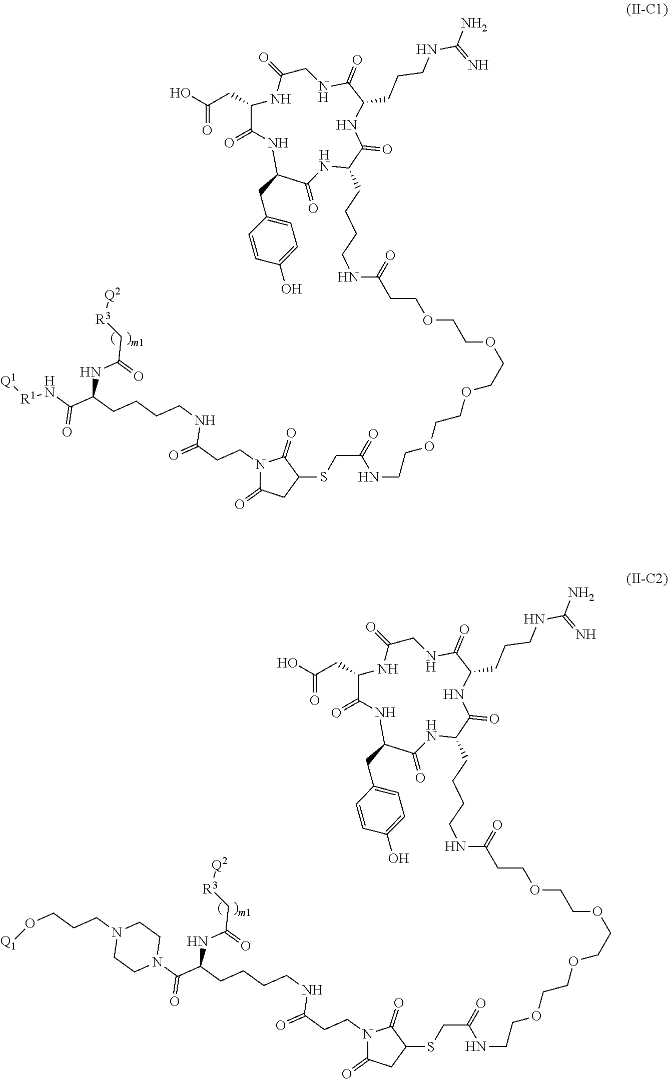

in formulas II-C1 and II-C2, each symbol is as defined in Formula (D), preferably, Q¹ is a FAP receptor-targeting moiety, Q² is a chelating group;

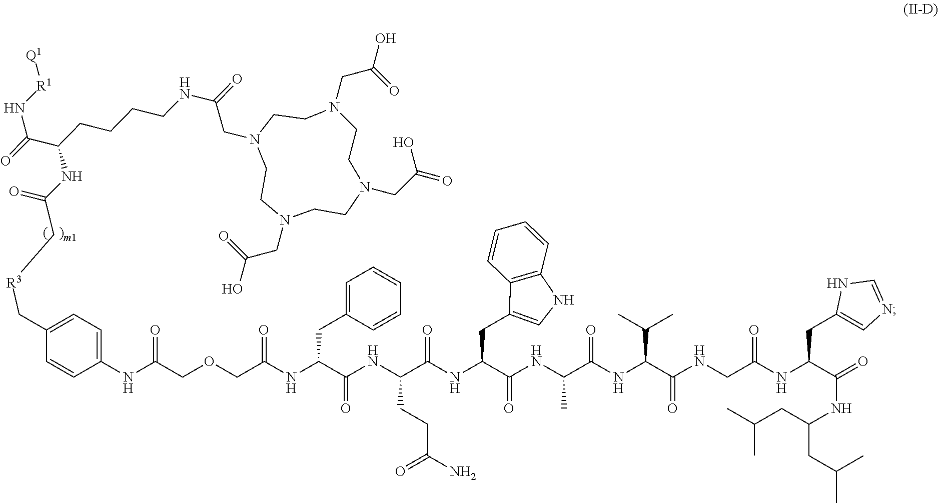

in formula II-D, each symbol is as defined in Formula (D), preferably, Q¹ is a FAP receptor-targeting moiety.

In some embodiments, the compound has a structure of formula III-1 or III-2, or a stereoisomer, tautomer, or pharmaceutically acceptable salt thereof:

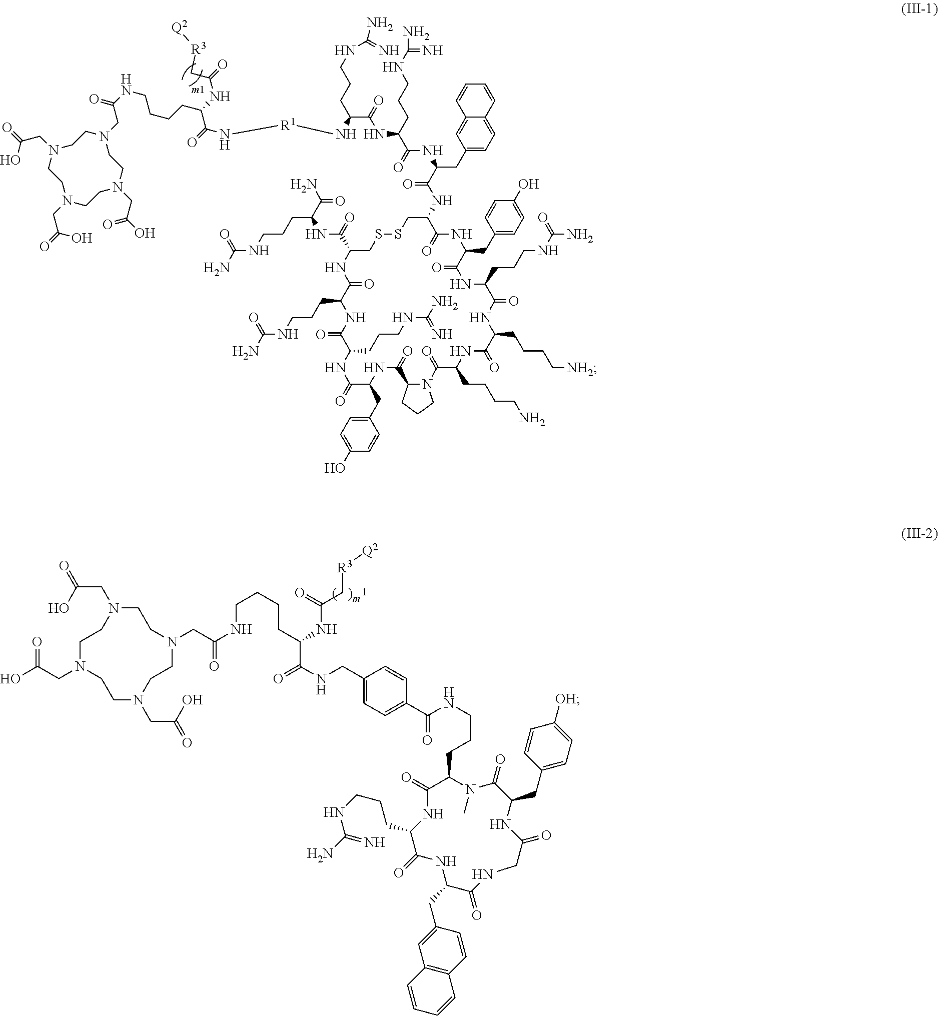

wherein, each symbol is as defined in Formula (I); preferably, Q² is a FAP receptor-targeting moiety.

In some embodiments, the compound has a structure of formula V:

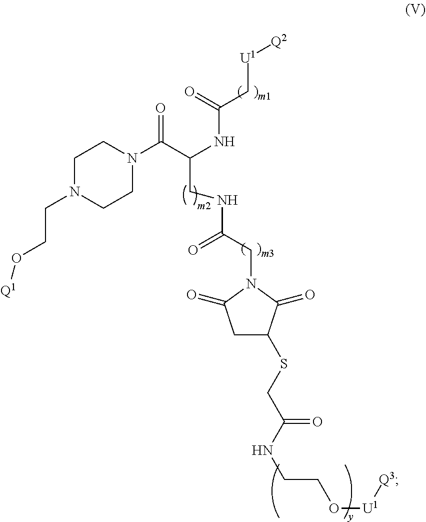

wherein, each U¹ is independently selected from the group consisting of a bond,

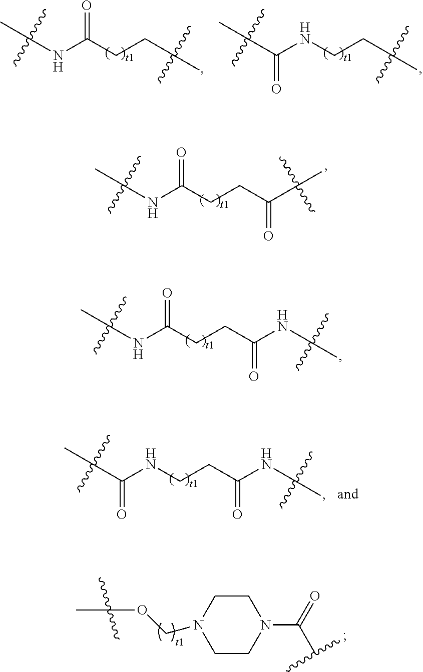

each t1 is independently selected from the group consisting of 1, 2, 3, and 4;
m1, m2, m3 and m4 are each independently selected from the group consisting of 0, 1, 2, 3, 4, and 5;
y is an integer selected from the group consisting of 3 to 10;
Q¹ is a FAP receptor-targeting moiety,
Q² is a chelating group,
Q³ is an αᵥβ₃ receptor-targeting moiety.

Preferably, Q¹ is selected from the group consisting of:

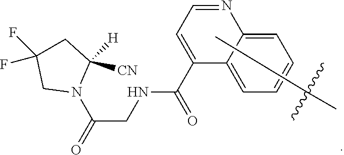

In some embodiments, the FAP receptor-targeting moiety has a structure of formula II-1:

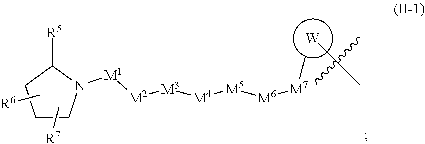

M¹, M², M³, M⁴, M⁵, M⁶, M⁷ are each independently selected from the group consisting of a bond, —O—, —CH₂—, —NR⁸—, —C(═O)—, —C(═S)—, —C(═NH)R⁸—, —CHR⁸—, and —C(R⁸)₂—, provided that: (i) two O atoms are not directly linked; and (ii) at most four of M1, M2, M3, M4, M5, M6, and M7 are bonds; R⁶ and R⁷ are each independently selected from the group consisting of —H, —OH, halogen atom, C₁₋₆alkyl, —O—C₁₋₆alkyl, and —S—C₁₋₆alkyl;
R⁵ is selected from the group consisting of —H, —CN, —B(OH)₂, —C(═O)-alkyl, —C(═O)-aryl-, —C═C—C(═O)-aryl, —C═C—S(═O)₂-aryl, —C(═O)OH, —S(═O)₂OH, —S(═O)₂NH₂, —P(═O)(OH)₂, and 5-tetrazolyl;
R⁸ is selected from the group consisting of —H, C₁₋₆alkyl, —O—C₁₋₆alkyl, —S—C₁₋₆alkyl, C₂₋₆alkenyl, C₂₋₆heteroalkenyl, C₅₋₆cycloalkenyl, C₄₋₆heterocycloalkenyl, C₂₋₆alkynyl, C₆₋₁₀aryl, and C₆₋₁₀arylC₁₋₆alkyl, wherein the C₁₋₆alkyl is optionally substituted by 1-3 substituents selected from the group consisting of —OH, oxygen, and halogen atom;
the ring W is selected from the group consisting of naphthyl, and 5-10-membered nitrogen-containing heteroaryl.

In some embodiments, -M¹-M²-M³-M⁴-M⁵-M⁶-M⁷- is —C(═O)—CH₂—NR⁸—C(═O)—.

In some embodiments, R⁶ and R⁷ are each independently selected from the group consisting of H and F.

In some embodiments, the ring W is quinolinyl; in some further embodiments, the ring W is 4-quinolinyl.

In some embodiments,

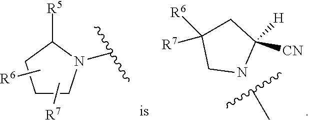

In some embodiments, the FAP receptor-targeting moiety has a structure of formula II-2:

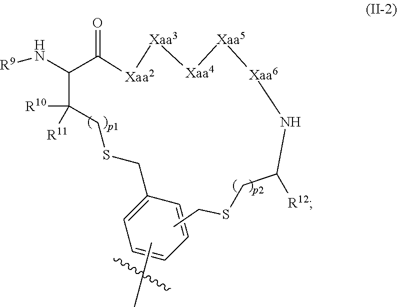

R⁹ is alkylacyl;
R¹⁰, R¹¹ are each independently selected from the group consisting of H and CH₃;
p1 is selected from the group consisting of 0 and 1; p2 is selected from the group consisting of 1 and 2;
Xaa², Xaa³, Xaa⁴, Xaa⁵ and Xaa⁶ are each independently selected from the group consisting of common amino acid residues and non-common amino acid residues;
preferably,
Xaa² is

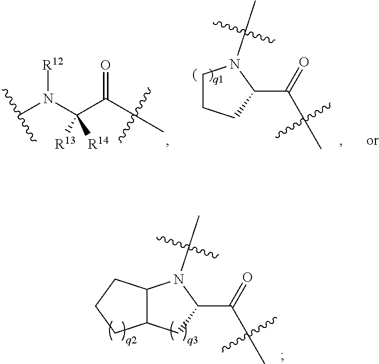

R¹², R¹³, R¹⁴ are each independently selected from the group consisting of C₁₋₂alkyl, carboxyl, and H, wherein the C₁₋₂alkyl is optionally substituted by 1 or 2 substituents selected from the group consisting of OH, NH₂, halogen atom, and C₅₋₇cycloalkyl;

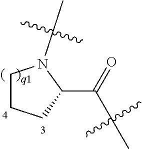

is optionally substituted at positions 3 and 4 by 1 or 2 substituents selected from the group consisting of methyl, OH, NH₂, and F;
q1 is selected from the group consisting of 0, 1, and 2;
q2 is selected from the group consisting of 1, 2, and 3;
q3 is selected from the group consisting of 1 and 2;
Xaa³ is

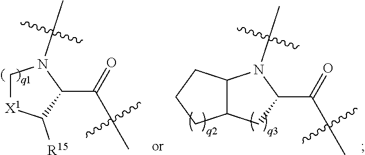

X¹ is selected from the group consisting of CH₂, CF₂, CHR¹⁶, S, O, and NH;
R¹⁵ is H, methyl, OH, NH₂, or F,
R¹⁶ is methyl, OH, NH₂, or F;
Xaa⁴ is

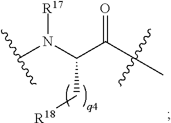

R¹⁷ is methyl or H;
R¹⁸ is selected from the group consisting of H, —OH, —C(═O)OH, —(C═O)NH₂, X², and —NH—C(═O)—X², wherein X² is selected from the group consisting of C₁₋₆alkyl, phenyl, and C₅₋₆heteroaryl, and X² is optionally substituted with 1 or 2 substituents selected from the group consisting of methyl, C(═O)NH₂, halogen atom, NH₂, and OH;
q4 is selected from the group consisting of 1, 2, and 3; wherein, in the (q4) number of CH₂ moieties, one or two hydrogen atoms are each independently optionally substituted with methyl, ethyl, phenyl, or C₅₋₆heteroaryl,
Xaa⁵ is

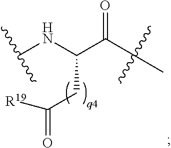

R¹⁹ is selected from the group consisting of OH and NH₂;
q⁵ is selected from the group consisting of 1, 2, and 3;
Xaa⁶ is selected from the group consisting of amino acid residues of aromatic L-α-amino acids and heteroaromatic L-α-amino acids.

In some embodiments, the FAP receptor-targeting moiety is:

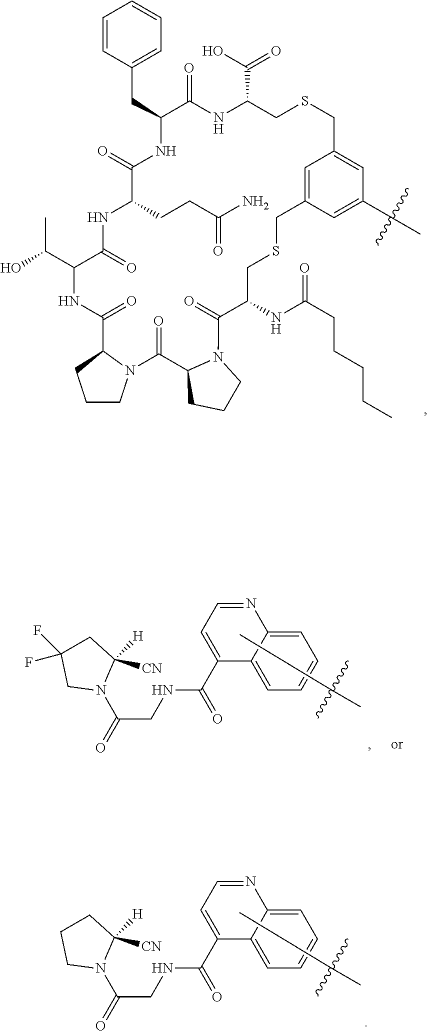

In some embodiments, the GRPR receptor-targeting moiety has a structure of formula III:

-Xaa⁷-Xaa⁸-Xaa⁹-Xaa¹⁰-Xaa¹¹-Xaa¹²-Xaa¹³-C(═O)—Z(III);
Xaa⁷, Xaa⁸, Xaa⁹, Xaa¹⁰, Xaa¹¹, Xaa¹², and Xaa¹³ are each independently selected from the group consisting of common amino acid residues and non-common amino acid residues;
preferably,
Xaa⁷ is absent or selected from the group consisting of amino acid residues of Asn, Thr, Phe, Thi, Cpa, naphthylalanine, β-naphthylalanine, Tpi, Tyr, o-I-Tyr, Trp, and 5F-Phe;
Xaa⁸ is selected from the group consisting of amino acid residues of Gin, Asn, and His;
Xaa⁹ is selected from the group consisting of amino acid residues of Trp and Tpi;
Xaa¹⁰ is selected from the group consisting of amino acid residues of Ala, Ser, and Val;
Xaa¹¹ is selected from the group consisting of amino acid residues of Val, Ser, and Thr;
Xaa¹² is selected from the group consisting of amino acid residues of Gly, Sar, D-Ala, and β-Ala;
Xaa¹³ is selected from the group consisting of amino acid residues of His. and (3-Me)His;
Z is selected from the group consisting of —NHOH, —NHNH₂, —NH-alkyl, —N(alkyl)₂, —O-alkyl, —NH—CHR²³R²⁴, and —O—CHR²³R²⁴; R²³ and R²⁴ are the same or different and are each independently selected from the group consisting of H, alkyl, alkyl ether, aryl, aryl ether, arylalkyl, halogen atom, hydroxy, and aryl substituted with hydroxyalkyl;
the GRPR receptor-targeting moiety is connected to remainder of the compound at Xaa⁷.

In some embodiments, the FAP receptor-targeting moiety is:

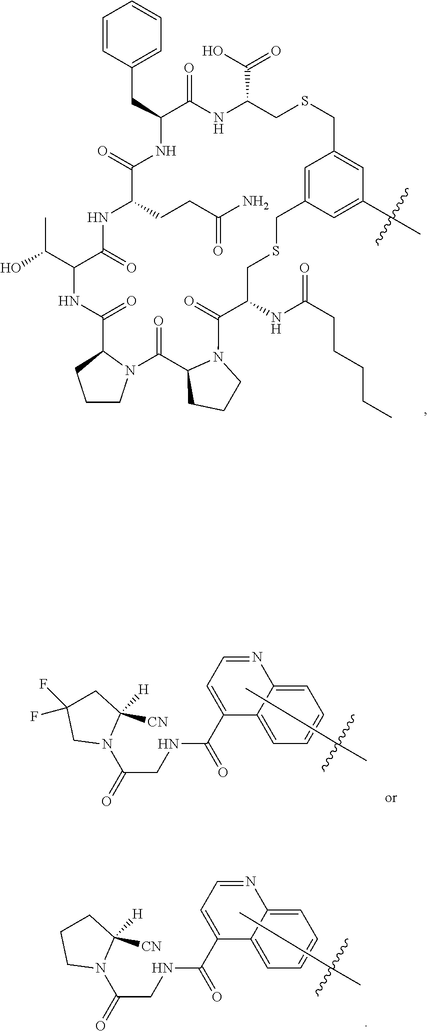

In some embodiments, the FAP receptor-targeting moiety is

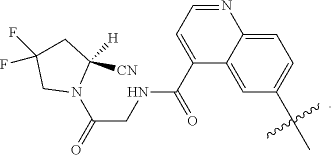 [SMILES: *c1ccc2nccc(C(=O)NCC(=O)N3CC(F)(F)C[C@H]3C#N)c2c1]

In some embodiments, the CXCR4 receptor-targeting moiety is selected from the group consisting of:

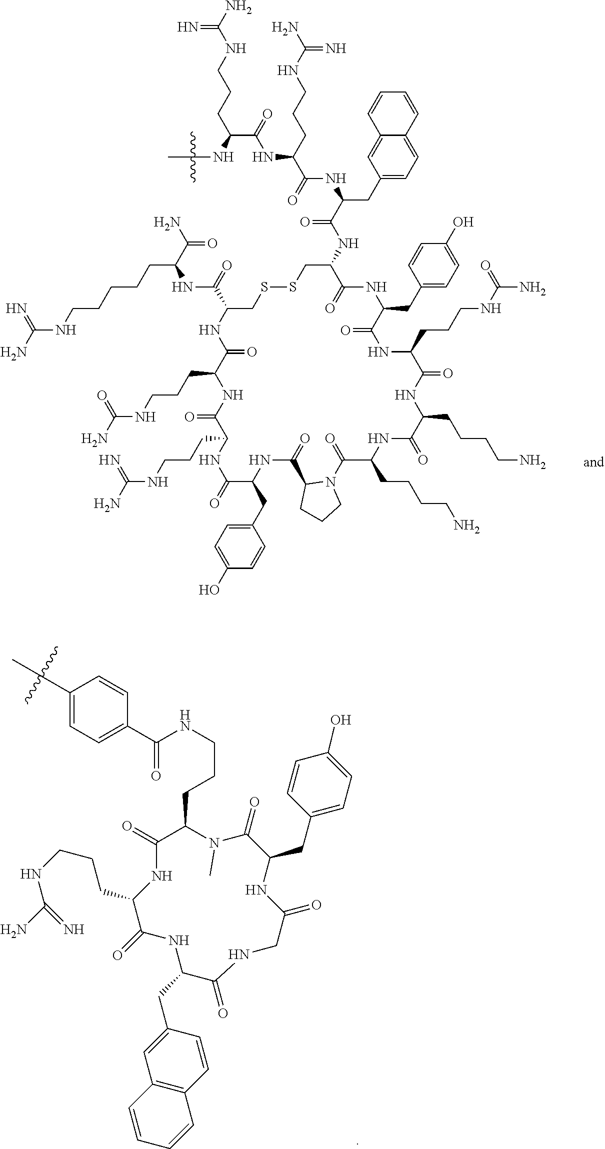 [SMILES: *N[C@@H](CCCNC(=N)N)C(=O)N[C@@H](CCCNC(=N)N)C(=O)N[C@@H](Cc1ccc2ccccc2c1)C(=O)N[C@H]1CSSC[C@@H](C(=O)N[C@@H](CCCCCNC(=N)N)C(N)=O)NC(=O)[C@H](CCCNC(N)=O)NC(=O)[C@H](CCCNC(=N)N)NC(=O)[C@H](Cc2ccc(O)cc2)NC(=O)[C@@H]2CCCN2C(=O)[C@H](CCCCN)NC(=O)[C@H](CCCCN)NC(=O)[C@H](CCCNC(N)=O)NC(=O)[C@H](Cc2ccc(O)cc2)NC1=O ; *c1ccc(C(=O)NCCC[C@@H]2C(=O)N[C@@H](CCCNC(=N)N)C(=O)N[C@@H](Cc3ccc4ccccc4c3)C(=O)NCC(=O)N[C@H](Cc3ccc(O)cc3)C(=O)N2C)cc1]

In some embodiments, the GRPR receptor-targeting moiety is:

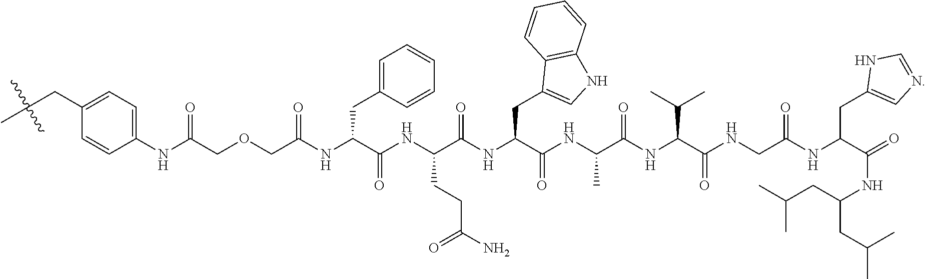 [SMILES: *Cc1ccc(NC(=O)COCC(=O)N[C@H](Cc2ccccc2)C(=O)N[C@@H](CCC(N)=O)C(=O)N[C@@H](Cc2c[nH]c3ccccc23)C(=O)N[C@@H](C)C(=O)N[C@H](C(=O)NCC(=O)NC(Cc2cnc[nH]2)C(=O)NC(CC(C)C)CC(C)C)C(C)C)cc1]

In some embodiments, the αᵥβ₃ receptor-targeting moiety is:

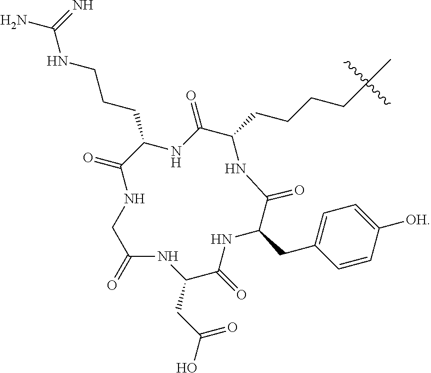 [SMILES: *CCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

In some embodiments, the chelating group is selected from the group consisting of 1,4,7,10-tetraazacyclododecane-1,4,7,10-tetraacetic acid (DOTA), 1,4,7,10-tetraazacyclododecane-1-(glutaric acid)-4,7,10-triacetic acid (DOTAGA), 1,4,7-triazacyclononane triacetic acid (NOTA), 1,4,7-triazacyclononane-N-glutaric acid-N′,N″-diacetic acid (NODAGA), 1,4,7-triazacyclononane-1,4-diacetic acid-methylphenylacetic acid (NODA-MPAA), bis-(2-hydroxybenzyl)ethylenediamine diacetic acid (HBED), 4,11-bis-(carboxymethyl)-1,4,8,11-tetraazabicyclo[6.6.2]-hexadecane (CB-TE2A), DFO, and a hexadentate tris(3,4-hydroxypyridinone) (THP)-derived group.

In some embodiments, the chelating group is one of the following groups:

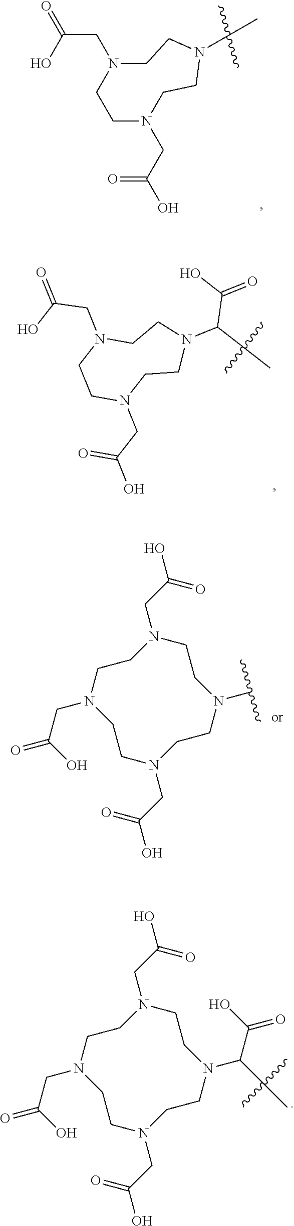 [SMILES: *N1CCN(CC(=O)O)CCN(CC(=O)O)CC1 ; *C(C(=O)O)N1CCN(CC(=O)O)CCN(CC(=O)O)CC1 ; *N1CCN(CC(=O)O)CCN(CC(=O)O)CCN(CC(=O)O)CC1 ; *C(C(=O)O)N1CCN(CC(=O)O)CCN(CC(=O)O)CCN(CC(=O)O)CC1]

In some embodiments, the chelating group is

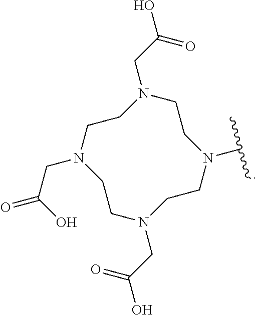 [SMILES: *N1CCN(CC(=O)O)CCN(CC(=O)O)CCN(CC(=O)O)CC1]

In some embodiments, the chelating group is

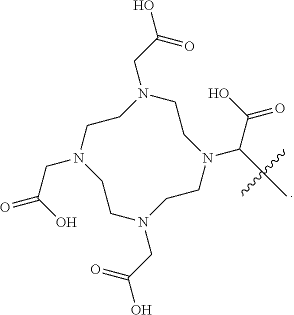 [SMILES: *C(C(=O)O)N1CCN(CC(=O)O)CCN(CC(=O)O)CCN(CC(=O)O)CC1]

In some embodiments, R¹ is a bond.

In some embodiments, R¹ is a heteroalkylene, for example, the heteroalkylene is —(CH₂)_{q}—S—(CH₂)ₚ—, and preferably, q is selected from the group consisting of 0, 1, and 2; p is selected from the group consisting of 0, 1, and 2.

In some embodiments, R¹ is —U¹-G¹-A¹-L-A²-G²-U²—, wherein: L is

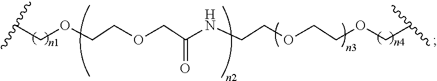

A² is —C(═O) NH—, and A¹, U¹, U², G¹, G² are all bonds. As can be easily understood by those skilled in the art, when A¹, U¹, U², G¹, G² are all bonds, R¹ is L-A².

In some embodiments, R³ is a bond.

In some embodiments, R³ is —U¹-G¹-A¹-L-A²-G²-U²—, wherein: U² is

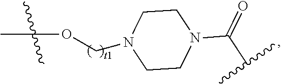

t1=3; A¹, A², L, U¹, G¹, G² are all bonds. As can be easily understood by those skilled in the art, when A¹, A², L, U¹, G¹, G² are all bonds, R³ is U².

In some embodiments, R³ is —U¹-G¹-A¹-L-A²-G²-U²—, wherein: L is

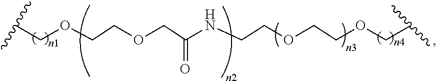

A² is-NHC(═O)—, G² is

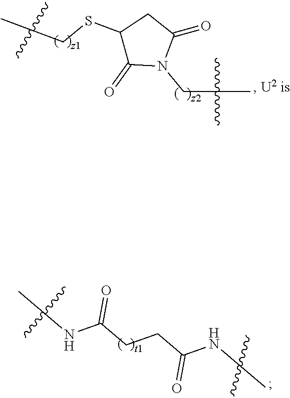

A¹, U′ and G¹ are all bonds.

In some embodiments, R⁴ is —U¹-G¹-A¹-L-A²-G²-U²—, wherein: G¹ is

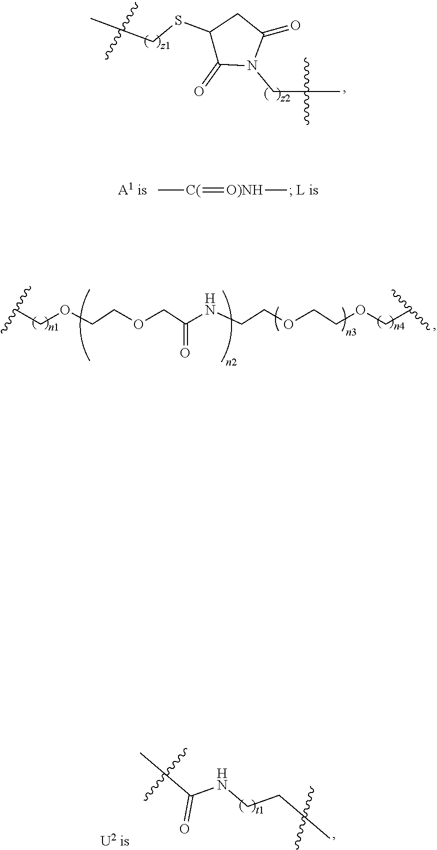

and U¹, A² and G² are all bonds.

In some embodiments, R⁴ is a bond.

In some embodiments, t1=3.

In some embodiments, m1=2.

In some embodiments, m2=4.

In some embodiments, m3=1.

In some embodiments, m4=2.

In some embodiments, n1=2, and/or n2=1, and/or n3=1, and/or n4=1.

In some embodiments, n1=2, and/or n2=0, and/or n3=2, and/or n4=2.

In some embodiments, U¹ is a bond and/or G¹ is a bond and/or A′ is a bond.

In some embodiments, R¹ is —U¹-G¹-A¹-L-A²-G²-U²—, wherein: L is

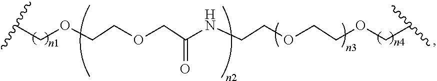

n1=2, n2=1, n3=1, n4=1, A² is —C(═O)NH—; A¹, U¹, U², G¹, G² are all bonds;
R³ is —U¹-G¹-A¹-L-A²-G²-U²—, wherein: U² is

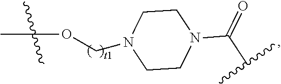

t1=3;
A¹, A², L, U¹, G¹, G² are all bonds; R⁴ is a bond; m1=2, m2=4, m3=1, Q¹ is a CXCR4 receptor-targeting moiety; Q² is a FAP receptor-targeting moiety; Q³ is a chelating group.

In some embodiments, R¹ is a bond; R³ is —U¹-G¹-A¹-L-A²-G²-U²—, wherein L is

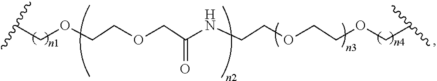

n1=2, n2=1, n3=1, n4=1; A² is —NHC(═O)—, G² is

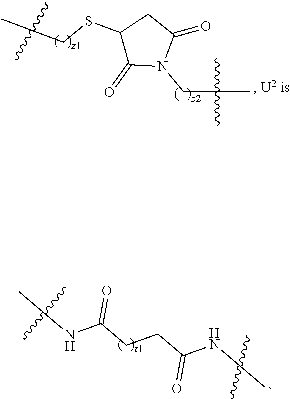

and U¹, G¹ and A¹ are all bonds; R⁴ is a bond;
m2=4, m3=1, m4=2; Q¹ is a FAP receptor-targeting moiety; Q² is a CXCR4 receptor-targeting moiety; Q³ is a chelating group.

In some embodiments, R¹ is —U¹-G¹-A¹-L-A²-G²-U²—; wherein L is

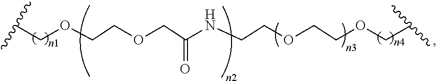

n1=2, n2=1, n3=1, n4=1, A² is —C(═O)NH—; U¹, G¹, A². G², U² are all bonds;
R³ is —U¹-G¹-A¹-L-A²-G²-U², wherein: U¹

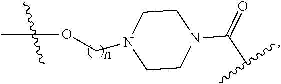

is t1=3, and G¹, A¹, L, A², G², U² are all bonds;
R⁴ is a bond; m1=2; m2=4, m3=1, Q¹ is a GRPR receptor-targeting moiety; Q² is a FAP receptor-targeting moiety; Q³ is a chelating group.

In some embodiments, R¹ is a bond;
R³ is —U¹-G¹-A¹-L-A²-G²-U², wherein: L is

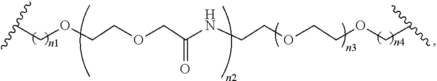

n1=2, n2=1, n3=1, n4=1; A¹ is —C(═O)NH—, U¹, G¹, A², G², U² are all bonds;
R⁴ is a bond; m2=4, m3=1, m4=2, Q¹ is a FAP receptor-targeting moiety; Q² is a GRPR receptor-targeting moiety; Q³ is a chelating group.

In some embodiments, R¹ is a bond;
R³ is —U¹-G¹-A¹-L-A²-G²-U²—, wherein: L is

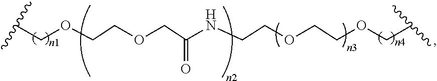

n1=2, n2=1, n3=1, n4=1; A¹ is —C(═O)NH—; U² is

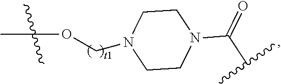

t1=3,
U¹, G¹, A², G² are all bonds; R⁴ is a bond; m4=1, m2=4, m3=1, m1=2,
Q¹ is a CXCR4 receptor-targeting moiety; Q² is a FAP receptor-targeting moiety; Q³ is a chelating group.

In some embodiments, R¹ is a bond;
R³ is —U¹-G¹-A¹-L-A²-G²-U²—, wherein: G¹ is

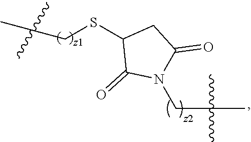

### A¹ is —C(═O) NH—;

L is

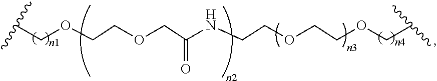

n1=2, n2=1, n3=1, n4=1;
U² is

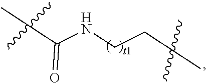

U¹, A² and G² are all bonds,
R⁴ is a bond; m4=1, m2=4, m3=1,
Q¹ is a FAP receptor-targeting moiety;
Q² is a CXCR4 receptor-targeting moiety;
Q³ is a chelating group.

In some embodiments,

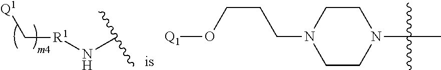

R³ is a bond;
R⁴ is —U¹-G¹-A¹-L-A²-G²-U²—, wherein: G¹ is

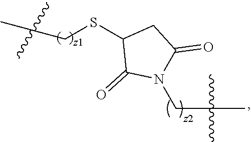

### A¹ is —C(═O) NH—;

L is

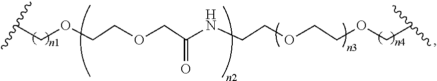

n1=2, n2-0, n3=2, n4=2;
U² is

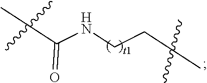

and U¹, A², G² are all bonds;
m1=2, m2=4;
Q¹ is a FAP receptor-targeting moiety;
Q² is a chelating group, for example, the chelating group is

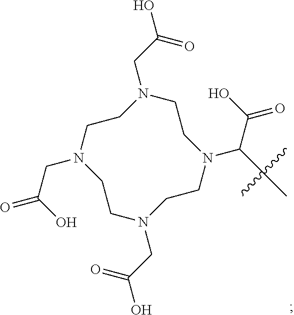 [SMILES: *C(C(=O)O)N1CCN(CC(=O)O)CCN(CC(=O)O)CCN(CC(=O)O)CC1]

Q³ is an αᵥβ₃ receptor-targeting moiety.

In such embodiments, the use of the above structures provide a longer linker between the targeting group Q³ and the chelating group Q², thereby enhancing dual-targeting capability.

In some embodiments, R¹ is heteroalkylene, such as —(CH₂)_{q}—S—(CH₂)ₚ—, preferably, q is selected from the group consisting of 0, 1, and 2; p is selected from the group consisting of 0, 1, and 2;
R³ is a bond;
R⁴ is —U¹-G¹-A¹-L-A²-G²-U²—, wherein: G¹ is

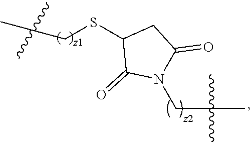

### A¹ is —C(═O) NH—;

L is

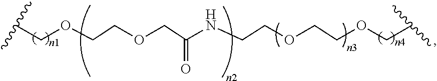

n1=2, n2=0, n3=2, n4=2;
U² is

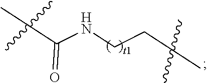

and U¹, A², and G² are bonds;
m1=2, m2=4; Q¹ is a FAP receptor-targeting moiety; Q² is a chelating group; Q³ is an αᵥβ₃ receptor-targeting moiety.

In some embodiments, the compound is selected from the group consisting of the following structures, or a stereoisomer, tautomer, or pharmaceutically acceptable salt thereof:

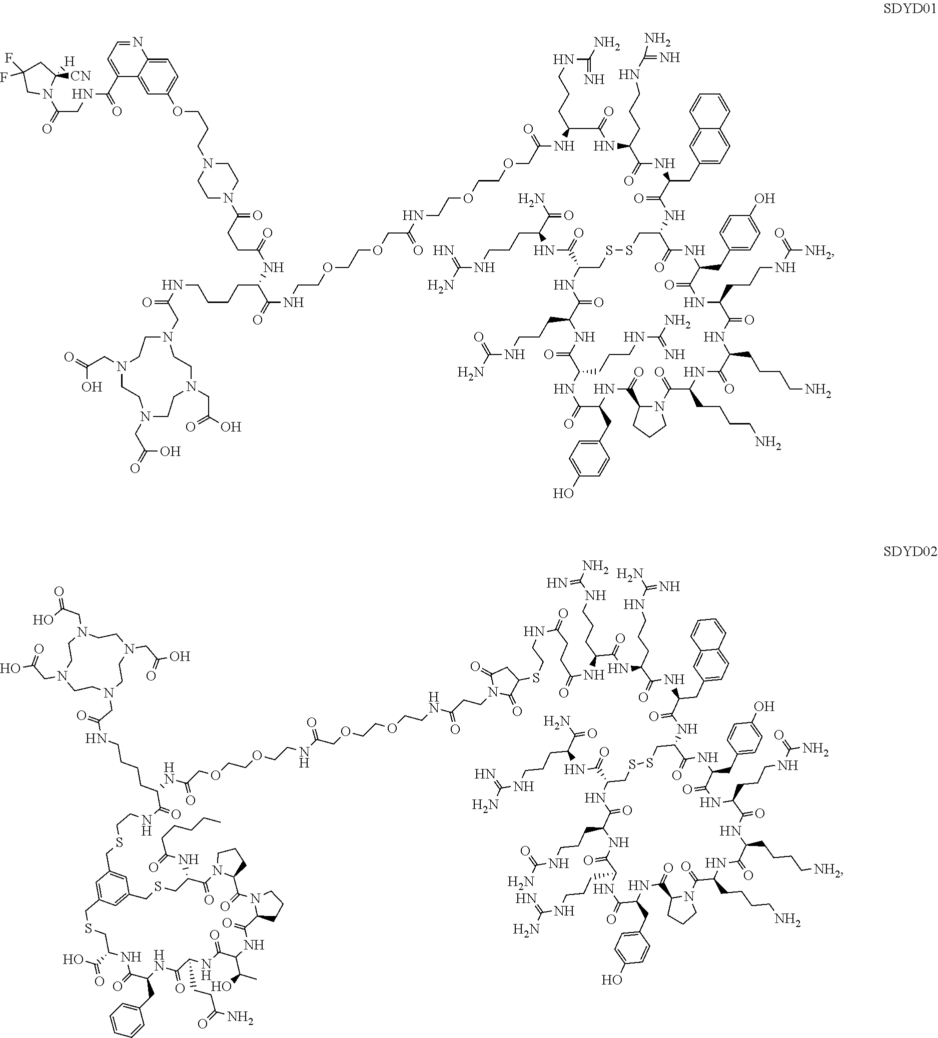

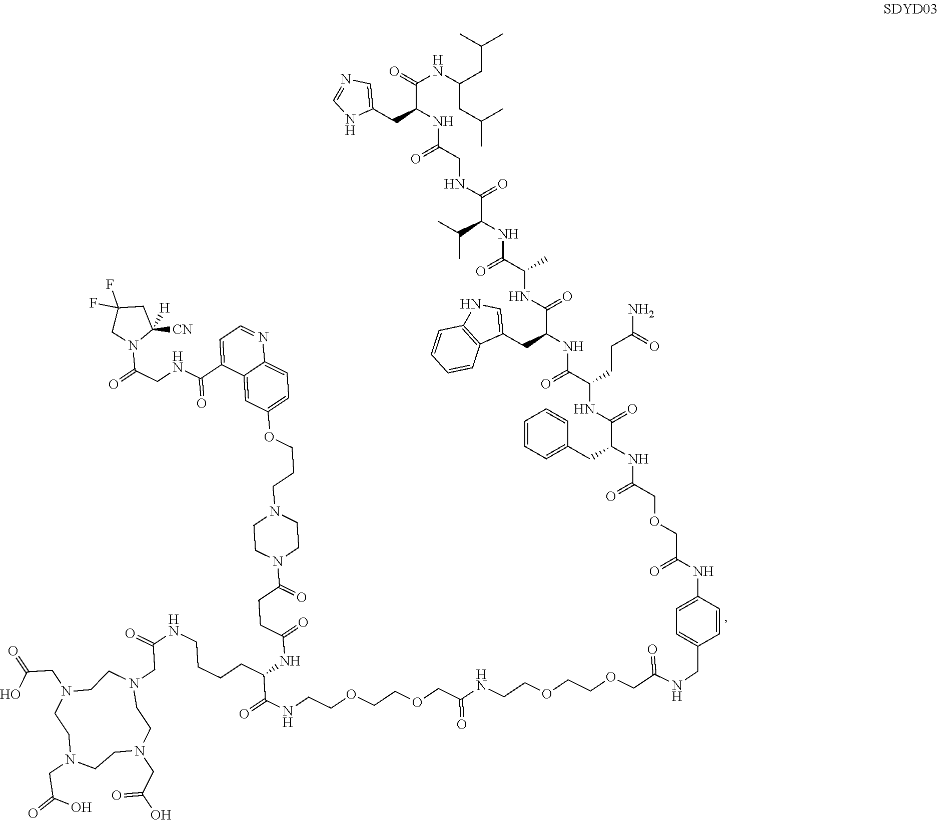 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)COCCOCCNC(=O)COCCOCCNC(=O)[C@H](CCCCNC(=O)CN2CCN(CC(=O)O)CCN(CC(=O)O)CCN(CC(=O)O)CC2)NC(=O)CCC(=O)N2CCN(CCCOc3ccc4nccc(C(=O)NCC(=O)N5CC(F)(F)C[C@H]5C#N)c4c3)CC2)cc1)C(C)C]

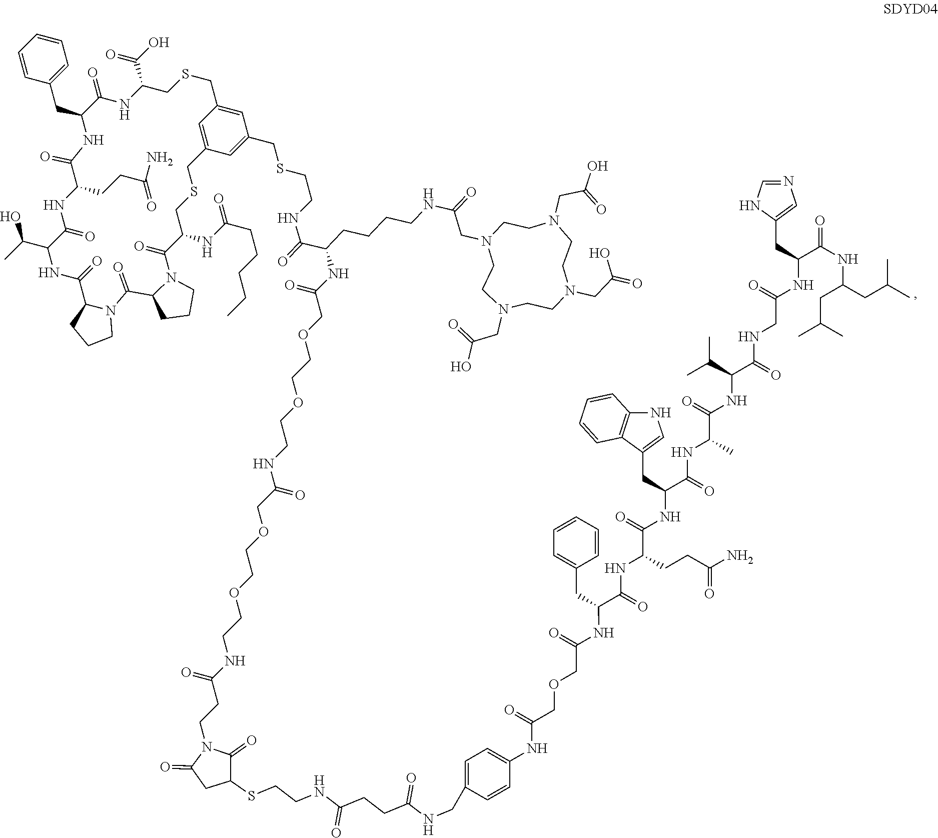 [SMILES: CCCCCC(=O)N[C@H]1CSCc2cc(CSCCNC(=O)[C@H](CCCCNC(=O)CN3CCN(CC(=O)O)CCN(CC(=O)O)CCN(CC(=O)O)CC3)NC(=O)COCCOCCNC(=O)COCCOCCNC(=O)CCN3C(=O)CC(SCCNC(=O)CCC(=O)NCc4ccc(NC(=O)COCC(=O)N[C@H](Cc5ccccc5)C(=O)N[C@@H](CCC(N)=O)C(=O)N[C@@H](Cc5c[nH]c6ccccc56)C(=O)N[C@@H](C)C(=O)N[C@H](C(=O)NCC(=O)N[C@@H](Cc5cnc[nH]5)C(=O)NC(CC(C)C)CC(C)C)C(C)C)cc4)C3=O)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O]

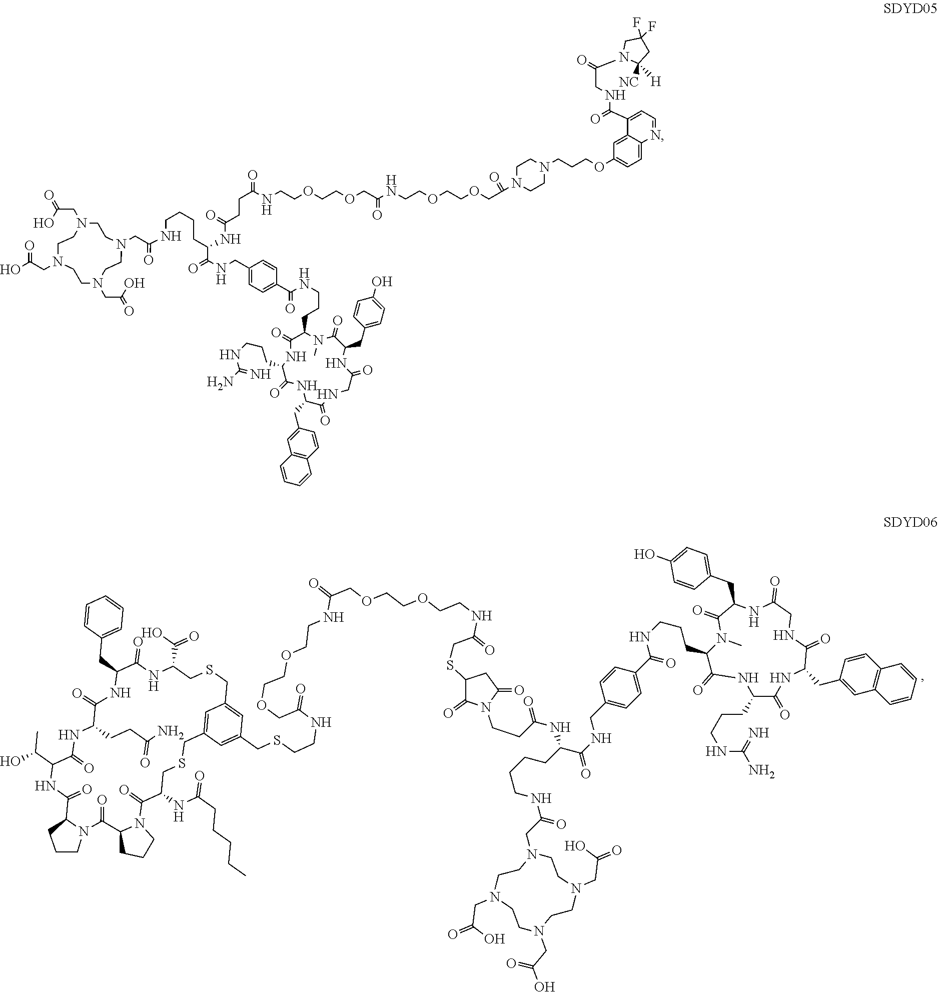

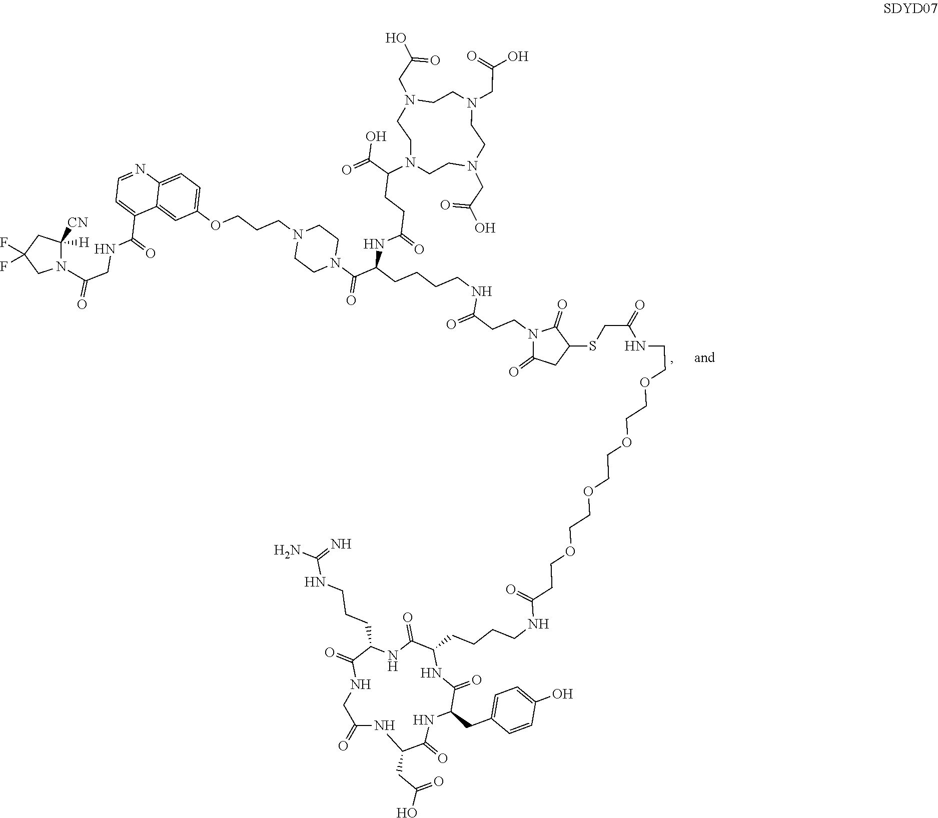 [SMILES: N#C[C@@H]1CC(F)(F)CN1C(=O)CNC(=O)c1ccnc2ccc(OCCCN3CCN(C(=O)[C@H](CCCCNC(=O)CCN4C(=O)CC(SCC(=O)NCCOCCOCCOCCOCCC(=O)NCCCC[C@@H]5NC(=O)[C@@H](Cc6ccc(O)cc6)NC(=O)[C@H](CC(=O)O)NC(=O)CNC(=O)[C@H](CCCNC(=N)N)NC5=O)C4=O)NC(=O)CCC(C(=O)O)N4CCN(CC(=O)O)CCN(CC(=O)O)CCN(CC(=O)O)CC4)CC3)cc12]

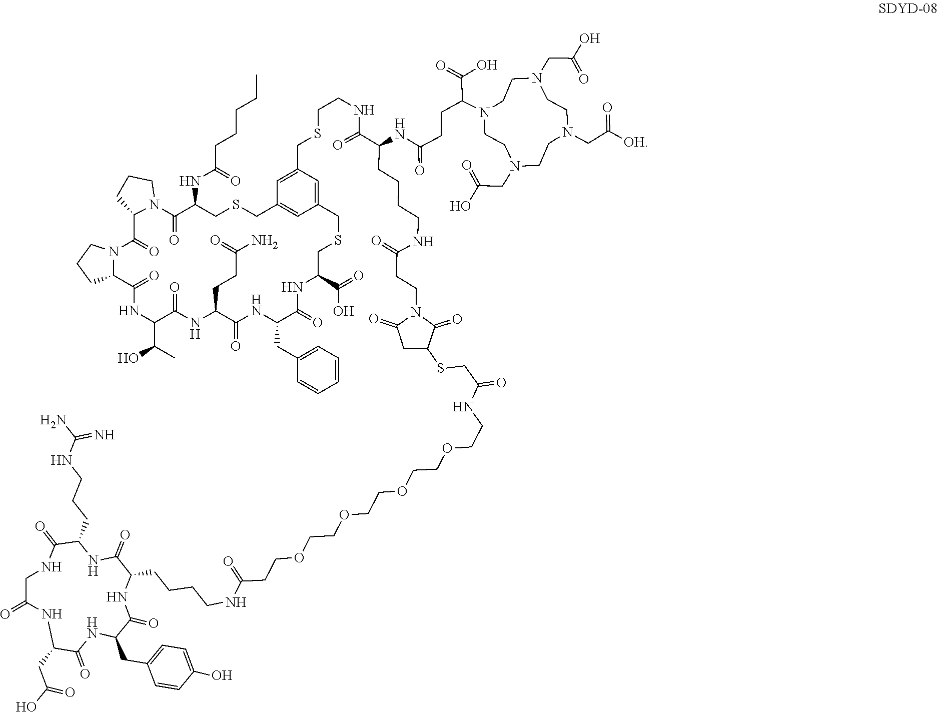 [SMILES: CCCCCC(=O)N[C@H]1CSCc2cc(CSCCNC(=O)[C@H](CCCCNC(=O)CCN3C(=O)CC(SCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C3=O)NC(=O)CCC(C(=O)O)N3CCN(CC(=O)O)CCN(CC(=O)O)CCN(CC(=O)O)CC3)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O]

In another aspect, the present invention provides a method for preparing compound SDYD01, comprising the steps of:

1) reacting compound 1-1 with compound 1-2 to give compound 1-3:

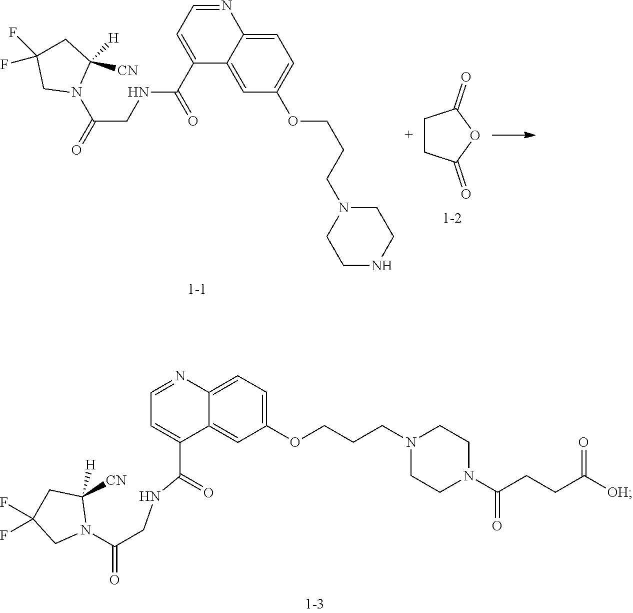 [SMILES: N#C[C@@H]1CC(F)(F)CN1C(=O)CNC(=O)c1ccnc2ccc(OCCCN3CCNCC3)cc12 ; O=C1CCC(=O)O1 ; N#C[C@@H]1CC(F)(F)CN1C(=O)CNC(=O)c1ccnc2ccc(OCCCN3CCN(C(=O)CCC(=O)O)CC3)cc12]

and
2) reacting compound 1-3 with compound 1-4 to give compound SDYD01:

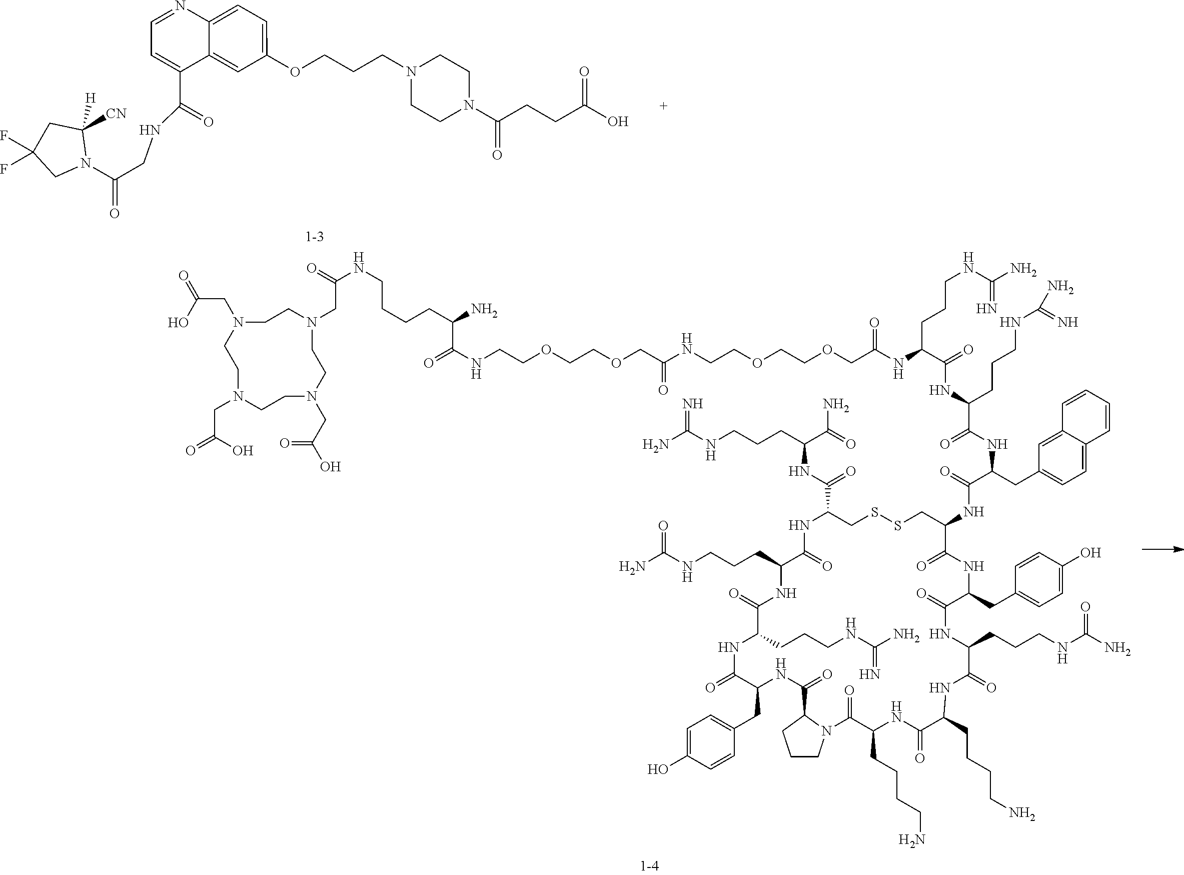 [SMILES: N#C[C@@H]1CC(F)(F)CN1C(=O)CNC(=O)c1ccnc2ccc(OCCCN3CCN(C(=O)CCC(=O)O)CC3)cc12 ; N=C(N)NCCC[C@H](NC(=O)[C@@H]1CSSC[C@@H](NC(=O)[C@H](Cc2ccc3ccccc3c2)NC(=O)[C@H](CCCNC(=N)N)NC(=O)[C@H](CCCNC(=N)N)NC(=O)COCCOCCNC(=O)COCCOCCNC(=O)[C@H](N)CCCCNC(=O)CN2CCN(CC(=O)O)CCN(CC(=O)O)CCN(CC(=O)O)CC2)C(=O)N[C@@H](Cc2ccc(O)cc2)C(=O)N[C@@H](CCCNC(N)=O)C(=O)N[C@@H](CCCCN)C(=O)N[C@@H](CCCCN)C(=O)N2CCC[C@H]2C(=O)N[C@@H](Cc2ccc(O)cc2)C(=O)N[C@@H](CCCNC(=N)N)C(=O)N[C@@H](CCCNC(N)=O)C(=O)N1)C(N)=O]

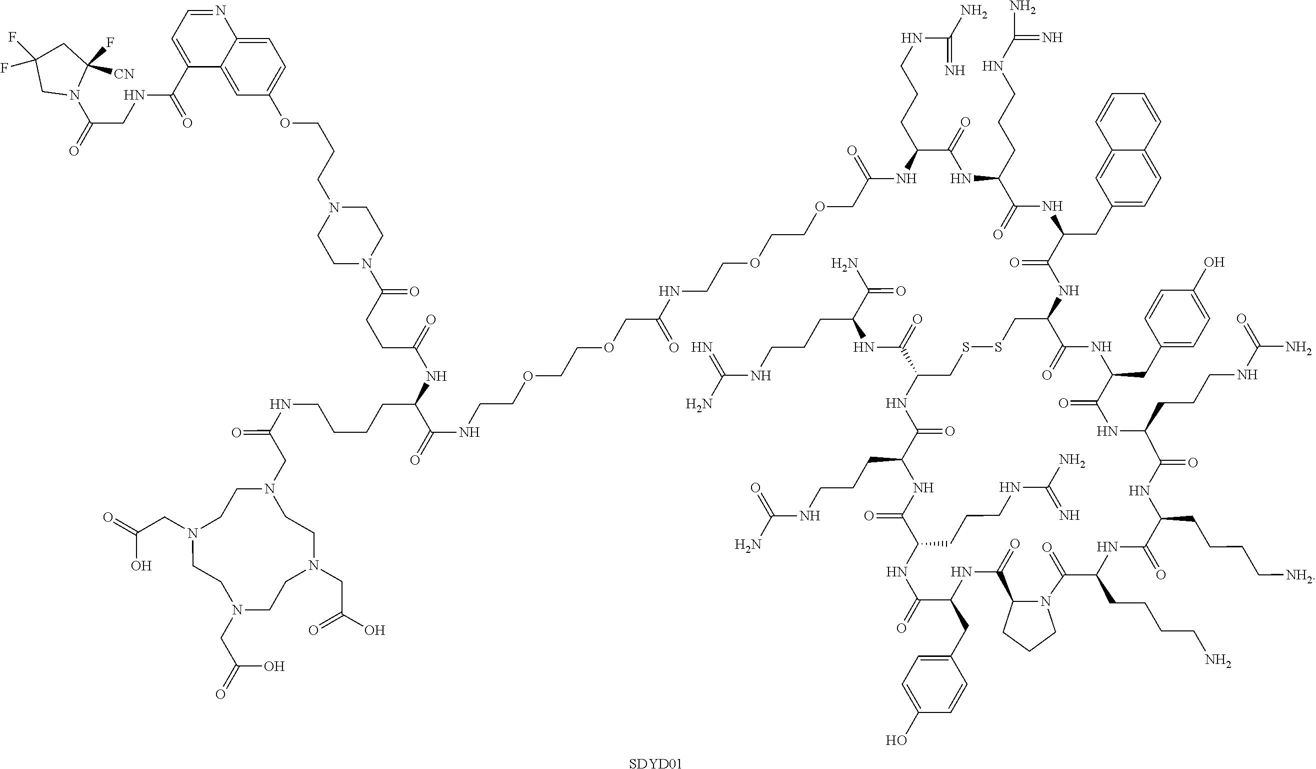 [SMILES: N#C[C@@]1(F)CC(F)(F)CN1C(=O)CNC(=O)c1ccnc2ccc(OCCCN3CCN(C(=O)CCC(=O)N[C@H](CCCCNC(=O)CN4CCN(CC(=O)O)CCN(CC(=O)O)CCN(CC(=O)O)CC4)C(=O)NCCOCCOCC(=O)NCCOCCOCC(=O)N[C@@H](CCCNC(=N)N)C(=O)N[C@@H](CCCNC(=N)N)C(=O)N[C@@H](Cc4ccc5ccccc5c4)C(=O)N[C@@H]4CSSC[C@@H](C(=O)N[C@@H](CCCNC(=N)N)C(N)=O)NC(=O)[C@H](CCCNC(N)=O)NC(=O)[C@H](CCCNC(=N)N)NC(=O)[C@H](Cc5ccc(O)cc5)NC(=O)[C@@H]5CCCN5C(=O)[C@H](CCCCN)NC(=O)[C@H](CCCCN)NC(=O)[C@H](CCCNC(N)=O)NC(=O)[C@H](Cc5ccc(O)cc5)NC4=O)CC3)cc12 ; "SDYD01"]

In another aspect, the present invention provides a method for preparing compound SDYD02, comprising the steps of:
1) reacting compound 2-1 with compound 2-2 to give compound 2-3:

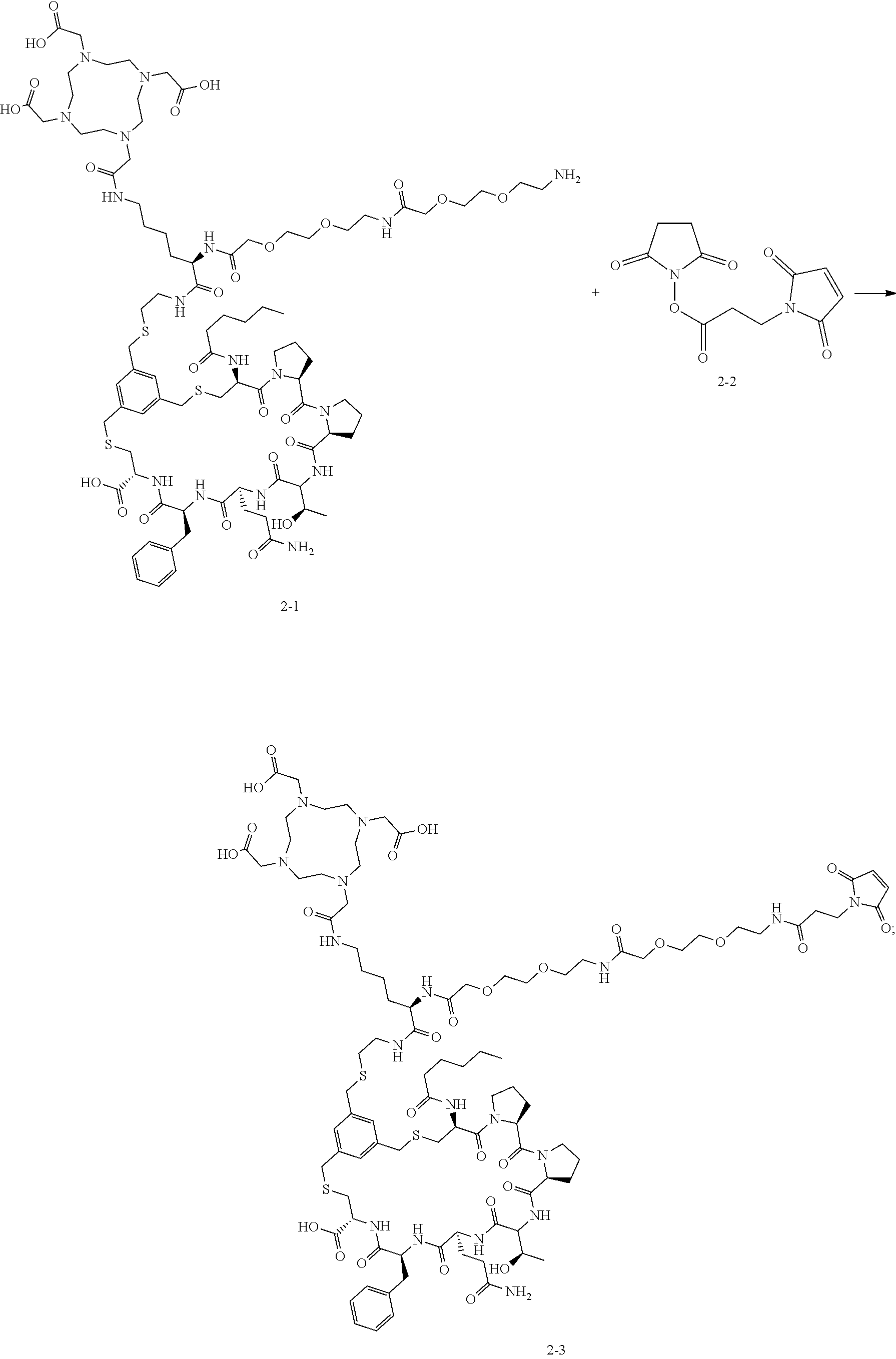

and
2) reacting compound 2-3 with compound 2-4 to give compound SDYD02:

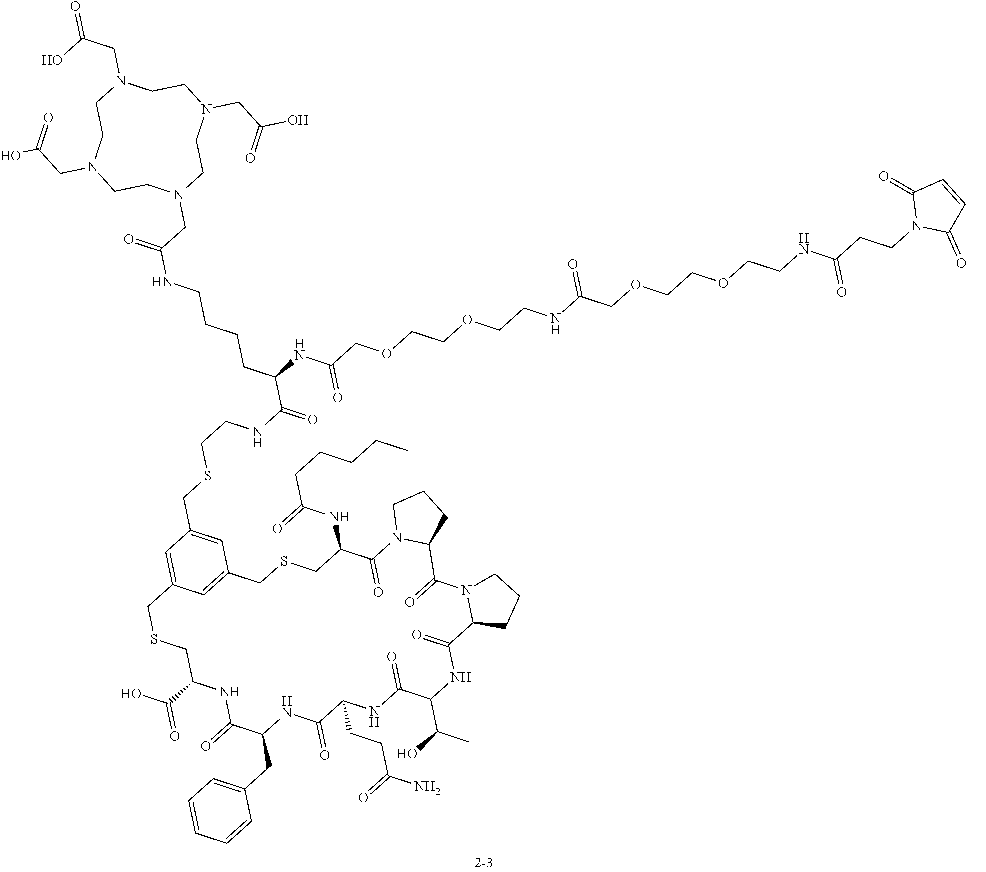 [SMILES: CCCCCC(=O)N[C@@H]1CSCc2cc(CSCCNC(=O)[C@@H](CCCCNC(=O)CN3CCN(CC(=O)O)CCN(CC(=O)O)CCN(CC(=O)O)CC3)NC(=O)COCCOCCNC(=O)COCCOCCNC(=O)CCN3C(=O)C=CC3=O)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O]

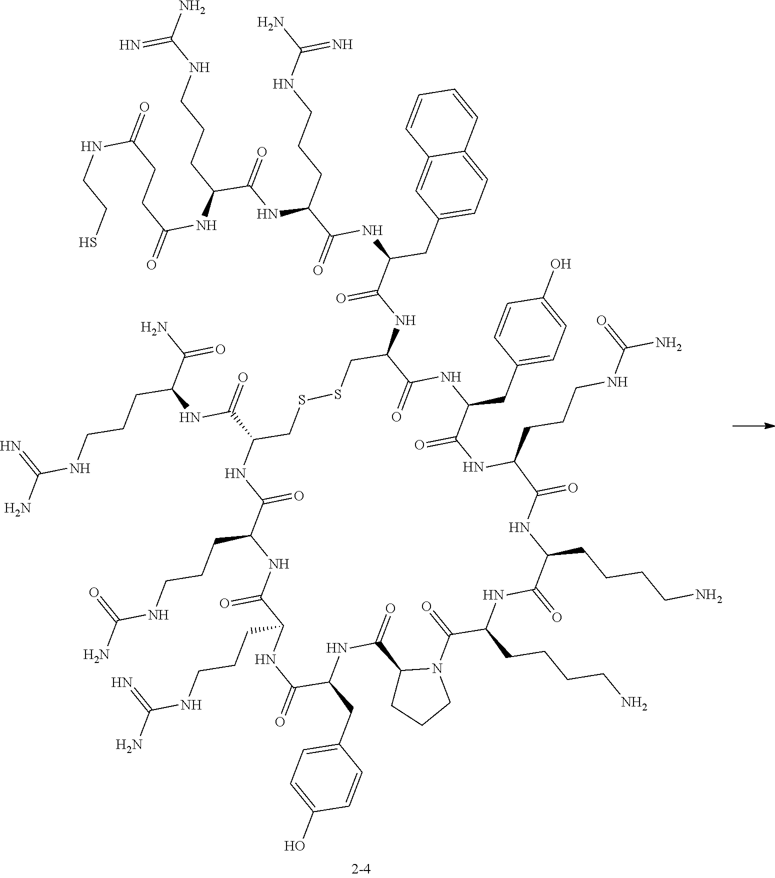 [SMILES: N=C(N)NCCC[C@H](NC(=O)[C@@H]1CSSC[C@@H](NC(=O)[C@H](Cc2ccc3ccccc3c2)NC(=O)[C@H](CCCNC(=N)N)NC(=O)[C@H](CCCNC(=N)N)NC(=O)CCC(=O)NCCS)C(=O)N[C@@H](Cc2ccc(O)cc2)C(=O)N[C@@H](CCCNC(N)=O)C(=O)N[C@@H](CCCCN)C(=O)N[C@@H](CCCCN)C(=O)N2CCC[C@H]2C(=O)N[C@@H](Cc2ccc(O)cc2)C(=O)N[C@@H](CCCNC(=N)N)C(=O)N[C@@H](CCCNC(N)=O)C(=O)N1)C(N)=O]

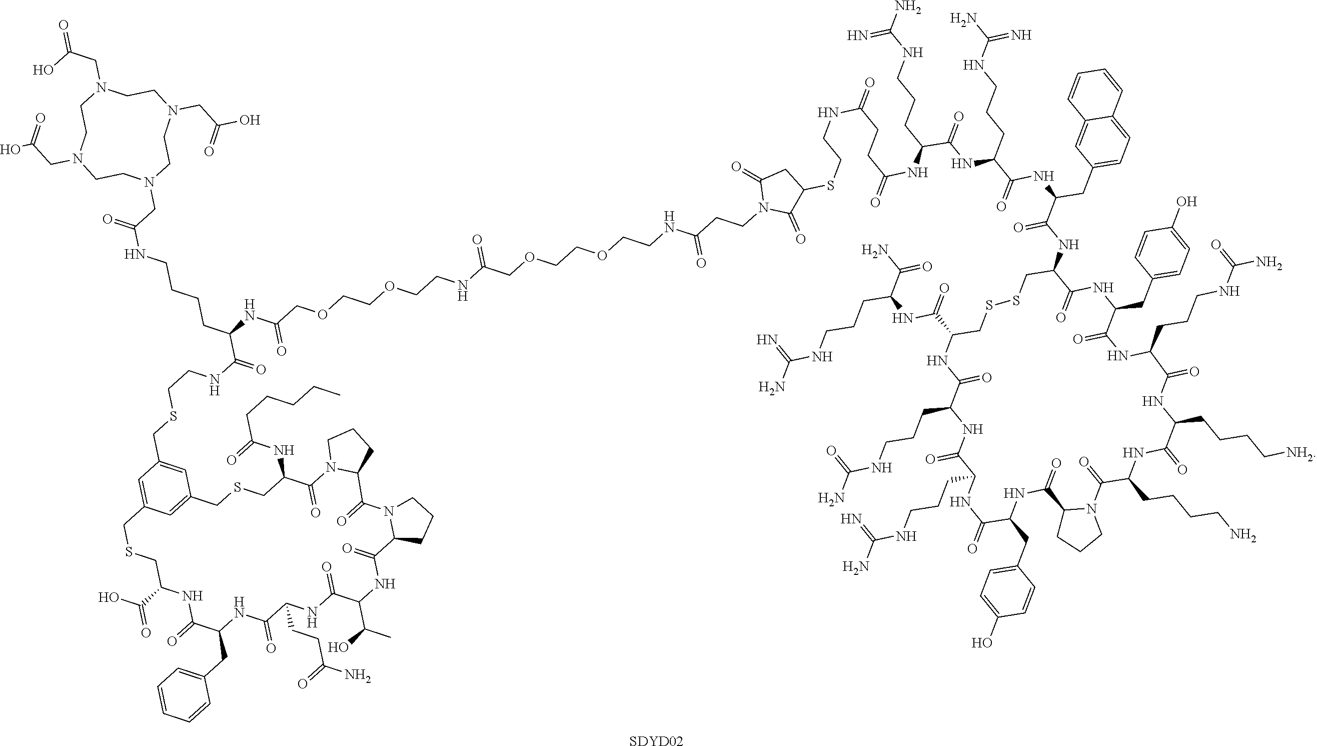

In another aspect, the present invention provides a method for preparing compound SDYD05, comprising the steps of:
1) reacting compound 5-1 with compound 5-2 to give compound 5-3:

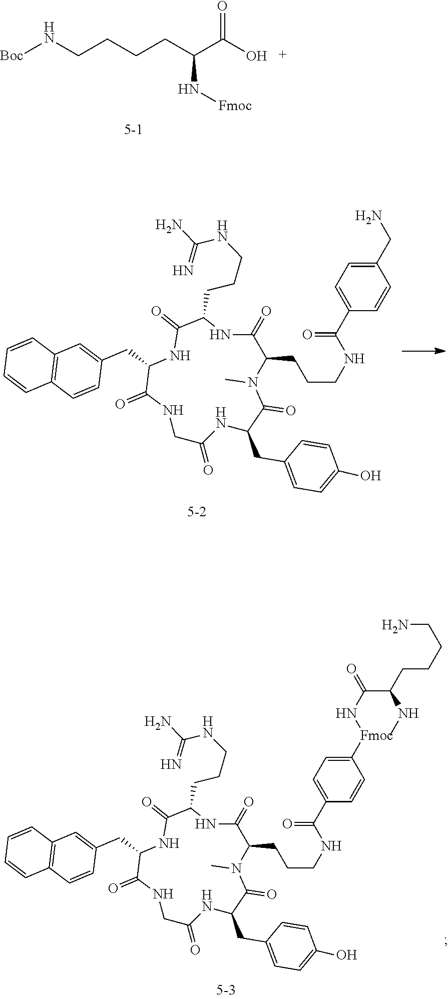

2) reacting compound 5-3 with compound 5-4 to give compound 5-5:

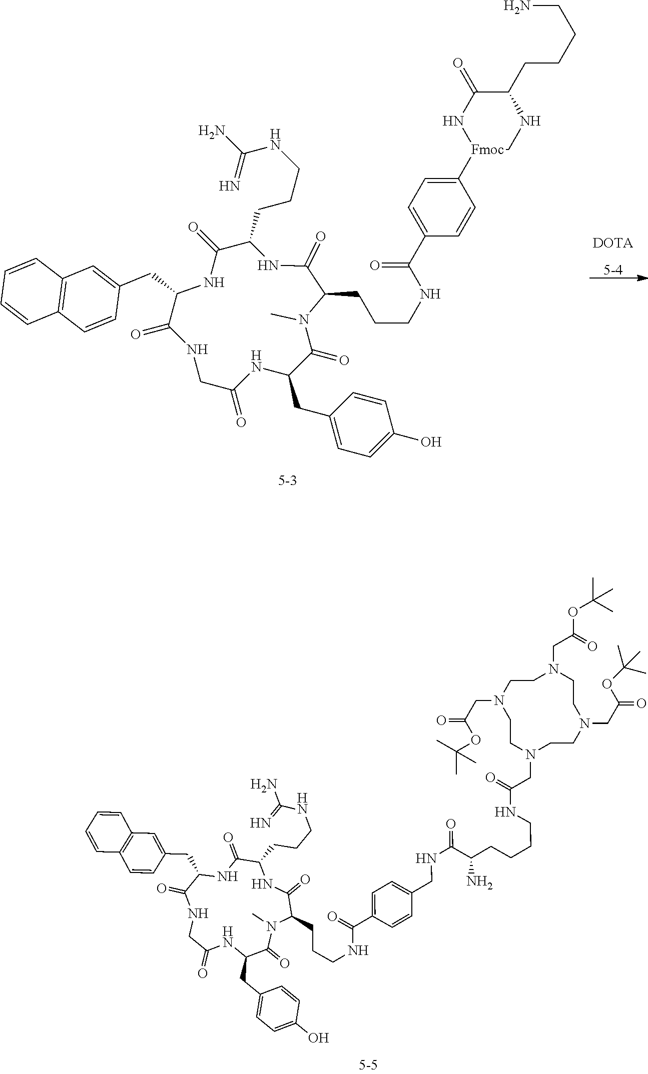

3) reacting compound 5-5 with compound 5-6 to give compound 5-7:

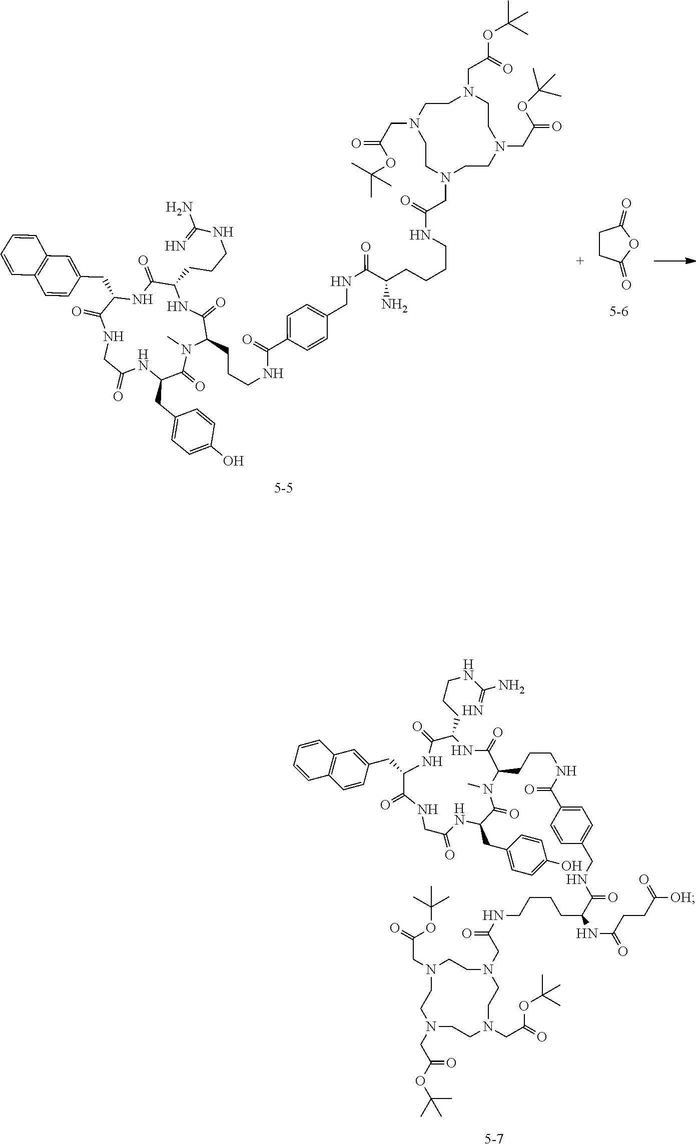 [SMILES: CN1C(=O)[C@@H](Cc2ccc(O)cc2)NC(=O)CNC(=O)[C@H](Cc2ccc3ccccc3c2)NC(=O)[C@H](CCCNC(=N)N)NC(=O)[C@H]1CCCNC(=O)c1ccc(CNC(=O)[C@@H](N)CCCCNC(=O)CN2CCN(CC(=O)OC(C)(C)C)CCN(CC(=O)OC(C)(C)C)CCN(CC(=O)OC(C)(C)C)CC2)cc1 ; O=C1CCC(=O)O1 ; CN1C(=O)[C@@H](Cc2ccc(O)cc2)NC(=O)CNC(=O)[C@H](Cc2ccc3ccccc3c2)NC(=O)[C@H](CCCNC(=N)N)NC(=O)[C@H]1CCCNC(=O)c1ccc(CNC(=O)[C@H](CCCCNC(=O)CN2CCN(CC(=O)OC(C)(C)C)CCN(CC(=O)OC(C)(C)C)CCN(CC(=O)OC(C)(C)C)CC2)NC(=O)CCC(=O)O)cc1]

and
4) reacting compound 5-7 with compound 5-8 to give compound SDYD05:

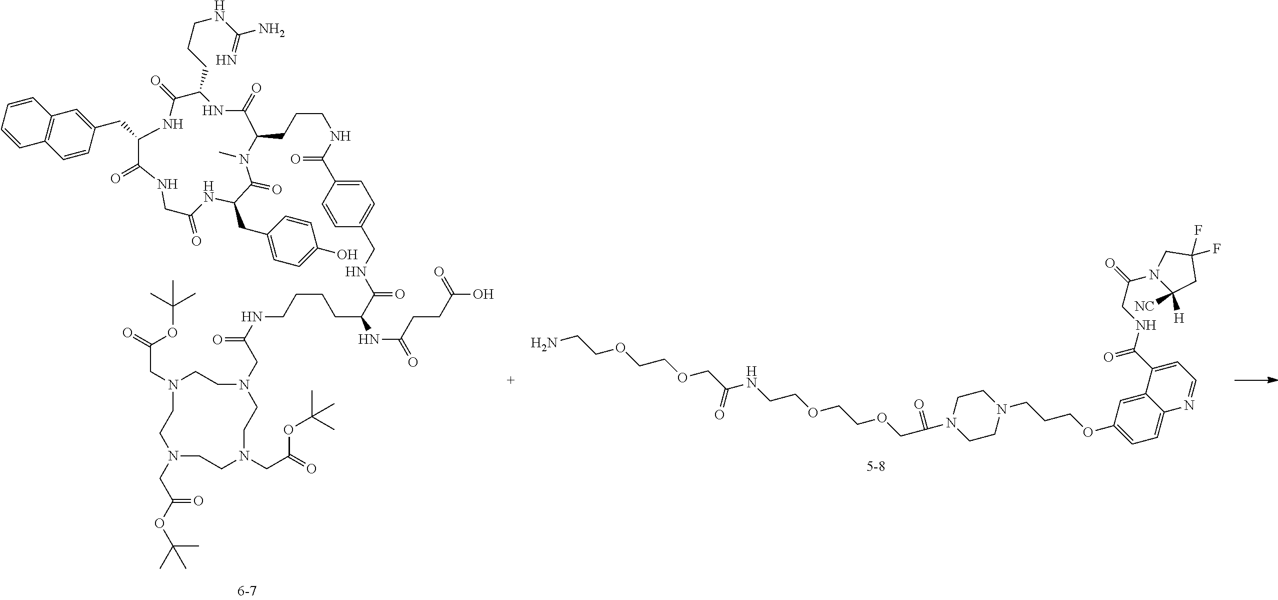 [SMILES: CN1C(=O)[C@@H](Cc2ccc(O)cc2)NC(=O)CNC(=O)[C@H](Cc2ccc3ccccc3c2)NC(=O)[C@H](CCCNC(=N)N)NC(=O)[C@H]1CCCNC(=O)c1ccc(CNC(=O)[C@H](CCCCNC(=O)CN2CCN(CC(=O)OC(C)(C)C)CCN(CC(=O)OC(C)(C)C)CCN(CC(=O)OC(C)(C)C)CC2)NC(=O)CCC(=O)O)cc1 ; N#C[C@H]1CC(F)(F)CN1C(=O)CNC(=O)c1ccnc2ccc(OCCCN3CCN(C(=O)COCCOCCNC(=O)COCCOCCN)CC3)cc12]

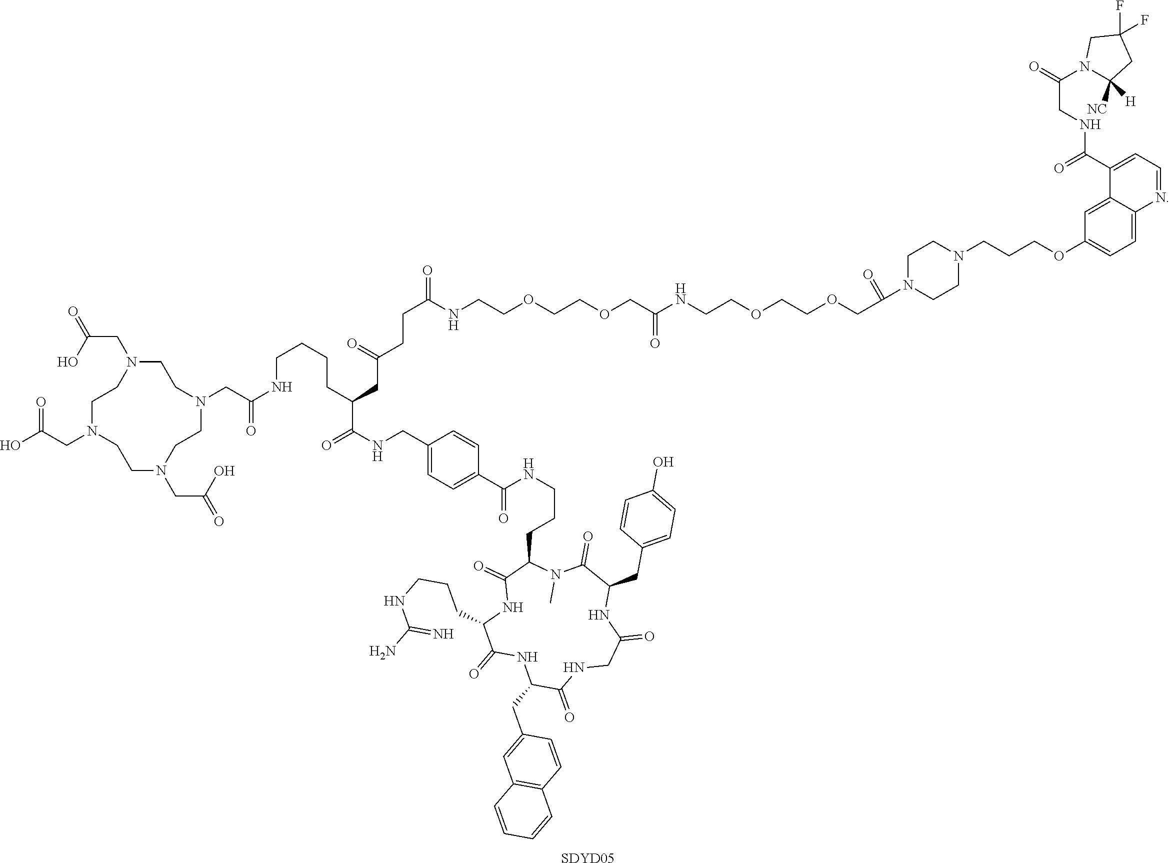

In another aspect, the present invention provides a method for preparing compound SDYD06, comprising the steps of:
1) reacting compound 6-1 with compound 6-2 to give compound 6-3:

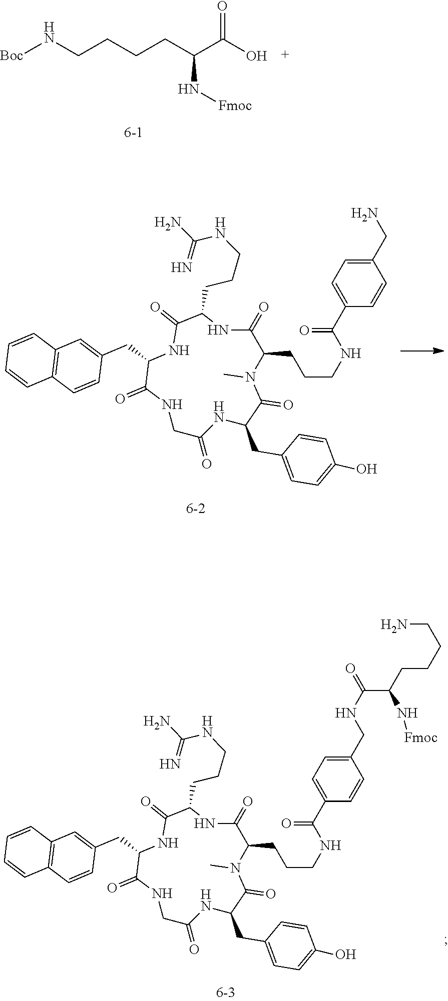

2) reacting compound 6-3 with compound 6-4 to give compound 6-5:

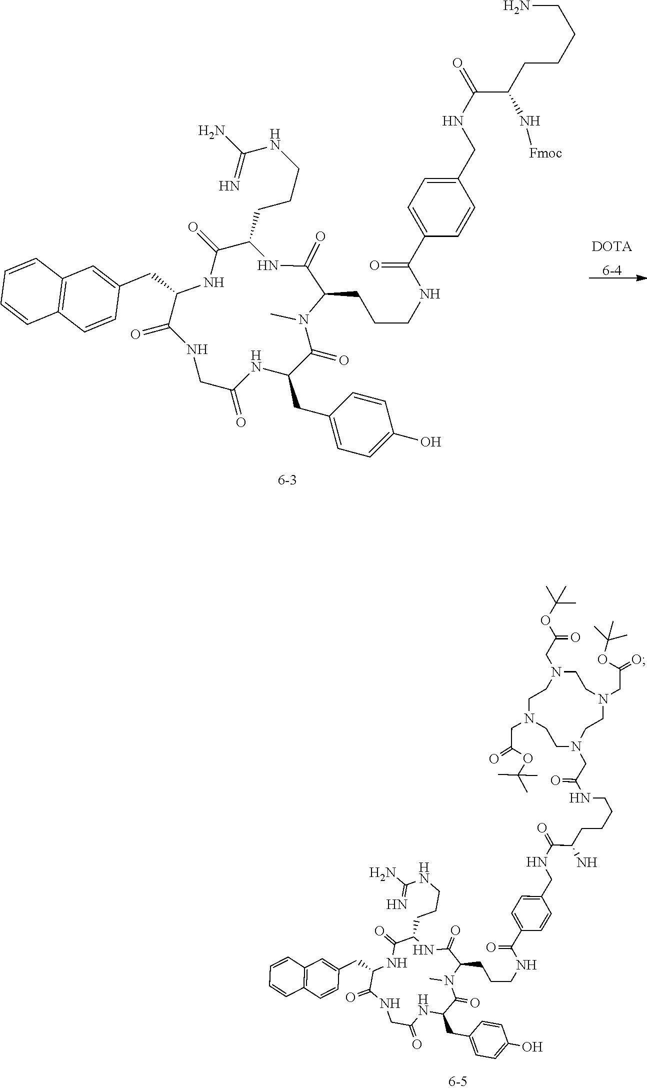

3) reacting compound 6-5 with compound 6-6 to give compound 6-7:

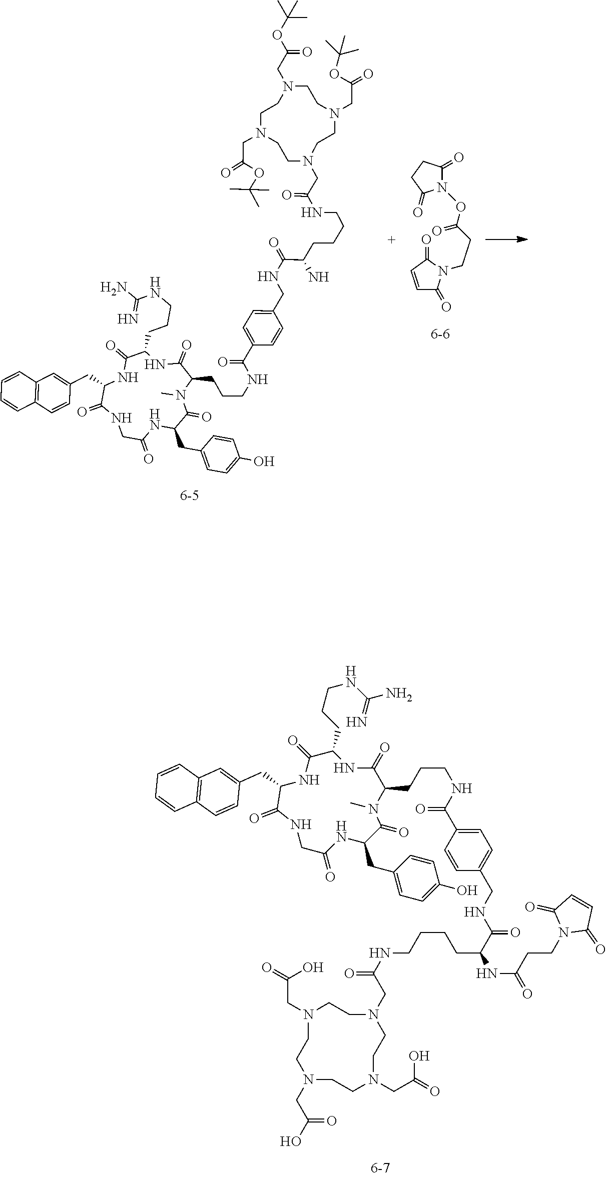 [SMILES: CN1C(=O)[C@@H](Cc2ccc(O)cc2)NC(=O)CNC(=O)[C@H](Cc2ccc3ccccc3c2)NC(=O)[C@H](CCCNC(=N)N)NC(=O)[C@H]1CCCNC(=O)c1ccc(CNC(=O)[C@@H](N)CCCCNC(=O)CN2CCN(CC(=O)OC(C)(C)C)CCN(CC(=O)OC(C)(C)C)CCN(CC(=O)OC(C)(C)C)CC2)cc1 ; O=C(CCN1C(=O)C=CC1=O)ON1C(=O)CCC1=O ; CN1C(=O)[C@@H](Cc2ccc(O)cc2)NC(=O)CNC(=O)[C@H](Cc2ccc3ccccc3c2)NC(=O)[C@H](CCCNC(=N)N)NC(=O)[C@H]1CCCNC(=O)c1ccc(CNC(=O)[C@H](CCCCNC(=O)CN2CCN(CC(=O)O)CCN(CC(=O)O)CCN(CC(=O)O)CC2)NC(=O)CCN2C(=O)C=CC2=O)cc1]

and
4) reacting compound 6-7 with compound 6-8 to give compound SDYD06:

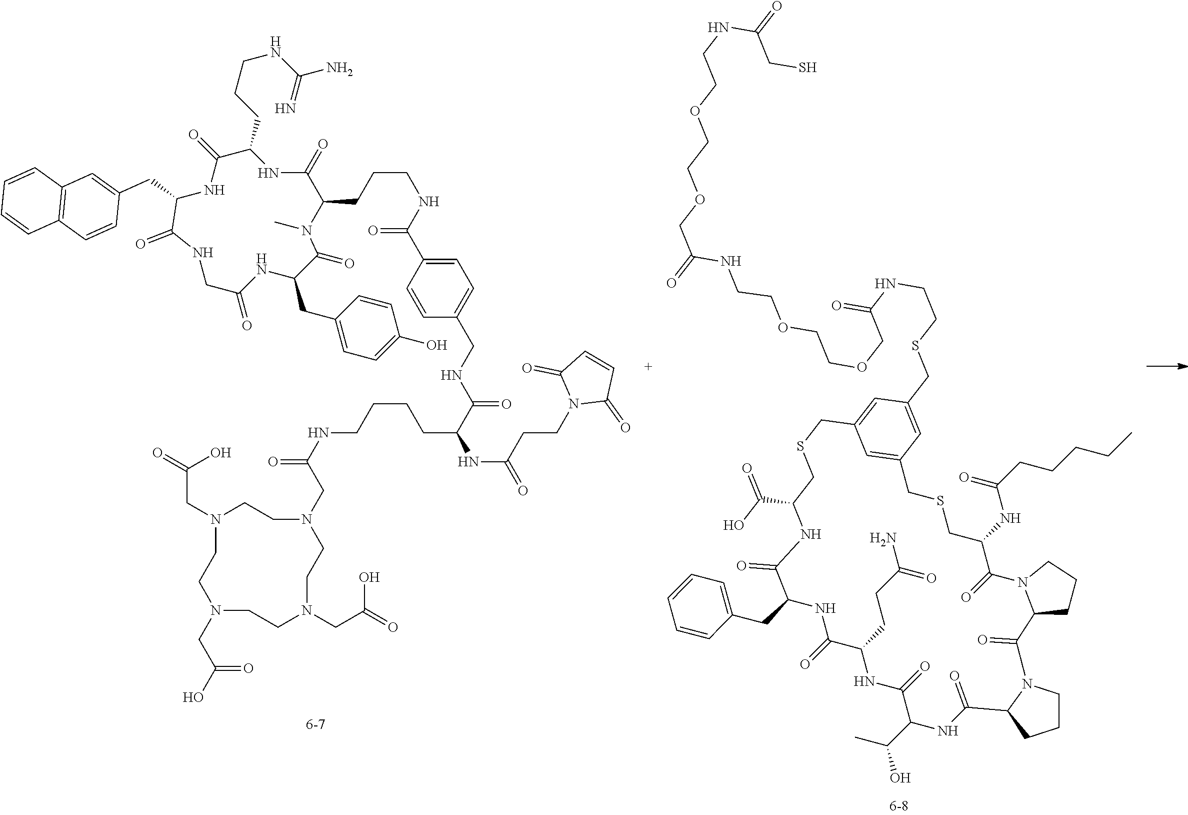 [SMILES: CN1C(=O)[C@@H](Cc2ccc(O)cc2)NC(=O)CNC(=O)[C@H](Cc2ccc3ccccc3c2)NC(=O)[C@H](CCCNC(=N)N)NC(=O)[C@H]1CCCNC(=O)c1ccc(CNC(=O)[C@H](CCCCNC(=O)CN2CCN(CC(=O)O)CCN(CC(=O)O)CCN(CC(=O)O)CC2)NC(=O)CCN2C(=O)C=CC2=O)cc1 ; CCCCCC(=O)N[C@H]1CSCc2cc(CSCCNC(=O)COCCOCCNC(=O)COCCOCCNC(=O)CS)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O]

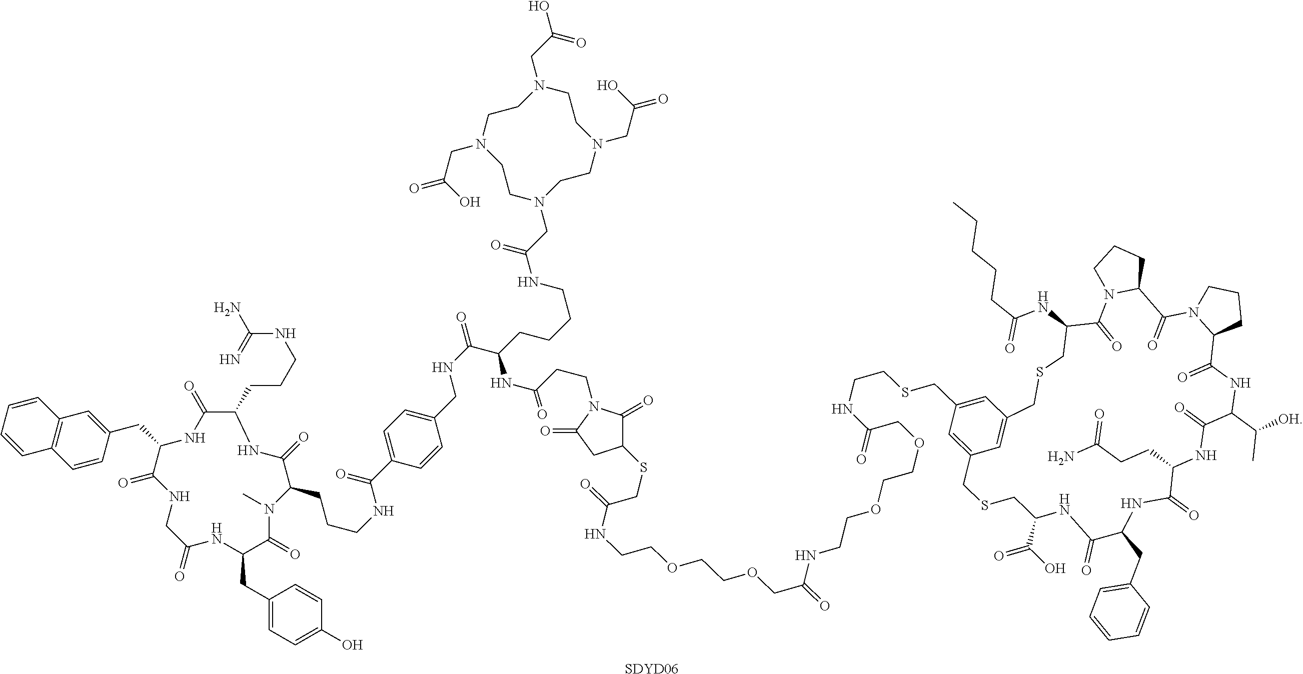 [SMILES: CCCCCC(=O)N[C@@H]1CSCc2cc(CSCCNC(=O)COCCOCCNC(=O)COCCOCCNC(=O)CSC3CC(=O)N(CCC(=O)N[C@H](CCCCNC(=O)CN4CCN(CC(=O)O)CCN(CC(=O)O)CCN(CC(=O)O)CC4)C(=O)NCc4ccc(C(=O)NCCC[C@@H]5C(=O)N[C@@H](CCCNC(=N)N)C(=O)N[C@@H](Cc6ccc7ccccc7c6)C(=O)NCC(=O)N[C@H](Cc6ccc(O)cc6)C(=O)N5C)cc4)C3=O)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O ; "SDYD06"]

In another aspect, the present invention provides a method for preparing compound SDYD03, comprising the steps of:
1) reacting compound 3-1 with compound 3-2 to give compound 3-3:

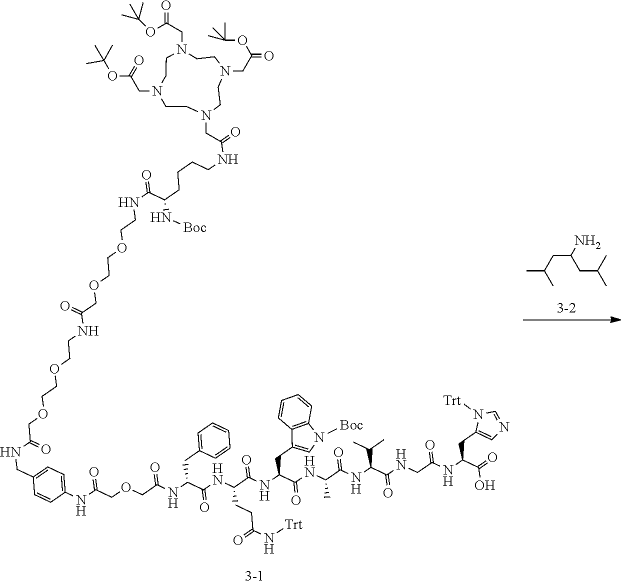

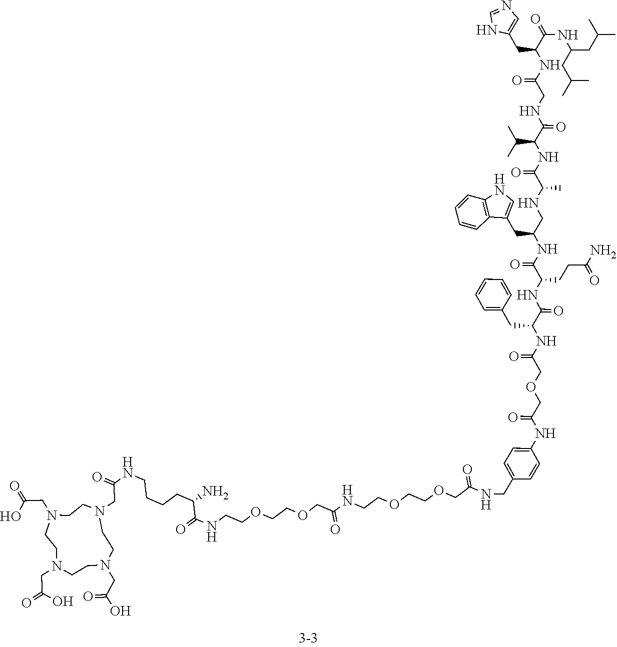 [SMILES: CC(C)CC(CC(C)C)NC(=O)[C@H](Cc1cnc[nH]1)NC(=O)CNC(=O)[C@@H](NC(=O)[C@H](C)NC[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)COCCOCCNC(=O)COCCOCCNC(=O)[C@@H](N)CCCCNC(=O)CN2CCN(CC(=O)O)CCN(CC(=O)O)CCN(CC(=O)O)CC2)cc1)C(C)C]

and
2) reacting compound 3-3 with compound 3-4 to give compound SDYD03:

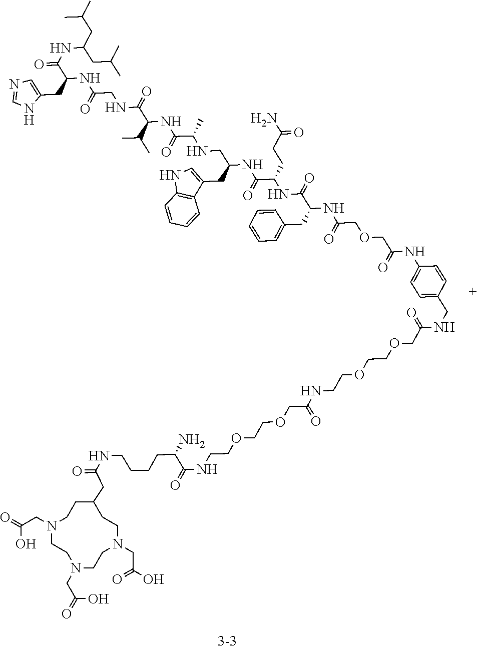 [SMILES: CC(C)CC(CC(C)C)NC(=O)[C@H](Cc1cnc[nH]1)NC(=O)CNC(=O)[C@@H](NC(=O)[C@H](C)NC[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)COCCOCCNC(=O)COCCOCCNC(=O)[C@@H](N)CCCCNC(=O)CC2CCN(CC(=O)O)CCN(CC(=O)O)CCN(CC(=O)O)CC2)cc1)C(C)C]

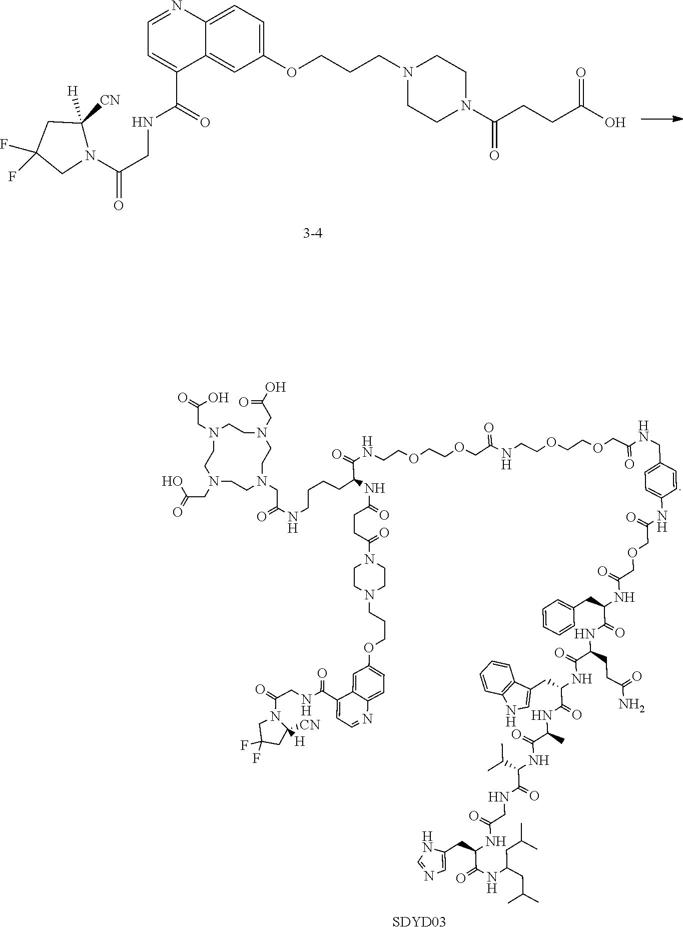 [SMILES: N#C[C@@H]1CC(F)(F)CN1C(=O)CNC(=O)c1ccnc2ccc(OCCCN3CCN(C(=O)CCC(=O)O)CC3)cc12 ; CC(C)CC(CC(C)C)NC(=O)[C@@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)COCCOCCNC(=O)COCCOCCNC(=O)[C@H](CCCCNC(=O)CN2CCN(CC(=O)O)CCN(CC(=O)O)CCN(CC(=O)O)CC2)NC(=O)CCC(=O)N2CCN(CCCOc3ccc4nccc(C(=O)NCC(=O)N5CC(F)(F)C[C@H]5C#N)c4c3)CC2)cc1)C(C)C ; "SDYD03"]

In another aspect, the present invention provides a method for preparing compound SDYD04, comprising the steps of:
1) reacting compound 4-1 with compound 4-2 to give compound 4-3:

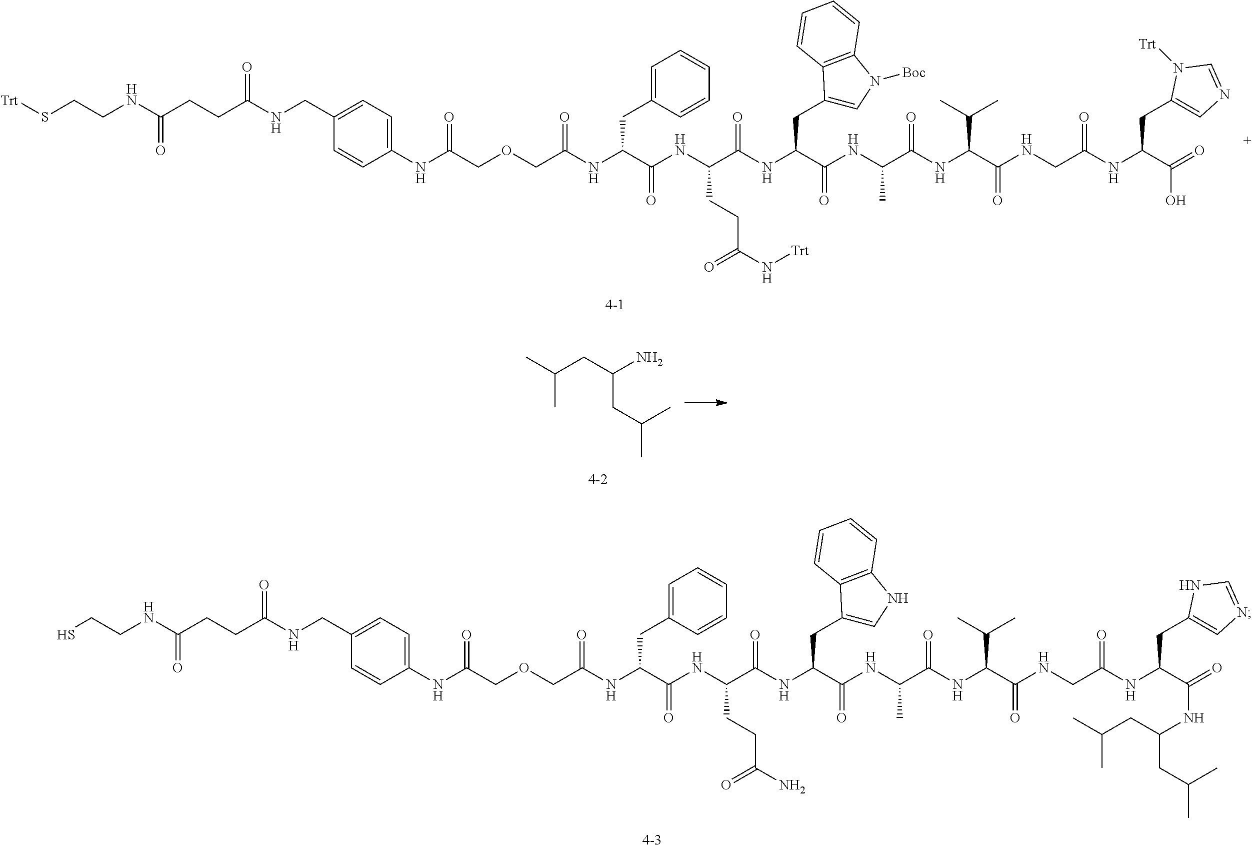

and
2) reacting compound 4-3 with compound 4-4 to give compound SDYD04:

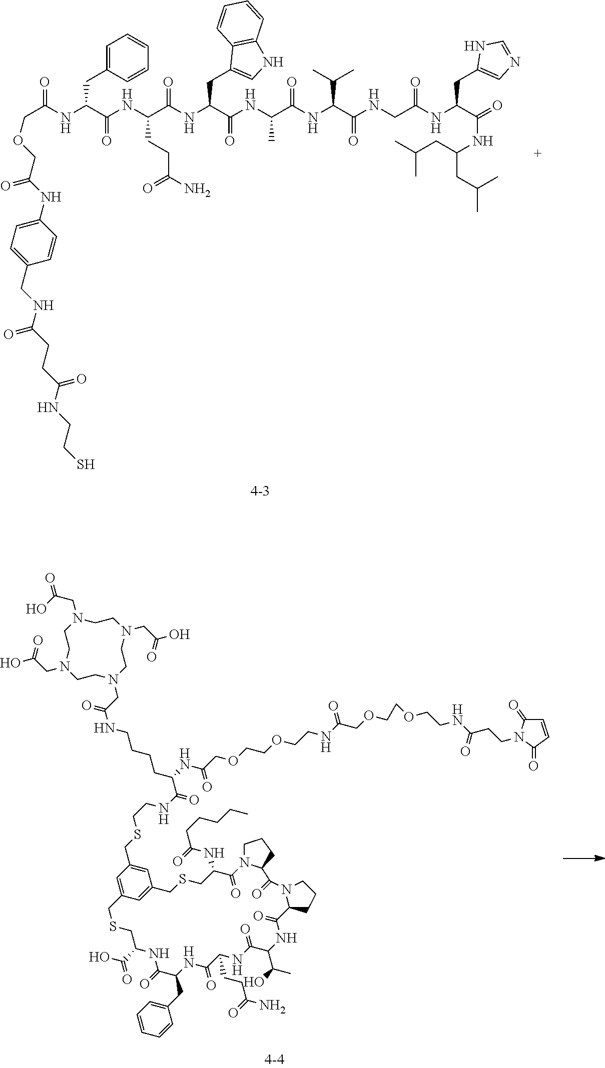 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CCC(=O)NCCS)cc1)C(C)C ; CCCCCC(=O)N[C@H]1CSCc2cc(CSCCNC(=O)[C@H](CCCCNC(=O)CN3CCN(CC(=O)O)CCN(CC(=O)O)CCN(CC(=O)O)CC3)NC(=O)COCCOCCNC(=O)COCCOCCNC(=O)CCN3C(=O)C=CC3=O)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O]

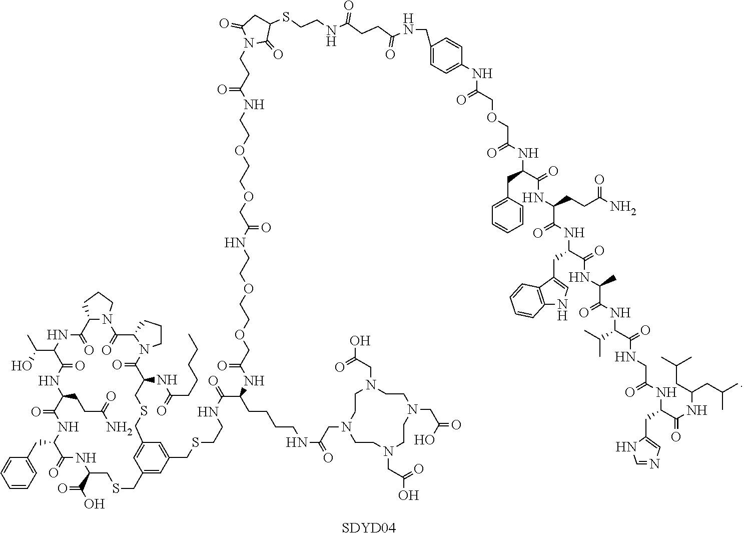 [SMILES: CCCCCC(=O)N[C@H]1CSCc2cc(CSCCNC(=O)[C@H](CCCCNC(=O)CN3CCN(CC(=O)O)CCN(CC(=O)O)CCN(CC(=O)O)CC3)NC(=O)COCCOCCNC(=O)COCCOCCNC(=O)CCN3C(=O)CC(SCCNC(=O)CCC(=O)NCc4ccc(NC(=O)COCC(=O)N[C@H](Cc5ccccc5)C(=O)N[C@@H](CCC(N)=O)C(=O)N[C@@H](Cc5c[nH]c6ccccc56)C(=O)N[C@@H](C)C(=O)N[C@H](C(=O)NCC(=O)N[C@@H](Cc5cnc[nH]5)C(=O)NC(CC(C)C)CC(C)C)C(C)C)cc4)C3=O)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O ; "SDYD04"]

In another aspect, the present invention provides a method for preparing compound SDYD07, comprising the steps of:
1) reacting compound 7-1 with compound 7-2 to give compound 7-3:

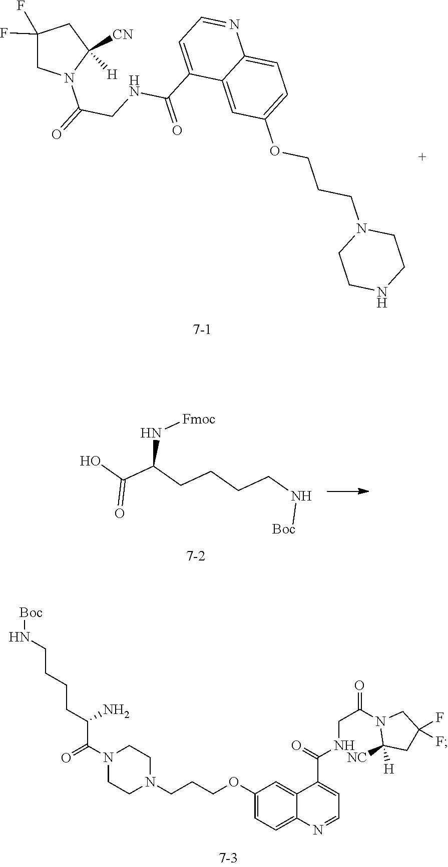

2) reacting compound 7-3 with compound 7-4 to give compound 7-5:

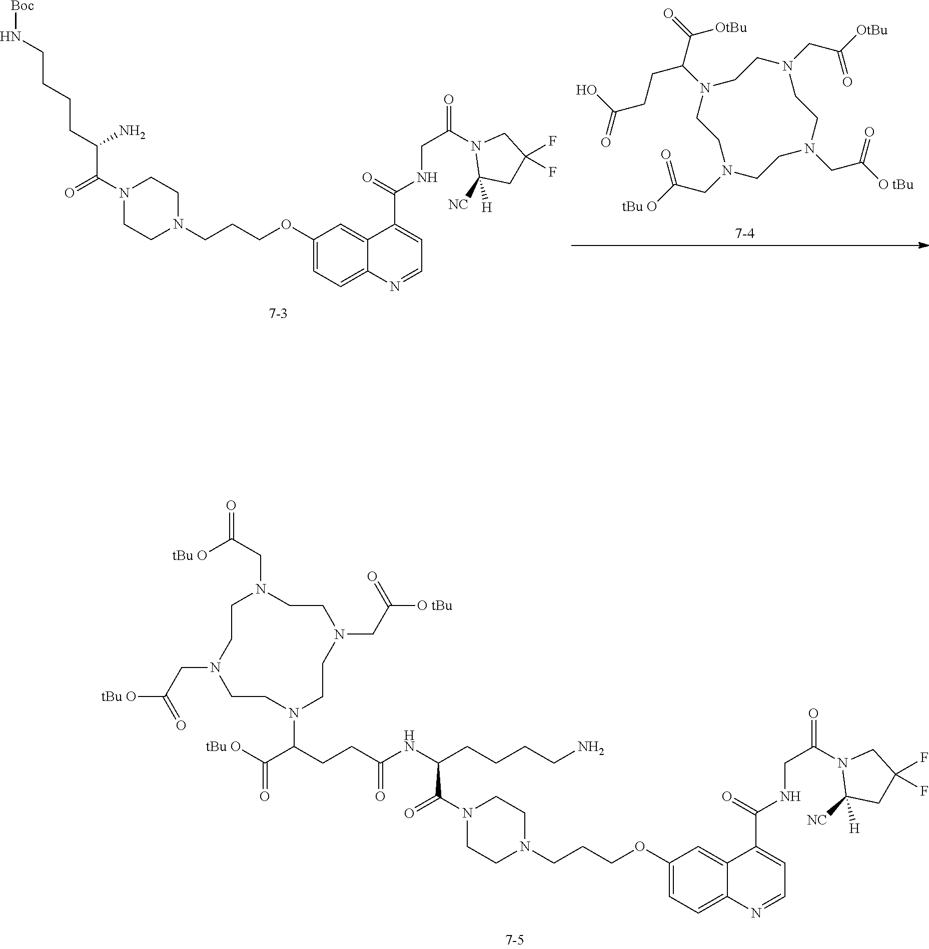

3) reacting compound 7-5 with compound 7-6 to give compound 7-7:

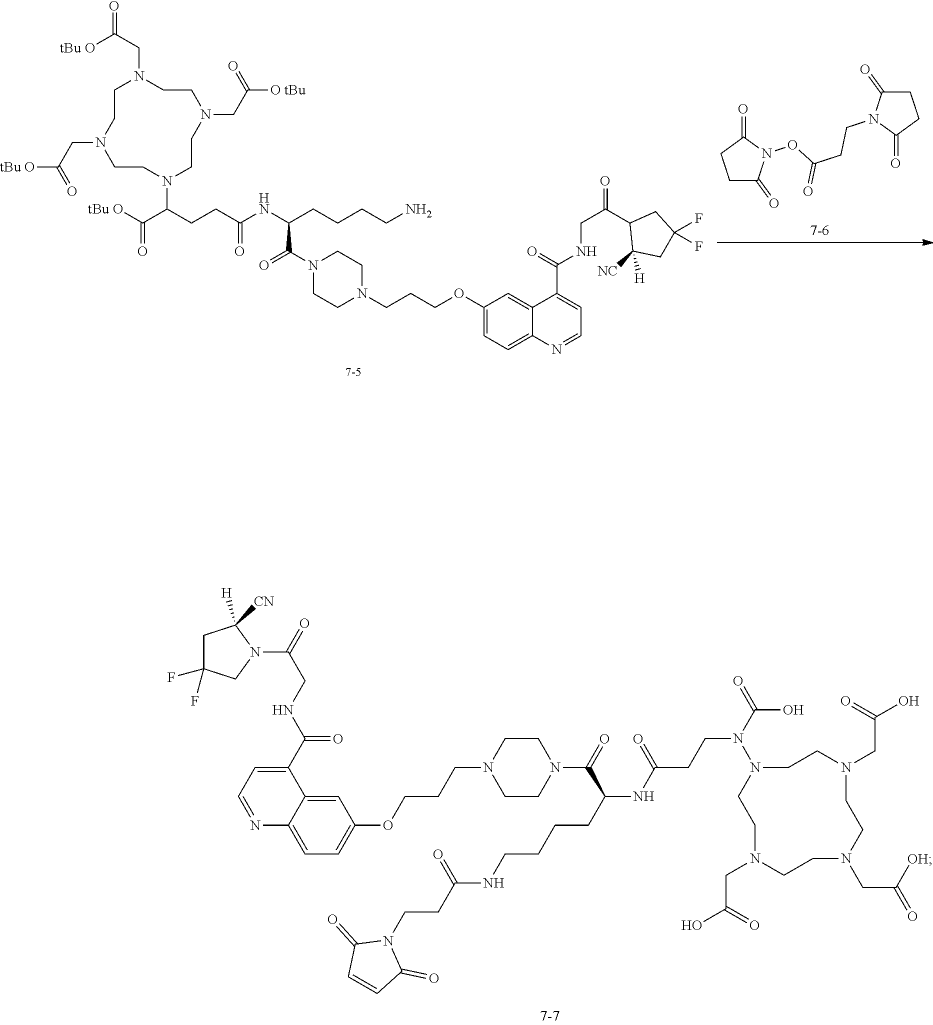

4) reacting compound 7-8 with compound 7-9 to give compound 7-10:

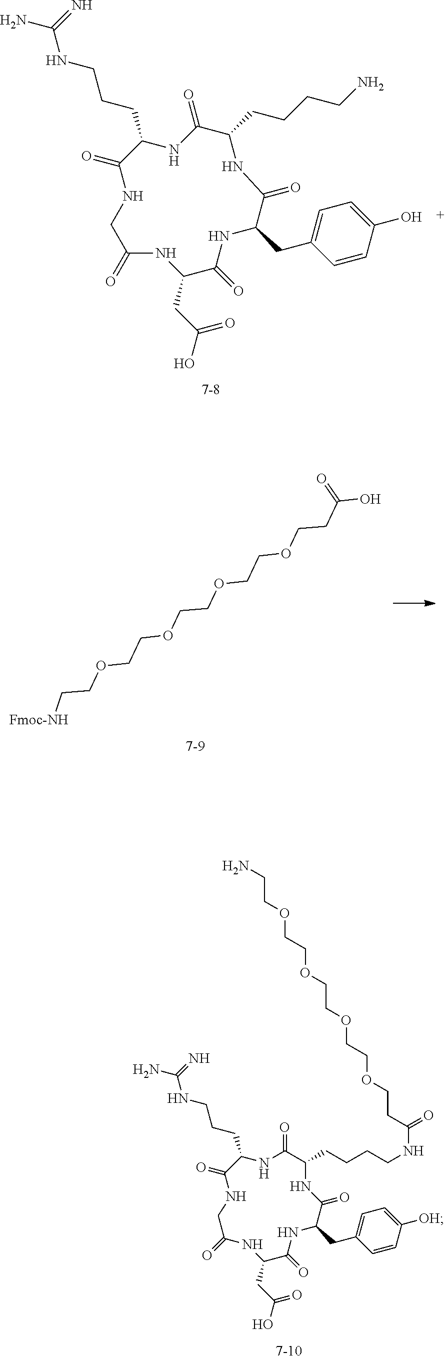

5) reacting compound 7-10 with compound 7-11 to give compound 7-12:

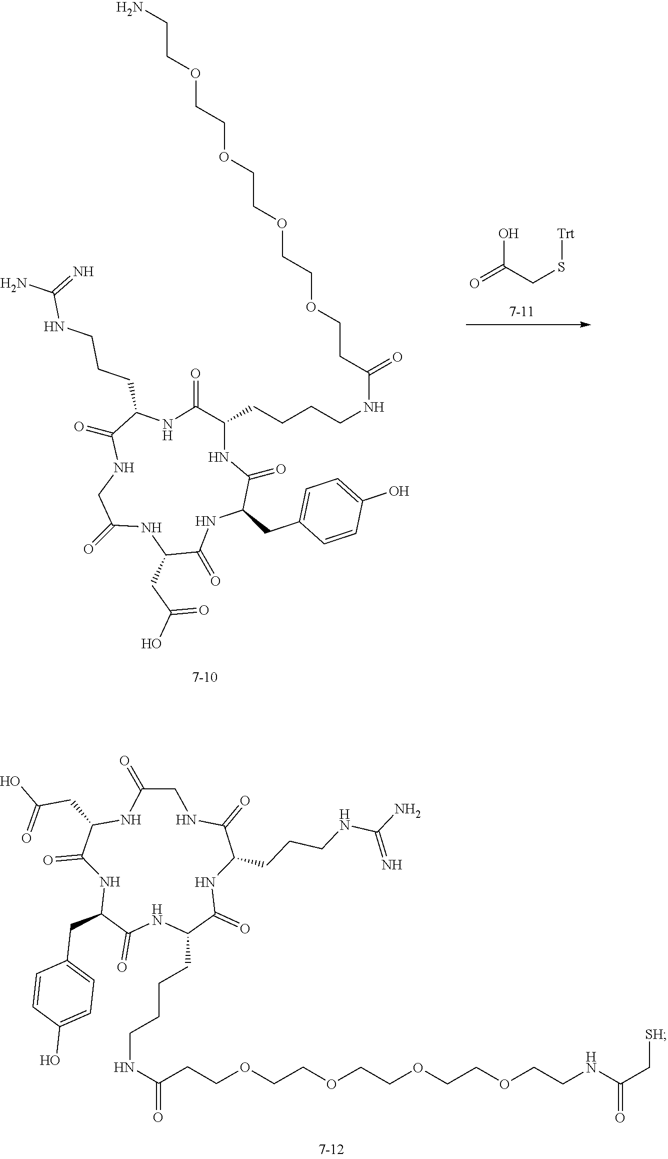

and
6) reacting compound 7-12 with compound 7-7 to give compound SDYD07:

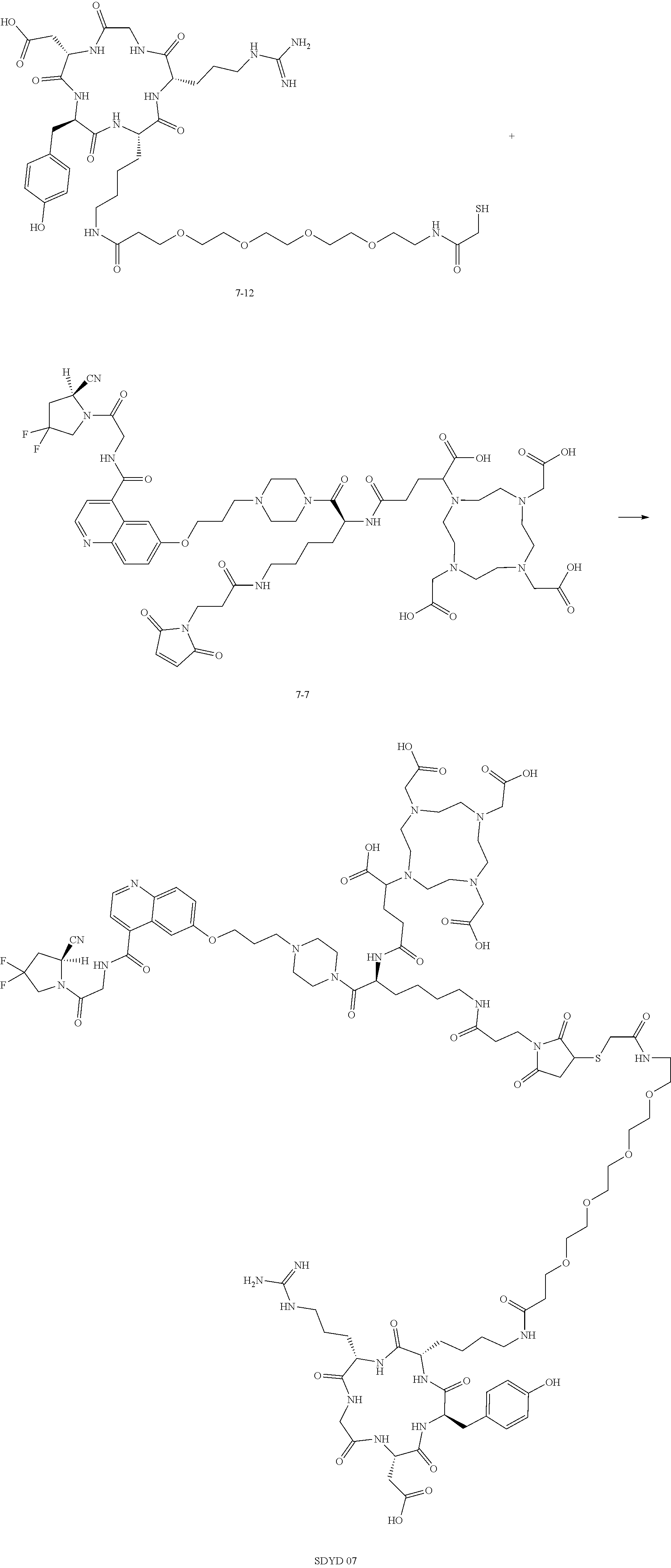

In another aspect, the present invention provides a method for preparing compound SDYD08, comprising the steps of:
1) reacting compound 8-1 with compound 8-2 to give compound 8-3:

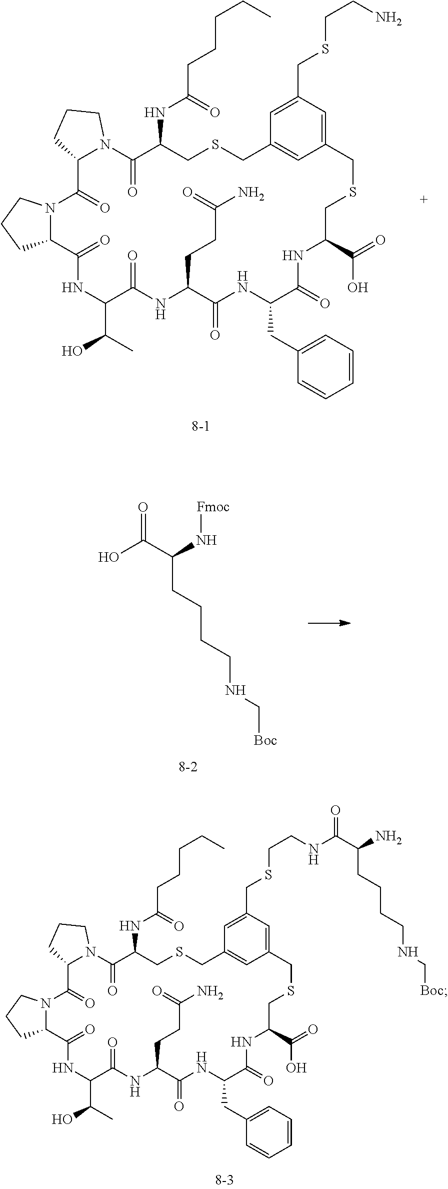

2) reacting compound 8-3 with compound 8-4 to give compound 8-5:

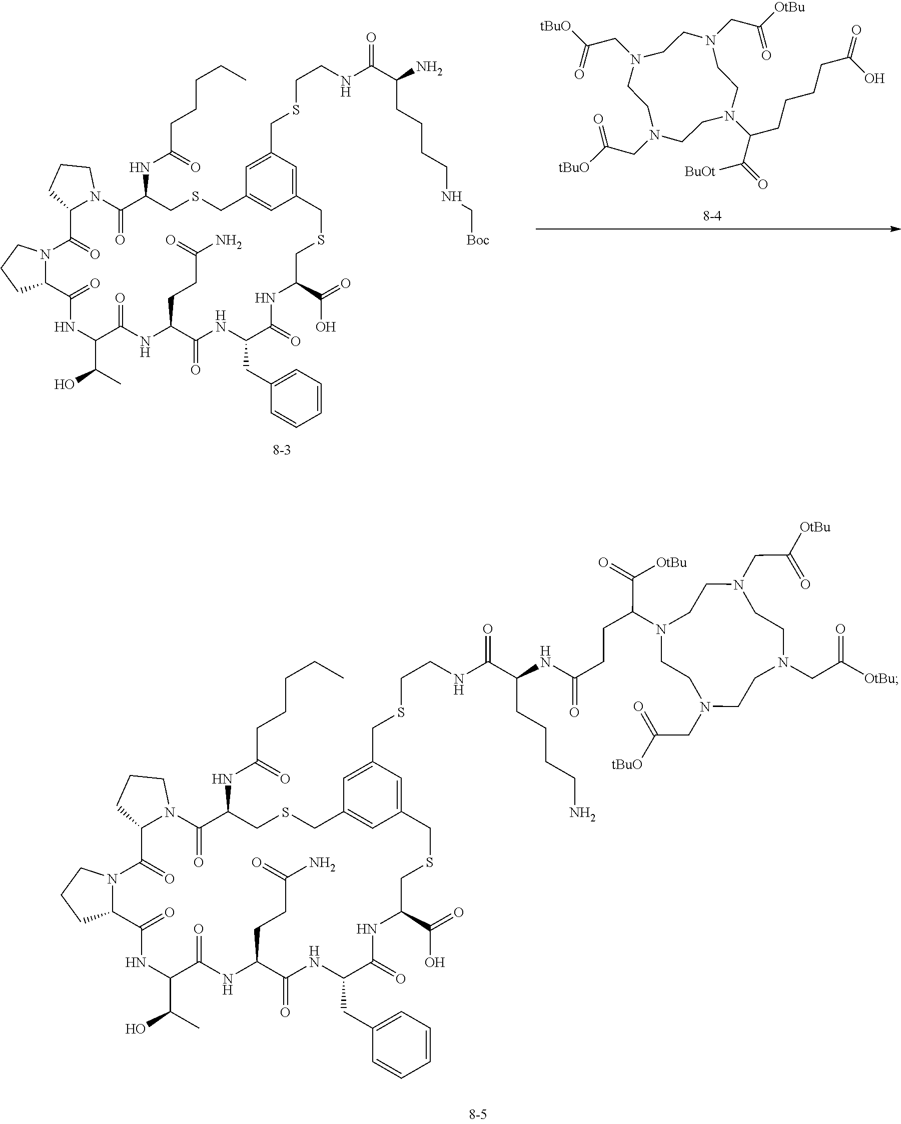

3) reacting compound 8-5 with compound 8-6 to give compound 8-7:

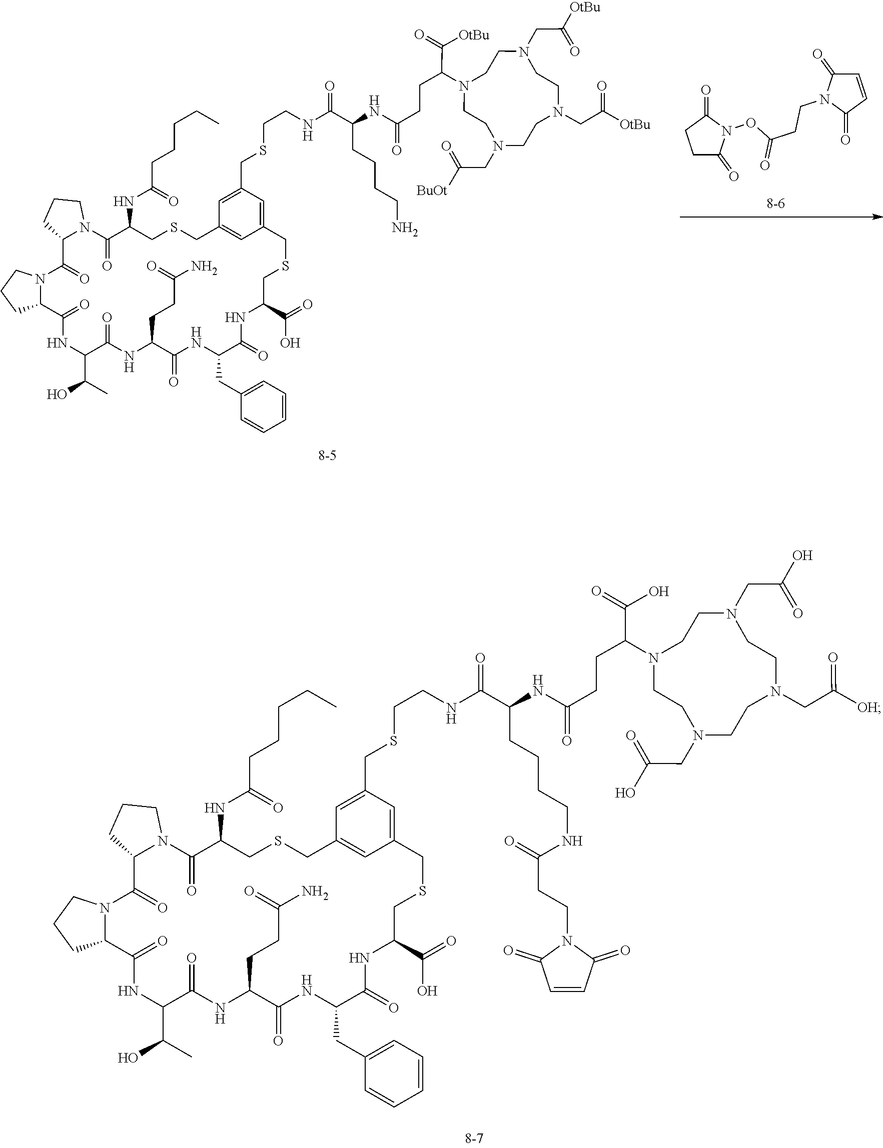

and
4) reacting compound 8-7 with compound 8-8 to give compound SDYD08:

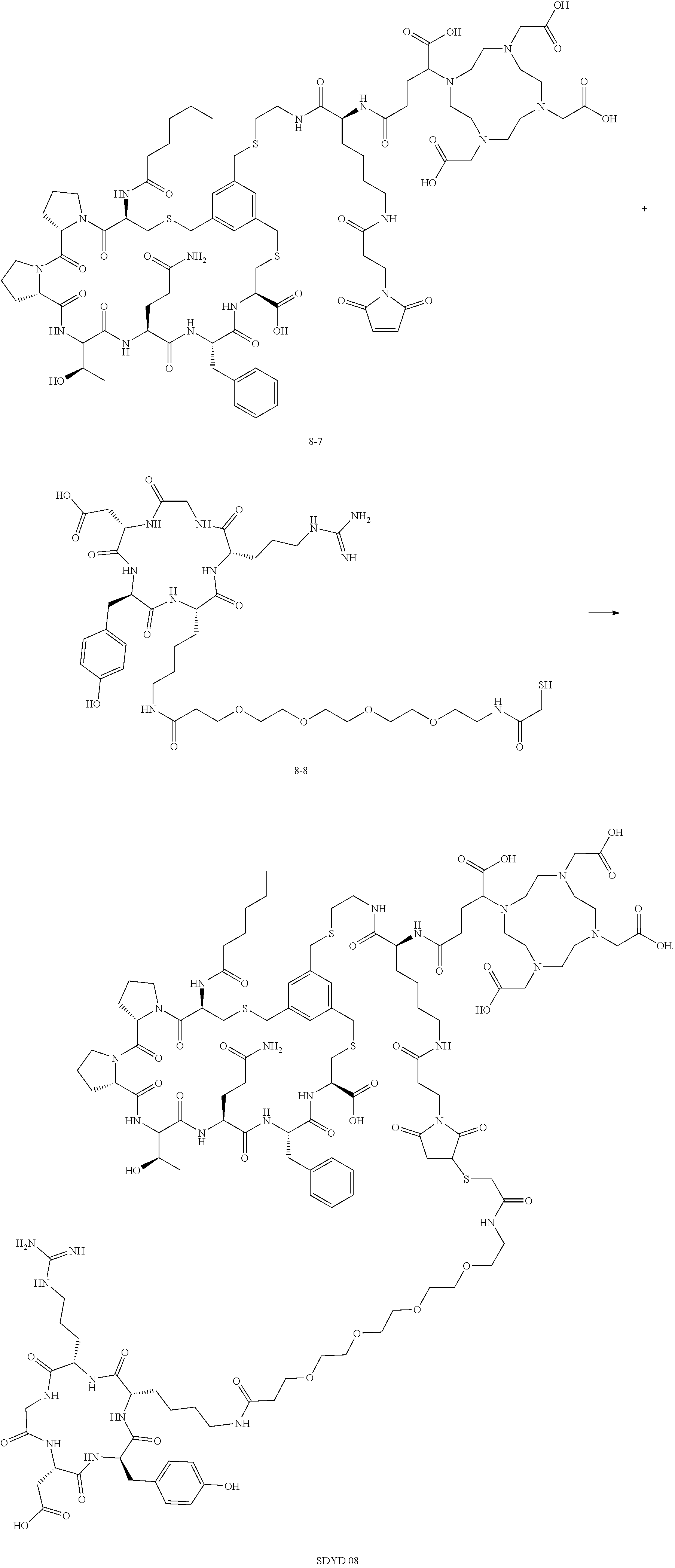

In another aspect, the present invention provides a radionuclide-labeled compound, obtained by labeling the compound of the present invention with a radionuclide.

In some embodiments, the radioactive moiety is a fluorescent isotope, a radioisotope, a radiopharmaceutical, or their combinations.

In some embodiments, wherein the radionuclide is selected from the group consisting of an isotope that emits alpha rays, an isotope that emits beta rays, an isotope that emits γ rays, an isotope that emits Auger electrons, an isotope that emits X-rays, and an isotope that emits fluorescence; preferably, wherein the radionuclide is selected from the group consisting of ¹⁸F, ⁵¹Cr, ⁶⁷Ga, ⁶⁸Ga, ¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁸Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ⁶⁴Cu, ⁶⁷Cu, ¹⁸⁸Re, ¹⁸⁶Re, ¹⁹⁸Au, ²²⁵Ac, ²²⁷Th, and ¹⁹⁹Ag.

In some embodiments, the radionuclide is selected from the group consisting of ¹⁸F, ⁵¹Cr, ⁶⁷Ga, ⁶⁸Ga, ¹¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁸Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ⁶⁴Cu, ⁶⁷Cu, ¹⁸⁸Re, ¹⁸⁶Re, ¹⁹⁸Au, ²²⁵Ac, ²²⁷TH, and ¹⁹⁰Ag.

In some embodiments, the radionuclide is selected from the group consisting of ⁶⁸Ga and ¹⁷⁷Lu.

In some embodiments, the radionuclide-labeled compound is selected from the group consisting of:

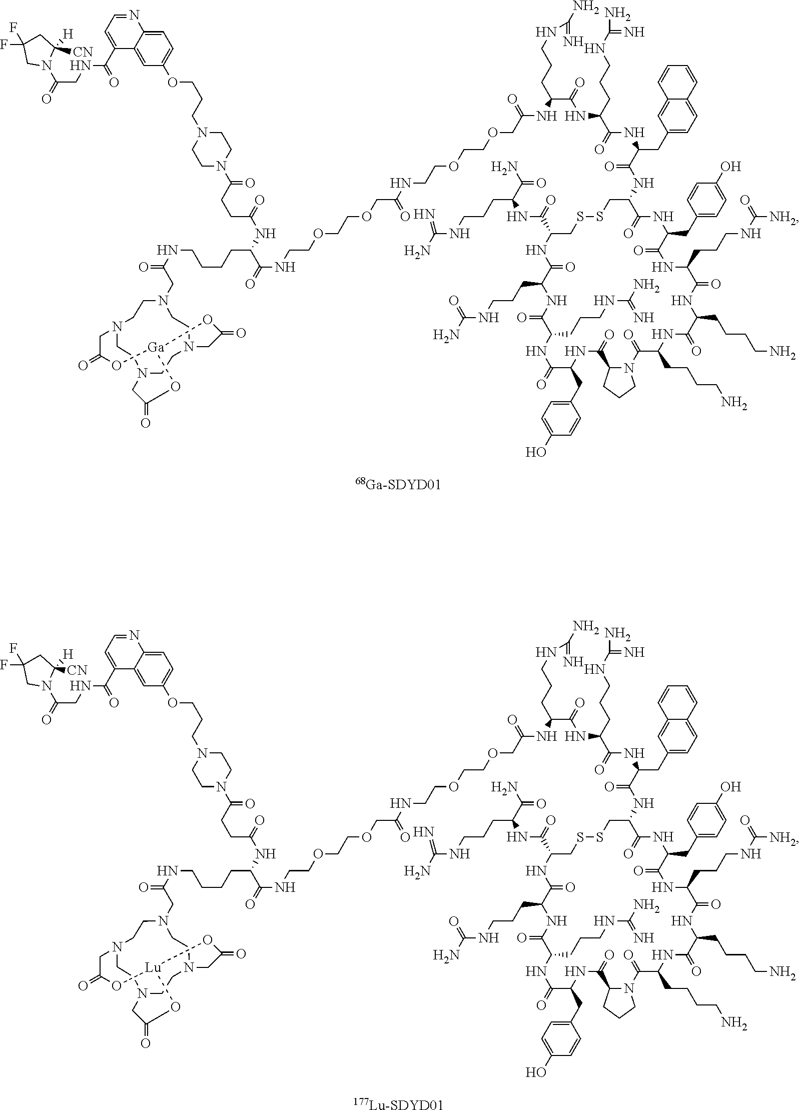

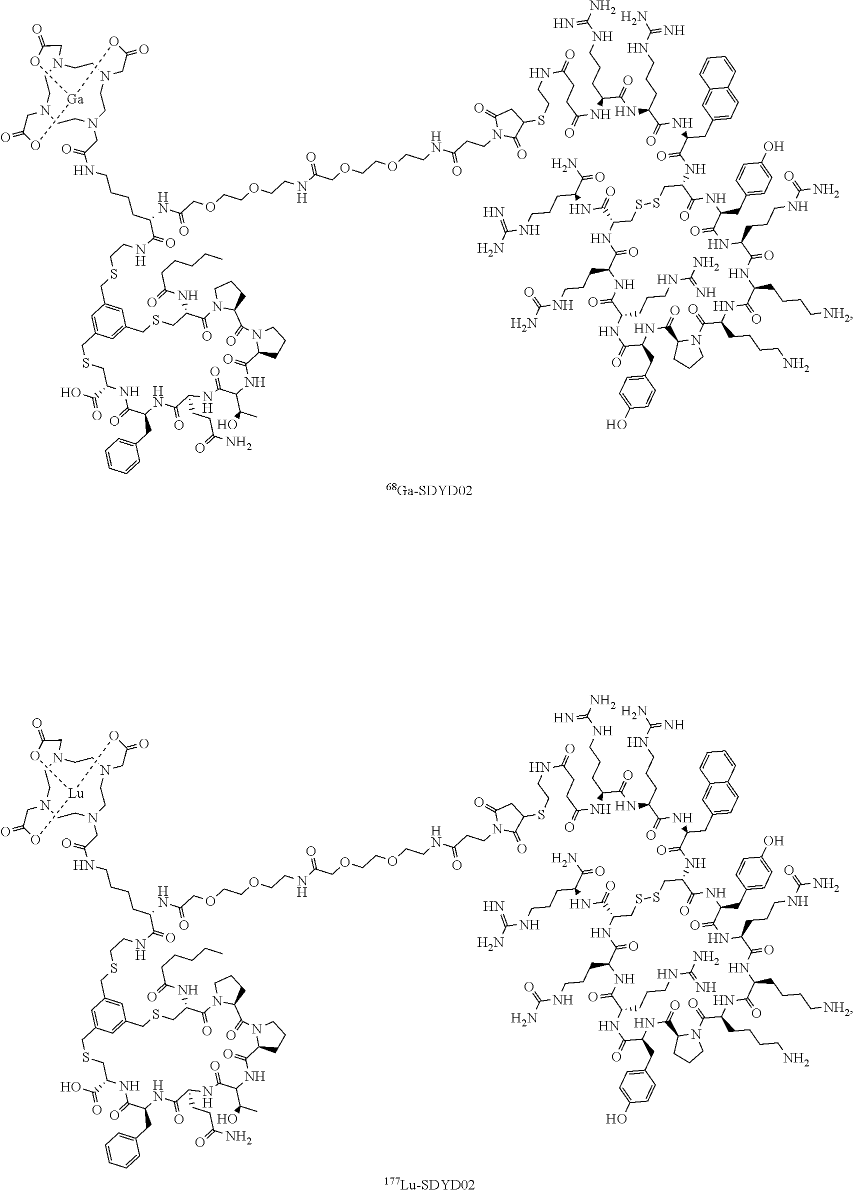

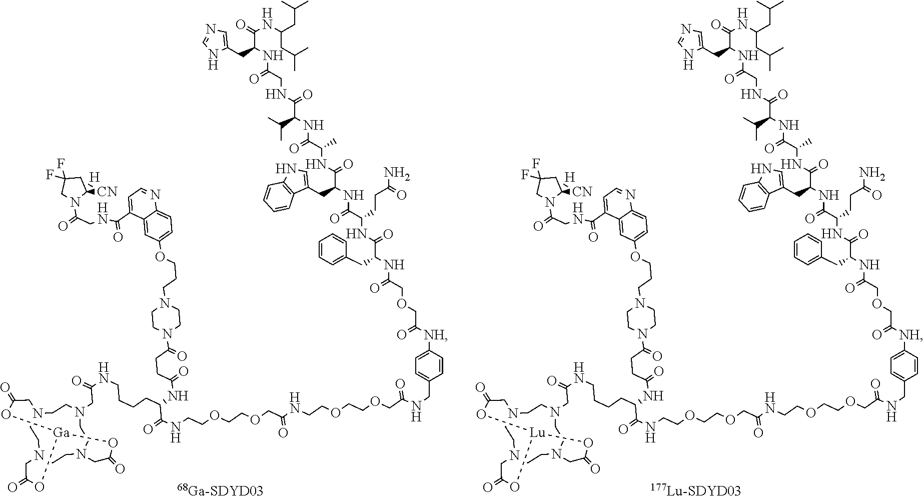

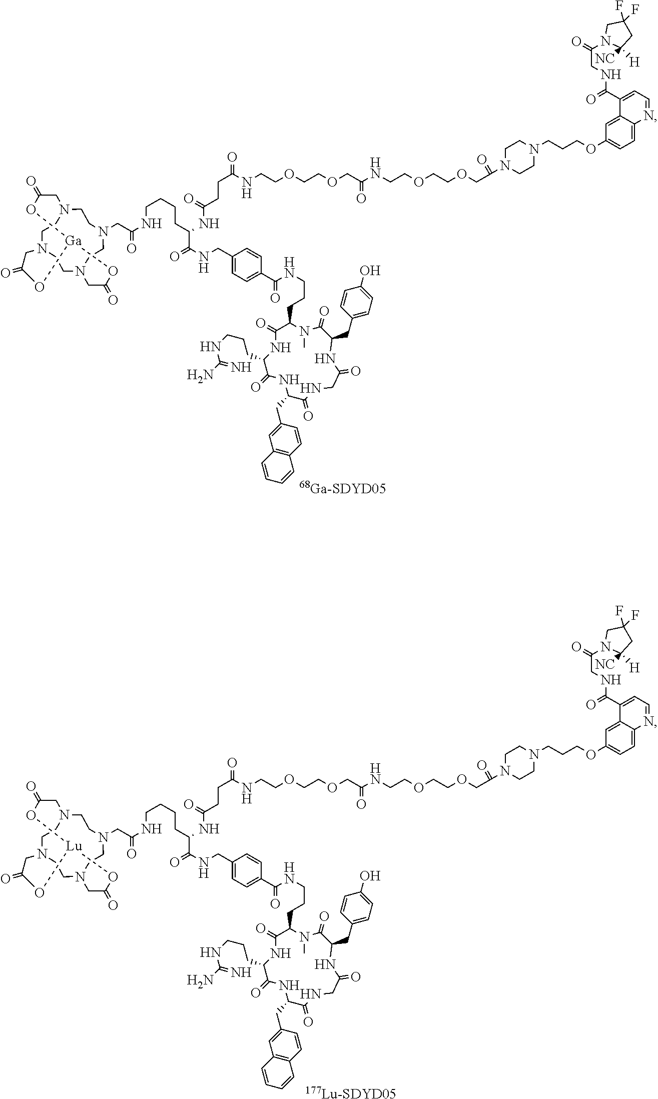

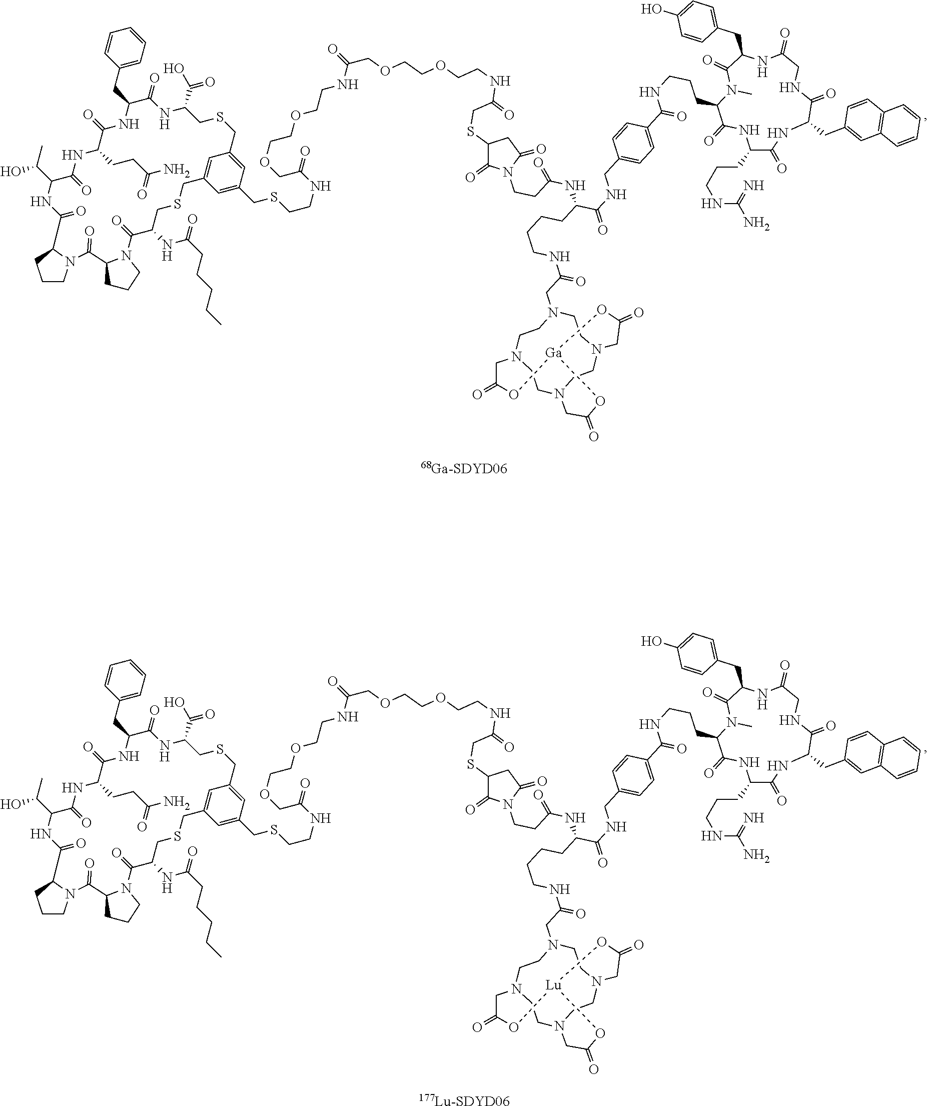

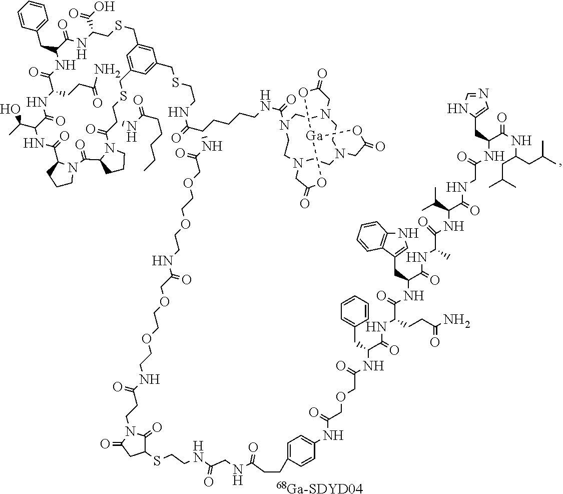

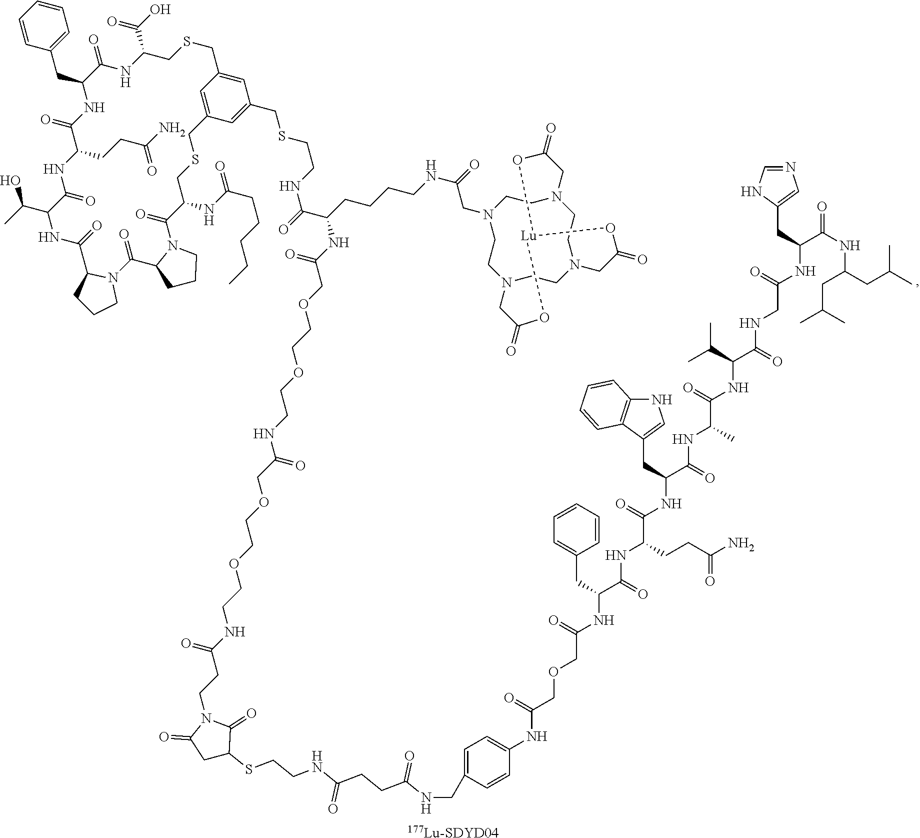

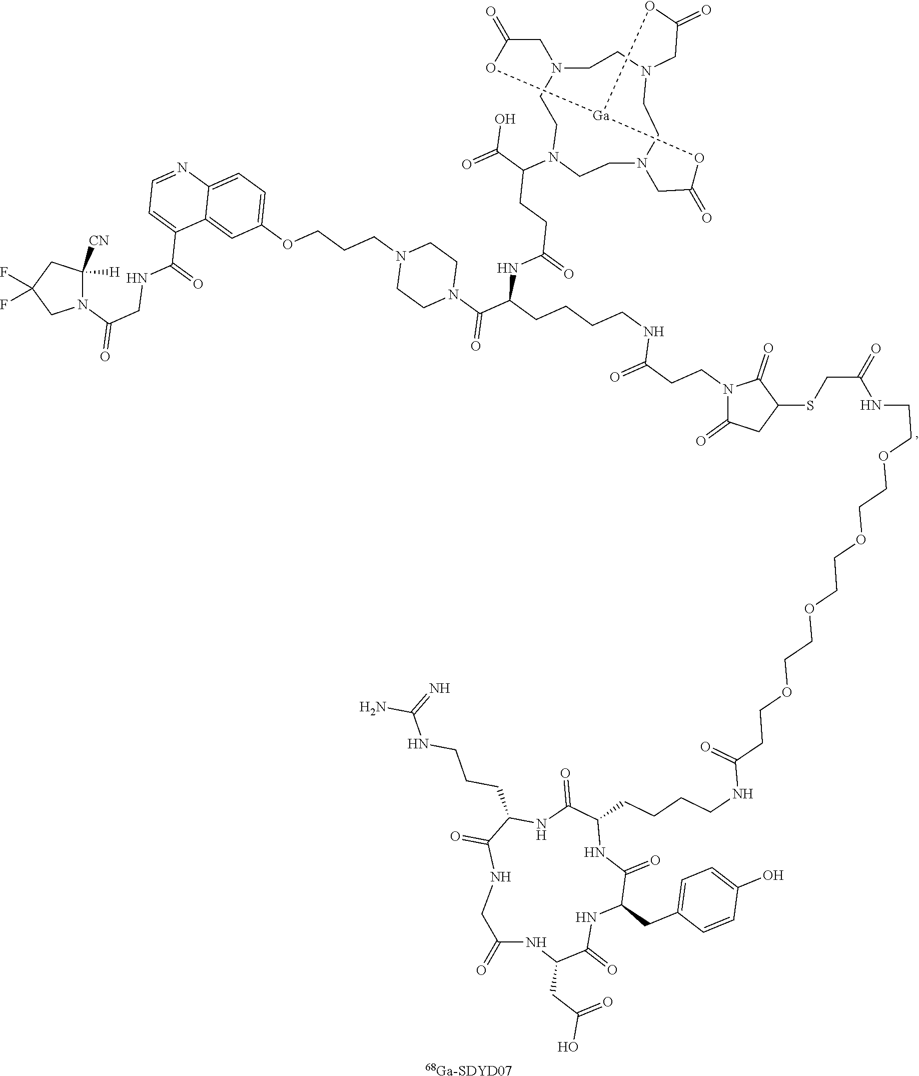

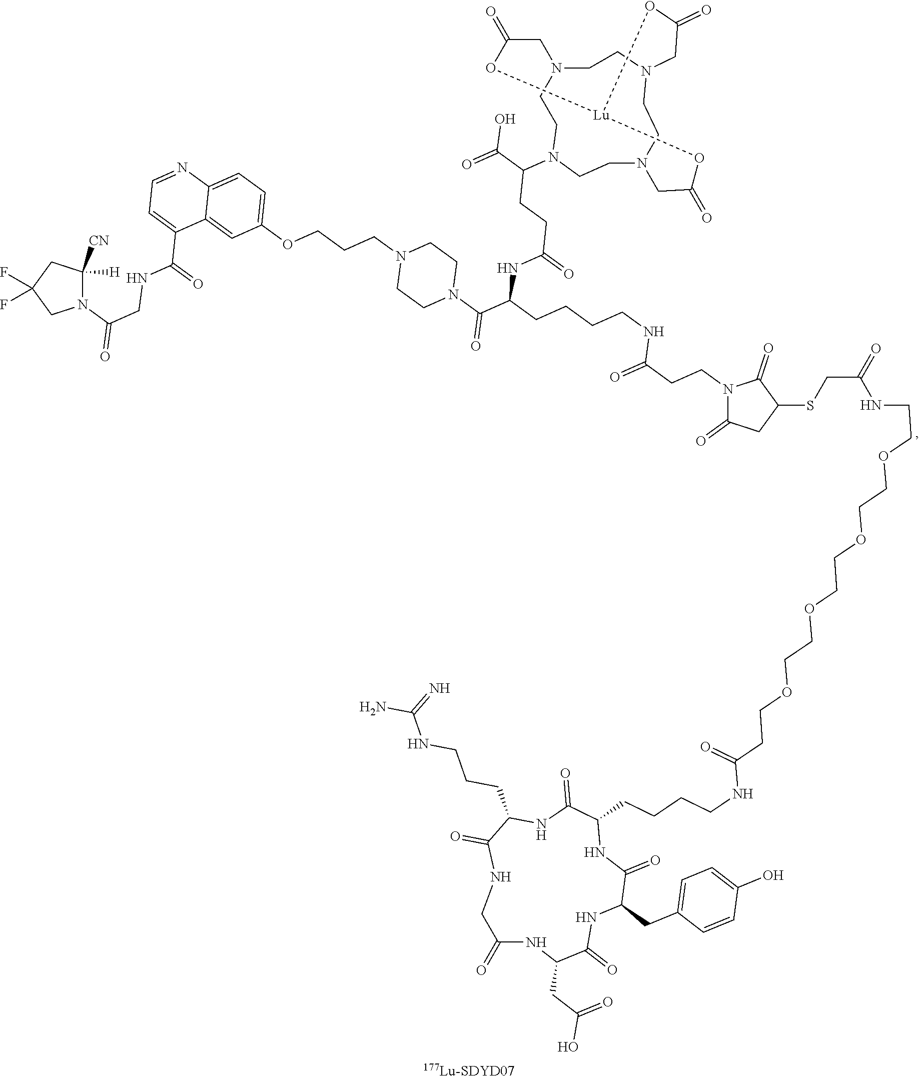

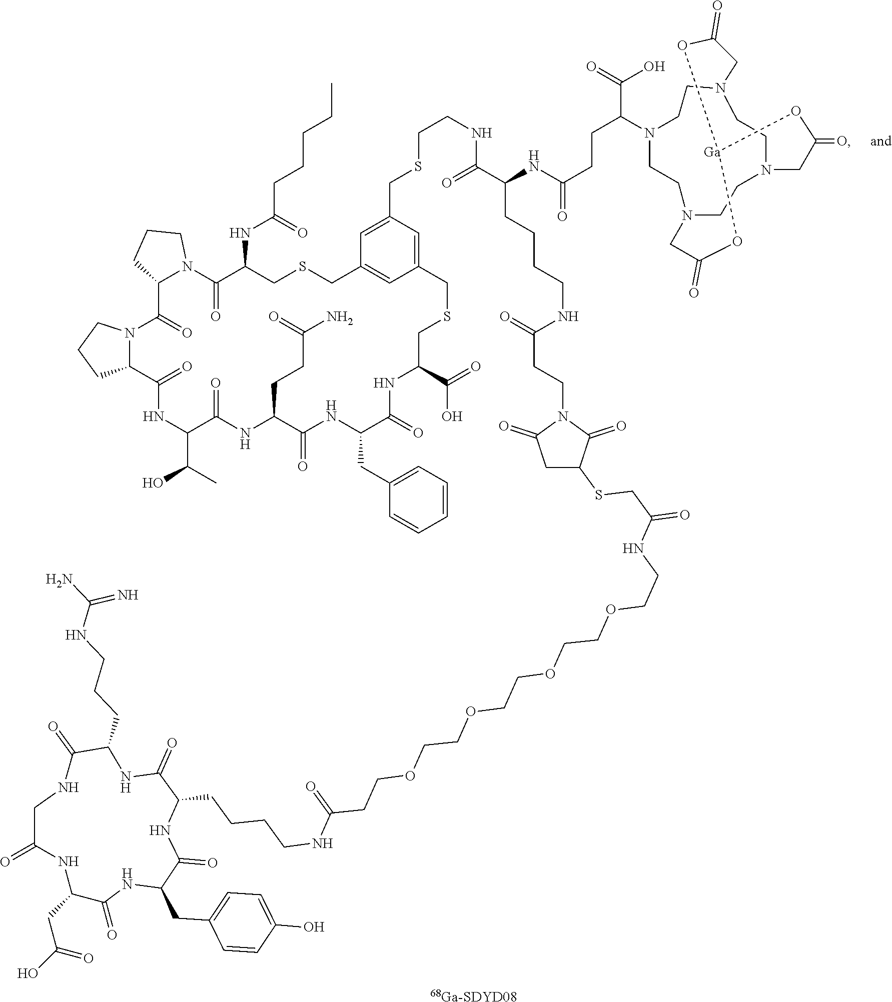

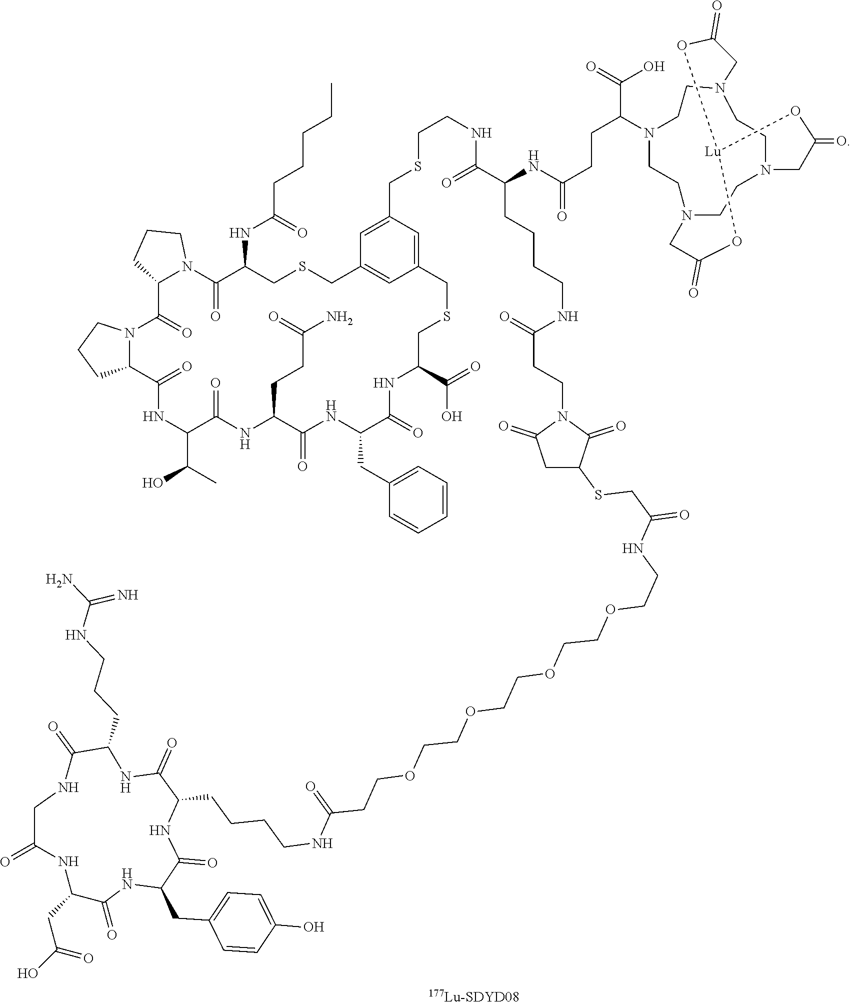

In another aspect, the present invention further provides a pharmaceutical composition, comprising or consisting of at least one compound of the present invention, and optionally, a pharmaceutically acceptable carrier and/or excipient.

In another aspect, the present invention further provides a use of the compound or pharmaceutical composition of the present invention in the diagnosis or treatment of a disease characterized by abnormal expression of one or two of FAP, CXCR4, GRPR, and αᵥβ₃ in an animal or human subject; or a method thereof.

In some embodiments, the disease is characterized by abnormal expression of one or both of FAP and CXCR4;

In some embodiments, the disease is characterized by abnormal expression of one or both of FAP and GRPR;

In some embodiments, the disease is characterized by abnormal expression of one or both of FAP and αᵥβ₃;

In some embodiments, the disease is selected from the group consisting of cancer, chronic inflammation, atherosclerosis, fibrosis, tissue remodeling and scarring disorder, preferably, the cancer is selected from the group consisting of breast cancer, pancreatic cancer, small bowel cancer, colon cancer, rectal cancer, lung cancer, head and neck cancer, ovarian cancer, hepatocellular carcinoma, esophageal cancer, hypopharyngeal cancer, nasopharyngeal cancer, laryngeal cancer, myeloma cells, bladder cancer, cholangiocarcinoma, clear cell renal cancer, neuroendocrine tumor, oncogenic osteomalacia, sarcoma. CUP (carcinoma of unknown primary), thymic carcinoma, glioma, neuroglioma, astrocytoma, cervical cancer, and prostate cancer.

In addition, the present invention further provides a kit comprising or consisting of the compound or pharmaceutical composition of the present invention.

The structure of compounds disclosed in the present invention are capable of synergistically targeting specific pairs of targeting of the FAP and CXCR4 targets, the FAP and GRPR targets, or the FAP and αᵥβ₃ targets in tumors, which increases number and utilization efficiency of effective receptors within tumors. The compounds of the present invention exhibit enhanced targeting specificity, higher tumor uptake efficiency, and improved tumor retention capability, while being cleared more rapidly from non-tumor tissues. The radiolabeled compound further provided based on such structure is expected to be applicable in diagnosing or treating diseases characterized by abnormal expression of FAP, CXCR4, GRPR, or αᵥβ₃.

### DESCRIPTION OF DRAWINGS

FIG. 1 illustrates the high performance liquid chromatogram of SDYD01.

FIG. 2 illustrates the mass spectrum of SDYD01.

FIG. 3 illustrates the radiochemical purity (RCP) analysis of radiolabeled SDYD01: (a) RCP of ⁶⁸Ga-SDYD01; (b) RCP of ¹⁷⁷Lu-SDYD01.

FIG. 4 illustrates the stability study of ⁶⁸Ga-SDYD01 in buffer: (a) 0.5 hours: (b) 1 hour; (c) 2 hours.

FIG. 5 illustrates the stability study of ¹⁷⁷Lu-SDYD01 in buffer: (a) 24 hours; (b) 48 hours; (c) 120 hours.

FIG. 6 illustrates the radioligand competitive assay of the ⁶⁸Ga-SDYD01 in histidine and cysteine solutions: (a) in 10 mM cysteine solution at 2 hours; (b) in 10 mM histidine solution at 2 hours.

FIG. 7 illustrates the high performance liquid chromatogram of SDYD02.

FIG. 8 illustrates the mass spectrum of SDYD02.

FIG. 9 illustrates the radiochemical purity (RCP) analysis of radiolabeled compound SDYD02: (a) RCP of 68Ga-SDYD02: (b) RCP of 177Lu-SDYD02.

FIG. 10 illustrates the stability study of ⁶⁸Ga-SDYD02 in buffer: (a) 0.5 hours; (b) 1 hour; (c) 2 hours.

FIG. 11 illustrates the stability study of ¹⁷⁷Lu-SDYD02 in buffer: (a) 24 hours; (b) 48 hours; (c) 120 hours.

FIG. 12 illustrates the radioligand competitive assay of the ⁶⁸Ga-SDYD02 in histidine and cysteine solutions: (a) in 10 mM cysteine solution at 2 hours: (b) in 10 mM histidine solution at 2 hours.

FIG. 13 illustrates the high performance liquid chromatogram of SDYD05.

FIG. 14 illustrates the mass spectrum of SDYD05.

FIG. 15 illustrates the radiochemical purity (RCP) analysis of radiolabeled SDYD05: (a) RCP of ⁶⁸Ga-SDYD05; (b) RCP of ¹⁷⁷Lu-SDYD05.

FIG. 16 illustrates the stability study of ⁶⁸Ga-SDYD05 in buffer: (a) 0.5 hours; (b) 1 hour; (c) 2 hours.

FIG. 17 illustrates the stability study of ¹⁷⁷Lu-SDYD05 in buffer: (a) 24 hours: (b) 48 hours; (c) 120 hours.

FIG. 18 illustrates the radioligand competitive assay of the ⁶⁸Ga-SDYD05 in histidine and cysteine solutions: (a) in 10 mM cysteine solution at 2 hours; (b) in 10 mM histidine solution at 2 hours.

FIG. 19 illustrates the high performance liquid chromatogram of SDYD06.

FIG. 20 illustrates the mass spectrum of SDYD06.

FIG. 21 illustrates the radiochemical purity (RCP) analysis of radiolabeled SDYD06: (a) RCP of ⁶⁸Ga-SDYD06: (b) RCP of ¹⁷⁷Lu-SDYD06.

FIG. 22 illustrates the stability study of ⁶⁸Ga-SDYD06 in buffer: (a) 0.5 hours: (b) 1 hour; (c) 2 hours.

FIG. 23 illustrates the radioligand competitive assay of the ⁶⁸Ga-SDYD06 in histidine and cysteine solutions: (a) in 10 mM cysteine solution at 2 hours; (b) in 10 mM histidine solution at 2 hours.

FIG. 24 illustrates the high performance liquid chromatogram of SDYD03.

FIG. 25 illustrates the mass spectrum of SDYD03.

FIG. 26 illustrates the radiochemical purity (RCP) analysis of radiolabeled compound SDYD03: (a) RCP of ⁶⁸Ga-SDYD03; (b) RCP of ¹⁷⁷Lu-SDYD03.

FIG. 27 illustrates the stability study of ⁶⁸Ga-SDYD03 in buffer: (a) 0.5 hours; (b) 1 hour; (c) 2 hours.

FIG. 28 illustrates the stability study of ¹⁷⁷Lu-SDYD03 in buffer: (a) 24 hours; (b) 48 hours; (c) 120 hours.

FIG. 29 illustrates the high performance liquid chromatogram of SDYD04.

FIG. 30 illustrates the mass spectrum of SDYD04.

FIG. 31 illustrates the radiochemical purity (RCP) analysis of radiolabeled compound SDYD04: (a) RCP of ⁶⁸Ga-SDYD04; (b) RCP of ¹⁷⁷Lu-SDYD04.

FIG. 32 illustrates the stability study of ⁶Ga-SDYD04 in buffer: (a) 0.5 hours; (b) 1 hour; (c) 2 hours.

FIG. 33 illustrates the stability study of ¹⁷⁷Lu-SDYD04 in buffer: (a) 24 hours: (b) 48 hours; (c) 120 hours.

FIG. 34 illustrates the high performance liquid chromatogram of SDYD07.

FIG. 35 illustrates the mass spectrum of SDYD07.

FIG. 36 illustrates the radiochemical purity (RCP) analysis of radiolabeled SDYD07: (a) RCP of ⁶⁸Ga-SDYD07; (b) RCP of ¹⁷⁷Lu-SDYD07.

FIG. 37 illustrates the stability study of ⁶⁸Ga-SDYD07 in buffer: (a) 0.5 hours; (b) 1 hour; (c) 2 hours.

FIG. 38 illustrates the stability study of ¹⁷⁷Lu-SDYD07 in buffer: (a) 24 hours; (b) 48 hours; (c) 120 hours.

FIG. 39 illustrates the high performance liquid chromatogram of SDYD08.

FIG. 40 illustrates the mass spectrum of SDYD08.

FIG. 41 illustrates the radiochemical purity (RCP) analysis of radiolabeled SDYD08: (a) RCP of ⁶⁸Ga-SDYD08; (b) RCP of ¹⁷⁷Lu-SDYD08.

FIG. 42 illustrates the stability study of ⁶⁸Ga-SDYD08 in buffer: (a) 0.5 hours; (b) 1 hour; (c) 2 hours.

FIG. 43 illustrates the stability study of ¹⁷⁷Lu-SDYD08 in buffer: (a) 24 hours; (b) 48 hours: (c) 120 hours.

FIG. 44 illustrates the cell uptake results of ⁶⁸Ga-SDYD01: (a) radioactive count of cell uptake; (b) inhibition rate at each time point.

FIG. 45 illustrates the cell uptake results of ⁶⁸Ga-SDYD03: (a) radioactive count of cell uptake: (b) inhibition rate at each time point.

FIG. 46 illustrates the cell uptake results of ⁶⁸Ga-SDYD04: (a) radioactive count of cell uptake: (b) inhibition rate at each time point.

FIG. 47 illustrates the cell uptake results of ⁶⁸Ga-SDYD05: (a) radioactive count of cell uptake; (b) inhibition rate at each time point.

FIG. 48 illustrates the cell uptake results of ⁶⁸Ga-SDYD06: (a) radioactive count of cell uptake; (b) inhibition rate at each time point.

FIG. 49 illustrates the cell uptake results of ⁶⁸Ga-SDYD07: (a) radioactive count of cell uptake: (b) inhibition rate at each time point.

FIG. 50 illustrates the cell uptake results of ⁶⁸Ga-SDYD08: (a) radioactive count of cell uptake; (b) inhibition rate at each time point.

FIG. 51 illustrates the animal experimental results of ⁶⁸Ga-SDYD01: (a) PET/CT imaging of tumor-bearing mice; (b) biodistribution data in tumor-bearing mice.

FIG. 52 illustrates the animal experimental results of ⁶⁸Ga-SDYD02: (a) PET/CT imaging of tumor-bearing mice; (b) biodistribution data in tumor-bearing mice.

FIG. 53 illustrates the animal experimental results of ⁶⁸Ga-SDYD03: (a) PET/CT imaging of tumor-bearing mice; (b) biodistribution data in tumor-bearing mice.

FIG. 54 illustrates the animal experimental results of ⁶⁸Ga-SDYD04: (a) PET/CT imaging of tumor-bearing mice; (b) biodistribution data in tumor-bearing mice.

FIG. 55 illustrates the animal experimental results of ⁶⁸Ga-SDYD05: (a) PET/CT imaging of tumor-bearing mice; (b) biodistribution data in tumor-bearing mice.

FIG. 56 illustrates the animal experimental results of ⁶⁸Ga-SDYD06: (a) PET/CT imaging of tumor-bearing mice; (b) biodistribution data in tumor-bearing mice.

FIG. 57 illustrates the animal experimental results of ⁶⁸Ga-SDYD07: (a) PET/CT imaging of tumor-bearing mice: (b) biodistribution data in tumor-bearing mice; (c) relative ratio of tumor-to-normal tissue radioactive uptake in tumor-bearing mice.

FIG. 58 illustrates the animal experimental results of ⁶⁸Ga-SDYD08: (a) PET/CT imaging of tumor-bearing mice; (b) biodistribution data in tumor-bearing mice; (c) relative ratio of tumor-to-normal tissue radioactive uptake in tumor-bearing mice.

FIG. 59 illustrates the animal experimental results of ⁶⁸Ga-FAPI04: (a) PET/CT imaging of tumor-bearing mice; (b) biodistribution data in tumor-bearing mice.

FIG. 60 illustrates the animal experimental results of ⁶⁸Ga-FAP2286: (a) PET/CT imaging of tumor-bearing mice; (b) biodistribution data in tumor-bearing mice.

FIG. 61 illustrates the biodistribution data of ⁶⁸Ga-SDYD07-1 in tumor-bearing mice.

### DETAILED DESCRIPTION

In order to make the objectives, technical solutions, and advantages of the present invention clearer and more explicit, the invention is described in further detail below in conjunction with specific Examples. The specific Examples described herein are merely for explaining the present invention and are not intended to limit the invention in any way. Furthermore, descriptions of well-known structures and techniques are omitted in the following description to avoid unnecessarily obscuring the concepts of the present disclosure. Such structures and techniques have been described in many publications.

### Definitions

Unless otherwise specified, all technical and scientific terms used herein have the same meaning as commonly understood by those skilled in the art to which this invention belongs. For the purpose of interpreting this specification, the following definitions will apply, and where appropriate, terms used in the singular will also include the plural, and vice versa.

Unless the context clearly indicates otherwise, the expressions “a” and “an” as used herein include plural referents. For example, reference to “a cell” includes a plurality of such cells and equivalents known to those skilled in the art, and the like.

The term “pharmaceutically acceptable salt” refers to a salt of a compound of the present invention. Suitable pharmaceutically acceptable salts of the compounds of the present invention include acid addition salts, which may be formed, for example, by mixing a solution of choline or its derivatives with a solution of a pharmaceutically acceptable acid such as hydrochloric acid, sulfuric acid, fumaric acid, maleic acid, succinic acid, acetic acid, benzoic acid, citric acid, tartaric acid, carbonic acid, or phosphoric acid. Furthermore, when a compound of the present invention carries an acidic moiety, its suitable pharmaceutically acceptable salts may include alkali metal salts (e.g., sodium or potassium salts); alkaline earth metal salts (e.g., calcium or magnesium salts); and salts formed with suitable organic ligands (e.g., formed with ammonium, quaternary ammonium, and amine cations by using counter-anions such as halide, hydroxide, carboxylate, sulfate, phosphate, nitrate, alkylsulfonate, and arylsulfonate). Illustrative examples of pharmaceutically acceptable salts include, but are not limited to: acetate, adipate, alginate, ascorbate, aspartate, besylate, benzoate, bicarbonate, bisulfate, bitartrate, borate, bromide, butyrate, edetate calcium, camphorate, camphorsulfonate, camsylate, carbonate, chloride, citrate, clavulanate, cyclopentanepropionate, digluconate, dihydrochloride, dodecyl sulfate, edetate, edisylate, estolate, esylate, ethanesulfonate, formate, fumarate, gluconate, gluceptate, glucoheptonate, glutamate, glycerophosphate, glycollylarsanilate, hemisulfate, heptanoate, hexanoate, hexylresorcinate, hydrabamine, hydrobromide, hydrochloride, hydroiodide, 2-hydroxyethanesulfonate, hydroxynaphthoate, iodide, isothionate, lactate, lactobionate, laurate, lauryl sulfate, malate, maleate, malonate, mandelate, mesylate, methanesulfonate, methyl sulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al., “Pharmaceutical Salts,” Journal of Pharmaceutical Science, 1977, 66, pp 1-19). Certain specific compounds of the invention contain both basic and acidic functional groups, enabling the compound to be converted into either a base addition salt or an acid addition salt.

The neutral form of the compound may be regenerated by contacting its salt with a base or an acid and then isolating the parent compound in a conventional manner. The parent form of the compound may differ from the various salt forms in certain physical properties such as solubility in polar solvents, but for the purposes of the present invention, these salts are equivalent to the parent form of the compound.

In addition to salt forms, the present invention provides compounds in prodrug forms. Prodrugs of the compounds described herein are those compounds that readily undergo chemical changes under physiological conditions to provide the compound of Formula (I). A prodrug is an active or inactive compound that can be chemically modified into the compound of the present invention through in vivo physiological action, such as hydrolysis, metabolism, or the like, after the prodrug is administered to a patient. Furthermore, prodrugs can be converted to the compounds of the invention in an ex vivo environment through chemical or biochemical methods. For instance, when placed in a transdermal patch reservoir with a suitable enzyme, a prodrug may be converted slowly into the compound of the present invention. The applicability and techniques involved in the preparation and use of prodrugs are well known to those skilled in the art. For A general discussion on prodrugs involving esters, refer to Svensson and Tunek, Drug Metabolism Reviews 16.5 (1988); and Bundgaard, Design of Prodrugs, Elsevier (1985). Examples of masked carboxylate anions include various esters, such as those formed with alkyl (e.g., methyl, ethyl), cycloalkyl (e.g., cyclohexyl), aralkyl (e.g., benzyl, p-methoxybenzyl), and alkylcarbonyloxyalkyl (e.g., pivaloyloxymethyl). Amines may be masked to form aryl carbonyloxymethyl-substituted derivatives, which are cleaved by esterases in vivo, releasing the free drug and formaldehyde (Bundgaard, J. Med. Chem., 25, 1989). Similarly, drugs containing acidic NH groups, such as imidazole, imide, indole, etc., may be masked by N-acyloxymethyl group (Bundgaard, Design of Prodrugs, Elsevier, 1985). Hydroxy groups may be masked as esters and ethers. EP 0 039 051 (Sloan and Little, Apr. 11, 1981) discloses Mannich-based hydroxamic acid prodrugs, their preparation, and use.

The term “alkyl” refers to a saturated, straight-chain or branched carbon chain. Preferably, the chain contains from 1 to 10 carbon atoms, i.e., 1, 2, 3, 4, 5, 6, 7, 8, 9, or 10 carbon atoms, such as methyl, ethyl, propyl, isopropyl, butyl, isobutyl, tert-butyl, pentyl, hexyl, heptyl, or octyl. The alkyl group is optionally substituted.

The term “cycloalkyl” refers to a cyclized alkyl group, including monocyclic, bicyclic, or polycyclic ring systems, which contain no unsaturated bonds such as double bonds and contain no heteroatoms, for example, C₅₋₇ cycloalkyl, C₃₋₇ cycloalkyl, or C₃₋₆ cycloalkyl, C₅₋₇ cycloalkyl includes C₅, C₆, and C₇ cycloalkyl. Examples of cycloalkyl include, but are not limited to, cyclopropyl, cyclobutyl, cyclopentyl, cyclohexyl, cycloheptyl, and the like.

The term “heteroalkylene” refers to a group obtained by replacing one or more carbon atoms in a divalent, saturated, straight-chain or branched alkyl group by a heteroatom. Preferably, the heteroatom is selected from the group consisting of N. O, and S. Preferably, the group contains from 1 to 9 carbon atoms, i.e., 1, 2, 3, 4, 5, 6, 7, 8, or 9 carbon atoms. Examples include —O—CH₂—, —S—CH₂—, —CH₂—O—CH₂—, —CH₂—O—C₂H₄—, —CH₂—S—CH₂—, —CH₂—S—C₂H₄—, —C₂H₄—O—CH₂—, —C₂H₄—O—C₂H₄—, —C₂H₄—S—CH₂—, —C₂H₄—S—C₂H₄—, and the like. The heteroalkylene group is optionally substituted.

The terms “alkenyl” and “cycloalkenyl” refer to a linear or cyclic group containing olefinically unsaturated carbon atoms having one or more double bonds. Examples are propenyl and cyclohexenyl. Preferably, an alkenyl chain contains from 2 to 8 carbon atoms, i.e., 2, 3, 4, 5, 6, 7, or 8 carbon atoms, such as vinyl, 1-propenyl, 2-propenyl, isopropenyl, 1-butenyl, 2-butenyl, 3-butenyl, isobutenyl, sec-butenyl, 1-pentenyl, 2-pentenyl, 3-pentenyl, 4-pentenyl, hexenyl, heptenyl, or octenyl. Preferably, a cycloalkenyl ring contains from 3 to 8 carbon atoms. i.e., 3, 4, 5, 6, 7, or 8 carbon atoms, such as 1-cyclopropenyl, 2-cyclopropenyl, 1-cyclobutenyl, 2-cyclobutenyl, 1-cyclopentenyl, 2-cyclopentenyl, 3-cyclopentenyl, cyclohexenyl, cycloheptenyl, or cyclooctenyl. The terms “heteroalkenyl” or “heterocycloalkenyl” refer to an “alkenyl” or “cycloalkenyl” into which one or more heteroatoms are inserted; preferably, the heteroatom is selected from the group consisting of N, O, and S.

The term “alkynyl” refers to a linear or cyclic group containing unsaturated carbon atoms having one or more triple bonds. An example is propargyl. Preferably, an alkynyl chain contains from 2 to 8 carbon atoms. i.e., 2, 3, 4, 5, 6, 7, or 8 carbon atoms, such as ethynyl, 1-propynyl, 2-propynyl, 1-butynyl, 2-butynyl, 3-butynyl, 1-pentynyl, 2-pentynyl, 3-pentynyl, 4-pentynyl, hexynyl, heptynyl, or octynyl.

The term “aryl” preferably refers to an aromatic monocyclic or polycyclic system containing 5-16 carbon atoms, wherein the polycyclic system may be an aromatic group comprising linked, fused, or spiro rings. Examples are phenyl, biphenyl, naphthyl, or anthryl. The aryl group is optionally substituted.

The term “aralkyl” refers to an alkyl moiety substituted by an aryl group, wherein alkyl and aryl have the meanings given above. An example is benzyl. Preferably, in the context of this text, an alkyl chain contains from 1 to 8 carbon atoms, i.e., 1, 2, 3, 4, 5, 6, 7, or 8 carbon atoms, such as methyl, ethyl, propyl, isopropyl, butyl, isobutyl, sec-butyl, tert-butyl, pentyl, hexyl, heptyl, or octyl. The alkyl and/or aryl portions of the aralkyl group are optionally substituted.

The term “heteroaryl” refers to a monocyclic or polycyclic aromatic compound in which one or more ring-forming carbon atoms are replaced by heteroatoms. For example, a heteroaryl may be a five-membered or six-membered aromatic monocycle in which at least one ring-forming carbon atom is replaced by 1, 2, 3, or 4 (for a five-membered ring) or 1, 2, 3, 4, or 5 (for a six-membered ring) identical or different heteroatoms, with the heteroatoms being preferably selected from the group consisting of O, N, and S; or an aromatic bicyclic system in which 1, 2, 3, 4, 5, or 6 of the 8, 9, 10, 11, or 12 ring-forming carbon atoms are replaced by identical or different heteroatoms, with the heteroatoms being preferably selected from the group consisting of O, N. and S; or an aromatic tricyclic system in which 1, 2, 3, 4, 5, or 6 of the 13, 14, 15, or 16 ring-forming carbon atoms are replaced by identical or different heteroatoms, with the heteroatoms being preferably selected from the group consisting of O, N. and S. A heteroaryl may also be a polycyclic system having more than three rings. The bicyclic, tricyclic, or polycyclic system can be a linked, fused, or spiro aromatic group.

The term “heteroaralkyl” refers to an alkyl moiety substituted by a heteroaryl group, wherein the alkyl and heteroaryl have the meanings as described above. Examples are 2-alkylpyridyl, 3-alkylpyridyl, or 2-methylpyridyl. Preferably, in the context of this text, an alkyl chain contains from 1 to 8 carbon atoms, i.e., 1, 2, 3, 4, 5, 6, 7, or 8 carbon atoms, such as methyl, ethyl, propyl, isopropyl, butyl, isobutyl, sec-butyl, tert-butyl, pentyl, hexyl, heptyl, or octyl. The alkyl and/or heteroaryl portions of the heteroaralkyl group are optionally substituted.

The term “N-containing aromatic or non-aromatic, monocyclic or bicyclic heterocycle” refers to a cyclic saturated or unsaturated hydrocarbon compound containing at least one nitrogen atom as a ring-forming constituent unit.

The term “halogen” refers to a halogen residue selected from the group consisting of F, Br, I, and Cl. Preferably, the halogen is F.

The term “hydroxy” refers to —OH.

The expression “optionally substituted” means that one, two, three, or more than three hydrogen atoms in the group may each be replaced, independently, by a substituent.

In this application, regarding —U¹-G¹-A¹-L-A²-G²-U²—, when one or several of U¹, G¹, A¹, L, A², G², U² is/are bond(s), it means that the one or several unit(s) is/are absent. For clarity, examples are provided herein. For instance, when U¹ is a bond, —U¹-G¹-A¹-L-A²-G²-U²— has the same meaning as (i.e., equals to)-G¹-A¹-L-A²-G²-U²—; when L is a bond, —U¹-G¹-A¹-L-A²-G²-U²— has the same meaning as (i.e., equals to)-UL-G¹-A¹-A²-G²-U²; when both U¹ and G¹ are bonds, —U¹-G¹-A¹-L-A²-G²-U²— has the same meaning as (i.e., equals to)-A¹-L-A²-G²-U²—; when both U¹ and G² are bonds, —U¹-G¹-A¹-L-A²-G²-U²— has the same meaning as (i.e., equals to)-G¹-A¹-L-A²-U²—; when U¹. G¹, and A¹ are all bonds, —U¹-G¹-A¹-L-A²-G²-U²— is the same as (i.e., equals to) -L-A²-G²-U²—; when U¹. G¹, A¹, L, A², G² are all bonds, —U¹-G¹-A¹-L-A²-G²-U²— has the same meaning as (i.e., equals to) —U²—.

As used herein, the term “amino acid” refers to any organic acid containing one or more amino substituents, such as α-, β-, or γ-amino derivatives of an aliphatic carboxylic acid. In the polypeptide notation used herein, such as Xaa1-Xaa2-Xaa3-Xaa4-Xaa5, wherein Xaa1 to Xaa5 each independently represent an amino acid as defined, and according to standard usage and convention, the left-hand direction is the amino-terminal direction and the right-hand direction is the carboxy-terminal direction.

The term “common amino acid” refers to the twenty naturally occurring amino acids, including all their stereoisomers, i.e., D,L-, D- and L-amino acids. These common amino acids may also be referred to herein by their conventional three-letter or one-letter abbreviations, and their abbreviations follow the conventions in the art (see, e.g., Immunology-A Synthesis, 2^{nd} Edition, by E. S. Golub and D. R. Gren, Sinauer Associates, Sunderland Mass (1991)).

The term “non-common amino acid” refers to non-naturally occurring amino acids or chemical amino acid analogs, such as α,α-disubstituted amino acids, N-alkyl amino acids, homo-amino acids, dehydroamino acids, aromatic amino acids (other than phenylalanine, tyrosine, and tryptophan) and ortho-aminobenzoic acid, meta-aminobenzoic acid, or para-aminobenzoic acid. Non-common amino acids also include compounds having amine and carboxyl functional groups separated in a 1,3-, or even greater, substitution pattern, such as β-alanine. γ-aminobutyric acid, Freidinger lactams, bicyclic dipeptides (BTD), amino-methylbenzoic acids, and others known in the art. Isosteres of the statine type, hydroxyethylene isosteres, reduced amide bond isosteres, thioamide isosteres, urea isosteres, carbamate isosteres, thioether isosteres, vinyl isosteres, and other amide bond isosteres known in the art may also be used. The use of analogs or non-common amino acids can improve the stability and biological half-life of the incorporated peptide, as they are more resistant to breakdown under physiological conditions. Those skilled in the art will recognize that substitutions of similar types can be made. A non-limiting list of non-common amino acids suitable for use as structural units in peptides and their standard abbreviations (in parentheses) is as follows: α-aminobutyric acid (Abu), L-N-methylalanine (Nmala), α-amino-α-methylbutyrate (Mgabu), L-N-methylarginine (Nmarg), aminocyclopropane (Cpro), L-N-methylasparagine (Nmasn), L-N-methylaspartate (Nmasp), anilineisobutyric acid (Aib), L-N-methylcysteine (Nmcys), aminonorbornyl (Norb), L-N-methylglutamine (Nmgln), L-N-methylglutamate (Nmglu), cyclohexylalanine (Chexa), L-N-methylhistidine (Nmhis), cyclopentylalanine (Cpen), L-N-methylisoleucine (Nmile), L-N-methylleucine (Nmleu), L-N-methyllysine (Nmlys), L-N-methylmethionine (Nmmet), L-N-methylnorleucine (Nmnle), L-N-methylnorvaline (Nmnva), L-N-methylornithine (Nmorn), L-N-methylphenylalanine (Nmphe), L-N-methylproline (Nmpro), L-N-methylserine (Nmser), L-N-methylthreonine (Nmthr), L-N-methyltryptophan (Nmtrp), D-ornithine (Dorn), L-N-methyltyrosine (Nmtyr), L-N-methylvaline (Nmval), L-N-methylethylglycine (Nmetg), L-N-methyl-tert-butylglycine (Nmtbug), L-norleucine (Nle), L-norvaline (Nva), α-methylaminoisobutyrate (Maib), α-methyl-γ-aminobutyrate (Mgabu), D-α-methylalanine (Dmala), α-methylcyclohexylalanine (Mchexa), D-α-methylarginine (Dmarg), α-methylcyclopentylalanine (Mcpen), D-α-methylasparagine (Dmasn), α-methyl-α-naphthylalanine (Manap), D-α-methylaspartate (Dmasp), α-methylpenicillamine (Mpen), D-α-methylcysteine (Dmcys), N-(4-aminobutyl)glycine (Nglu), D-α-methylglutamine (Dmgln), N-(2-aminoethyl)glycine (Naeg), D-α-methylhistidine (Dmhis), N-(3-aminopropyl)glycine (Norn), D-α-methylisoleucine (Dmile), N-amino-α-methylbutyrate (Nmaabu), D-α-methylleucine (Dmleu), α-naphthylalanine (Anap), D-α-methyllysine (Dmlys), N-benzylglycine (Nphe), D-α-methylmethionine (Dmmet), N-(2-carbamoylethyl)glycine (Ngln), D-α-methylornithine (Dmorn), N-(carbamoylmethyl)glycine (Nasn), D-α-methylphenylalanine (Dmphe), N-(2-carboxyethyl)glycine (Nglu), D-α-methylproline (Dmpro), N-(carboxymethyl)glycine (Nasp), D-α-methylserine (Dmser), N-cyclobutylglycine (Ncbut), D-α-methylthreonine (Dmnthr), N-cycloheptylglycine (Nchep), D-α-methyltryptophan (Dmtrp), N-cyclohexylglycine (Nchex), D-α-methyltyrosine (Dmty), N-cyclodecylglycine (Nedec), D-α-methylvaline (Dmval), N-cyclododecylglycine (Ncdod), D-N-methylalanine (Dnmala), N-cyclooctylglycine (Ncoct), D-N-methylarginine (Dnmarg), N-cyclopropylglycine (Nepro), D-N-methylasparagine (Dnmasn), N-cycloundecylglycine (Ncund), D-N-methylaspartate (Dnmasp), N-(2,2-diphenylethyl)glycine (Nbhm), D-N-methylcysteine (Dnmcys), N-(3,3-diphenylpropyl)glycine (Nbhe), D-N-methylglutamine (Dnmgln), N-(3-guanidinopropyl)glycine (Narg), D-N-methylglutamate (Dnmglu), N-(1-hydroxyethyl)glycine (Ntbx), D-N-methylhistidine (Dnmhis), N-(hydroxyethyl)glycine (Nser), D-N-methylisoleucine (Dnmile), N-(imidazolylethyl)glycine (Nhis), D-N-methylleucine (Dnmleu), N-(3-indolylethyl)glycine (Nhtrp), D-N-methyllysine (Dnnilys), N-methyl-γ-aminobutyrate (Nmgabu), N-methylcyclohexylalanine (Nmchexa), D-N-methylmethionine (Dnmmet), D-N-methylornithine (Dnmorn). N-methylcyclopentylalanine (Nmcpen), N-methylglycine (Nala), D-N-methylphenylalanine (Dnmphe), N-methylaminoisobutyrate (Nmaib), D-N-methylproline (Dnmpro), N-(1-methylpropyl)glycine (Nile), D-N-methylserine (Dnmser), N-(2-methylpropyl)glycine (Nleu), D-N-methylthreonine (Dnmthr), D-N-methyltryptophan (Dnmtrp), N-(1-methylethyl)glycine (Nval), D-N-methyltyrosine (Dnmtyr), N-methyl-α-naphthylalanine (Nmanap), D-N-methylvaline (Dnmval), N-methylpenicillamine (Nmpen), γ-aminobutyric acid (Gabu), N-(p-hydroxyphenyl)glycine (Nhtyr), L-tert-butylglycine (Tbug), N-(thiomethyl)glycine (Ncys), L-ethylglycine (Etg), Penicillamine (Pen), L-homophenylalanine (Hphe), L-α-methylalanine (Mala), L-α-methylarginine (Marg), L-α-methylasparagine (Masn), L-α-methylaspartic acid (Masp), L-α-methyltert-butylglycine (Mtbug), L-α-methylcysteine (Mcys), L-methylethylglycine (Metg), L-α-methylglutamine (MgIn), L-α-methylglutamic acid (Mglu), L-α-methylhistidine (Mhis), L-α-methylhomophenylalanine (Mhphe), L-α-methylisoleucine (Mile), N-(2-methylthioethyl)glycine (Nmet), L-α-methylleucine (Mleu), L-α-methyllysine (Mlys), L-α-methylmethionine (Mmet), L-α-methylnorleucine (Mnle), L-α-methylnorvaline (Mnva), L-α-methylornithine (Morn), L-α-methylphenylalanine (Mphe), L-α-methylproline (Mpro), L-α-methylserine (Mser), L-α-methylthreonine (Mthr), L-α-methyltryptophan (Mtrp), L-α-methyltyrosine (Mtyr), L-α-methylvaline (Mval), L-N-methylhomophenylalanine (Nmhphe), N—(N-(2,2-diphenylethyl) carbamoylmethyl)glycine (Nnbhm), N—(N-(3,3-(Nnbhe), 1-carboxy-1-(2,2-diphenylpropyl) carbamoylmethyl)glycine diphenylethylamino)cyclopropane (Nmbc), L-O-methylserine (Omser), L-O-methylhomoserine (Omhser).

The term “radioactive moiety” refers to a molecular assembly carrying a radionuclide. The nuclide is bound via covalent or coordination bonds that are stable under physiological conditions. Examples are [¹³¹I]-3-iodobenzoic acid or ⁶⁸Ga-DOTA.

A “fluorescent isotope” emits electromagnetic radiation after being excited by electromagnetic radiation of a shorter wavelength.

A “radioisotope” is a radioactive isotope of an element (included in the term “radionuclide”) that emits α-, β-, and/or γ-radiations.

The term “radiopharmaceutical” as used in the context of the present invention refers to a biologically active compound modified with a radioisotope. In particular, intercalating substances can be used to deliver radioactivity in direct proximity to DNA (e.g., a ¹³¹I-carrying derivative of Hoechst-33258).

A chelating group is part of the compound of the invention, wherein the chelating group is connected to the compound of the invention directly or indirectly (e.g., via a linker). Preferred chelating groups are chelating agents capable of forming metal chelates, preferably, the metal is at least one radiometal. The at least one radiometal is preferably usable or applicable for diagnostic and/or therapeutic and/or theranostic use, more preferably usable or applicable for imaging and/or radiotherapy.

The term “chelating group” is derived from a chelating agent compound. The terms “chelating agent” and “chelator” are used interchangeably in the context of the present invention and refer to a molecule, typically an organic molecule, usually a Lewis base, having two or more unshared electron pairs available for a metal ion. The metal ion is typically coordinated with the chelator via two or more electron pairs. The terms “bidentate chelator”, “tridentate chelator” and “tetradentate chelator” refer to chelators having two, three, and four electron pairs, respectively, which are readily provided simultaneously to the metal ion coordinated by the chelator. Typically, the electron pairs of the chelator form coordinate bonds with a single metal ion. However, in some instances, the chelator can form coordinate bonds with more than one metal ion, and various binding modes are possible.
Chelators that can in principle be used and/or are suitable for practice of the present invention (including diagnosis and/or treatment of diseases) are known to those skilled in the art. A wide variety of corresponding chelators is available and has been reviewed, for example, by Banerjee et al. (Banerjee, et al., Dalton Trans, 2005, 24:3886) and references therein (Price, et al., Chem Soc Rev, 2014, 43:260; Wadas, et al., Chem Rev, 2010, 110:2858). Such chelators include, but are not limited to, linear, cyclic, macrocyclic, tetrapyridinyl, N3S, N2S2, and N4 chelators, as described in U.S. Pat. Nos. 5,367,080 A, 5,364,613 A, 5,021,556 A, 5,075,099 A, and 5,886,142 A.

Representative chelators and their derivatives include, but are not limited to: AAZTA, BAT, CDTA, DTA, DTPA, CY-DTA, DTCBP, CTA, cyclam, cyclen, TETA, Sarcophagine, CPTA, TEAMA, Cyclen, DO3A, DO2A, TRITA, DATA, DFO, DATA (M), DATA (P), DATA (Ph), DATA (PPh), DEDPA, H4octapa, H2dedpa, H5decapa, H2azapa, H2CHXDEDPA, DFO-Chx-MAL, DFO-p-SCN, DFO-1AC, DFO-BAC, p-SCN-Bn-DFO, DFO-pPhe-NCS, DFO-HOPO, DFC, Diphosphine, DOTA, DOTAGA, DOTA-MFCO, DOTAM-monoacid, nitro-DOTA, nitro-PA-DOTA, p-NCS-Bz-DOTA, PA-DOTA, DOTA-NCS, DOTA-NHS, CB-DO2A, PCTA, p-NH2-Bn-PCTA, p-SCN-Bn-PCTA, p-SCN-Bn-DOTA, DOTMA, NB-DOTA, H4NB-DOTA, H4TCE-DOTA, 3,4,3-(Li-1,2-HOPO), TREN (Me-3,2-HOPO), TCE-DOTA, DOTP, DOXP, p-NCS-DOTA, p-NCS-TRITA, TRITA, TETA, 3p-C-DEPA, 3p-C-DEPA-NCS, p-NH2-BN—OXO-DO3A, p-SCN—BN-TCMC, TCMC, 4-aminobutyl-DOTA, azido-monoamide-DOTA, BCN-DOTA, butynyl-DOTA, BCN-DOTA-GA, DOA³P, DO2a2p, DO2A (trans-H2do2a), DO3A, DO3A-thiol, DO3AtBu-N-(2-aminoethyl) acetamide, DO2AP, CB-DO2A, C3B-DO2A, HP-DO3A, DOTA-NHS-ester, maleimido-DOTA-GA, maleimido-mono-amide-DOTA, maleimido-DOTA, NH2-DOTA-GA, NH2-PEG4-DOTA-GA, GA, p-NH2-Bn-DOTA, p-NO2-Bn-DOTA, p-SCN-Bn-DOTA, p-SCN-Bz-DOTA, TA-DOTA, TA-DOTA-GA, OTTA, DOXP, TSC, DTC, DTCBP, PTSM, ATSM, H2ATSM, H2PTSM, Dp44mT, DpC, Bp44mT, QT, hybrid thiosemicarbazone-benzothiazole), tetradentate thiosemicarbazone-styrylpyridinyl ligand H2L2-4, HBED, HBED-CC, dmHBED, dmEHPG, HBED-nn, SHBED, Br-Me2HBED, BPCA, HEHA, BF-HEHA, deferiprone, THP, HYNIC (2-hydrazinonicotinamide), NHS—HYNIC, HYNIC-Kp-DPPB, HYNIC-Ko-DPPB, (HYNIC) (tricine)2, (HYNIC) (EDDA)Cl, p-EDDHA, AIM, AIM A, IAM B, MAMA, MAMA-DGal, MAMA-MGal, MAMA-DA, MAMA-HAD, Macropa, Macropaquin, Macroquin-SO3, NₓS₄₋ₓ, N2S2, N3S, N4, MAG3B, NOTA, NODAGA, SCN-Bz-NOTA-R, NOT-P (NOTMP), NOTAM, p-NCS-NOTA, TACN, TACN-TM, NETA, NETA-monoamine, p-SCN-PhPr-NE3TA, C-NE3TA-NCS, C-NETA-NCS, 3p-C-NETA, NODASA, NOPO, NODA, NO2A, N-Benzyl-NODA, C-NOTA, BCNOT-monoamine, maleimido-mono-amide-NOTA, NO2A-azide, NO2A-butyne, NO2AP, NO3AP, N-NOTA, oxo-DO3A, p-NH2-Bn-NOTA, p-NH2-Bn-oxo-DO3A, p-NO2-Bn-Cyclen, p-SCN-Bn-NOTA, p-SCN-Bn-oxo-DO3A, TRAP, PEPA, BF-PEPA, Pycup, Pycup2A, pycup1AlBn, pycup2Bn, SarAr-R, Diamsar, AmBaSar-R, siamSar, Sar, Tachpyr, tachpyr-(6-Me), TAM A, TAM B, TAME, TAME-Hex, THP-Ph-NCS, THP-NCS, THP-TATE, NTP, H3THP, THPN, CB-TE2A, PCB-TE1A1P, TETA-NHS, CPTA, CPTA-NHS, CB-TE1KIP, CB-TE2A, TE2A, H2CB-TE2A, TE2P, CB-TE2P, MM-TE2A, DM-TE2A, 2C-TETA, 6C-TETA, BAT, BAT-6, NHS-BAT ester, SSBAT, SCN—CHX-A-DTPA-P, SCN-TETA, TMT-amine, p-BZ-HTCPP.

HYNIC, DTPA, EDTA, DOTA, TETA, bis(aminoethanethiol)-based chelators are disclosed in U.S. Pat. No. 5,720,934; desferrioxamines (DFO) are disclosed in Doulias, et al., Free Radic Biol Med, 2003, 35:719; tetrapyridinyl and N3S, N2S2 and N4 chelators are disclosed in U.S. Pat. Nos. 5,367,080A, 5,364,613 A, 5,021,556 A, 5,075,099 A, 5,886,142 A, wherein all references are incorporated by reference in their entirety, 6-amino-6-methyl perhydro-1,4-diazepane-N,N′,N″,N″-tetraacetic acid (AAZTA) is disclosed in Pfister, et al., EJNMMI Res, 2015, 5:74; deferiprone (namely, 1,2-dimethyl-3,4-hydroxy pyridinone), and hexadentate tris(3,4-hydroxy pyridinone) (namely, THP) are disclosed in Cusnir, et al., Int J Mol Sci. 2017, 18: monoamine-monoamide dithiol (MAMA)-based chelators are disclosed in Demoin, et al., Nucl Med Biol, 2016, 43:802; MACROPA and its analogues are disclosed in Thiele, et al., Angew Chem Int Ed Engl, 2017, 56:14712; 1,4,7,10,13,16-hexaazacyclohexadecane-N,N′,N″,N′″,N″″,N′″″-hexaacetic acid (HEHA) and PEPA analogues are disclosed in Price and Orvig (Price, et al., ChemSoc Rev, 2014, 43:260); Pycup and its analogues are disclosed in Boros, et al., Mol Pharm, 2014, 11:617; N,N-bis-(2-hydroxybenzyl)ethylenediamine-N,N-diacetic acid (HBED), 1,4,7,10-tetra(carbamoylmethyl)-1,4,7,10-tetraazacyclododecane (TCM), 2-[(carboxylmethyl)]-[5-(4-nitrophenyl-1-[4,7,10-tris(carboxylmethyl)-1,4,7,10-tetraazacyclododecane-1-yl]pent-2-yl)-amino]acetic acid (3p-C-DEPA), CB-TE2A, TE2A, TE1A1P, Diamsar, 1-N-(4-aminobenzyl)-3,6,10,13,16,19-hexaazabicyclo[6.6.6]-eicosan-1,8-diamine (SarAr), NETA, N,N0,N00-tris(2-thioethyl)-1,4,7-triazacycononane (TACN-TM), {4-[2-(bis-carboxylmethyl-amino)-ethyl]-7-carboxylmethyl-[1,4,7]triazanon-1-yl}-acetic acid (NETA), diethylenetriamine pentaacetic acid (DTP), 3-({4,7-bis-[(2-carboxyl-ethyl)-hydroxy-phosphoryl methyl]-[1,4,7]triazene-1-ylmethyl}-hydroxy-phosphoryl) propionic acid (TRAP), NOPO, H4octapa, SHBED, BPCA, 3,6,9,15-tetraazabicyclo [9.3.1]-pentadecane-1(15), 11,13-tiene-3,6,9, -triacetic acid (PCTA) and 1,4,7,10,13-pentaazacyclopentadecane-N,N′,N″,N′″,N″″-pentaacetic acid (PEPA) are disclosed in Price and Orvig (Price, et al., Chem Soc Rev, 2014, 43: 260; 1-hydroxy-2-pyridinone ligands (HOPO) are disclosed in Allott, et al., Chem Commun (Camb), 2017, 53:8529; [4-carboxylmethyl-6-(carboxylmethyl-methyl-amino)-6-methyl-[1,4]diazohept-1-yl]-acetic acid (DATA) is disclosed in Tornesello, et al., Molecules, 2017, 22:1282; tetra(aminomethyl)methane (TAM) and analogues are disclosed in McAuley, et al., Canadian Journal of Chemistry, 1989, 67:1657; hexadentate tris(3,4-hydroxypyridinone) (THP) and analogues are disclosed in Ma, et al., Dalton Trans, 2015, 44:4884.

Diagnostic and/or therapeutic applications of some of the aforementioned chelators have been described in the prior art. For example, 2-hydrazinonicotinamide (HYNIC) has been extensively used for incorporating ^{99m}Tc and ^{186,188}Re in the presence of co-ligands (Schwartz, et al., Bioconjug Chem, 1991, 2:333; Babich, et al., J Nucl Med, 1993, 34:1964; Babich, et al., Nucl Med Biol, 1995, 22:25). DTPA is used in Octreoscan® to complex ¹¹¹In, and some modifications have been described in the literatures (Li, et al., Nucl Med Biol, 2001, 28:145; Brechbiel, et al., Bioconjug Chem, 1991, 2:187). The use of DOTA-type chelators in radiotherapy was described by Tweedle et al. (U.S. Pat. No. 4,885,363). Other polyaza macrocycles for chelating trivalent isotopic metals were described by Eisenwiener et al. (Eisenwiener, et al., Bioconjug Chem, 2002, 13:530). N⁴-chelators, such as ^{99m}Tc-N4-chelators, have been used for peptide labeling in the context of minigastrin targeting the CCK-2 receptor (Nock, et al., J Nucl Med, 2005, 46:1727).

In some embodiments, the metal chelator is selected from the group consisting of, but not limited to, DOTA, DOTAGA, NOTA, NODAGA, NODA-MPAA, HBED, TETA, CB-TE2A, DTPA, DFO, Macropa, HOPO, TRAP, THP, DATA, NOTP, Sarcophagine, FSC, NETA, H4octapa, Pycup, NₓS₄₋ₓ (N4, N2S2. N3S), Hynic, ^{99m}Tc(CO)₃-chelators, and analogs thereof, wherein:

DOTA denotes 1,4,7,10-tetraazacyclododecane-1,4,7,10-tetraacetic acid; DOTAGA denotes 1,4,7,10-tetraazacyclododecane, 1-(glutaric acid)-4,7,10-triacetic acid; NOTA denotes 1,4,7-triazacycononanetriacetic acid; NODAGA denotes 1,4,7-triazacycononane-N-glutaric acid-N′,N″-diacetic acid: NODA-MPAA denotes 1,4,7-triazacycononane-1,4-diacetic acid-methylphenylacetic acid; HBED denotes bis-(2-hydroxybenzyl)ethylenediamine diacetic acid; TETA denotes 1,4,8,11-tetraazacyclododecane-1,4,8,11-tetraacetic acid: CB-TE2A denotes 4,11-bis-(carboxymethyl)-1,4,8,11-tetraazabicyclo[6.6.2]-hexadecane; DTPA denotes diethylenetriamine pentaacetic acid; DFO denotes Desferal- or Desferrioxamine-type chelators, a non-limiting example has the chemical name of N-[5-({3-[5-(acetyl-hydroxy-amino)-pentylcarbamoyl]-propionyl}-hydroxy-amino)-pentyl]-N-(5-aminopentyl)-N-hydroxy-succinamide; Macropa denotes N,N′-bis [(6-carboxyl-2-pyridyl)methyl]-4,13-diaza-18-crown-6; HOPO denotes octadecane hydroxy pyridinone chelators, the structure of a non-limiting example is shown below; TRAP denotes 3-({4,7-bis[(2-carboxyl-ethyl)-hydroxy-phosphorylmethyl]-[1,4,7]triazonan-1-ylmethyl}-hydroxy-phosphoryl)-propionic acid; THP denotes hexadentate tris(3,4-hydroxypyridinone; DATA denotes [4-carboxylmethyl-6-(carboxylmethyl-methyl-amino)-6-methyl-[1,4]diazepan-1-y]-acetic acid; NOTP denotes 1,4,7-triazacycononane-N,N′N″-tris(methylenephosphonic acid); Sarcophagine denotes 3,6,10,13,16,19-hexaazabicyclo[6.6.6]icosan; FSC denotes 3,15,27-triamino-7,19,31-trihydroxy-10,22,34-trimethyl-1,13,25-trioxa-7,19,31-triaza-cyclohexatriacontane-9,21,33-triene-2,8,14,20,26,32-hexaone; NETA, {4-[2-(bis-carboxylmethyl-amino)-ethyl]-7-carboxylmethyl-[1,4,7]triazanon-1-yl}-acetic acid: H4octapa denotes N,N′-(6-carboxy-pyridin-2-yl-methyl)-N,N′-diacetic acid-1,2-ethylenediamine; Pycup denotes 1,8-(2,6-Pyridinedimethylene)-1,4,8,11-tetraazacyclotetradecane; NₓS₄₋ₓ (N4, N2S2, N3S) denotes a group of tetradentate chelators with N atoms (basic amine or non-basic amide) and thiols as donors, which form stable Tc-complexes, especially Tc(V)-oxo complexes, a representative non-limiting example is MAG3, whose structure is shown below; and MAG3 denotes {2-[2-(3-mercapto-propionylamino)-acetylamino]-acetylamino}-acetic acid; HYNIC denotes 6-hydrazinonicotinic acid; ^{99m}Tc(CO)₃-chelators denote bidentate or tridentate chelators that can form stable complexes with the technetium tricarbonyl fragment;

The chemical structures of these types of chelators are as follows:

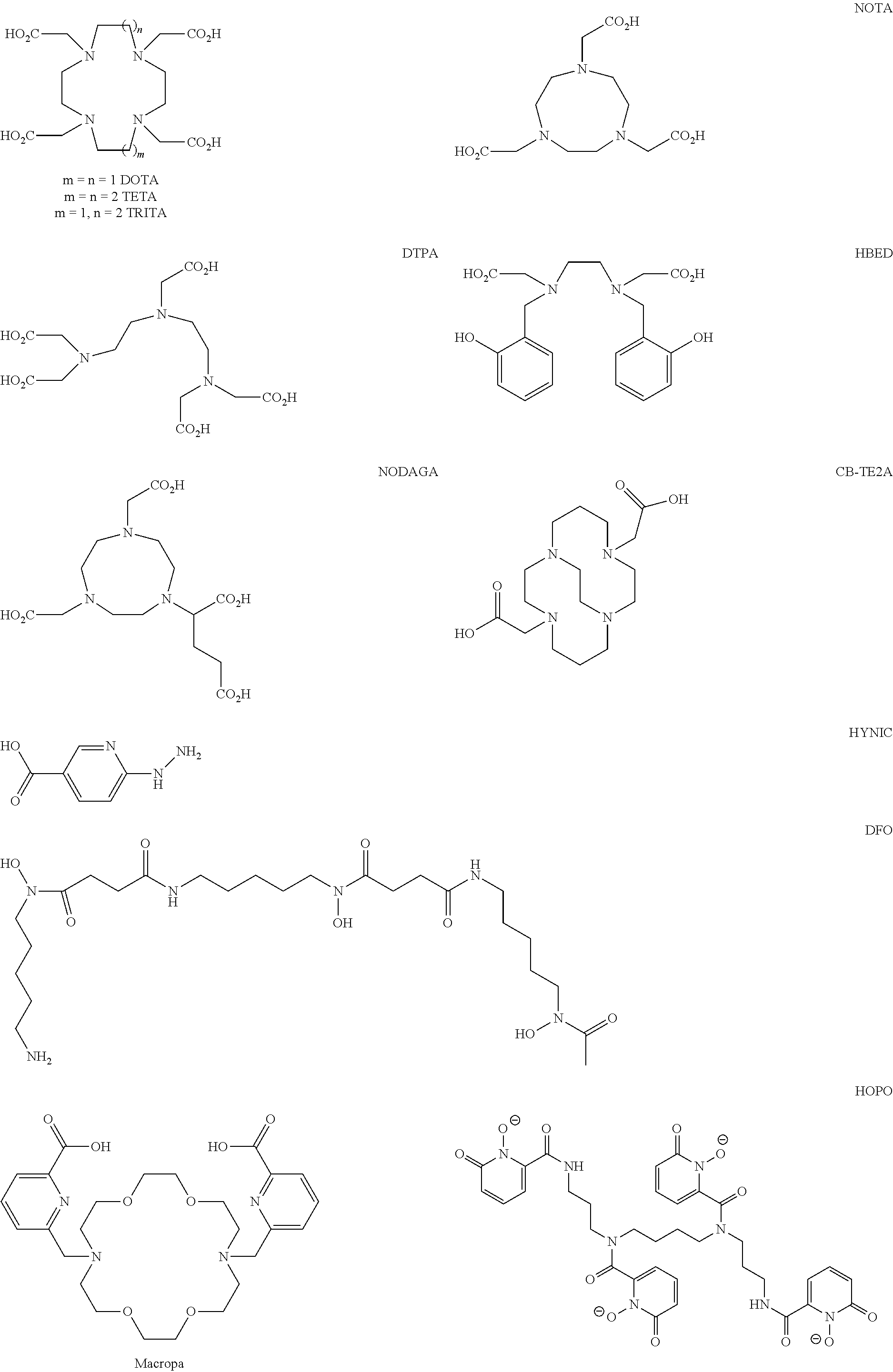

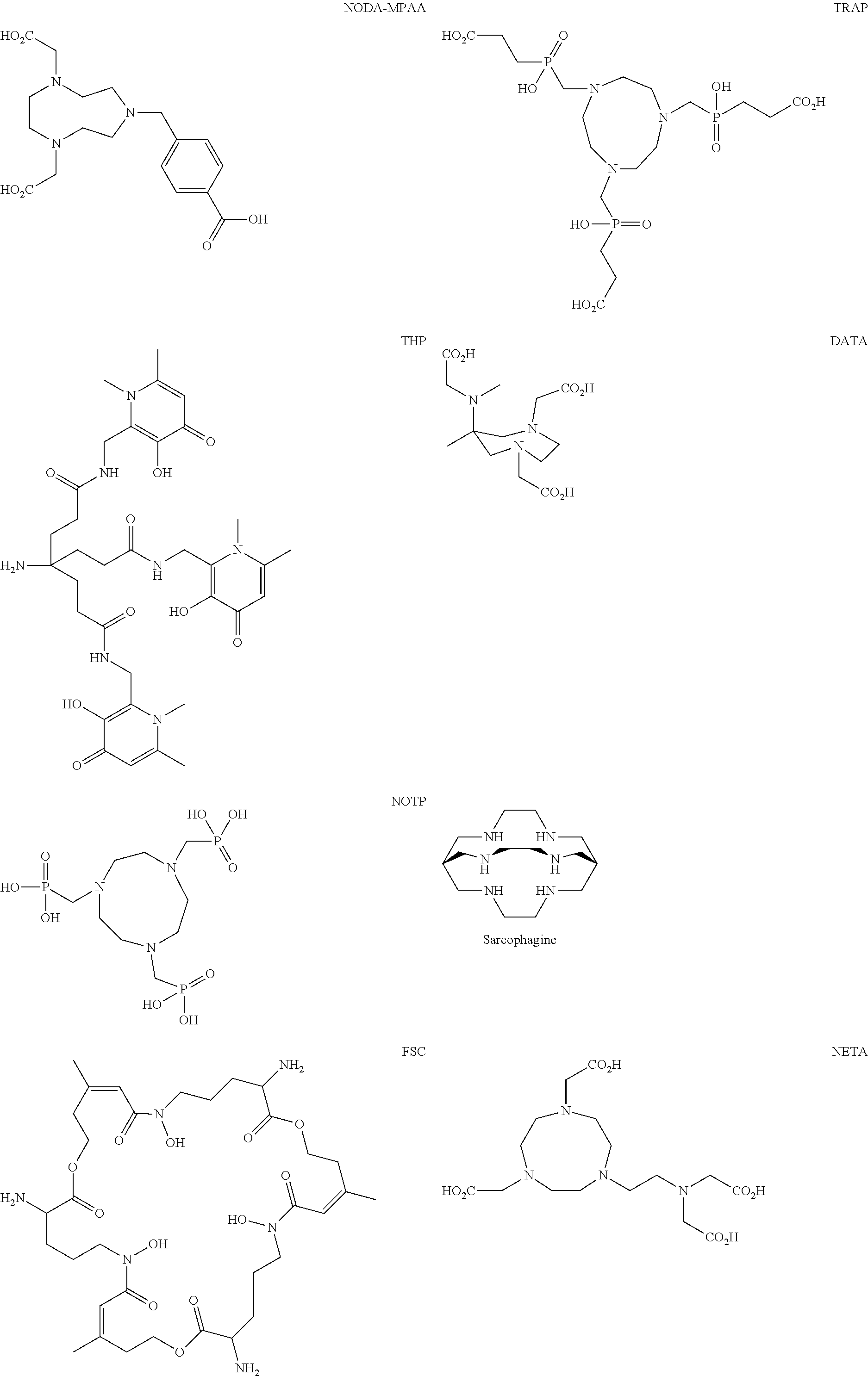

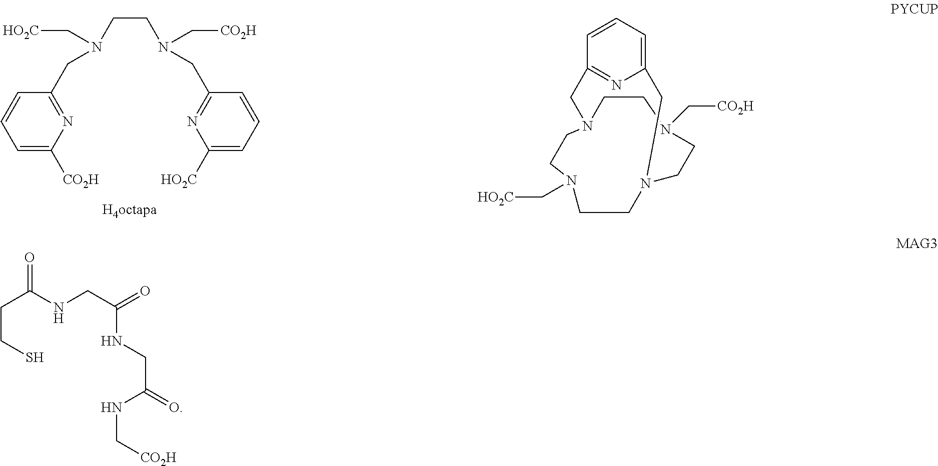 [SMILES: O=C(O)CN(CCN(CC(=O)O)Cc1cccc(C(=O)O)n1)Cc1cccc(C(=O)O)n1 ; O=C(O)CN1CCN2CCN(CC(=O)O)CCN(CC1)Cc1cccc(n1)C2 ; O=C(O)CNC(=O)CNC(=O)CNC(=O)CCS]

In some embodiments, the metal chelator is selected from the group consisting of DOTA, DOTAGA, NOTA, NODAGA, NODA-MPAA, HBED, CB-TE2A, DFO, THP, N4, and their analogues.

In some embodiments, the metal chelator is selected from the group consisting of DOTA, DOTAGA, NOTA, N4Ac and NODAGA, and their analogues.

A person skilled in the art will further appreciate that, unless otherwise specified, the presence of the chelator in the compound of the present invention includes the possibility of the chelator being complexed with any metal complex partner (i.e., any metal that can theoretically be complexed by the chelator). Explicit reference to a chelator of a compound of the invention, or the general term chelator relating to a compound of the invention refers either to the uncomplexed chelator itself, or to the chelator complexed with any metal complex partner, wherein the metal complex partner is any radioactive or non-radioactive metal complex partner. Preferably, the chelator-metal complex, i.e., the chelator complexed with the metal complex partner, is a stable chelator-metal complex.

Non-radioactive chelator-metal complexes have various applications, for example, for evaluating properties that are difficult to determine, such as stability or activity. One aspect is that cold variants of the radioactive forms of the metal complex partner (e.g., the non-radioactive gallium, lutetium, or indium complexes described in the examples) can serve as surrogates for the radioactive compounds. Furthermore, they are valuable tools for identifying metabolites in vitro or in vivo and for evaluating the toxicological properties of the compounds of the invention. Additionally, chelator-metal complexes can be used in binding assays, utilizing the fluorescent properties of some metal complexes with different ligands (e.g., europium salts).

Chelators can be synthetic or commercially available and possess various (potentially pre-activated) groups for conjugation to peptides or amino acids. Direct conjugation of the chelator to the nitrogen in the amino of the corresponding compound of the invention is entirely possible for chelators selected from the group consisting of: DOTA, DOTAGA, NOTA, NODAGA, NODA-MPAA, HBED, TETA, CB-TE2A, DTPA, DFO, DATA, Sarcophagine, N4, MAG3, and Hynic, preferably DOTA, DOTAGA, NOTA, NODAGA, NODA-MPAA, CB-TE2A, and N4. A preferred linkage in this regard is amide bond.

A person skilled in the art is aware of functional groups on chelators that are ideal precursors for direct conjugation of the chelator to the nitrogen in the amino, including but not limited to carboxylic acids: activated carboxylic acids such as active esters like NHS-esters, pentafluorophenol-esters, HOBt-esters, and HOAt-esters; and isothiocyanates.

A person skilled in the art is aware of functional groups on chelators that are ideal precursors for direct conjugation of the chelator to the carboxyl group of a peptide, including but not limited to nitrogens in alkylamino and arylamino moieties. Corresponding chelator reagents are some commercially available chelators, for example, DOTA possessing alkylamino or arylamino nitrogen.

A person skilled in the art will recognize that the radionuclide being linked or to be linked to the compound of the invention is selected taking into account the disease to be treated and/or diagnosed and/or the characteristics of the patient and patient group to be treated and diagnosed, respectively.

In some embodiments of the invention, the radioactive nuclide is also referred to as a radionuclide. Radioactive decay is the process by which an unstable atomic nucleus loses energy by emitting ionizing particles (ionizing radiation). There are different types of radioactive decay. Energy attenuation or loss occurs when an atom with one type of nucleus (called the parent radionuclide) transforms into a nucleus in a different state or containing different numbers of protons and neutrons. Any of these products is named the daughter nuclide. In certain decays, the parent and daughter are different chemical elements, so the decay process results in a nuclear transmutation (producing an atom of a new element). For instance, radioactive decay can be α decay, β decay, and γ decay. α decay occurs when the nucleus ejects an α particle (a helium nucleus). This is the most common process of emitting nucleons. Nevertheless, in more rare types of decay, atomic nuclei can eject protons or specific nucleons of other elements (referred to as cluster decay in this process). During the process of a proton turning into a neutron or vice versa, beta decay occurs when an atomic nucleus emits electrons (β⁻-decay) or positrons (B⁺-decay) and a type of neutrino. In contrast, there are radioactive decay processes that do not result in transmutation, wherein energy of an excited nucleus can be emitted as γ rays in γ decay, or used to eject an orbital electron through interaction with the excited nucleus in a process called internal conversion, or used for absorbing internal atomic electrons from the electron shell, thereby converting nuclear protons into neutrons, leading to a process of electron capture (EC) that emits electron neutrinos, or alternatively, can be emitted without changing the number of protons and neutrons in a process called isomeric transition (IT). One form of radioactive decay, known as spontaneous fission (SF), is found only in very heavy chemical elements and results in their spontaneous disintegration into smaller atomic nuclei and some isolated nuclear particles. The compounds according to the present invention may be synthesized using one or more of the methods described below. It should be noted that the general procedures outlined herein are illustrated using compounds with unspecified stereochemistry. Nonetheless, these procedures are generally applicable to compounds with defined stereochemistry, e.g., where a group has the (S) or (R) configuration. Furthermore, using well-established methods such as configuration inversion, compounds with one stereochemistry (e.g., (R)) can typically be used to readily access those with the opposite stereochemistry (i.e., (S)).

Certain compounds of the present invention may exist in unsolvated forms as well as solvated forms, including hydrated forms. In general, the solvated forms are considered equivalent to the unsolvated forms and are intended to be encompassed within the scope of the present invention. Certain compounds of the invention may exist in multiple crystalline or amorphous forms. In general, all such physical forms are considered equivalent for the intended purposes of the present invention and are intended to be encompassed within its scope.

Certain compounds of the present invention possess asymmetric carbon atom (optical center) or double bond. All racemates, diastereomers, geometric isomers, and individual isomers of the compound are encompassed within the scope of the invention.

Compounds of the present invention may further incorporate atoms having a non-natural abundance of isotopes at one or more positions. For example, the compounds may be radiolabeled with radioisotopes, such as tritium (³H), iodine-125 (¹²⁵I), or carbon-14 (¹⁴C). All isotopic variations of the compounds of the present invention, regardless of whether they are radioactive, are intended to be encompassed within the scope of the invention.

The term “pharmaceutical composition” as used in this application refers to a substance and/or combination of substances for identifying, preventing, or treating a tissue condition or disease. Pharmaceutical compositions are formulated to be suitable for administration to a patient for preventing and/or treating a disease. Additionally, a pharmaceutical composition refers to the combination of an active agent with an inert or active carrier, making the composition suitable for therapeutic use. Depending on their chemical and physical properties, pharmaceutical compositions can be formulated for oral, parenteral, topical, inhalational, rectal, sublingual, transdermal, subcutaneous, or vaginal routes of administration. Pharmaceutical compositions may be in the form of solid, semi-solid, liquid, and transdermal therapeutic system (TTS). Solid composition may be selected from the group consisting of tablet, coated tablet, powder, granule, pill, capsule, effervescent tablet, and transdermal therapeutic system. Liquid composition may be selected from the group consisting of solution, syrup, infusion, extract, solution for intravenous administration, solution for infusion, and solution of the carrier system of the invention. Semi-solid compositions usable in the context of the invention include emulsion, suspension, cream, lotion, gel, microsphere, buccal lozenge, and suppository.

“Pharmaceutically acceptable” means approved by a federal or state government regulatory agency or listed in the United States Pharmacopeia or other generally recognized pharmacopeia for animals, and especially for humans.

The term “carrier” refers to a diluent, adjuvant, excipient, or vehicle administered with the therapeutic agent. Such pharmaceutical carriers can be sterile liquids, such as a saline solution in water and oils, including those of petroleum, animal, vegetable, or synthetic origin, such as peanut oil, soybean oil, mineral oil, sesame oil, etc. Saline solution is a preferred carrier when the pharmaceutical composition is administered intravenously. Saline solutions, as well as aqueous dextrose and glycerol solutions, can also be used as liquid carriers, particularly for injectable solutions. Suitable pharmaceutical excipients include starch, glucose, lactose, sucrose, gelatin, malt, rice, flour, chalk, silica gel, sodium stearate, glyceryl monostearate, talc, sodium chloride, skimmed milk powder, glycerol, propylene glycol, water, ethanol, etc. If desired, the composition can also contain small amounts of wetting agents or emulsifying agents or pH buffering agents. Examples of suitable pharmaceutical carriers are described in “Remington's Pharmaceutical Sciences” by E. W. Martin.

Amino acid residues and abbreviations: Glutamine, Gln; Asparagine, Asn; Histidine, His; Tryptophan, Trp; Tpi; Ala; Serine, Ser; Valine, Val; Threonine, Thr; Glycine, Glyl Sarosine, Sar; D-alanine, D-Ala; β-alanine, β-Ala; 3-Methylhistidine, (3-Me)His; Phenylalanine, Phe; 3-(2-Thienyl) alanine, Thi; 4-Chlorophenylalanine, Cpa; Tyrosine, Tyr; 3-iodo-tyrosine, o-I-Tyr; Pentafluorophenylalanine, 5F-Phe.

The term “FAP receptor-targeting moiety” refers to one or more molecular structure capable of binding to FAP and inhibiting FAP biological activity and/or mediated by FAP signaling.

The term “CXCR4 receptor-targeting moiety” refers to one or more molecular structure capable of binding to CXCR4 and inhibiting CXCR4 biological activity and/or mediated by CXCR4 signaling.

When a substituent is described by the conventional chemical formula written from left to right, the substituent also includes the chemically equivalent substituent obtained when the structural formula is written from right to left. For example,

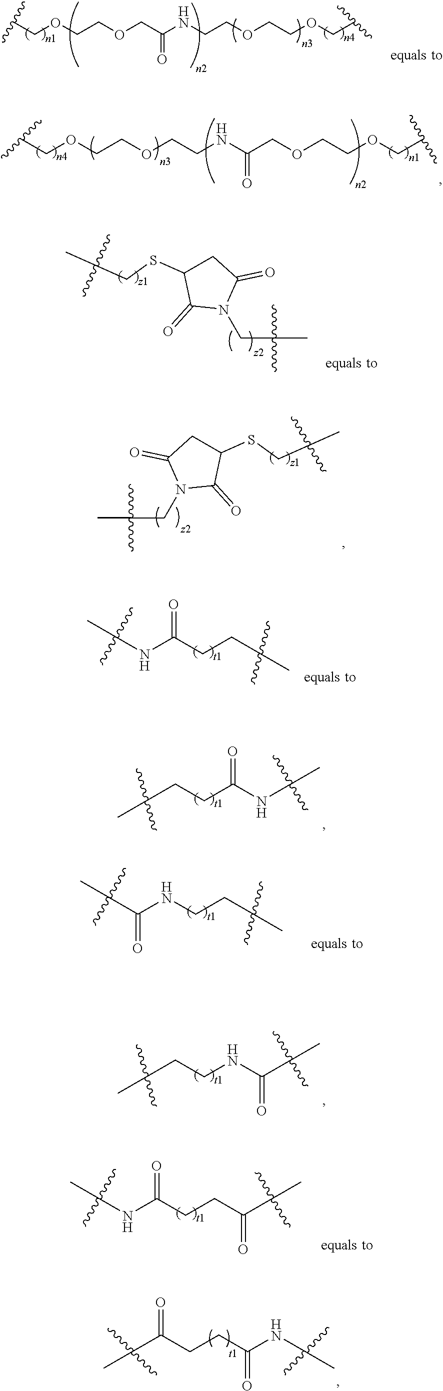

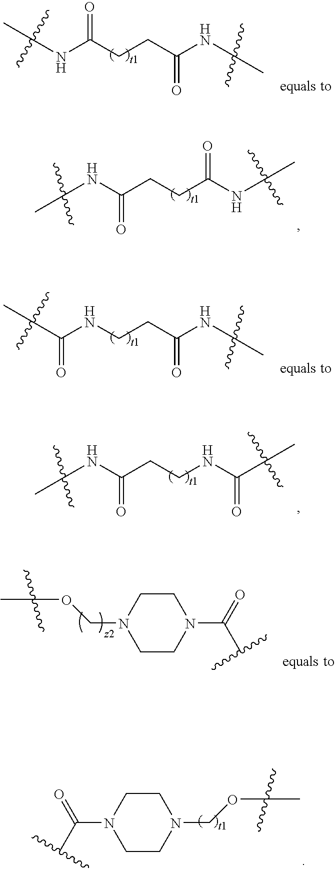

As described in the present invention, a ring system (as shown in the figure below) formed by connecting a substituent R to the center of a ring via a bond means that the substituent R is only substituted at any substitutable or any reasonable position on the ring A. For example, formula f represents any possible position on the ring A that can be substituted, as shown in formulas f¹-f⁴:

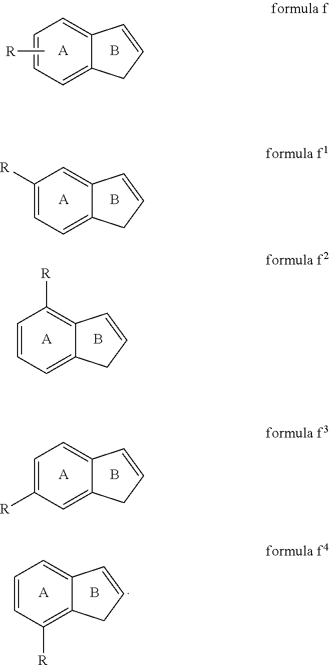

In the present invention, when a connecting bond traverses a ring system, with the other end connected to the remainder of the compound molecule, it indicates that the connection can be made at any possible position within the ring system.

For example, the structure

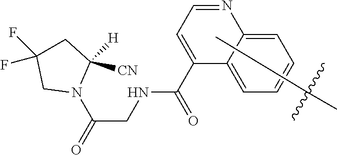

denotes any one of the following structures:

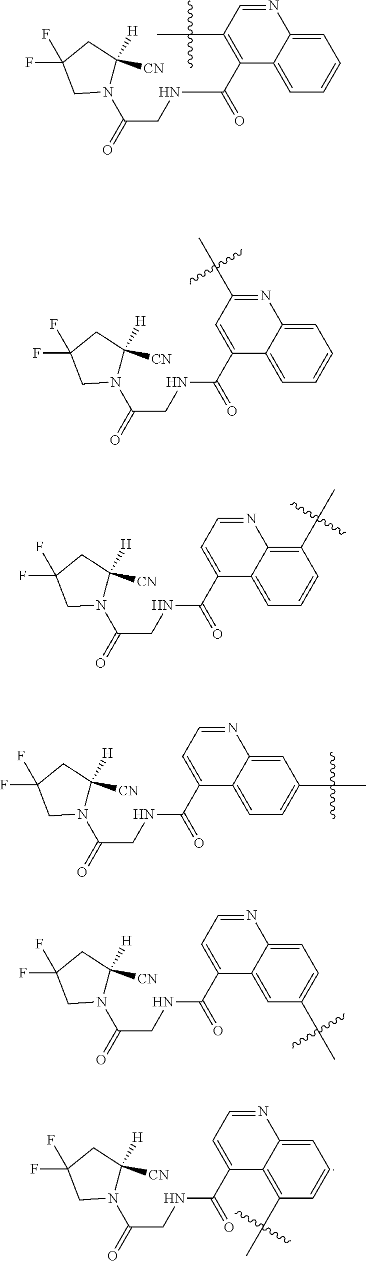 [SMILES: *c1cnc2ccccc2c1C(=O)NCC(=O)N1CC(F)(F)C[C@H]1C#N ; *c1cc(C(=O)NCC(=O)N2CC(F)(F)C[C@H]2C#N)c2ccccc2n1 ; *c1cccc2c(C(=O)NCC(=O)N3CC(F)(F)C[C@H]3C#N)ccnc12 ; *c1ccc2c(C(=O)NCC(=O)N3CC(F)(F)C[C@H]3C#N)ccnc2c1 ; *c1ccc2nccc(C(=O)NCC(=O)N3CC(F)(F)C[C@H]3C#N)c2c1 ; *c1cccc2nccc(C(=O)NCC(=O)N3CC(F)(F)C[C@H]3C#N)c12]

As described in the present invention, a ring system formed by connecting a substituent to the center of a ring via a bond such as (R^{x})ₙ means that n substituents R^{x} can be substituted at any substitutable position on the ring. For example, formula a represents that the benzene ring can be substituted with n R^{x}.

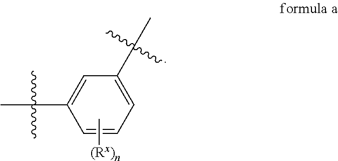

In the description of this specification, references to terms such as “an embodiment.” “some embodiments,” “an example,” “a specific example,” or “some examples” mean that specific features, structures, materials, or characteristics described in connection with the embodiment(s) or example(s) are included in at least one embodiment or example of the present invention. In this specification, schematic representations of the aforementioned terms do not necessarily refer to the same embodiment or example. Furthermore, the described specific features, structures, materials, or characteristics may be combined in any suitable manner in one or more embodiments or examples. Additionally, without mutual contradiction, those skilled in the art can combine and integrate the different embodiments or examples and the features of different embodiments or examples described in this specification.

The term “about” as used herein indicates a range of ±20% of the subsequent numerical value. In some embodiments, the term “about” indicates a range of ±10% of the subsequent numerical value. In some embodiments, the term “about” indicates a range of ±5% of the subsequent numerical value.

Examples and figures are provided below for better understanding the invention. However, it should be understood that these examples and figures are provided merely to illustrate the invention and do not constitute any limitation. The actual scope of protection for the invention is set forth in the claims. It should be understood that any modifications and alterations can be made without departing from the spirit of the invention.

### Methods and Materials

### Experimental Materials and Instruments

Unless otherwise specified, all chemicals are commercially purchased and used without further purification. Table 1 lists the instruments and key chemicals used in this experiment.

TABLE 1
Experimental Instruments and Materials

| Instrument/Material | Manufacturer | Model |
|---|---|---|
| Chromatography Grade | J&K Scientific Ltd., China | |
| Methanol | | |
| Chromatography Grade | Adamas-beta company | |
| Acetonitrile | | |
| High Performance Liquid | Thermo Fisher Scientific, | Ultimate |
| Chromatograph | USA | 3000 |
| ⁶⁸Ge/⁶⁸Ga generator | China Isotope & Radiation | 22-060 |
| | Corporation | |
| PBS Buffer | Servicebio, Wuhan | |
| Capillary Electrophoresis | Agilent Technologies, USA | CE 7100 |
| System | | |
| Intermediates or starting | Nanchang Tanzhen | |
| materials (e.g., 1-4, 2-1, 2-4, | Biotechnology Co., Ltd. | |
| etc.) | | |

### Chromatographic Conditions

The chromatographic conditions used for the analysis of precursors and radiotracers are listed in Table 2.

TABLE 2
Chromatographic Conditions

| | Chemical Purity | |
|---|---|---|
| | Determination | Radiolabeling |
| Column | Sepax GP-C18 (5 μm, | Agilent ZORBAX SB-C18 |
| | 4.6 mm × 250 mm) | (5 μm, 4.6 mm × 250 mm) |
| Mobile | A: Pure water with 0.1% TFA | A: Pure water with 0.1% TFA |
| Phase | B: Acetonitrile with | B: Acetonitrile with |
| | 0.1% TFA | 0.1% TFA |
| Elution | 0~20.0 min, 85%~65% A | 0~5.0 min, 95% A |
| Program | | 5.0~19.0 min, 95%~5% A |
| | | 19.0~20.0 min, 5%~95% A |

### Example 1: Synthesis of SDYD01 and Related Radioactive Experimental Studies

### Synthesis and Analysis of SDYD01:

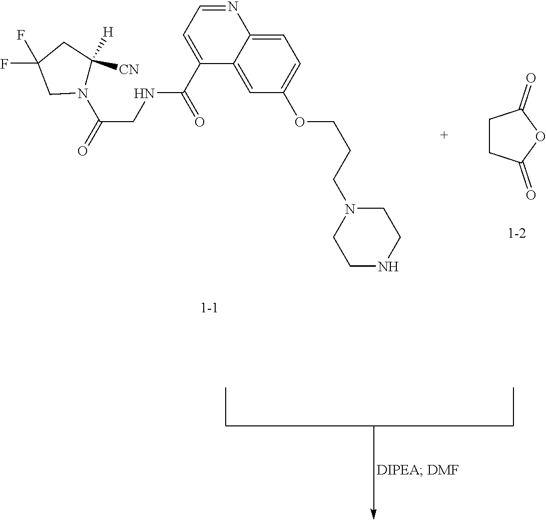

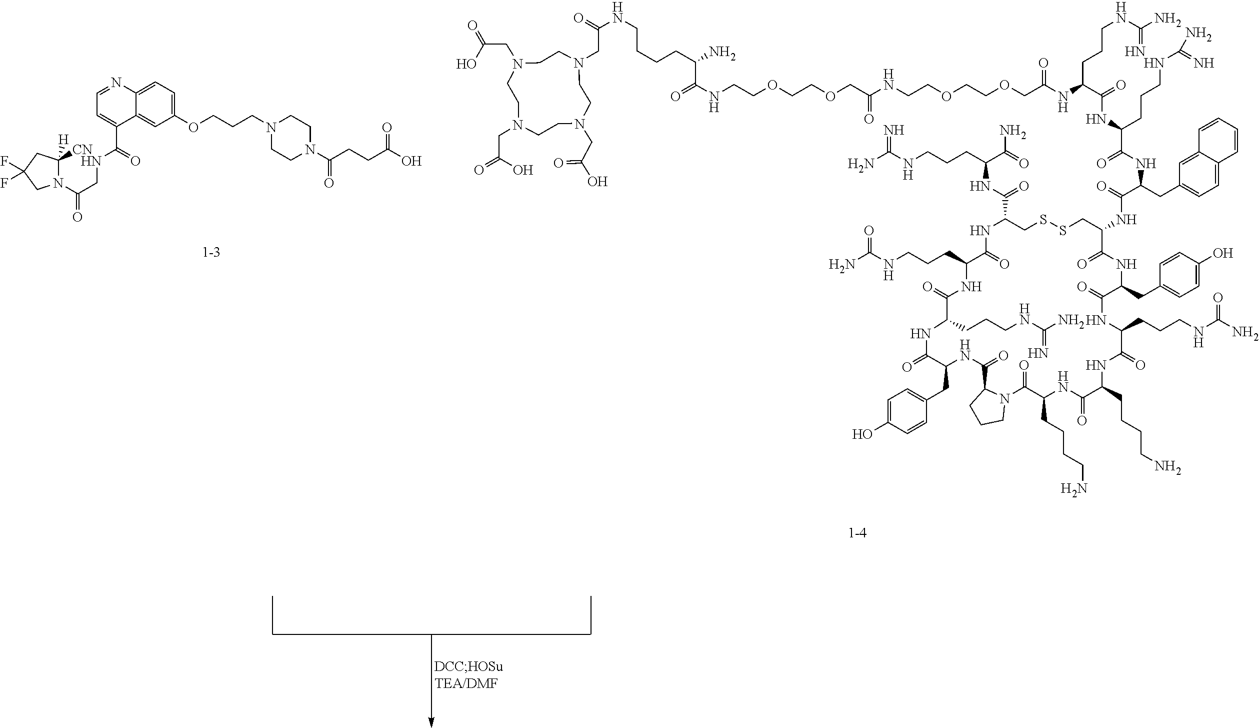

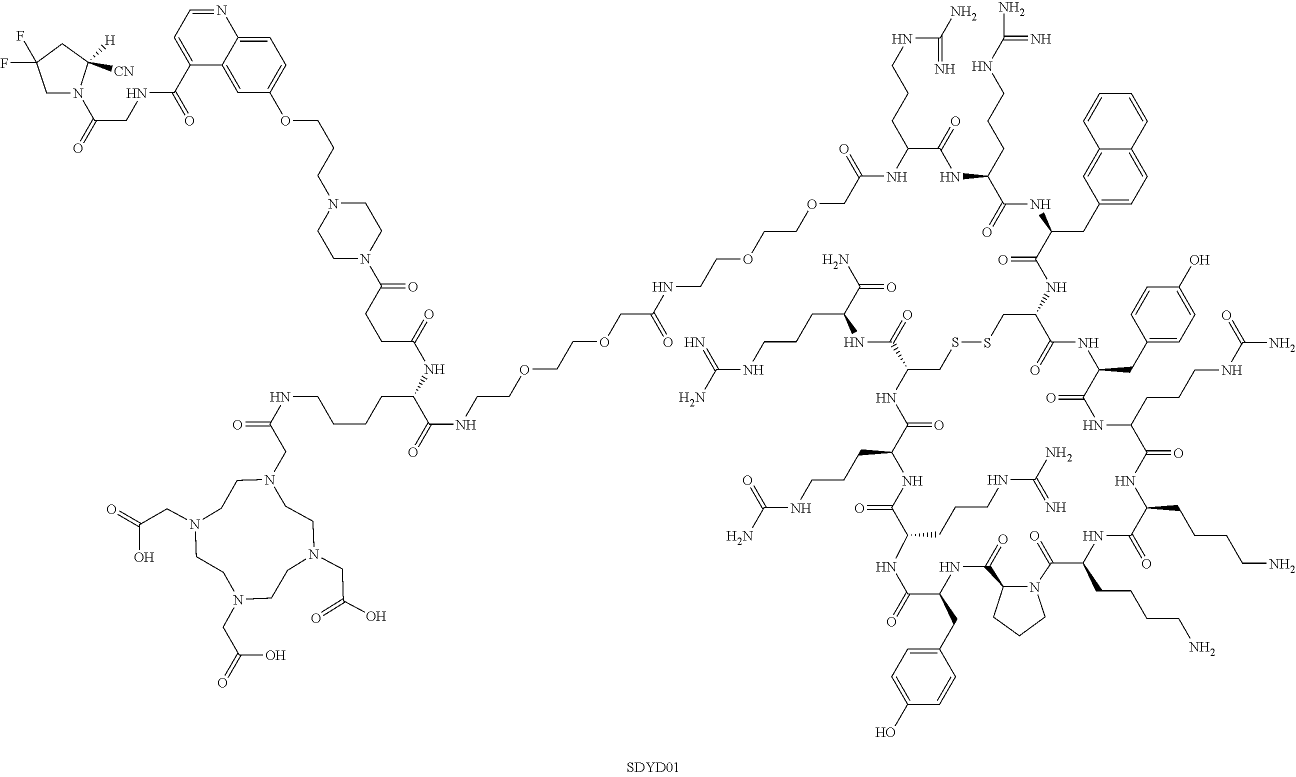 [SMILES: N#C[C@@H]1CC(F)(F)CN1C(=O)CNC(=O)c1ccnc2ccc(OCCCN3CCN(C(=O)CCC(=O)N[C@@H](CCCCNC(=O)CN4CCN(CC(=O)O)CCN(CC(=O)O)CCN(CC(=O)O)CC4)C(=O)NCCOCCOCC(=O)NCCOCCOCC(=O)NC(CCCNC(=N)N)C(=O)N[C@@H](CCCNC(=N)N)C(=O)N[C@@H](Cc4ccc5ccccc5c4)C(=O)N[C@H]4CSSC[C@@H](C(=O)N[C@@H](CCCNC(=N)N)C(N)=O)NC(=O)[C@H](CCCNC(N)=O)NC(=O)[C@H](CCCNC(=N)N)NC(=O)[C@H](Cc5ccc(O)cc5)NC(=O)[C@@H]5CCCN5C(=O)[C@H](CCCCN)NC(=O)[C@H](CCCCN)NC(=O)C(CCCNC(N)=O)NC(=O)[C@H](Cc5ccc(O)cc5)NC4=O)CC3)cc12 ; "SDYD01"]

S1. Compound 1-1 (100 mg) was dissolved in 20 mL of DMF, added with 6.0 eq DIPEA, followed by 5.0 eq compound 1-2. The reaction was allowed to proceed at room temperature for 3 hours. Upon LC-MS confirmation of reaction completion, DMF was removed via rotary evaporation. Purification via reversed-phase preparative LC afforded 61 mg of compound 1-3 (yield: 51%).

S2. Compound 1-3 (61 mg) was dissolved in 10 mL of DMF, followed by the addition of 1.2 eq DCC and 1.2 eq HOSu (N-Hydroxy succinimide). The reaction was allowed to proceed at room temperature for 6 hours. The reaction mixture was filtered to remove precipitated solids. 296 mg of compound 1-4 and 2 eq TEA were added to the filtrate. The reaction was allowed to proceed at room temperature for 3 hours. Upon LC-MS confirmation of reaction completion, DMF was removed via rotary evaporation. Purification via reversed-phase preparative LC afforded 188 mg of target product SDYD01 (yield: 53%).

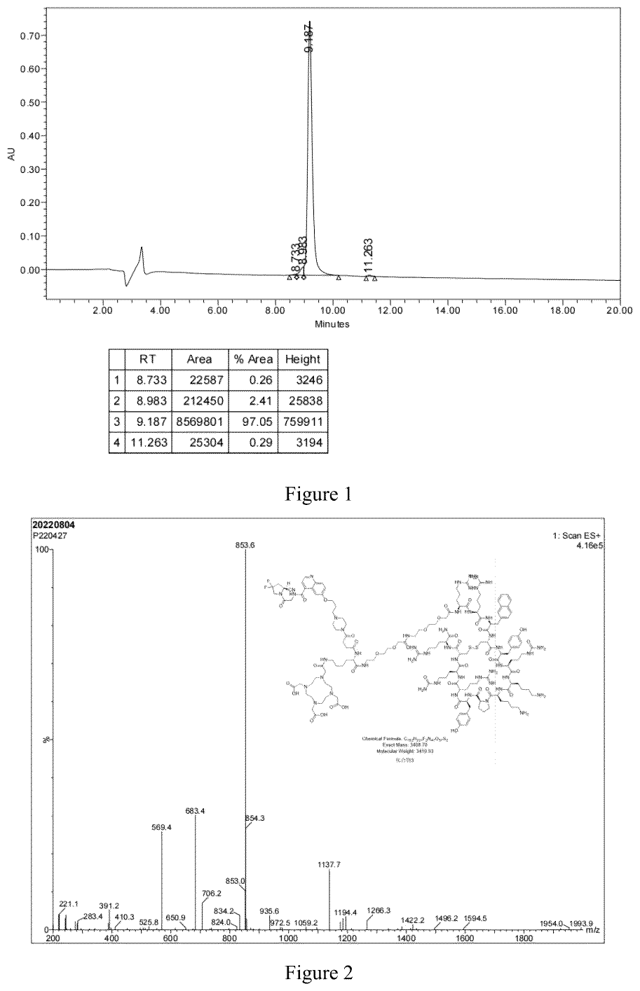

HPLC purity: 97.05% (FIG. 1). Molecular weight: theoretical 3410.93, measured 853.6 for [M+4H]/4 (FIG. 2). Elemental analysis: N: 14.65%. C: 50.05%, H: 5.89%, which are consistent with the theoretical values. Main peak's PI is 12.474. Specific rotatory power is −9.8°.

### Radiolabeling of SDYD01 and Determination of Radiochemical Purity

Radiochemical purity refers to the percentage of radionuclide in the specified chemical form relative to the total radionuclide present in a radioactive sample. In this experiment, it specifically refers to the percentage of radioactivity successfully attached to the compound SDYD01 relative to the total radioactivity after purification.

1. ⁶⁸Ga Labeling: 10 μg of compound SDYD01 was added to 100 μL of NaOAc (0.25 M) buffer, followed by addition of 400 μL of ⁶⁸GaCl₃ solution (by rinsing the ⁶⁸Ge-⁶⁸Ga generator using 0.05 M HCl). The reaction mixture was shaken at 100° C. for 15 min. The crude product was purified using a C18 cartridge. The radiochemical purity of ⁶⁸Ga-SDYD01 was determined by radio-HPLC.

2. ¹⁷⁷Lu Labeling: 10 μg of compound SDYD01 was added to 150 μL of sodium acetate buffer (pH=4.6), followed by addition of 20 MBq of ¹⁷⁷LuCl₃ solution. The reaction mixture was shaken at 100° C. for 50 min. The crude product was purified using a C18 cartridge. The radiochemical purity of ¹⁷⁷Lu-SDYD01 was determined by radio-HPLC.

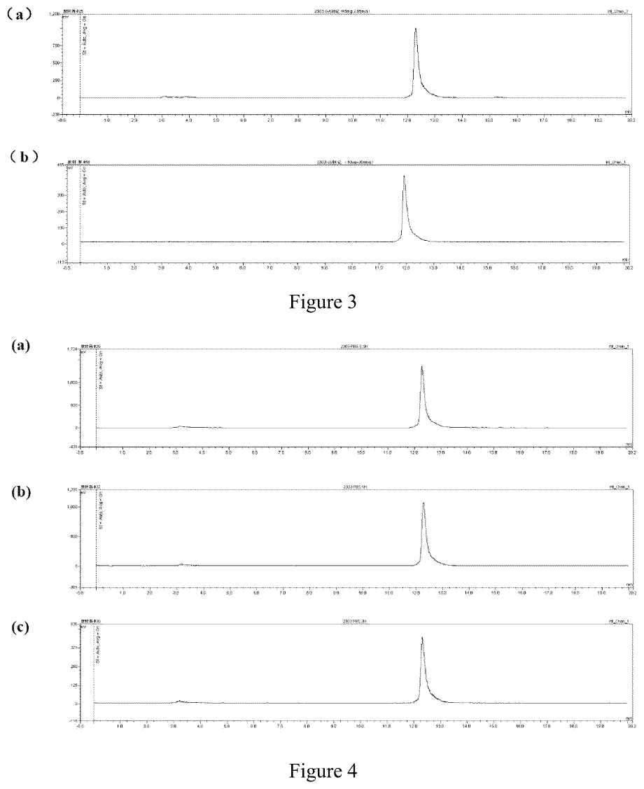

The radiochemical purity of ⁶⁸Ga/¹⁷⁷Lu-SDYD01 was determined by radio-HPLC. Results showed 99% for ⁶⁸Ga-SDYD01 and 98% for ¹⁷⁷Lu-SDYD01 (FIG. 3).

### Stability of SDYD01 Radioligands in PBS Buffer

The purpose of this experiment was to test the stability of the radioligands under buffer conditions simulating the physiological environment. This stability is a prerequisite for further animal experiments. If no significant degradation of the ligand occurs, it indicates that the metal chelation and peptide bonds are stable in the buffer.

1. ⁶⁸Ga-SDYD01: ⁶⁸Ga-SDYD01 was added to PBS buffer (pH=7.4) and incubated at 37° C. The mixture samples were taken for radio-HPLC analysis after 0.5 hours, 1 hour, and 2 hours of incubation. The stability of ⁶⁸Ga-SDYD01 in PBS buffer: the radiochemical purities of ⁶⁸Ga-SDYD01 after incubation in PBS for 0.5 hours, 1 hour, and 2 hours were 98%, 97%, and 97%, respectively (FIG. 4). The results indicate that ⁶⁸Ga-SDYD01 is quite stable in PBS buffer.

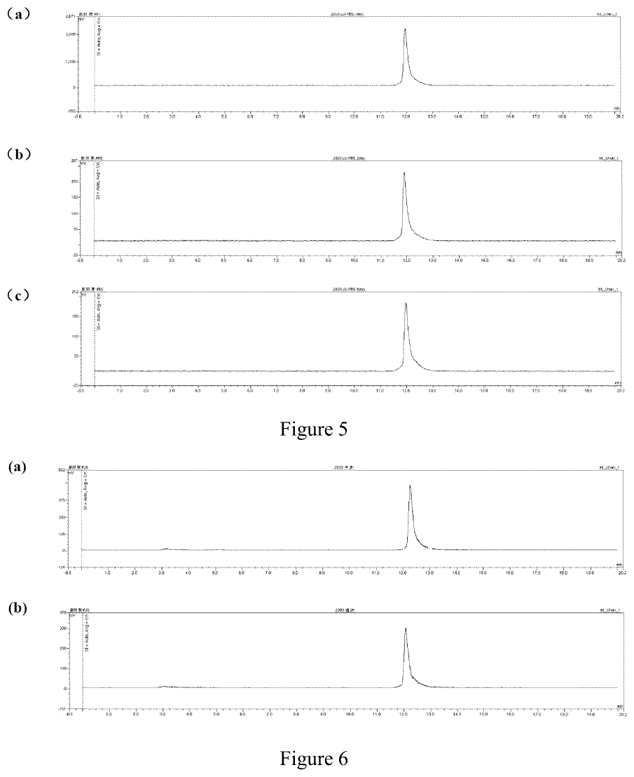

2. ¹⁷⁷Lu-SDYD01: ¹⁷⁷Lu-SDYD01 was added to PBS buffer (pH=7.4) and incubated at 37° C. The mixture samples were taken for radio-HPLC analysis after 24 hours, 48 hours, and 120 hours of incubation. The stability of ¹⁷⁷Lu-SDYD01 in PBS buffer: the radiochemical purities of ¹⁷⁷Lu-SDYD01 after incubation in PBS for 24 hours, 48 hours, and 120 hours were 97%, 96%, and 94%, respectively (FIG. 5). The results indicate that 177Lu-SDYD01 is quite stable in PBS buffer.

### Competitive Experiment of SDYD01 Radioligand Against Endogenous Free Amino Acids

To an EP tube containing 150 μL of PBS buffer, 150 μL of H₂O, and 50 μL of ⁶⁸Ga-SDYD01 was added 50 μL of cysteine hydrochloride solution (10 mM) or histidine solution (10 mM). The mixture was vortexed and incubated at 37° C. for 2 hours, after which samples were taken for radio-HPLC analysis.

This study utilized different concentrations of histidine and cysteine solutions to investigate whether histidine (or cysteine) competes with ⁶⁸Ga-SDYD01 for metal chelation. The study showed that after incubation with histidine and cysteine for 2 hours, the radiochemical purity of ⁶⁸Ga-SDYD01 remained at 93% (FIG. 6), indicating that ⁶⁸Ga-SDYD01 did not undergo competitive chelation in the presence of histidine and cysteine.

### Determination of Hydrophilicity/Lipophilicity of SDYD01 Radioligands

10 μL of Purified ⁶⁸Ga-SDYD01 was diluted to 500 μL with HEPES buffer (pH=7.4), then 500 μL of n-octanol was added. The mixture was vortexed for 30 min and then centrifuged. Subsequently, 450 μL each of the aqueous and organic phases were taken and centrifuged again. Then, 100 μL each of the aqueous and organic phases were taken, and the radioactivity was measured using a γ counter to calculate the n-octanol/water distribution coefficient (Log Do/w). The measured Log Do/w value for ⁶⁸Ga-SDYD01 was −2.54±0.01, and for ¹⁷⁷Lu-SDYD01 was −2.55±0.05.

### Example 2: Synthesis of SDYD02 and Related Radioactive Experimental Studies

### Synthesis and Analysis of SDYD02:

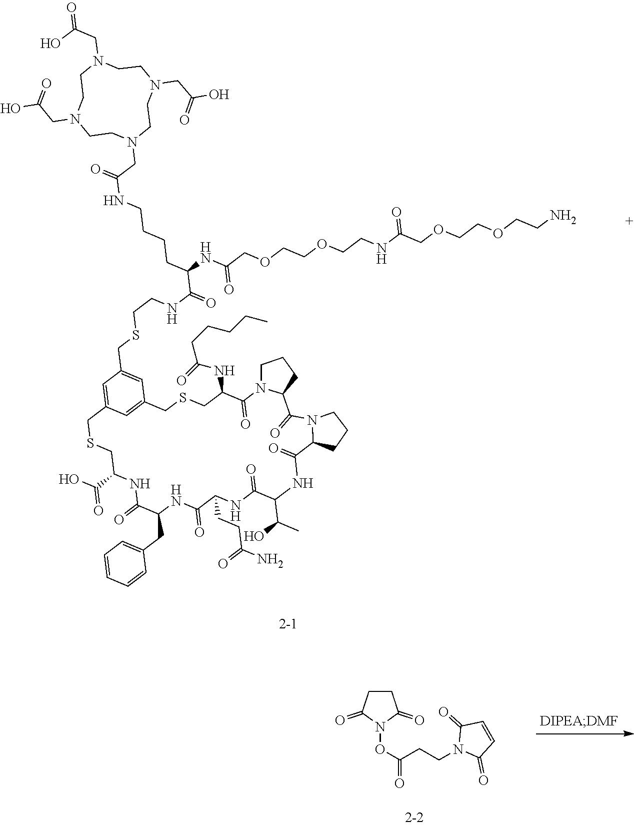

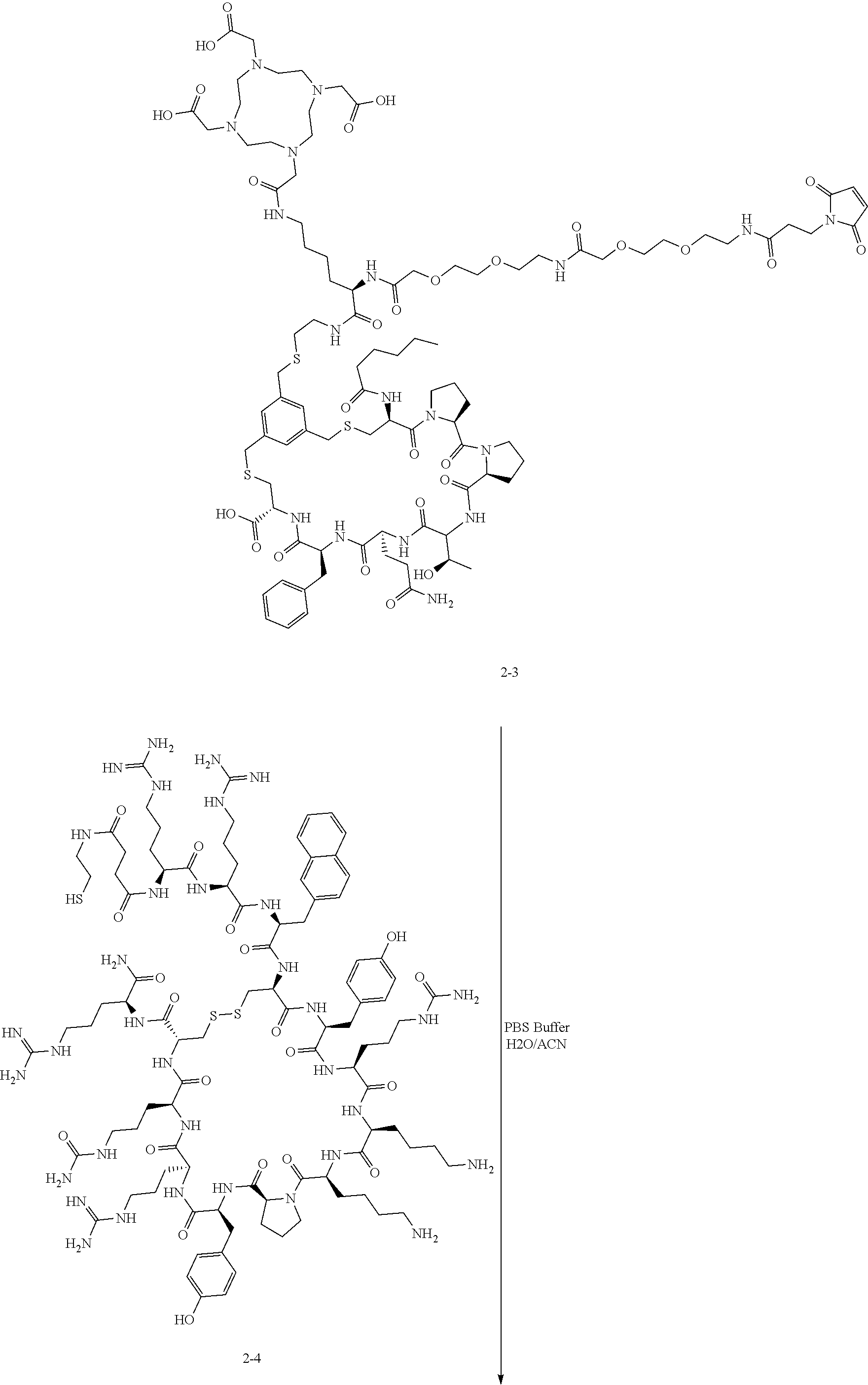

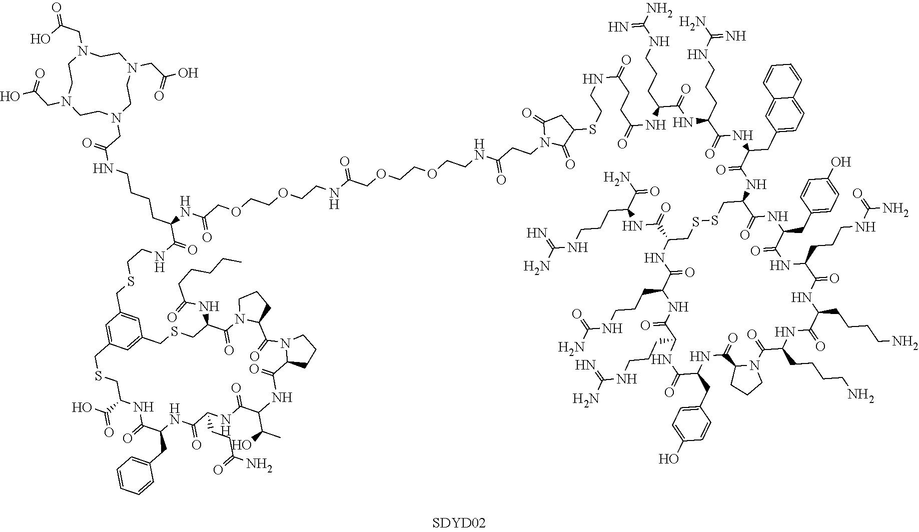

S1. Compound 2-1 (500 mg) and compound 2-2 (140 mg) were dissolved in 5 mL DMF, added with 3 eq DIPEA. The reaction was allowed to proceed at room temperature for 3 hours. Upon LC-MS confirmation of reaction completion, DMF was removed via rotary evaporation. Purification via reversed-phase preparative LC afforded 320 mg of compound 2-3 (yield: 64%).

S2. Compound 2-3 (320 mg) and compound 2-4 (344 mg) were dissolved in a mixture of acetonitrile and water (1:1, 5 mL). This solution was added to a 0.2 M PBS buffer solution (pH=7.2) under a nitrogen atmosphere. The reaction was allowed to proceed at room temperature for 1 hour. The reaction was monitored by LC-MS. After completion, purification via reversed-phase chromatography afforded 338 mg of the target product SDYD02 (yield: 51%).

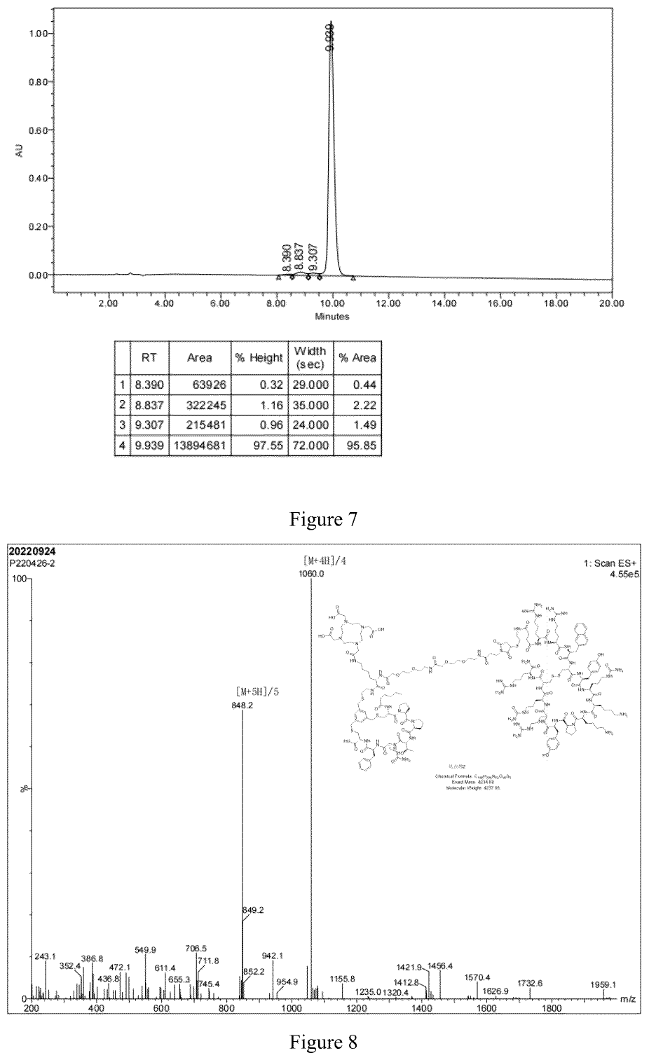

HPLC purity: 95.85% (FIG. 7). Molecular weight: theoretical 4237.01, measured 1060.0 for [M+4H]/4, 848.2 for [M+5H]/5 (FIG. 8). Elemental analysis: N: 14.29%, C: 52.55%, H: 6.125%, which are consistent with the theoretical values. Main peak's PI is 11.183. Specific rotatory power is 6.6°.

### Radiolabeling of SDYD02 and Determination of Radiochemical Purity

Radiochemical purity refers to the percentage of radionuclide in the specified chemical form relative to the total radionuclide present in a radioactive sample. In this experiment, it specifically refers to the percentage of radioactivity successfully attached to the compound SDYD02 relative to the total radioactivity after purification.

1. ⁶⁸Ga Labeling: 10 μg of compound SDYD02 was added to 100 μL of NaOAc (0.25M) buffer, followed by addition of 400 μL of ⁶⁸GaCl₃ solution (by rinsing the ⁶⁸Ge-⁶⁸Ga generator using 0.05 M HCl). The reaction mixture was shaken at 100° C. for 15 min. The crude product was purified using a C18 cartridge. The radiochemical purity of ⁶⁸Ga-SDYD02 was determined by radio-HPLC.

2. ¹⁷⁷Lu Labeling: 20 μg of compound SDYD02 was added to 150 μL of sodium acetate buffer (pH=4.6), followed by addition of 20 MBp of LuCl₃ solution containing ¹⁷⁷Lu. The reaction mixture was shaken at 100° C. for 50 minutes. The crude product was purified using a C18 cartridge. The radiochemical purity of ¹⁷⁷Lu-SDYD02 was determined by radio-HPLC.

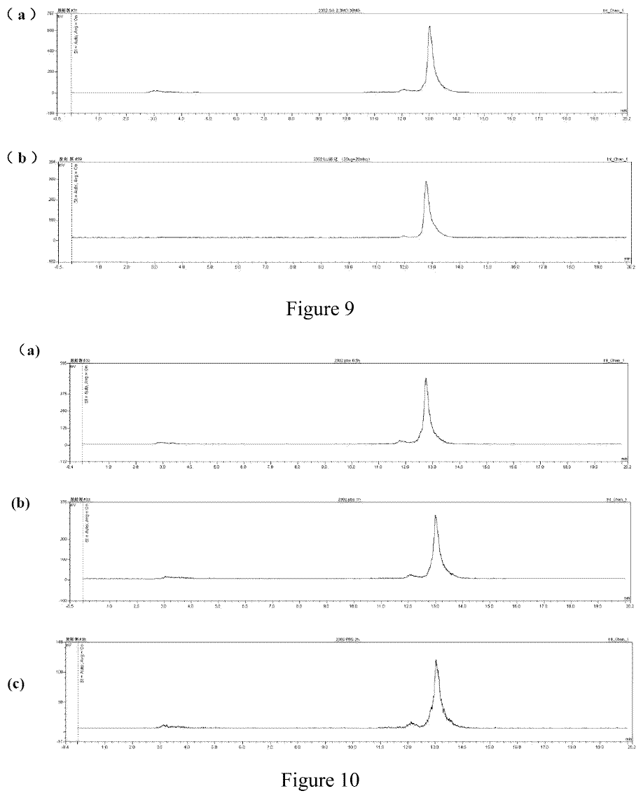

After purification through a C18 cartridge, the radiochemical purity of ⁶⁸Ga/¹⁷⁷Lu-SDYD02 was determined by radio-HPLC. Results showed 96% for ⁶⁸Ga-SDYD02 and 98% for ¹⁷⁷Lu-SDYD02 (FIG. 9).

### Stability of SDYD02 Radioligands in PBS Buffer

The purpose of this experiment was to test the stability of the radioligands under buffer conditions simulating the physiological environment. This stability is a prerequisite for further animal experiments. If no significant degradation of the ligand occurs, it indicates that the metal chelation and peptide bonds are stable in the buffer.

1. ⁶⁸Ga-SDYD02: ⁶⁸Ga-SDYD02 was added to PBS buffer (pH=7.4) and incubated at 37° C. The mixture samples were taken for radio-HPLC analysis after 0.5 hours, 1 hour, and 2 hours of incubation. The stability of ⁶⁸Ga-SDYD02 in PBS buffer: the radiochemical purities of ⁶⁸Ga-SDYD02 after incubation in PBS for 0.5 hours, 1 hour, and 2 hours were 96%, 95% and 93%, respectively (FIG. 10). The results indicate that ⁶⁸Ga-SDYD02 is quite stable in PBS buffer.

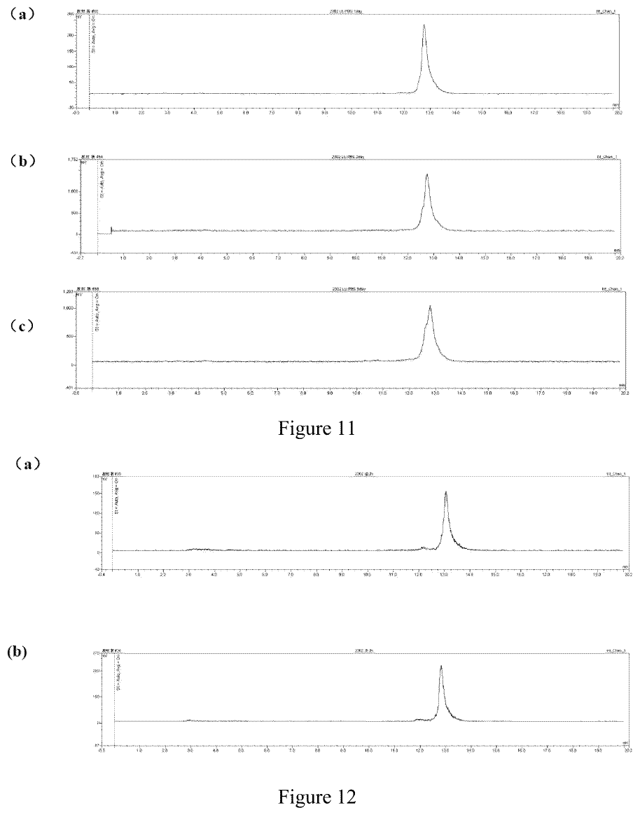

2. ¹⁷⁷Lu-SDYD02: ¹⁷⁷Lu-SDYD02 was added to PBS buffer (pH=7.4) and incubated at 37° C. The mixture samples were taken for radio-HPLC analysis after 24 hours, 48 hours, and 120 hours of incubation. The stability of ¹⁷⁷Lu-SDYD02 in PBS buffer: the radiochemical purities of ¹⁷⁷Lu-SDYD02 after incubation in PBS for 24 hours, 48 hours, and 120 hours were 95%, 91% and 87%, respectively (FIG. 11). The results indicate that ¹⁷⁷Lu-SDYD02 is quite stable in PBS buffer.

Competitive Experiment of SDYD02 Radioligand against Endogenous Free Amino Acids

To an EP tube containing 150 μL of PBS buffer, 150 μL of H₂O, and 50 μL of ⁶⁸Ga-SDYD02 was added 50 μL of cysteine hydrochloride solution (10 mM) or histidine solution (10 mM). The mixture was vortexed and incubated at 37° C. for 2 hours, after which samples were taken for radio-HPLC analysis.

This study utilized different concentrations of histidine and cysteine solutions to investigate whether histidine (or cysteine) competes with ⁶⁸Ga-SDYD02 for metal chelation. The study showed that after incubation with histidine and cysteine for 2 hours, the radiochemical purities of ⁶⁸Ga-SDYD02 remained at 92% and 91% respectively (FIG. 12), indicating that ⁶⁸Ga-SDYD02 did not undergo competitive chelation in the presence of histidine and cysteine.

### Determination of Hydrophilicity/Lipophilicity of SDYD02 Radioligands

10 μL of purified ⁶⁸Ga-SDYD02 was diluted to 500 μL with HEPES buffer (pH=7.4), then 500 μL of n-octanol was added. The mixture was vortexed for 30 min and then centrifuged. Subsequently, 450 μL each of the aqueous and organic phases were taken and centrifuged again. Then, 100 μL each of the aqueous and organic phases were taken, and the radioactivity was measured using a γ counter to calculate the n-octanol/water distribution coefficient (Log Do/w). The measured Log Do/w value for ⁶⁸Ga-SDYD02 was −2.47±0.03, and for ¹⁷⁷Lu-SDYD02 was −2.35±0.02.

### Example 3: Synthesis of SDYD05 and Related Radioactive Experimental Studies

### Synthesis and Analysis of SDYD05:

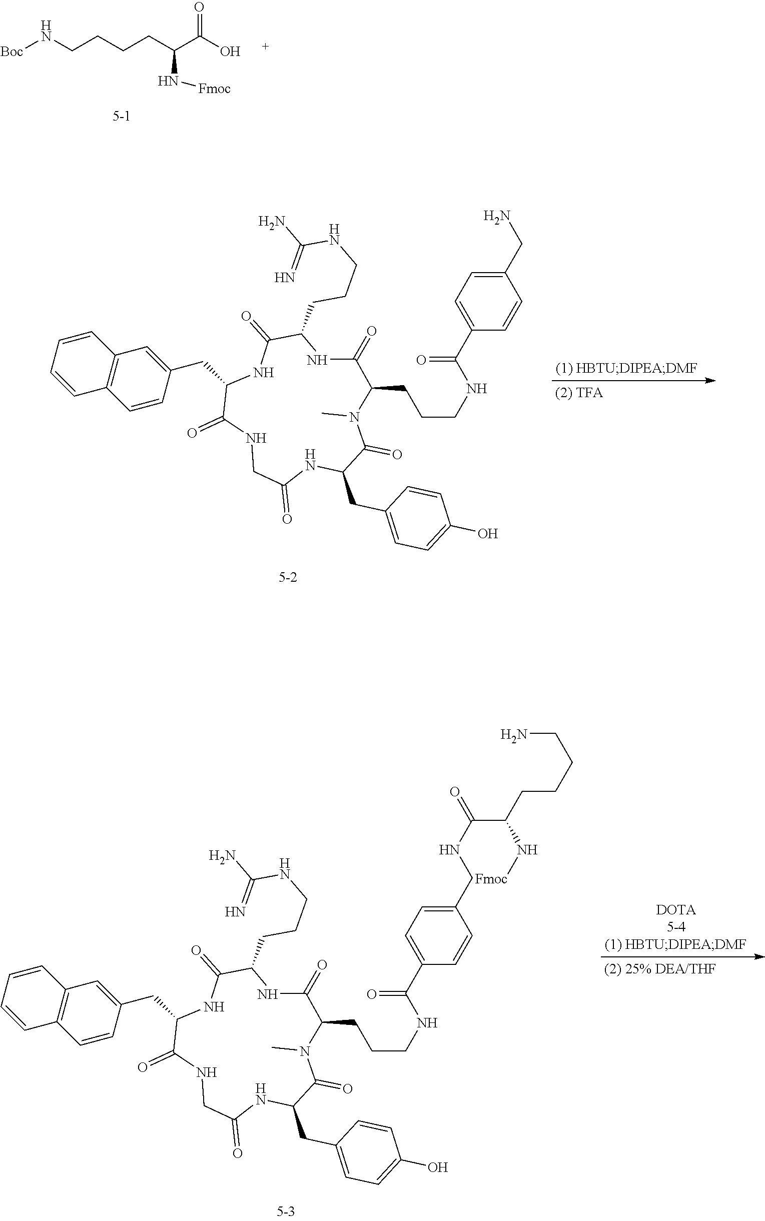

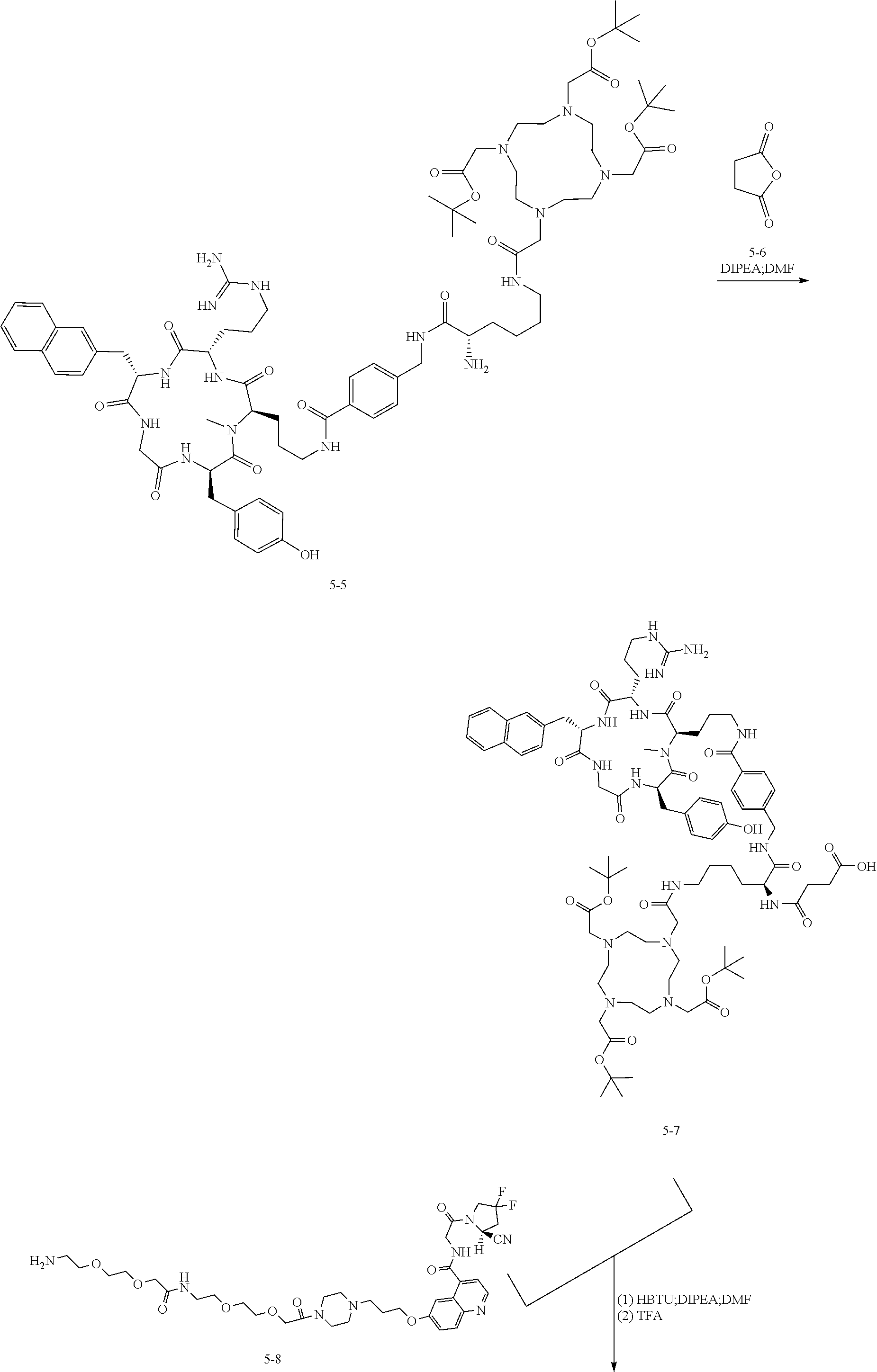

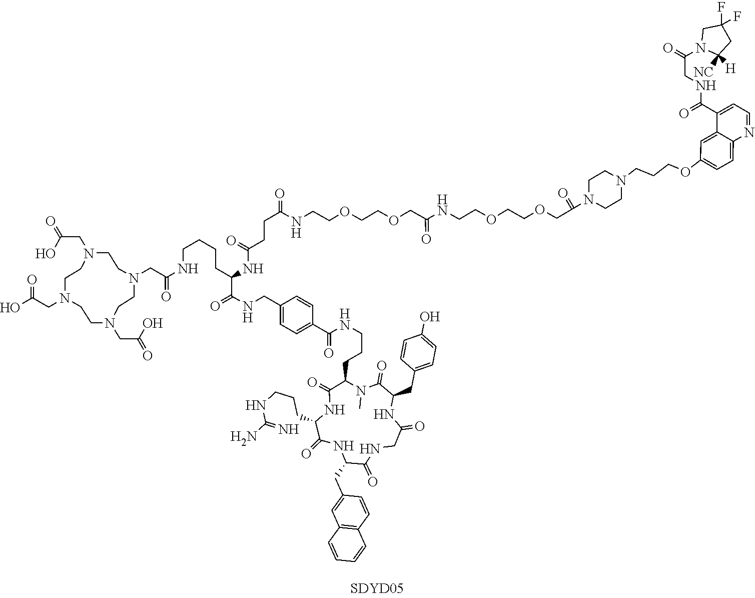

S1. Compound 5-1 (124 mg) and compound 5-2 (218 mg) were dissolved in 10 mL of DMF, added with 3 eq DIEA, followed by rapid addition of 1.1 eq HBTU. The reaction was allowed to proceed at room temperature for 10 minutes. Upon LC-MS confirmation of reaction completion, DMF was removed via rotary evaporation. 30 mL of TFA was added, and stirred at room temperature for 2 hours. The reaction solution was added slowly into 50 mL of ice-cold diethyl ether. Large amounts of solid precipitated out and were filtered to afford a crude product, which was purified by reversed-phase preparative LC to afford 186 mg of compound 5-3 (yield: 60%).

S2. Compound 5-3 (186 mg) and compound 5-4 (91 mg) were dissolved in 5 mL of DMF, added with 3 eq DIEA, followed by rapid addition of 1.1 eq HBTU. The reaction was allowed to proceed at room temperature for 10 minutes. Upon LC-MS confirmation of reaction completion, DMF was removed via rotary evaporation. 50 mL of 25% DEA/THF was added, and stirred at room temperature for 2 hours. The solvent was removed by rotary evaporation to give a crude product, which was purified by reversed-phase preparative HPLC to afford 129 mg of compound 5-5 (yield: 54%).

S3. Compound 5-5 (129 mg) and compound 5-6 (9 mg) were dissolved in 3 mL of DMF, followed by addition of 3 eq DIEA. The reaction was allowed to proceed at room temperature for 3 hours. Upon LC-MS confirmation of reaction completion, DMF was removed via rotary evaporation. Purification by reversed-phase preparative LC afforded 74 mg of compound 5-7 (yield: 53%).

S4. Compound 5-7 (74 mg) and compound 5-8 (35 mg) were dissolved in 3 mL of DMF, added with 3 eq DIEA, followed by rapid addition of 1.1 eq HBTU. The reaction was allowed to proceed at room temperature for 10 minutes. Upon LC-MS confirmation of reaction completion, DMF was removed via rotary evaporation. 5 mL of TFA was added, and stirred at room temperature for 30 minutes, and the reaction solution was added slowly into 50 mL of ice-cold diethyl ether. Large amounts of solid precipitated out and were filtered to afford a crude product, which was purified by reversed-phase preparative LC to afford 50 mg of target product, SDYD05 (DOTA-FAPI-Pentixafor) (yield: 50%).

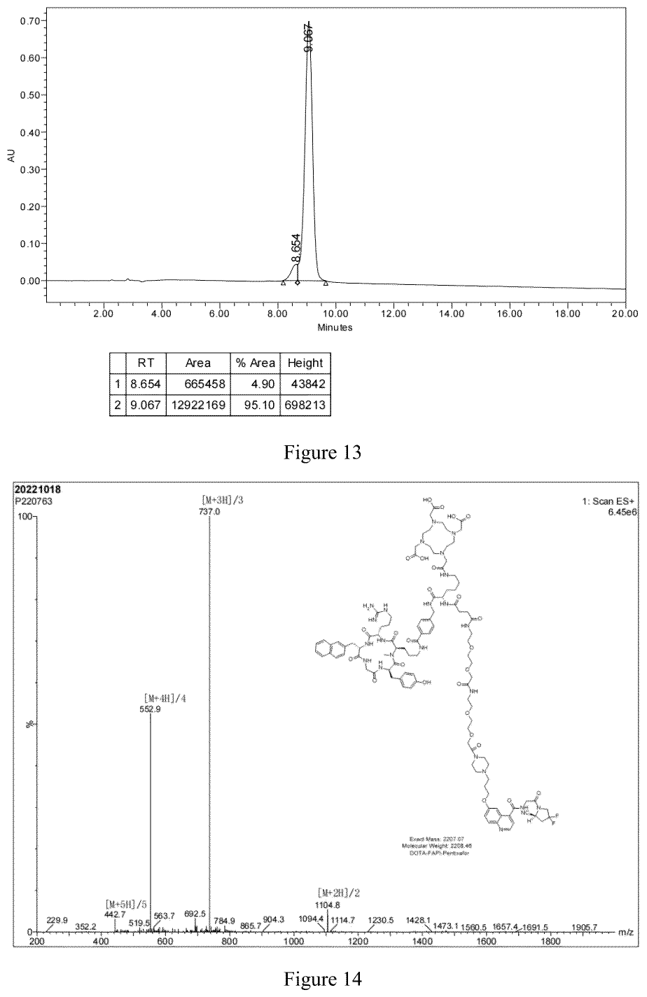

HPLC purity: 95.10% (FIG. 13). Molecular weight: theoretical 2207.07, measured 737.0 for [M+3H]/3, 552.9 for [M+4H]/4 (FIG. 14). Elemental analysis: N: 13.36%, C: 53.37%, H: 5.93%, which are consistent with the theoretical values. PI range of series of peaks is 6.617-7.126. Specific rotatory power is 3.8°.

### Radiolabeling of SDYD05 and Determination of Radiochemical Purity

Radiochemical purity refers to the percentage of radionuclide in the specified chemical form relative to the total radionuclide present in a radioactive sample. In this experiment, it specifically refers to the percentage of radioactivity successfully attached to the compound SDYD05 relative to the total radioactivity after purification.

1. ⁶⁸Ga Labeling: 10 μg of compound SDYD05 was added to 100 μL of NaOAc (0.25 M) buffer, followed by addition of 400 μL of ⁶⁸GaCl₃ solution (by rinsing the ⁶⁸Ge-⁶⁸Ga generator using 0.05 M HCl). The reaction mixture was shaken at 100° C. for 15 min. The crude product was purified using a C18 cartridge. The radiochemical purity of ⁶⁸Ga-SDYD05 was determined by radio-HPLC.

2. ¹⁷⁷Lu Labeling: 20 μg of compound SDYD05 was added to 150 μL of sodium acetate buffer (pH=4.6), followed by addition of 20 MBq of LuCl₃ solution containing ¹⁷⁷Lu. The reaction mixture was shaken at 100° C. for 50 minutes. The crude product was purified using a C18 cartridge. The radiochemical purity of 177Lu-SDYD05 was determined by radio-HPLC.

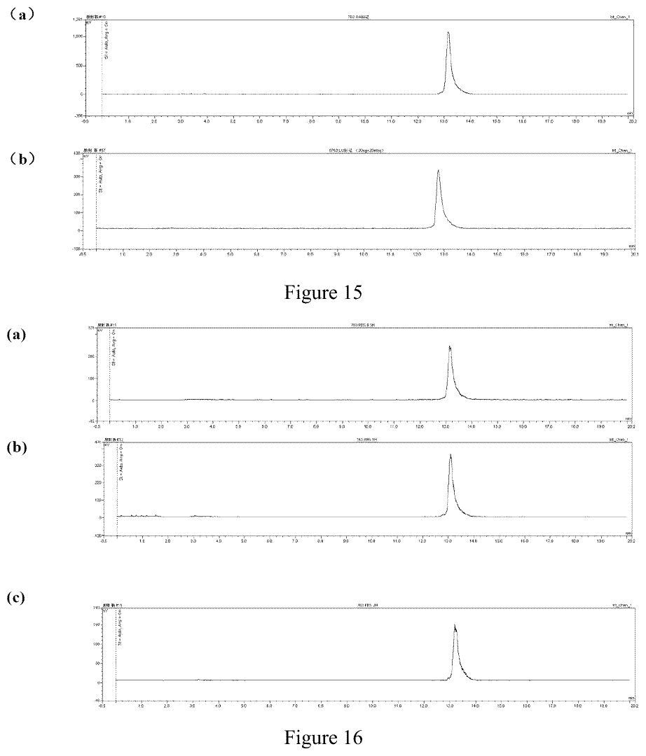

After purification through a C18 cartridge, the radiochemical purity of ⁶⁸Ga/¹⁷⁷Lu-SDYD05 was determined by radio-HPLC. Results showed 99% for ⁶⁸Ga-SDYD05 and 98% for ¹⁷⁷Lu-SDYD05 (FIG. 15).

### Stability of SDYD05 Radioligands in PBS Buffer

The purpose of this experiment was to test the stability of the radioligands under buffer conditions simulating the physiological environment. This stability is a prerequisite for further animal experiments. If no significant degradation of the ligand occurs, it indicates that the metal chelation and peptide bonds are stable in the buffer.

1. ⁶⁸Ga-SDYD05: ⁶⁸Ga-SDYD05 was added to PBS buffer (pH=7.4) and incubated at 37° C. The mixture samples were taken for radio-HPLC analysis after 0.5 hours, 1 hour, and 2 hours of incubation. The stability of ⁶⁸Ga-SDYD05 in PBS buffer: the radiochemical purities of ⁶⁸Ga-SDYD05 after incubation in PBS for 0.5 hours, 1 hour, and 2 hours were 98%, 96% and 95%, respectively (FIG. 16). The results indicate that ⁶⁸Ga-SDYD05 is quite stable in PBS buffer.

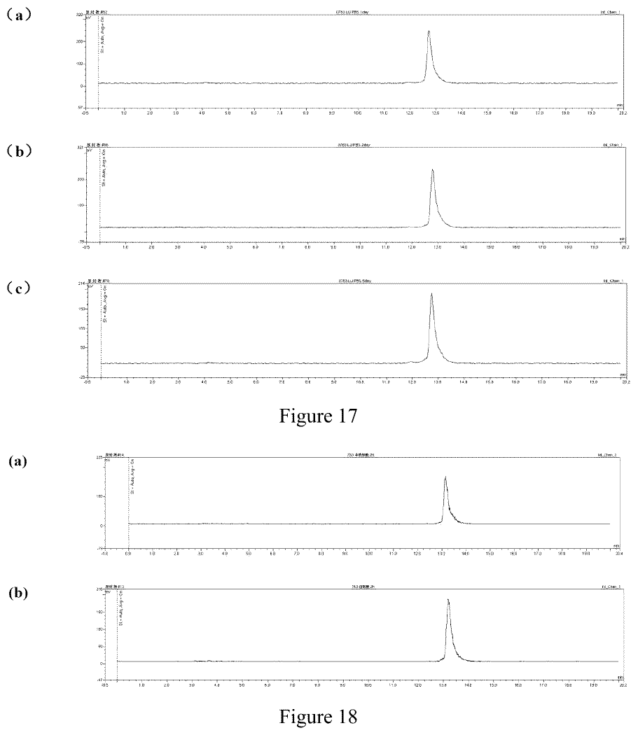

2. ¹⁷⁷Lu-SDYD05: ¹⁷⁷Lu-SDYD05 was added to PBS buffer (pH=7.4) and incubated at 37° C. The mixture samples were taken for radio-HPLC analysis after 24 hours, 48 hours, and 120 hours of incubation. The stability of ¹⁷⁷Lu-SDYD05 in PBS buffer: the radiochemical purities of ¹⁷⁷Lu-SDYD05 after incubation in PBS for 24 hours, 48 hours, and 120 hours were 97%, 96% and 96%, respectively (FIG. 17). The results indicate that ¹⁷⁷Lu-SDYD05 is quite stable in PBS buffer.

### Competitive Experiment of SDYD05 Radioligand Against Endogenous Free Amino Acids

To an EP tube containing 150 μL of PBS buffer, 150 μL of H₂O, and 50 μL of ⁶⁸Ga-SDYD05 was added 50 μL of cysteine hydrochloride solution (10 mM) or histidine solution (10 mM). The mixture was vortexed and incubated at 37° C. for 2 hours, after which samples were taken for radio-HPLC analysis. This study utilized different concentrations of histidine and cysteine solutions to investigate whether histidine (or cysteine) competes with ⁶⁸Ga-SDYD05 for metal chelation. The study showed that after incubation with histidine and cysteine for 2 hours, the radiochemical purity of ⁶⁸Ga-SDYD05 remained at 94% and 95% (FIG. 18), indicating that ⁶⁸Ga-SDYD05 did not undergo competitive chelation in the presence of histidine and cysteine.

### Determination of Hydrophilicity/Lipophilicity of SDYD05 Radioligands

10 μL of purified ⁶⁸Ga-SDYD05 was diluted to 500 μL with HEPES buffer (pH=7.4), then 500 μL of n-octanol was added. The mixture was vortexed for 30 min and then centrifuged. Subsequently, 450 μL each of the aqueous and organic phases were taken and centrifuged again. Then, 100 μL each of the aqueous and organic phases were taken, and the radioactivity was measured using a γ counter to calculate the n-octanol/water distribution coefficient (Log Do/w). The measured Log Do/w value for ⁶⁸Ga-SDYD05 was −2.37±0.02, and for ¹⁷⁷Lu-SDYD05 was −2.46±0.05.

### Example 4: Synthesis of SDYD06 and Related Radioactive Experimental Studies

### Synthesis and Analysis of SDYD06:

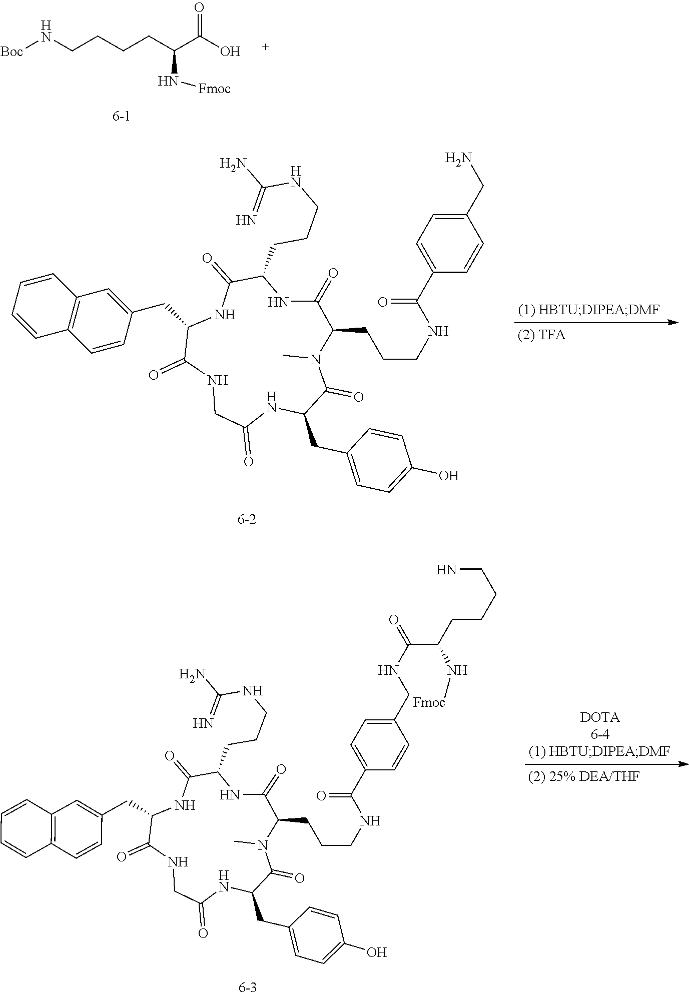

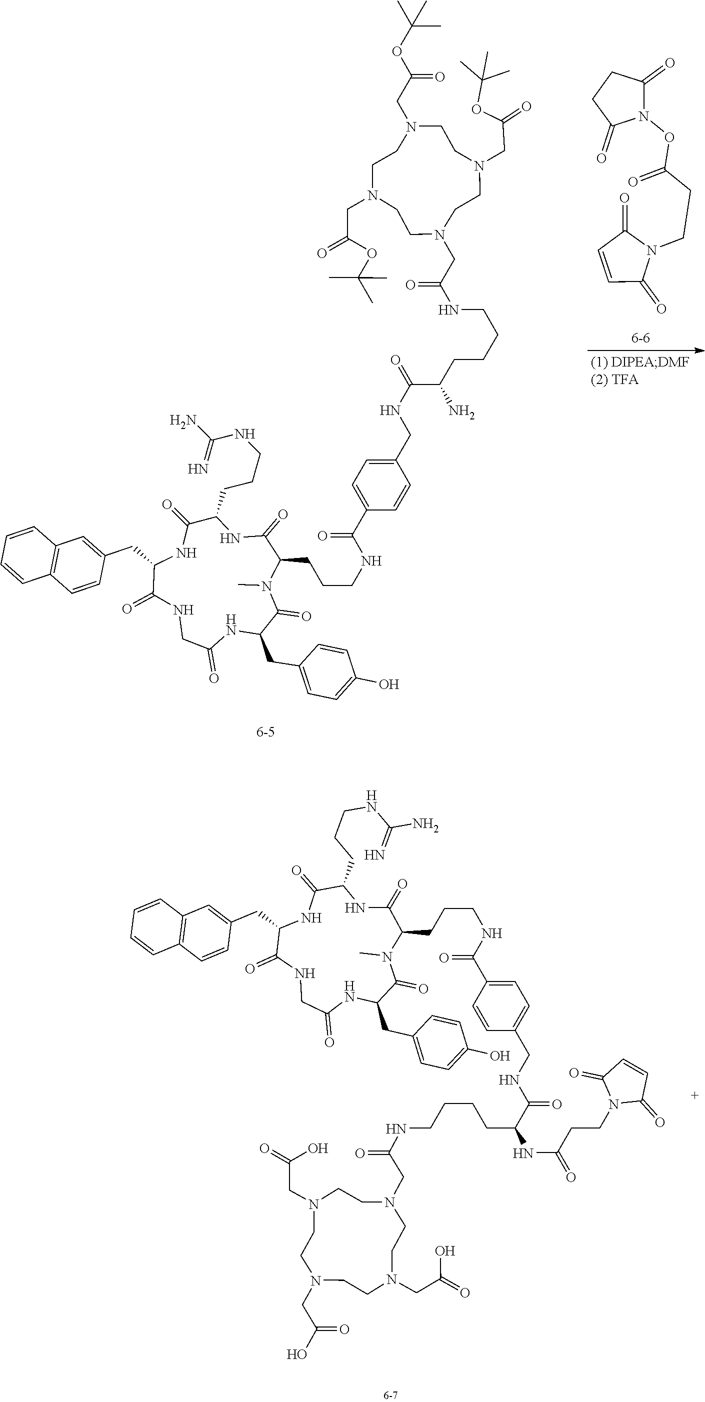

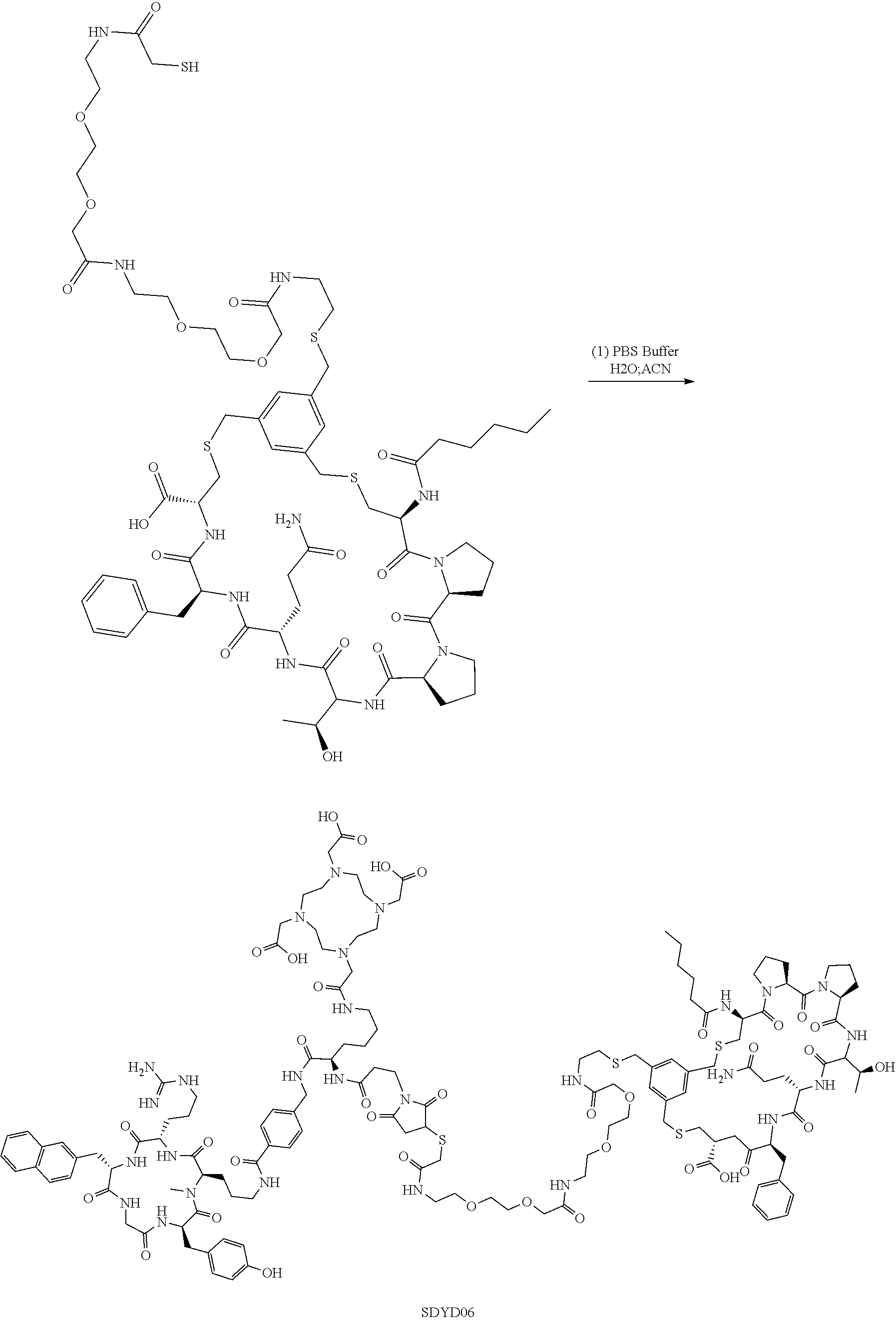

S1. Compound 6-1 (100 mg) was dissolved in 20 mL of DMF, added with 1.2 eq HBTU, 3.0 eq DIPEA, followed by 1.0 eq (178 mg) compound 6-2. The reaction was allowed to proceed at room temperature for 2 hours. Upon LC-MS confirmation of reaction completion, DMF was removed via rotary evaporation. 20 mL of TFA was added and stirred at room temperature for 30 min. Upon LC-MS confirmation of reaction completion, the reaction solution was added into ice-cold diethyl ether. Large amounts of solid precipitated out and were centrifuged. The solid was purified by reversed-phase preparative LC to afford 179.8 mg of compound 6-3 (yield: 71%).

S2. Compound 6-3 (179.8 mg) was dissolved in 20 mL of DMF, added with 1.2 eq HBTU and 3.0 eq DIPEA, followed by 1.0 eq (173.6 mg) Cmpd4. The reaction was allowed to proceed at room temperature for 2 hours. Upon LC-MS confirmation of reaction completion, DMF was removed via rotary evaporation. 15 mL of THF and 5 mL of DEA were added and stirred at room temperature for 2 hours. Upon LC-MS confirmation of reaction completion, the reaction solution was concentrated, and then purified by reversed-phase preparative LC to afford 168 mg of compound 6-5 (yield: 73%).

S3. Compound 6-5 (168 mg) was dissolved in 20 mL of DMF, added with 2.0 eq DIPEA, followed by 3.0 eq (88.4 mg) compound 6-6. The reaction was allowed to proceed at room temperature for 2 hours. Upon LC-MS confirmation of reaction completion, DMF was removed via rotary evaporation. 20 mL of TFA was added and stirred at room temperature for 2 hours. Upon LC-MS confirmation of reaction completion, the reaction solution was added into ice-cold diethyl ether. Large amounts of solid precipitated out and were centrifuged. The solid was purified by reversed-phase preparative LC to afford 127.9 mg of compound 6-7 (yield: 77%).

S4. Compound 6-7 (127.9 mg) was dissolved in 20 mL of H₂O/ACN=1:1, added with 1.2 eq (148 mg) compound 6-8, followed by 5 mL of 0.2 mol PBS Buffer solution (pH=7.2). The reaction was allowed to proceed at room temperature for 1 hour. Upon LC-MS confirmation of reaction completion, the reaction solution was directly purified by reversed-phase preparative LC to afford 150 mg of compound SDYD06 (yield: 60%).

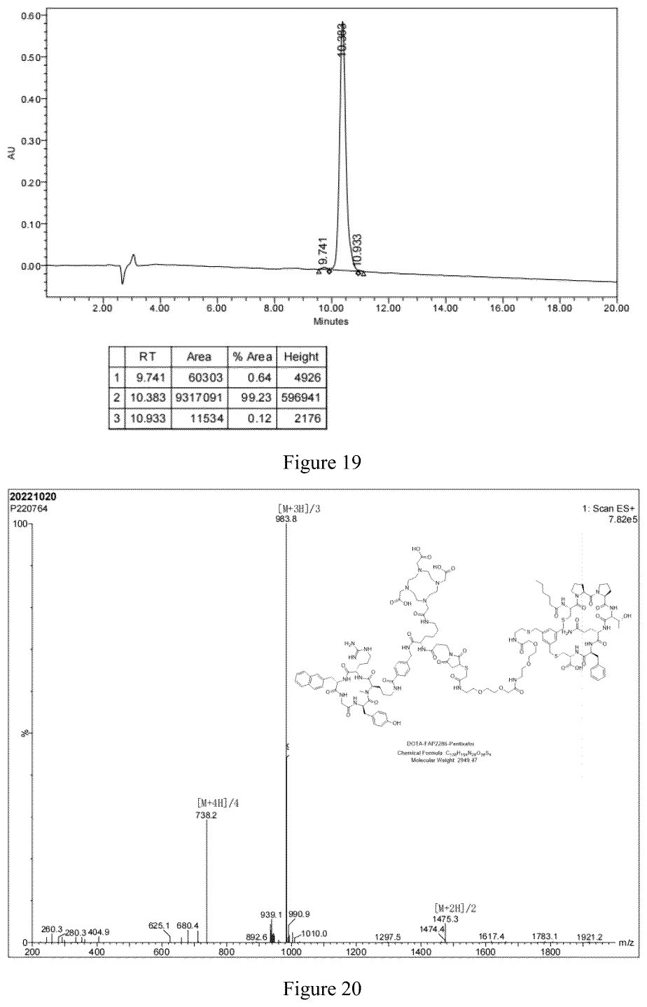

HPLC purity: 99.23% (FIG. 19). Molecular weight: theoretical 2949.44, measured 983.8 for [M+3H]/3, 738.2 for [M+4H]/4 (FIG. 20). Elemental analysis: N: 12.11%, C: 51.09%. H: 5.76%, which are consistent with the theoretical values. Specific rotatory power is −13.5°. During isoelectric point measurement, insoluble substance formed when the sample was prepared in pure water. Subsequent analysis of the supernatant after centrifugation showed no detectable signal.

### Radiolabeling of SDYD06 and Determination of Radiochemical Purity

Radiochemical purity refers to the percentage of radionuclide in the specified chemical form relative to the total radionuclide present in a radioactive sample. In this experiment, it specifically refers to the percentage of radioactivity successfully attached to the compound SDYD06 relative to the total radioactivity after purification.

1. ⁶⁸Ga Labeling: 10 μg of compound SDYD06 was added to 100 μL of NaOAc (0.25 M) buffer, followed by addition of 400 μL of ⁶⁸GaCl₃ solution (by rinsing the ⁶⁸Ge-⁶⁸Ga generator using 0.05 M HCl). The reaction mixture was shaken at 100° C. for 15 min. The crude product was purified using a C18 cartridge. The radiochemical purity of ⁶⁸Ga-SDYD06 was determined by radio-HPLC.

2. ¹⁷⁷Lu Labeling: 10 μg of compound SDYD06 was added to 150 μL of sodium acetate buffer (pH=4.6), followed by addition of 20MBq of LuCH, solution containing ¹⁷⁷Lu. The reaction mixture was shaken at 100° C. for 50 minutes. The crude product was purified using a C18 cartridge. The radiochemical purity of ¹⁷⁷Lu-SDYD06 was determined by radio-HPLC.

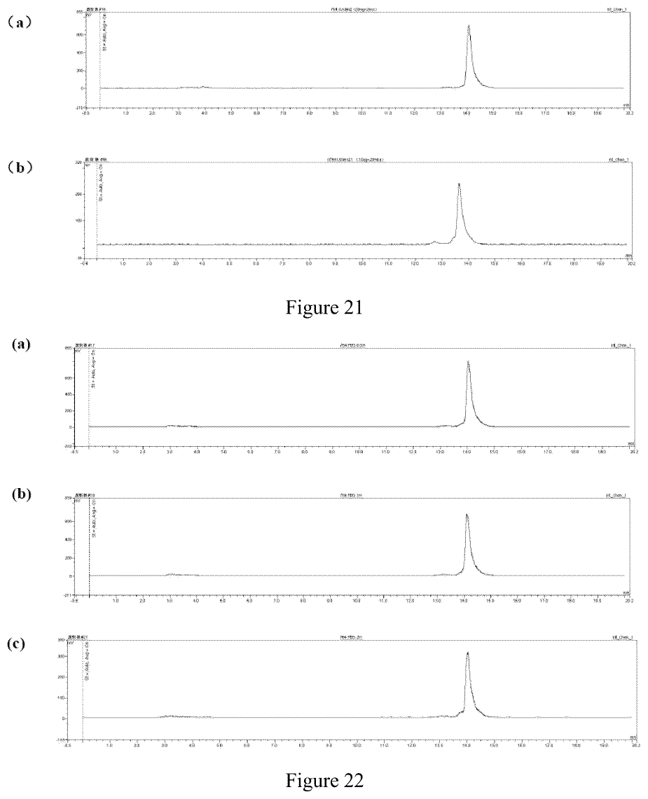

After purification through a C18 cartridge, the radiochemical purity of ⁶⁸Ga/¹⁷⁷Lu-SDYD06 was determined by radio-HPLC. Results showed 97% for ⁶⁸Ga-SDYD06 and 98% fovr 177Lu-SDYD06 (FIG. 21).

### Stability of SDYD06 Radioligands in PBS Buffer

The purpose of this experiment was to test the stability of the radioligands under buffer conditions simulating the physiological environment. This stability is a prerequisite for further animal experiments. If no significant degradation of the ligand occurs, it indicates that the metal chelation and peptide bonds are stable in the buffer.

1. ⁶⁸Ga-SDYD06: ⁶⁸Ga-SDYD06 was added to PBS buffer (pH=7.4) and incubated at 37° C. The mixture samples were taken for radio-HPLC analysis after 0.5, 1, and 2 hours of incubation. The stability of ⁶⁸Ga-SDYD06 in PBS buffer: the radiochemical purities of ⁶⁸Ga-SDYD06 after incubation in PBS for 0.5 hours, 1 hour, and 2 hours were 96%, 94% and 93%, respectively (FIG. 22). The results indicate that ⁶⁸Ga-SDYD06 is quite stable in PBS buffer.

2. ¹⁷⁷Lu-SDYD06: ¹⁷⁷Lu-SDYD06 was added to PBS buffer (pH=7.4) and incubated at 37° C. The mixture samples were taken for radio-HPLC analysis after 24 hours, 48 hours, and 120 hours of incubation. The stability of ¹⁷⁷Lu-SDYD06 in PBS buffer: the radiochemical purities of ¹⁷⁷Lu-SDYD05 after incubation in PBS for 24 hours, 48 hours, and 120 hours indicate that ¹⁷⁷Lu-SDYD06 is quite stable in PBS buffer.

### Competitive Experiment of SDYD06 Radioligand Against Endogenous Free Amino Acids

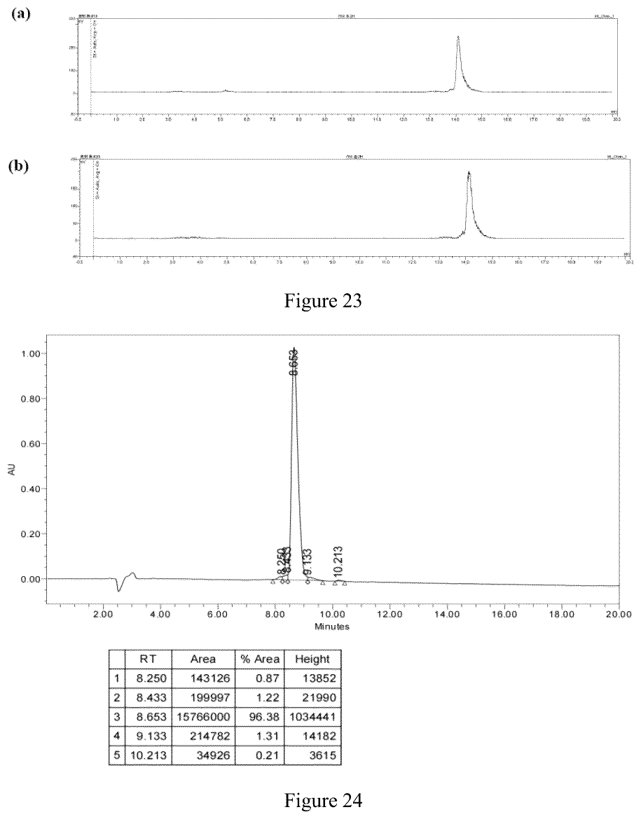

To an EP tube containing 150 μL of PBS buffer, 150 μL of H₂O, and 50 μL of ⁶⁸Ga-SDYD06 was added 50 μL of cysteine hydrochloride solution (10 mM) or histidine solution (10 mM). The mixture was vortexed and incubated at 37° C. for 2 hours, after which samples were taken for radio-HPLC analysis. This study utilized different concentrations of histidine and cysteine solutions to investigate whether histidine (or cysteine) competes with ⁶⁸Ga-SDYD06 for metal chelation. The study showed that after incubation with histidine and cysteine for 2 hours, the radiochemical purity of ⁶⁸Ga-SDYD06 remained at 92% and 93% (FIG. 23), indicating that ⁶⁸Ga-SDYD06 did not undergo competitive chelation in the presence of histidine and cysteine.

### Determination of Hydrophilicity/Lipophilicity of SDYD06 Radioligands

10 μL of purified ⁶⁸Ga-SDYD06 was diluted to 500 μL with HEPES buffer (pH=7.4), then 500 μL of n-octanol was added. The mixture was vortexed for 30 min and then centrifuged. Subsequently, 450 μL each of the aqueous and organic phases were taken and centrifuged again. Then, 100 μL each of the aqueous and organic phases were taken, and the radioactivity was measured using a γ counter to calculate the n-octanol/water distribution coefficient (Log Do/w). The measured Log Do/w value for ⁶⁸Ga-SDYD06 was −1.84±0.03, and for ¹⁷⁷Lu-SDYD06 was −1.94±0.08.

### Example 5: Synthesis of SDYD03 and Related Radioactive Experimental Studies

### Synthesis and Analysis of SDYD03:

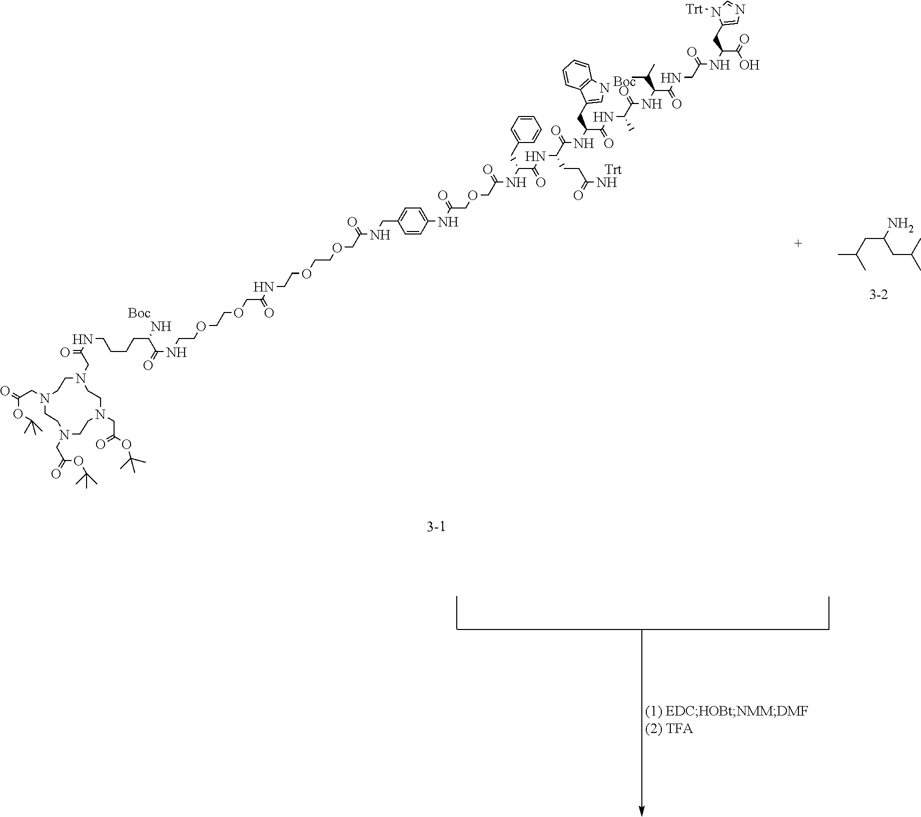

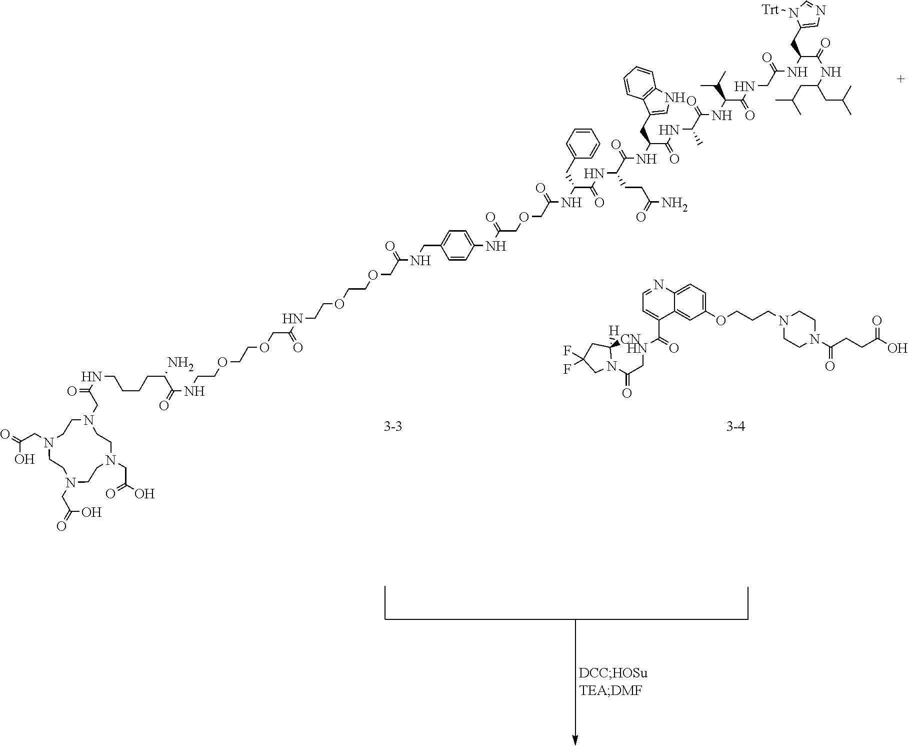

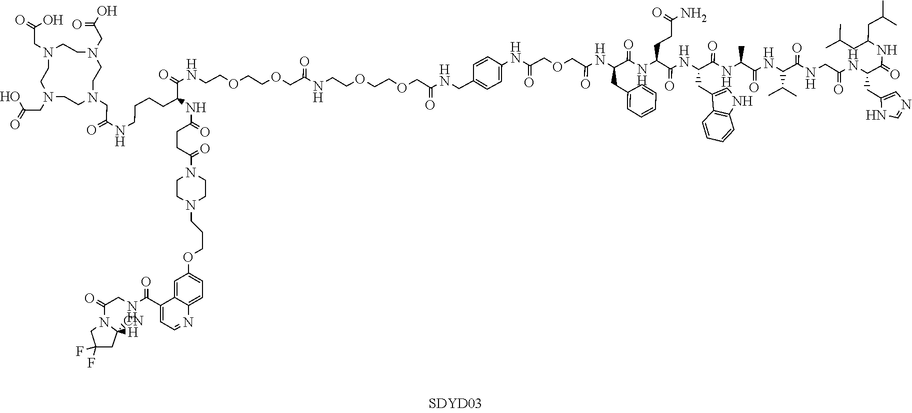

S1. Compound 3-1 (300 mg) was dissolved in 10 mL of DMF, added with 1.1 eq EDC and 1.1 eq HOBt. The reaction was allowed to proceed at room temperature for 10 min, and 2.0 eq (31.5 mg) compound 3-2 and 5.0 eq N-methylmorpholine were added. The reaction was allowed to proceed at room temperature for 2 hours. Upon LC-MS confirmation of reaction completion, DMF was removed via rotary evaporation. 10 mL of TFA was added, and stirred at room temperature for 2 hours. Large amounts of solid precipitated out with ice-cold diethyl ether and were centrifuged. The solid was purified by reversed-phase preparative LC to afford 127 mg of compound 3-3 (yield: 58%).

S2. Compound 3-3 (127 mg) was dissolved in 20 mL of DMF, followed by addition of 1.2 eq DCC and 1.2 eq HOSu. The reaction was allowed to proceed at room temperature for 6 hours. The precipitated solid was removed by filtering. To the filtrate were added 2.0 eq (75 mg) compound 3-4 and 2.0 eq TEA. The reaction was allowed to proceed at room temperature for 3 hours. Upon LC-MS confirmation of reaction completion, DMF was removed via rotary evaporation. Purification by reversed-phase preparative LC afforded 119 mg of target product SDYD03 (yield: 73%).

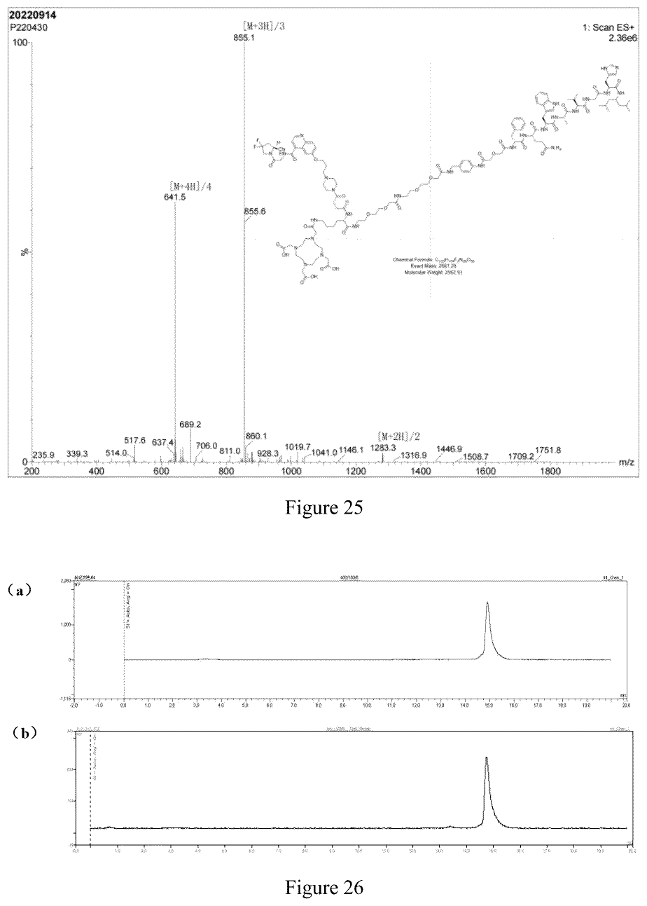

HPLC purity: 96.38% (FIG. 24). Molecular weight: theoretical 2562.91, measured 855.1 for [M+3H]/3, 641.5 for [M+4H]/4 (FIG. 25). Elemental analysis: N: 12.54%, C: 52.38%, H: 5.74%, which are consistent with the theoretical values. PI range of series of peaks is 5.992-7.095. Specific rotatory power is 32.3°.

### Radiolabeling of SDYD03 and Determination of Radiochemical Purity

Radiochemical purity refers to the percentage of radionuclide in the specified chemical form relative to the total radionuclide present in a radioactive sample. In this experiment, it specifically refers to the percentage of radioactivity successfully attached to the compound SDYD03 relative to the total radioactivity after purification.

1. ⁶⁸Ga Labeling: 10 μg of compound SDYD03 was added to 100 μL of NaOAc (0.25M) buffer, followed by addition of 400 μL ⁶⁸GaCl₃ solution (by rinsing the ⁶⁸Ge-⁶⁸Ga generator using 0.05 M HCl). The reaction mixture was shaken at 100° C. for 15 min. The crude product was purified using a C18 cartridge. The radiochemical purity of ⁶⁸Ga-SDYD03 was determined by radio-HPLC.

2. ¹⁷⁷Lu Labeling: 15 μg of compound SDYD03 was added to 150 μL of sodium acetate buffer (pH=4.6), followed by addition of 20MBq of LuCl₃ solution containing ¹⁷⁷Lu. The reaction mixture was shaken at 100° C. for 50 minutes. The crude product was purified using a C18 cartridge. The radiochemical purity of ¹⁷⁷Lu-SDYD03 was determined by radio-HPLC.

After purification through a C18 cartridge, the radiochemical purity of ⁶⁸Ga/¹⁷⁷Lu-SDYD03 was determined by radio-HPLC. Results showed 98% for ⁶⁸Ga-SDYD03 and 99% for ¹⁷⁷Lu-SDYD03 (FIG. 26).

### Stability of SDYD03 Radioligands in PBS Buffer

The purpose of this experiment was to test the stability of the radioligands under buffer conditions simulating the physiological environment. This stability is a prerequisite for further animal experiments. If no significant degradation of the ligand occurs, it indicates that the metal chelation and peptide bonds are stable in the buffer.

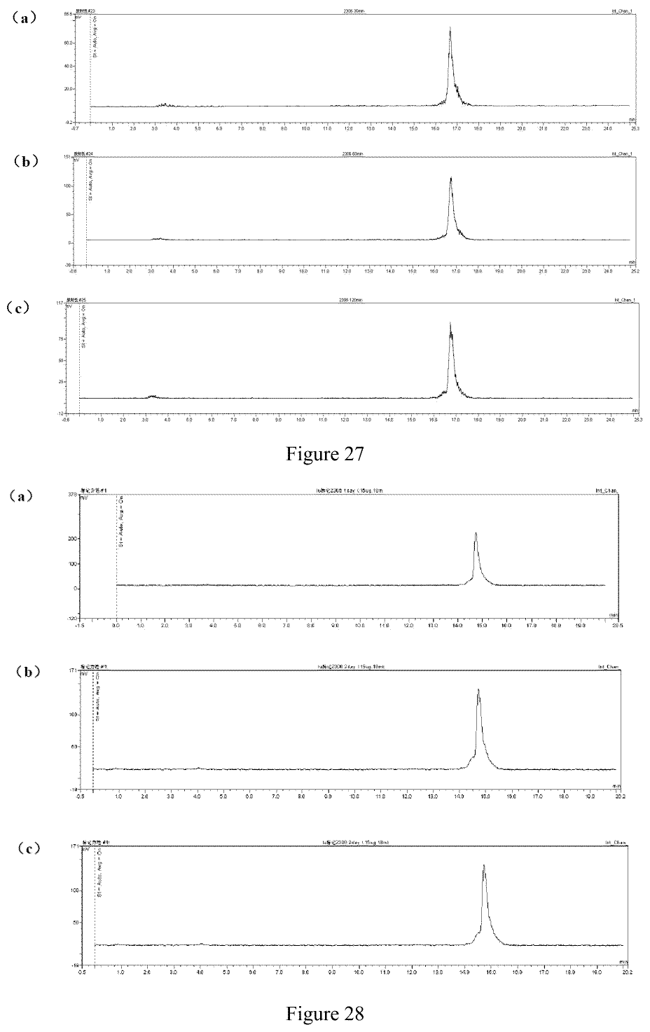

1. ⁶⁸Ga-SDYD03: ⁶⁸Ga-SDYD03 was added to PBS buffer (pH=7.4) and incubated at 37° C. The mixture samples were taken for radio-HPLC analysis after 0.5 hours, 1 hour, and 2 hours of incubation. The stability of ⁶⁸Ga-SDYD03 PBS in PBS buffer: the radiochemical purities of ⁶⁸Ga-SDYD03 after incubation in PBS for 0.5 hours, 1 hour, and 2 hours were 96%, 96% and 95%, respectively (FIG. 27). The results indicate that ⁶⁸Ga-SDYD03 is quite stable in PBS buffer.

2. ¹⁷⁷Lu-SDYD03: ¹⁷⁷Lu-SDYD03 was added to PBS buffer (pH=7.4) and incubated at 37° C. The mixture samples were taken for radio-HPLC analysis after 24 hours, 48 hours, and 120 hours of incubation. The stability of ¹⁷⁷Lu-SDYD03 in PBS buffer: the radiochemical purities of ¹⁷⁷Lu-SDYD03 after incubation in PBS for 24 hours, 48 hours, and 120 hours are 99%, 96% and 94%, respectively (FIG. 28). The results indicate that ¹⁷⁷Lu-SDYD03 is quite stable in PBS buffer.

### Determination of Hydrophilicity/Lipophilicity of SDYD03 Radioligands

10 μL of purified ⁶⁸Ga-SDYD03 was diluted to 500 μL with HEPES buffer (pH=7.4), then 500 μL of n-octanol was added. The mixture was vortexed for 30 min and then centrifuged. Subsequently, 450 μL each of the aqueous and organic phases were taken and centrifuged again. Then, 100 μL each of the aqueous and organic phases were taken, and the radioactivity was measured using a γ counter to calculate the n-octanol/water distribution coefficient (Log Do/w). The measured Log Do/w value for ⁶⁸Ga-SDYD03 was −1.72±0.04, and for ¹⁷⁷Lu-SDYD03 was −1.52±0.05.

### Example 6: Synthesis of SDYD04 and Related Radioactive Experimental Studies

### Synthesis and Analysis of SDYD04

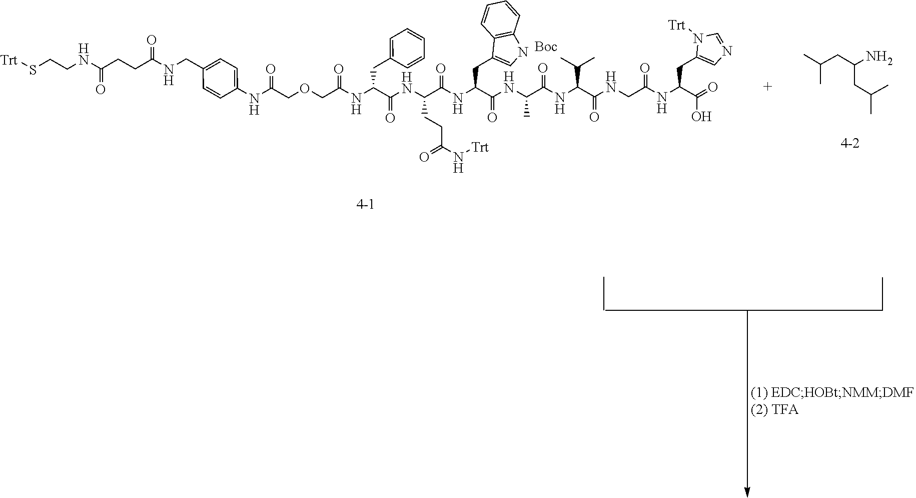

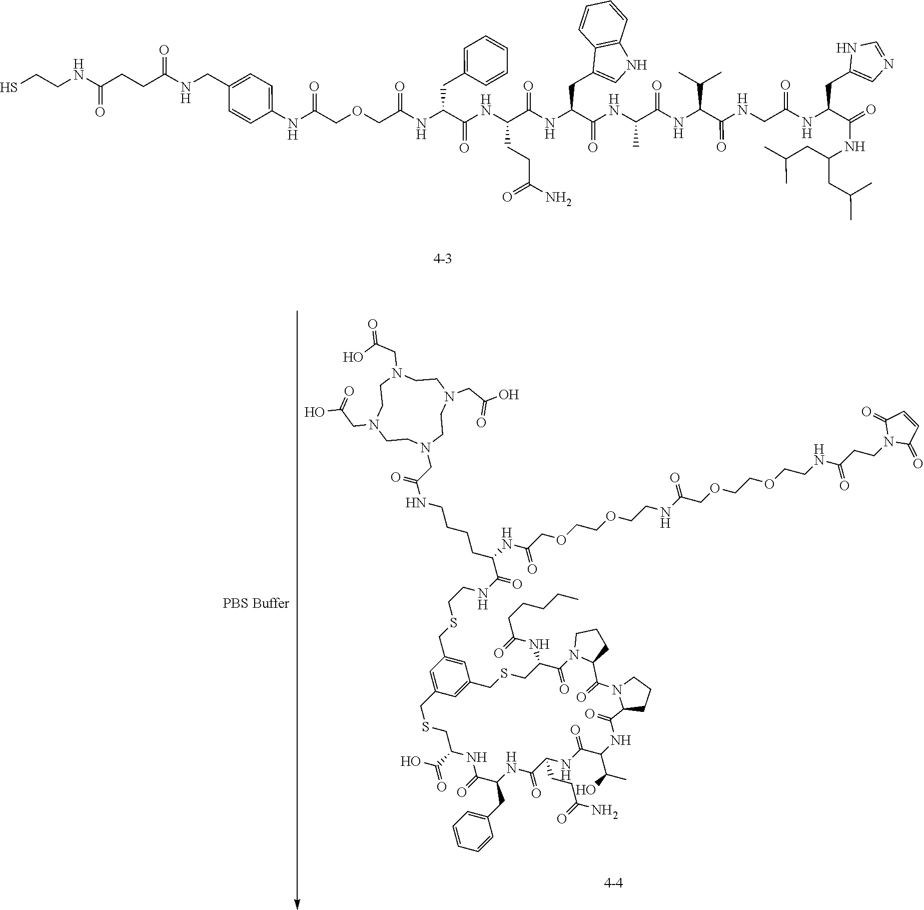

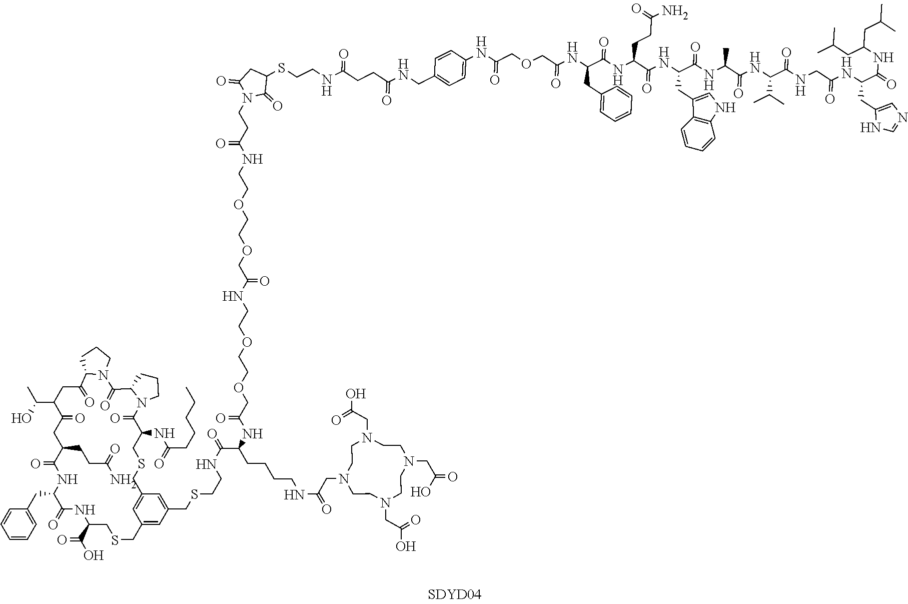

S1. Compound 4-1 (400 mg) was dissolved in 5 mL of DMF and stirred in ice bath for 5 minutes, followed by addition of 1.1 eq HOBt and 1.1 eq EDC. After stirring for 5 minutes, compound 4-2 (30 mg) was added, followed by addition of 3.0 eq NMM. The reaction was allowed to return to room temperature and proceed for 2 hours. Upon LC-MS confirmation of reaction completion, DMF was removed via rotary evaporation. The residue was dissolved in 5 mL of TFA, stirred at room temperature for 2.5 hours, and then the mixture was added into 50 mL of ice-cold diethyl ether. Large amounts of solid precipitated out and were centrifuged and dried. Purification by reversed-phase preparative LC afforded 158 mg of compound 4-3 (yield: 60%).

S2. Compound 4-3 (158 mg) and compound 4-4 (240 mg) were dissolved in a mixture of acetonitrile and water (1:1, 5 mL), and then added under a nitrogen atmosphere into 0.2 M PBS buffer solution (pH=7.2) (5 mL). The reaction was allowed to proceed at room temperature for 1 hour. Upon LC-MS confirmation of reaction completion, Purification by reversed-phase chromatography afforded 210 mg of target product SDYD04 (yield: 53%).

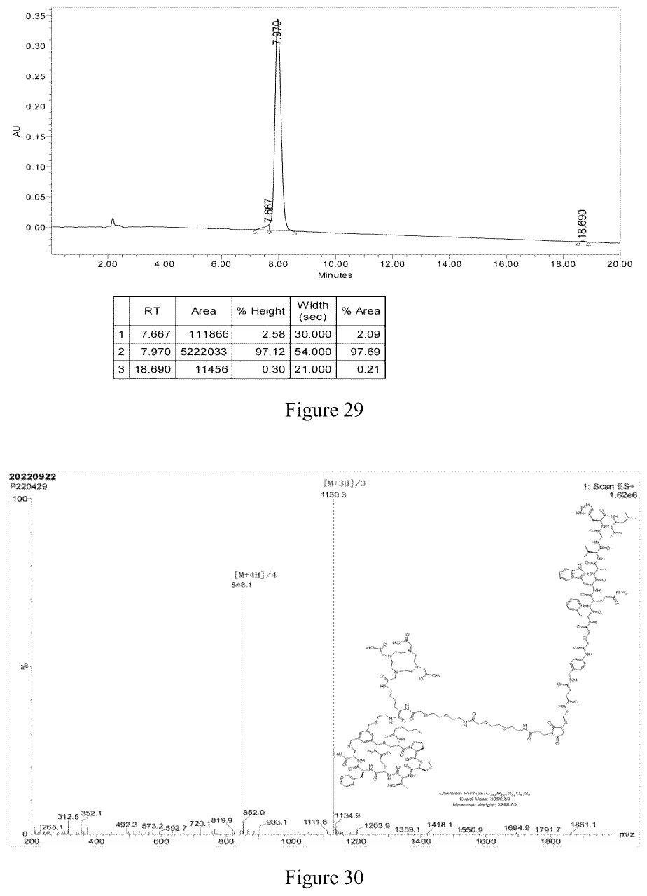

HPLC purity: 97.69% (FIG. 29). Molecular weight: theoretical 2562.91, measured 1130.3 for [M+3H]/3, 848.1 for [M+4H]/4 (FIG. 30). Elemental analysis: N: 12.98%, C: 50.59%, H: 5.57%, which are consistent with the theoretical values. During isoelectric point measurement, insoluble material formed when the sample was prepared in pure water. Subsequent analysis of the supernatant after centrifugation showed no detectable signal. Specific rotatory power is −22.2°.

### Radiolabeling of SDYD04 and Determination of Radiochemical Purity

Radiochemical purity refers to the percentage of radionuclide in the specified chemical form relative to the total radionuclide present in a radioactive sample. In this experiment, it specifically refers to the percentage of radioactivity successfully attached to the compound SDYD04 relative to the total radioactivity after purification.

1. ⁶⁸Ga Labeling: 10 μg of compound SDYD04 was added to 100 μL of NaOAc (0.25 M) buffer, followed by addition of 400 μL ⁶⁸GaCl₃ solution (by rinsing the 68Ge-68Ga generator using 0.05 M HCl). The reaction mixture was shaken at 100° C. for 15 min. The crude product was purified using a C18 cartridge. The radiochemical purity of ⁶⁸Ga-SDYD04 was determined by radio-HPLC.

2. ¹⁷⁷Lu Labeling: 20 μg of compound SDYD04 was added to 150 μL of sodium acetate buffer (pH=4.6), followed by addition of 20MBq of LuCl₃ solution containing ¹⁷⁷Lu. The reaction mixture was shaken at 100° C. for 50 minutes. The crude product was purified using a C18 cartridge. The radiochemical purity of 177Lu-SDYD04 was determined by radio-HPLC.

After purification through a C18 cartridge, the radiochemical purity of ⁶⁸Ga/¹⁷⁷Lu-SDYD04 was determined by radio-HPLC. Results showed 98% for ⁶⁸Ga-SDYD04 and 98% for ¹⁷⁷Lu-SDYD04 (FIG. 31).

### Stability of SDYD04 Radioligands in PBS Buffer

The purpose of this experiment was to test the stability of the radioligands under buffer conditions simulating the physiological environment. This stability is a prerequisite for further animal experiments. If no significant degradation of the ligand occurs, it indicates that the metal chelation and peptide bonds are stable in the buffer.

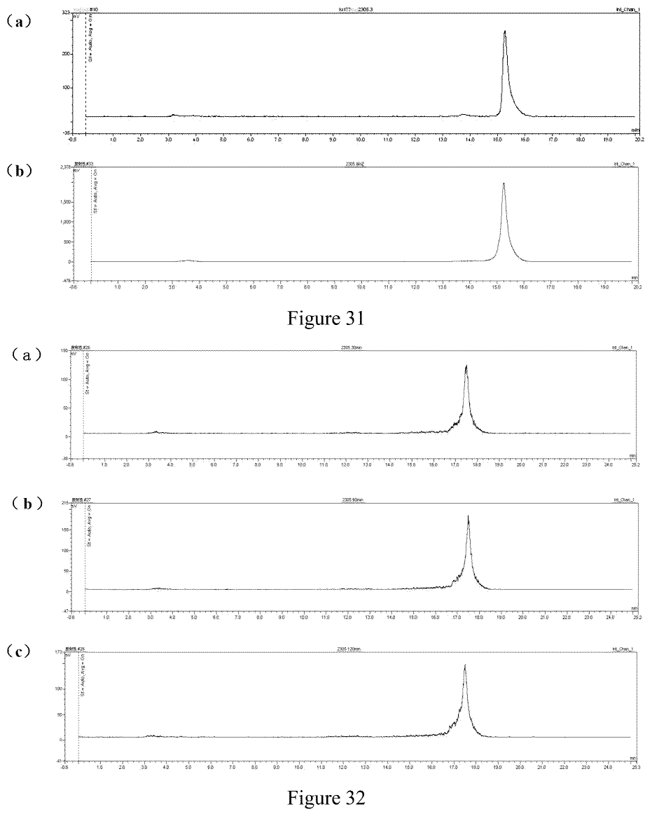

1. ⁶⁸Ga-SDYD04: ⁶⁸Ga-SDYD04 was added to PBS buffer (pH=7.4) and incubated at 37° C. The mixture samples were taken for radio-HPLC analysis after 0.5 hours, 1 hour, and 2 hours of incubation. The stability of ⁶⁸Ga-SDYD04 in PBS buffer: the radiochemical purities of ⁶⁸Ga-SDYD04 after incubation in PBS for 0.5 hours, 1 hour, and 2 hours were 97%, 97% and 95%, respectively (FIG. 32). The results indicate that ⁶⁸Ga-SDYD04 is quite stable in PBS buffer.

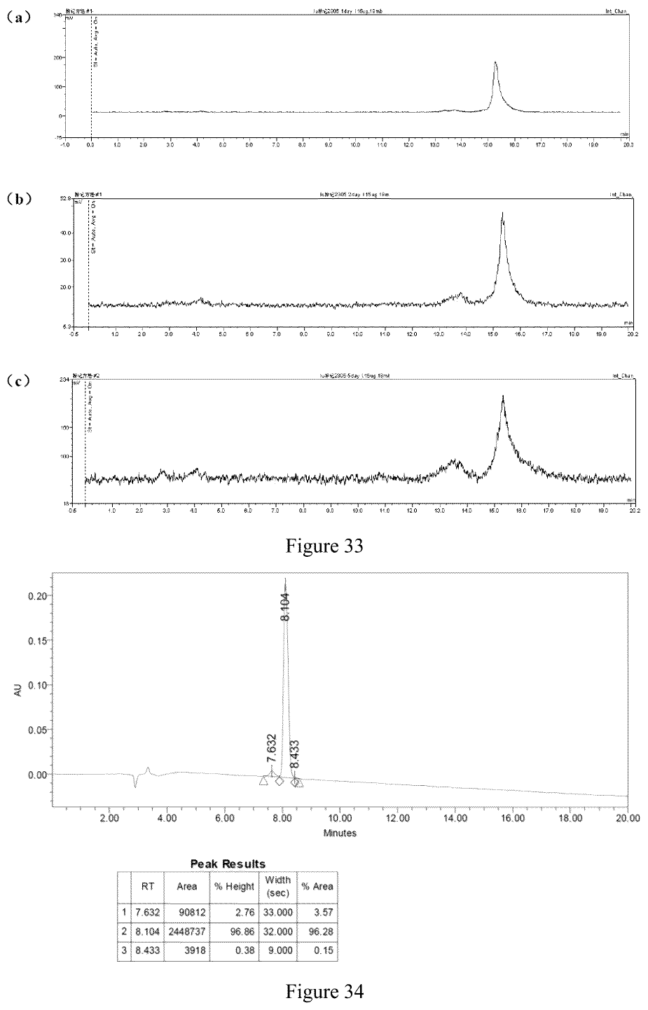

2, ¹⁷⁷Lu-SDYD04: ¹⁷⁷Lu-SDYD04 was added to PBS buffer (pH=7.4) and incubated at 37° C. The mixture samples were taken for radio-HPLC analysis after 24 hours, 48 hours, and 120 hours of incubation. The stability of ¹⁷⁷Lu-SDYD04 in PBS buffer: the radiochemical purities of ¹⁷⁷Lu-SDYD04 after incubation in PBS for 24 hours, 48 hours, and 120 hours were 97%, 84% and 63%, respectively (FIG. 33). The results indicate that ¹⁷⁷Lu-SDYD04 is quite stable in PBS buffer.

### Determination of Hydrophilicity/Lipophilicity of SDYD04 Radioligands

10 μL of purified ⁶⁸Ga-SDYD04 was diluted to 500 μL with HEPES buffer (pH=7.4), then 500 μL of n-octanol was added. The mixture was vortexed for 30 min and then centrifuged. Subsequently, 450 μL each of the aqueous and organic phases were taken and centrifuged again. Then, 100 μL each of the aqueous and organic phases were taken, and the radioactivity was measured using a γ counter to calculate the n-octanol/water distribution coefficient (Log Do/w). The measured Log Do/w value for ⁶⁸Ga-SDYD04 was −1.63±0.07, and for ¹⁷⁷Lu-SDYD04 was −1.39±0.03.

### Example 7: Synthesis of SDYD07 and Related Radioactive Experimental Studies

### Synthesis and Analysis of SDYD07:

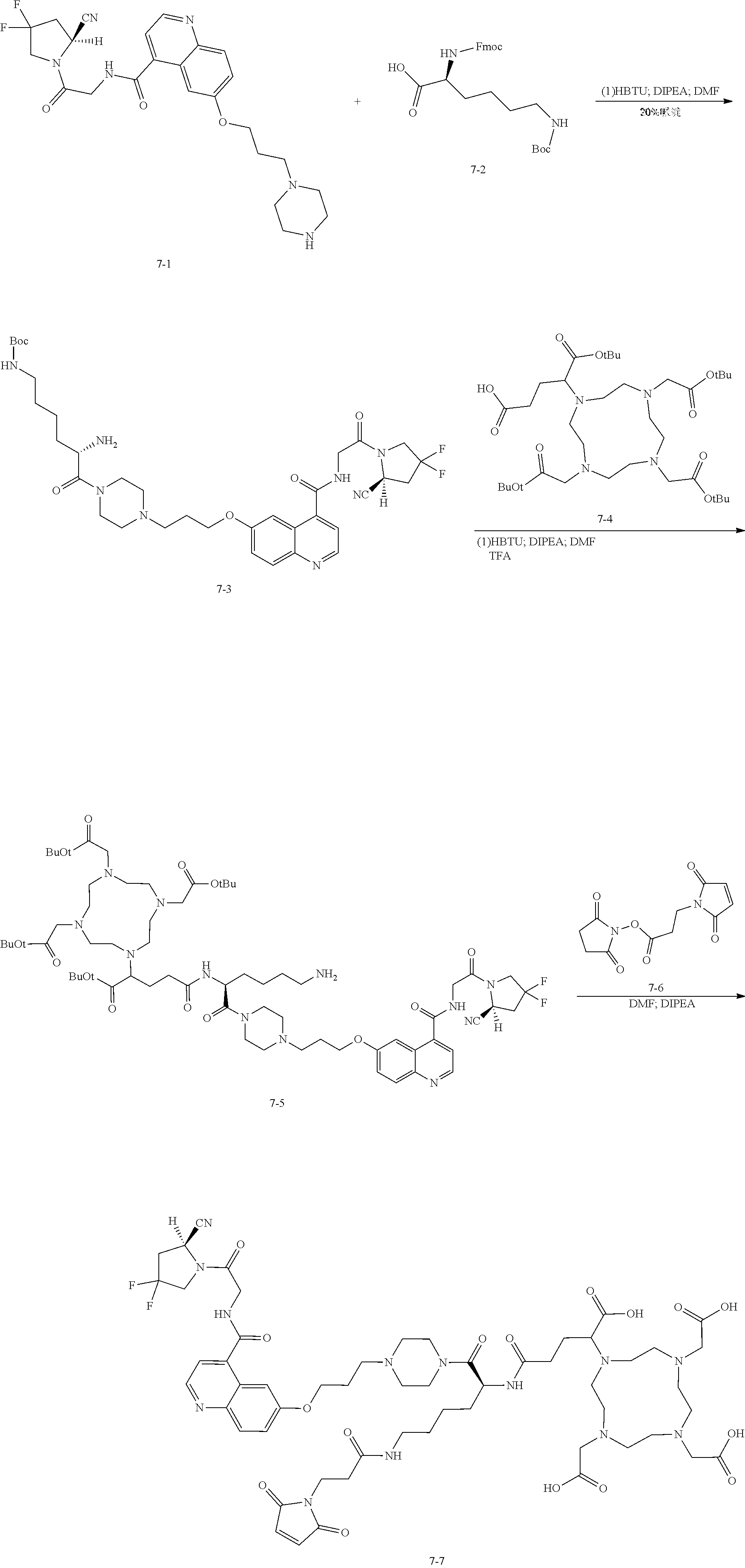

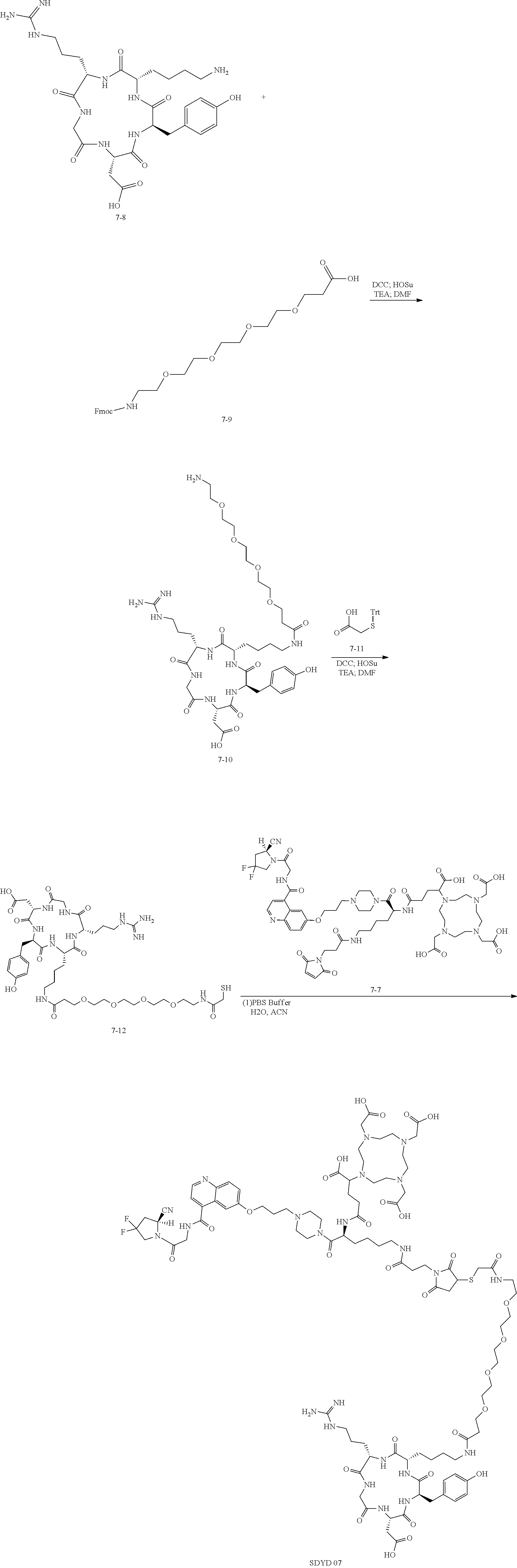

S1. Compound 7-1 (200 mg) was dissolved in 20 mL of DMF, added with 1.2 eq HBTU and 3.0 eq DIPEA, followed by addition of 1.1 eq (211 mg) compound 7-2. The reaction was allowed to proceed at room temperature for 2 hours. Upon LC-MS confirmation of reaction completion, 20 mL of 20% piperidine was added and the reaction mixture was then concentrated to dryness. The residue was washed with ice-cold diethyl ether 3 times and concentrated under reduced pressure to dryness. Purification by reversed-phase preparative LC afforded 190 mg of compound 7-3 (yield: 64.8%).

S2. Compound 7-4 (190 mg) was dissolved in 20 mL of DMF, added with 1.2 eq HBTU and 3.0 eq DIPEA, followed by addition of 1.0 eq (190 mg) 7-3. The reaction was allowed to proceed at room temperature for 2 hours. Upon LC-MS confirmation of reaction completion, DMF was removed via rotary evaporation. 20 mL of TFA was added and stirred at room temperature for 3 min. Upon LC-MS confirmation of reaction completion, the mixture was added into ice-cold diethyl ether. Large amounts of solid precipitated out and were centrifuged. The solid was purified by reversed-phase preparative LC to afford 210 mg of compound 7-5 (yield: 60.9%).

S3. Compound 7-5 (210 mg) was dissolved in 20 mL of DMF, added with 2.0 eq DIPEA, followed by 1.1 eq (47 mg) compound 7-6. The reaction was allowed to proceed at room temperature for 2 hours. Upon LC-MS confirmation of reaction completion, DMF was removed via rotary evaporation. 20 mL of TFA was added and stirred at room temperature for 2 hours. Upon LC-MS confirmation of reaction completion, ice-cold diethyl ether was added. Large amounts of solid precipitated out and were centrifuged. The solid was purified by reversed-phase preparative LC to afford 122.9 mg of compound 7-7 (yield: 62%).

S4. Compound 7-8 (300 mg) was dissolved in 20 mL of DMF, and added with 1.2 eq DCC and 1.2 eq HOSu. The reaction was allowed to proceed at room temperature for 6 hours. The precipitated solid was removed by filtering. To the filtrate were added 1.2 eq (283 mg) compound 7-9 and 2 eq TEA. The reaction was allowed to proceed at room temperature for 3 hours. Upon LC-MS confirmation of reaction completion, 20 mL 20% piperidine was added. DMF was removed via rotary evaporation. The residue was washed twice with diethyl ether, and dried under reduced pressure. Purification by reversed-phase preparative LC afforded 243 mg of compound 7-10 (yield: 58%).

S5. Compound 7-11 (103 mg) was dissolved in 20 mL of DMF, added with 1.2 eq DCC and 1.2 eq HOSu. The reaction was allowed to proceed at room temperature for 6 hours. The precipitated solid was removed by filtering. To the filtrate were added 0.9 eq (243 mg) compound 7-10 and 2 eq TEA. The reaction was allowed to proceed at room temperature for 3 hours. Upon LC-MS confirmation of reaction completion. DMF was removed via rotary evaporation. 20 mL of TFA was added and stirred at room temperature for 3 min. Upon LC-MS confirmation of reaction completion, ice-cold diethyl ether was added. Large amounts of solid precipitated out and were centrifuged. The solid was purified by reversed-phase preparative LC to afford 166 mg of compound 7-12 (yield: 63%).

S6. Compound 7-7 (216 mg) was dissolved in 20 mL of H₂O/ACN=1:1, added with 1.0 eq (166 mg) compound 7-12, followed by 5 mL of 0.2 mol PBS Buffer (pH=7.2). The reaction was allowed to proceed at room temperature for 1 hour. Upon LC-MS confirmation of reaction completion, the reaction mixture was directly purified by reversed-phase preparative LC to afford 194.8 mg of compound SDYD07 (yield: 51%).

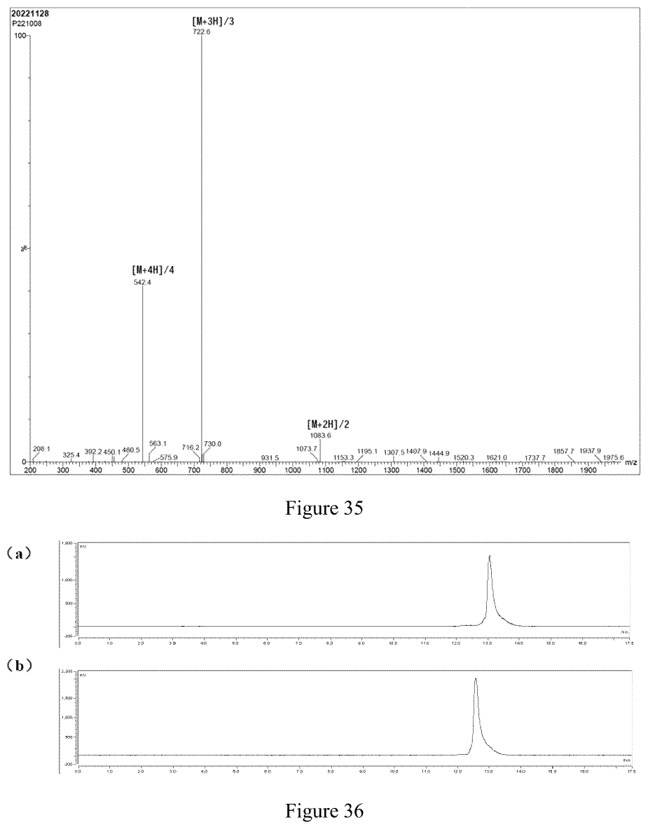

HPLC purity: 96.86% (FIG. 34). Molecular weight: theoretical 2165.36, measured 722.6 for [M+3H]/3, 542.4 for [M+4H]/4 (FIG. 35). Elemental analysis: N: 11.78%, C: 49.73%, H: 5.01%, which are consistent with the theoretical values. Main peak's PI is 4.809. Specific rotatory power is −32.2°.

### Radiolabeling of SDYD07 and Determination of Radiochemical Purity

Radiochemical purity refers to the percentage of radionuclide in the specified chemical form relative to the total radionuclide present in a radioactive sample. In this experiment, it specifically refers to the percentage of radioactivity successfully attached to the compound SDYD07 relative to the total radioactivity after purification.

1. ⁶⁸Ga Labeling: 2.3 nmol compound SDYD07 was added to 100 μL of NaOAc (0.25 M) buffer, followed by addition of 400 μL of ⁶⁸GaCl₃ solution (by rinsing the ⁶⁸Ge-⁶⁸Ga generator using 0.05 M HCl). The reaction mixture was shaken at 100° C. for 15 min. The crude product was purified using a C18 cartridge. The radiochemical purity of ⁶⁸Ga-SDYD07 was determined by radio-HPLC.

2. ¹⁷⁷Lu Labeling: 3.5 nmol compound SDYD07 was added to 130 μL of sodium acetate buffer (pH=5.6), followed by addition of 37MBq of LuCl₃ solution containing ¹⁷⁷Lu. The reaction mixture was shaken at 95° C. for 20 min. The crude product was purified using a C18 cartridge. The radiochemical purity of ¹⁷⁷Lu-SDYD07 was determined by radio-HPLC.

After purification through a C18 cartridge, the radiochemical purity of ⁶⁸Ga/¹⁷⁷Lu-SDYD07 was determined by radio-HPLC. Results showed 99% for ⁶⁸Ga-SDYD07 and 99% for ¹⁷⁷Lu-SDYD07 (FIG. 36).

### Stability of SDYD07 Radioligands in Phosphate Buffer Solution

The purpose of this experiment was to test the stability of the radioligands under buffer conditions simulating the physiological environment. This stability is a prerequisite for further animal experiments. If no significant degradation of the ligand occurs, it indicates that the metal chelation and peptide bonds are stable in the buffer.

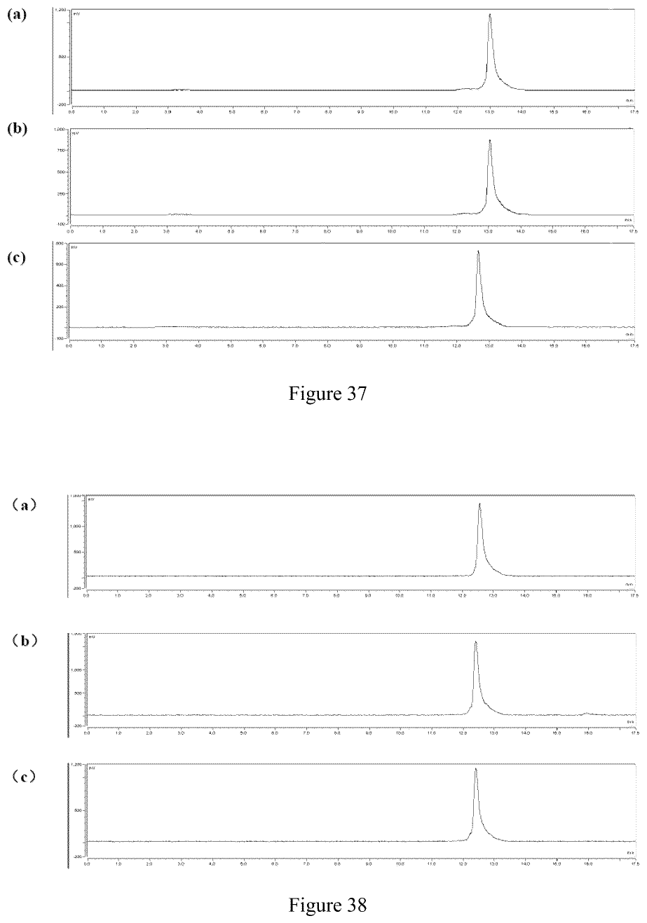

1. ⁶⁸Ga-SDYD07: ⁶⁸Ga-SDYD07 was added to PBS buffer (pH=7.4) and incubated at 37° C. The mixture samples were taken for radio-HPLC analysis after 0.5 hours, 1 hour, and 2 hours of incubation. The stability of ⁶⁸Ga-SDYD07 in PBS buffer: the radiochemical purities of ⁶⁸Ga-SDYD07 after incubation in PBS for 0.5 hours, 1 hour, and 2 hours were 99%, 98% and 98%, respectively (FIG. 37). The results indicate that ⁶⁸Ga-SDYD07 is quite stable in PBS buffer.

2. ¹⁷⁷Lu-SDYD07: ¹⁷⁷Lu-SDYD07 was added to PBS buffer (pH=7.4) and incubated at 37° C. The mixture samples were taken for radio-HPLC analysis after 24 hours, 48 hours, and 120 hours of incubation. The stability of ¹⁷⁷Lu-SDYD07 in PBS buffer: the radiochemical purities of ¹⁷⁷Lu-SDYD07 after incubation in PBS for 24 hours, 48 hours, and 120 hours were 99%, 98% and 97%, respectively (FIG. 38).

### Determination of Hydrophilicity/Lipophilicity of SDYD07 Radioligands

10 μL of purified ⁶⁸Ga-SDYD07 was diluted to 500 μL with HEPES buffer (pH=7.4), then 500 μL of n-octanol was added. The mixture was vortexed for 30 min and then centrifuged. Subsequently, 450 μL each of the aqueous and organic phases were taken and centrifuged again. Then, 100 μL each of the aqueous and organic phases were taken, and the radioactivity was measured using a γ counter to calculate the n-octanol/water distribution coefficient (Log Do/w). The measured Log Do/w value for ⁶⁸Ga-SDYD07 was −2.94±0.15, and for ¹⁷⁷Lu-SDYD07 was −2.71±0.15.

### Example 8: Synthesis of SDYD08 and Related Radioactive Experimental Studies

### Synthesis and Analysis of SDYD08:

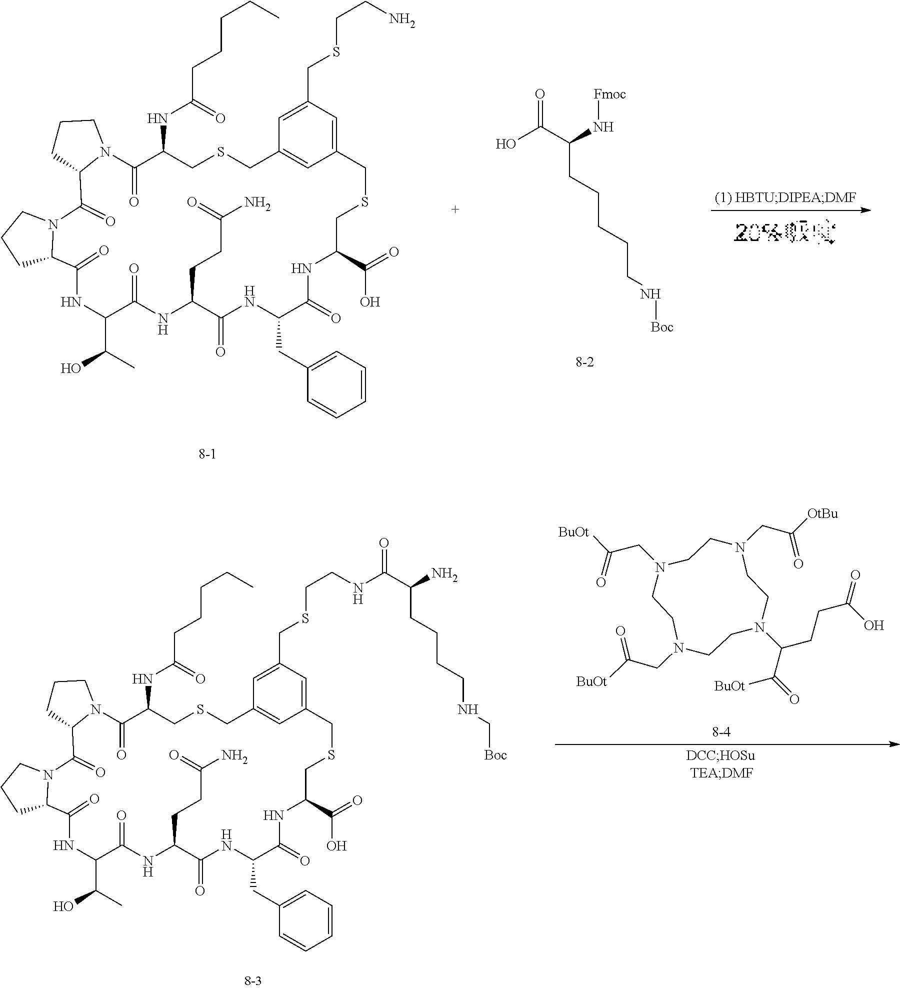

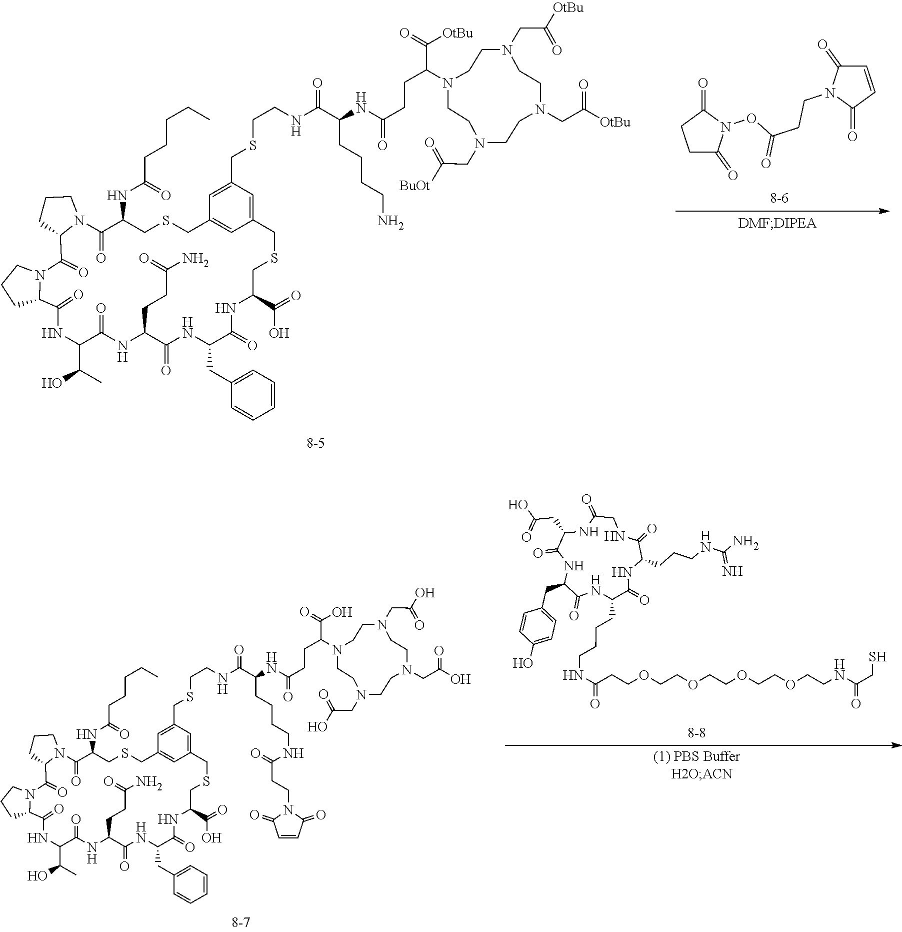

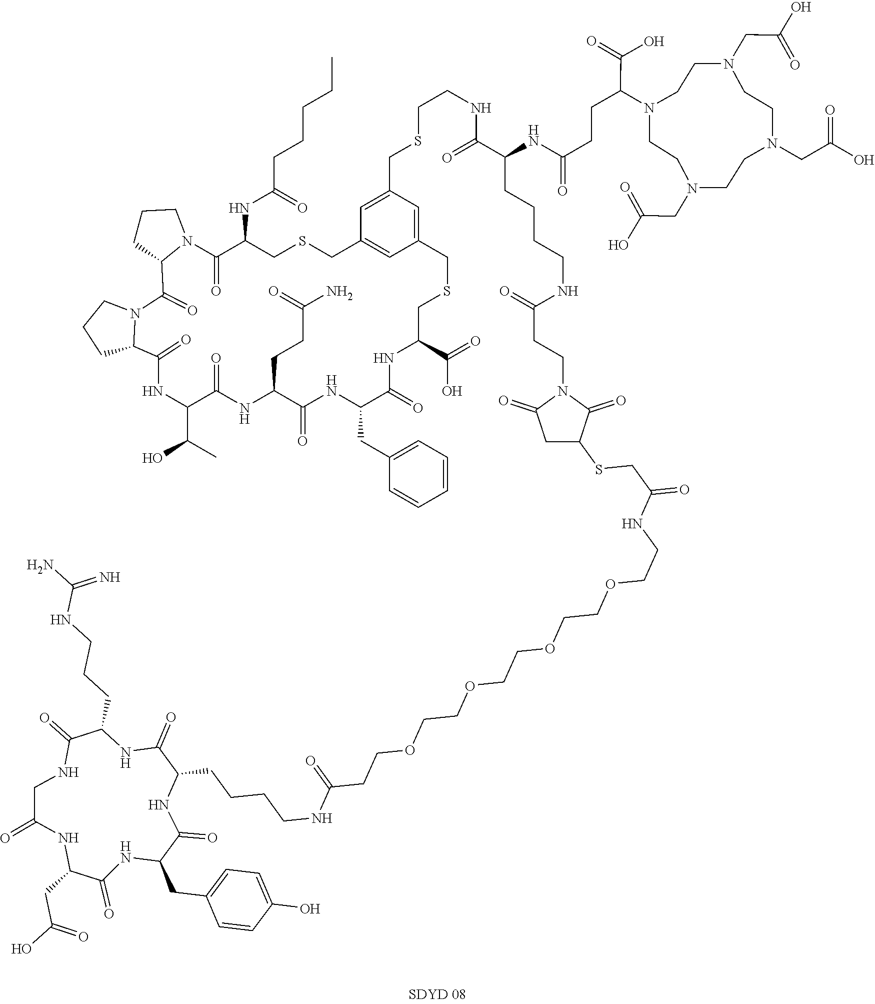

S1. Compound 8-1 (300 mg) was dissolved in 20 mL of DMF, added with 1.2 eq HBTU, 3.0 eq DIPEA, followed by 1.1 eq (142 mg) compound 8-2. The reaction was allowed to proceed at room temperature for 2 hours. Upon LC-MS confirmation of reaction completion, 20 mL of 20% piperidine was added, and then concentrated to dryness. The residue was washed with ice-cold diethyl ether 3 times and concentrated under reduced pressure to dryness. Purification by reversed-phase preparative LC afforded 210 mg of compound 8-3 (yield: 58%).

S2. Compound 8-4 (123 mg) was dissolved in 20 mL of DMF and added with 1.2 eq DCC and 1.2 eq HOSu. The reaction was allowed to proceed at room temperature for 6 hours. The precipitated solid was removed by filtering. To the filtrate were added 0.9 eq (210 mg) compound 8-3 and 2 eq TEA. The reaction was allowed to proceed at room temperature for 3 hours. Upon LC-MS confirmation of reaction completion, 20 mL of 20% piperidine was added. DMF was removed by rotary evaporation. The residue was washed twice with diethyl ether, and dried under reduced pressure. Purification by reversed-phase preparative LC afforded 186 mg of compound 8-5 (yield: 56%).

S3. Compound 8-5 (186 mg) was dissolved in 20 mL of DMF, added with 2.0 eq DIPEA, followed by 1.1 eq (29 mg) compound 8-6. The reaction was allowed to proceed at room temperature for 2 hours. Upon LC-MS confirmation of reaction completion, DMF was removed via rotary evaporation. 20 mL of TFA was added and stirred at room temperature for 2 hours. Upon LC-MS confirmation of reaction completion, ice-cold diethyl ether was added. Large amounts of solid precipitated out and were centrifuged. Purification by reversed-phase preparative LC afforded 119.8 mg of compound 8-7 (yield: 67%).

S4. Compound 8-7 (119.8 mg) was dissolved in 20 mL of H₂O/ACN=1:1, added with 1.0 eq (62 mg) compound 8-8, followed by 5 mL of 0.2 mol PBS Buffer (PH=7.2). The reaction was allowed to proceed at room temperature for 1 hour. Upon LC-MS confirmation of reaction completion, the reaction solution was directly purified by reversed-phase preparative LC to afford 121.7 mg of compound SDYD08 (yield: 67%).

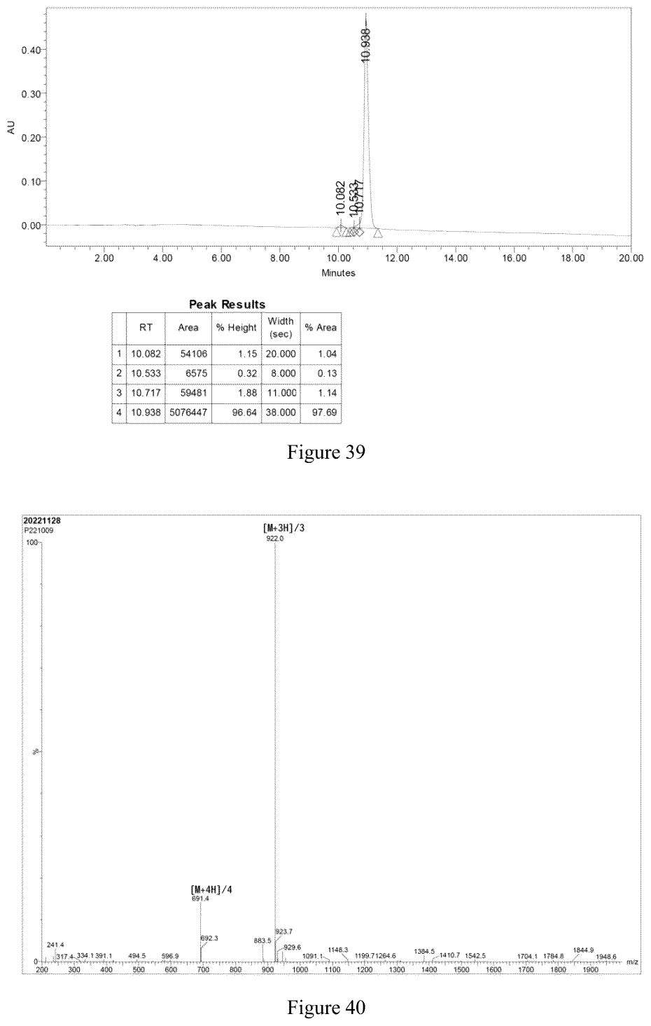

HPLC purity: 96.64% (FIG. 39). Molecular weight: theoretical 2763.21, measured 922.0 for [M+3H]/3, 691.4 for [M+4H]/4 (FIG. 40). Elemental analysis: N: 13.84%, C: 48.65%, H: 5.73%, which are consistent with the theoretical values. Main peak's PI is 2.916. Specific rotatory power is −24.3°.

### Radiolabeling of SDYD08 and Determination of Radiochemical Purity

Radiochemical purity refers to the percentage of radionuclide in the specified chemical form relative to the total radionuclide present in a radioactive sample. In this experiment, it specifically refers to the percentage of radioactivity successfully attached to the compound SDYD08 relative to the total radioactivity after purification.

1. ⁶⁸Ga Labeling: 3.0 nmol compound SDYD08 was added into 100 μL of NaOAc (0.25 M) buffer, followed by addition of 400 μL of ⁶⁸GaCl₃ solution (by rinsing the ⁶⁸Ge-⁶⁸Ga generator using 0.05 M HCl). The reaction mixture was shaken at 100° C. for 15 min. The crude product was purified using a C18 cartridge. The radiochemical purity of ⁶⁸Ga-SDYD08 was determined by radio-HPLC.

2. ¹⁷⁷Lu Labeling: 3.6 nmol compound SDYD08 was added into 130 μL of sodium acetate buffer (pH=5.6), followed by addition of 37MBq of LuCl₃ solution containing ¹⁷⁷Lu. The reaction mixture was shaken at 95° C. for 20 min. The crude product was purified using a C18 cartridge. The radiochemical purity of ¹⁷⁷Lu-SDYD08 was determined by radio-HPLC.

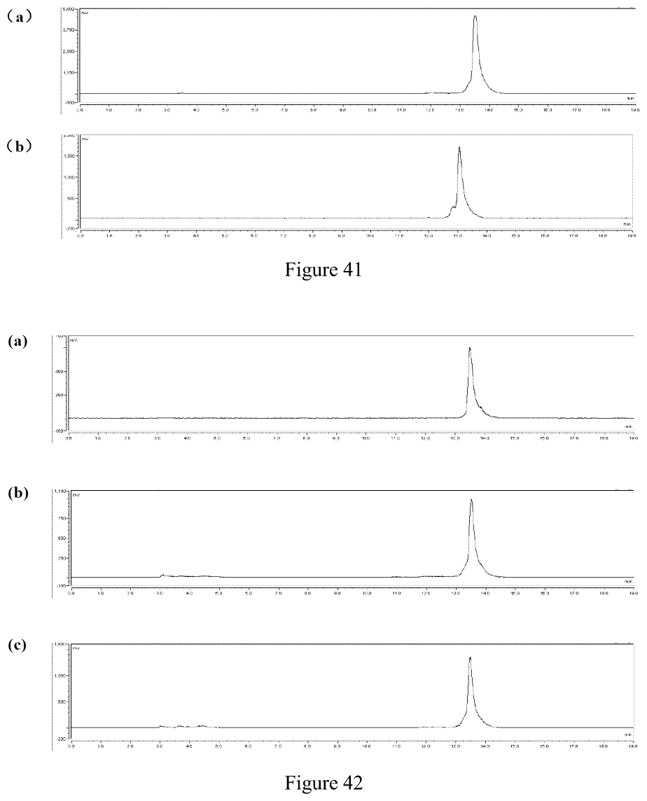

After purification through a C18 cartridge, the radiochemical purity of ⁶⁸Ga/¹⁷⁷Lu-SDYD08 was determined by radio-HPLC. Results showed 99% for ⁶⁸Ga-SDYD08 and 99% for ¹⁷⁷Lu-SDYD08 (FIG. 41).

### Stability of SDYD08 Radioligands in Phosphate Buffer

The purpose of this experiment was to test the stability of the radioligands under buffer conditions simulating the physiological environment. This stability is a prerequisite for further animal experiments. If no significant degradation of the ligand occurs, it indicates that the metal chelation and peptide bonds are stable in the buffer.

1. ⁶⁸Ga-SDYD08: ⁶⁸Ga-SDYD08 was added to PBS buffer (pH=7.4) and incubated at 37° C. The mixture samples were taken for radio-HPLC analysis after 0.5 hours, 1 hour, and 2 hours of incubation. The stability of ⁶⁸Ga-SDYD08 in PBS buffer: the radiochemical purities of ⁶⁸Ga-SDYD08 after incubation in PBS for 0.5 hours, 1 hour, and 2 hours were 99%, 97% and 96%, respectively (FIG. 42). The results indicate that ⁶⁸Ga-SDYD08 is quite stable in PBS buffer.

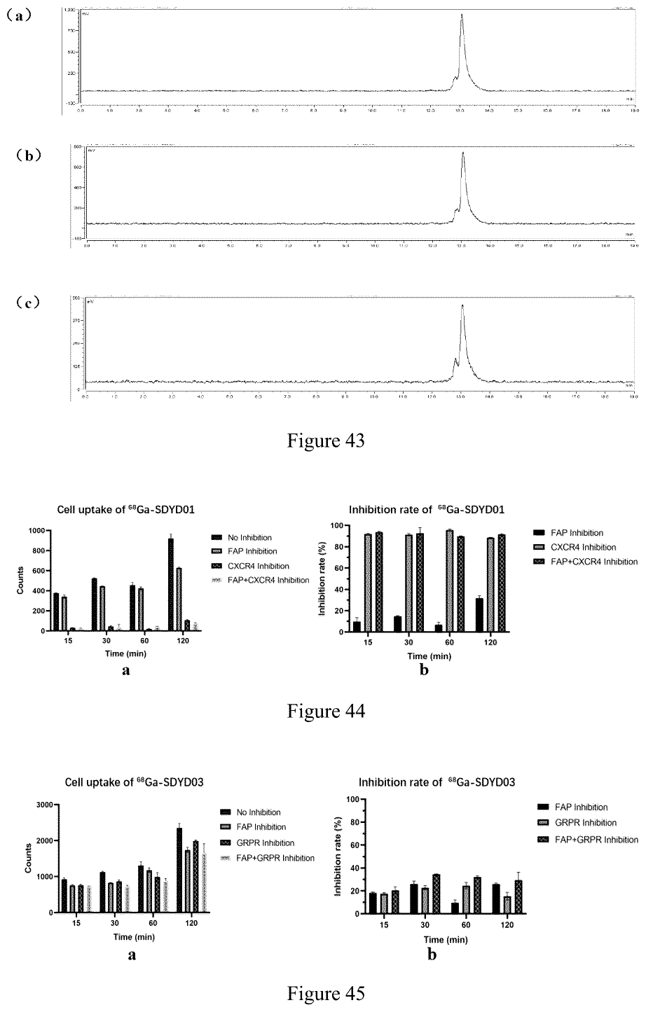

2. ¹⁷⁷Lu-SDYD08: ¹⁷⁷Lu-SDYD08 was added to PBS buffer (pH=7.4) and incubated at 37° C. The mixture samples were taken for radio-HPLC analysis after 24 hours, 48 hours, and 120 hours of incubation. The stability of ¹⁷⁷Lu-SDYD08 in PBS buffer: the radiochemical purities of ¹⁷⁷Lu-SDYD08 after incubation in PBS for 24 hours, 48 hours, and 120 hours were 98%, 97% and 95%, respectively (FIG. 43). The results indicate that ¹⁷⁷Lu-SDYD08 is quite stable in PBS buffer.

### Determination of Hydrophilicity/Lipophilicity of SDYD08 Radioligands

10 μL of purified ⁶⁸Ga-SDYD08 was diluted to 500 μL with HEPES buffer (pH=7.4), then 500 μL of n-octanol was added. The mixture was vortexed for 30 min and then centrifuged. Subsequently, 450 μL each of the aqueous and organic phases were taken and centrifuged again. Then, 100 μL each of the aqueous and organic phases were taken, and the radioactivity was measured using a γ counter to calculate the n-octanol/water distribution coefficient (Log Do/w). The measured Log Do/w value for ⁶⁸Ga-SDYD08 was −3.23±0.16, and for ¹⁷⁷Lu-SDYD08 was −2.82±0.13.

### Example 9: Synthesis of SDYD07-1

SDYD07-1 was synthesized by referring to the method described in Example 7, as shown in the following scheme:

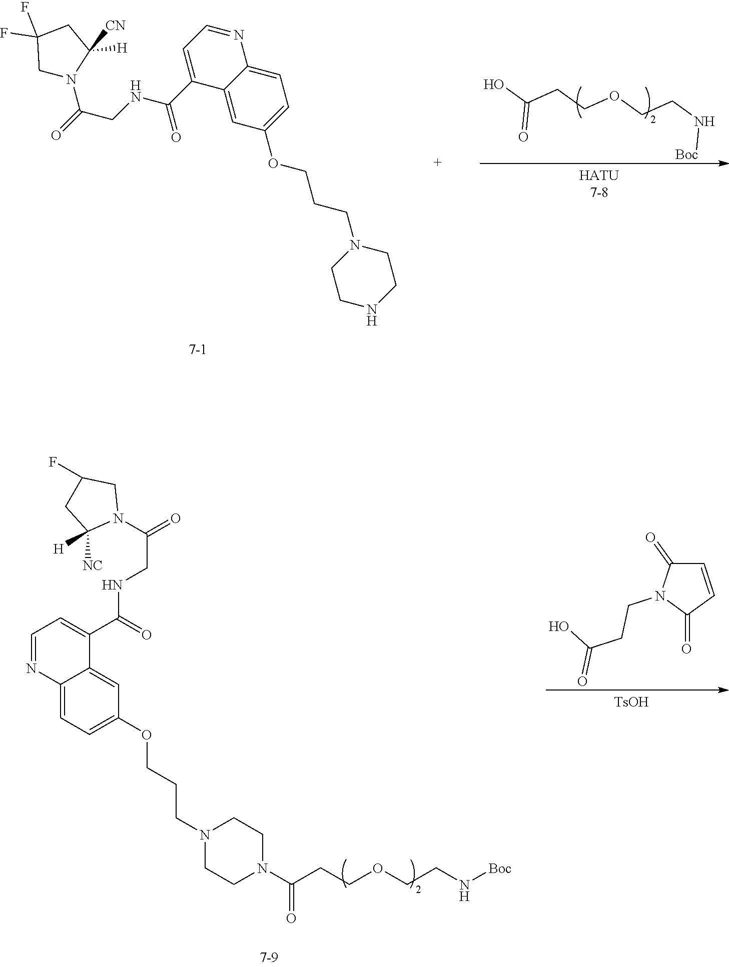

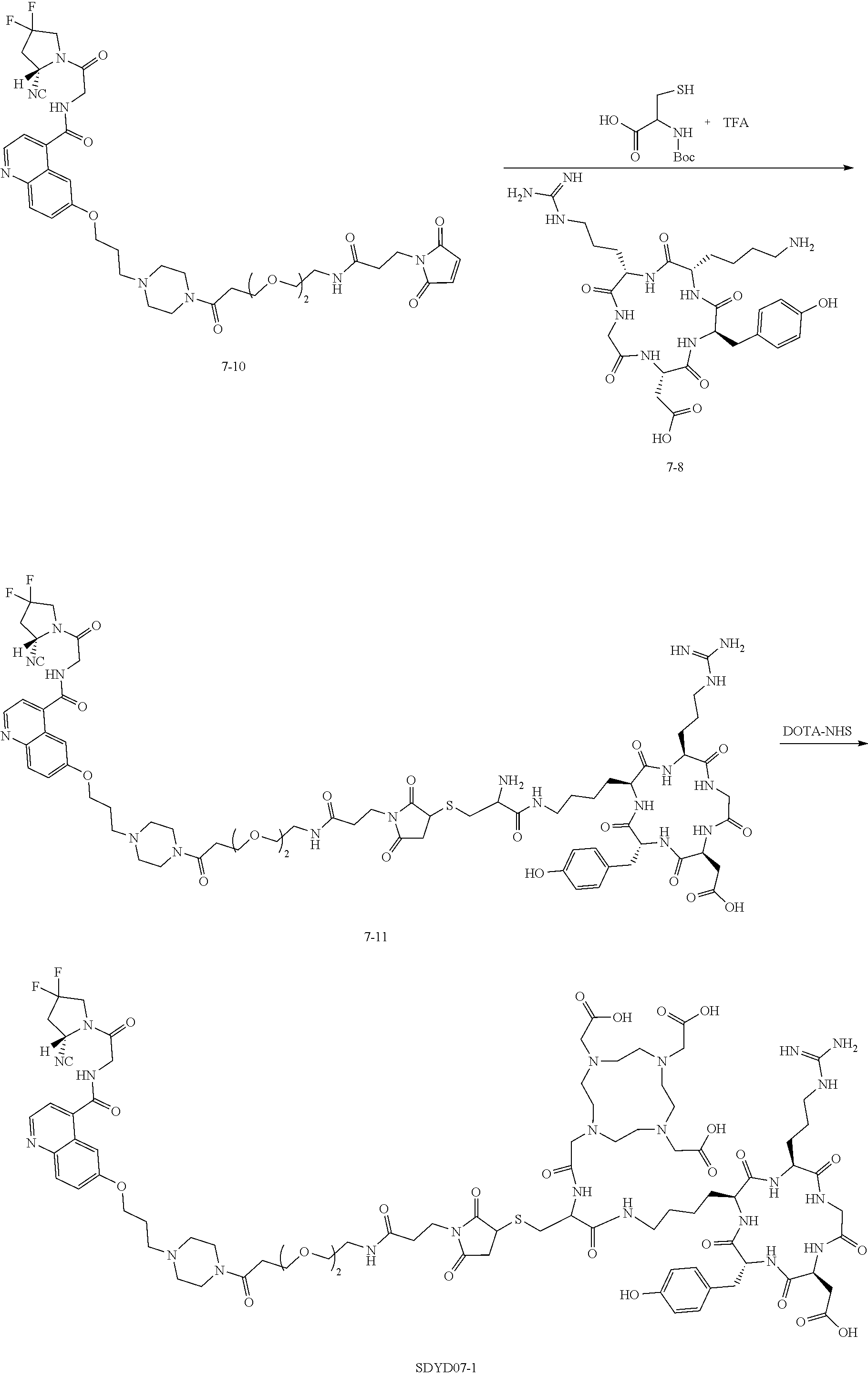

### Test Examples

The cell lines involved in the cell experiments in the test examples include: SUM149PT human breast cancer cell line, HUTU-80 human duodenal adenocarcinoma cell line, and U-118 MG human brain astrocytoma/glioblastoma cell line.

Each of cell lines was cultured under the following conditions:

SUM149PT: Complete medium was formulated using DMEM high-glucose medium, 10% FBS, and a penicillin-streptomycin solution. The cells were cultured in a constant temperature incubator at 37° C. with 5% CO₂. The culture medium was regularly replaced, and the cells were passaged. The culture medium was generally changed every 2 to 3 days. The growth of the tumor cells was observed daily under a microscope, including monitoring cell growth status, checking for adherence, examining cell morphology for any abnormalities, checking for contamination, and observing medium color for any abnormalities. Cells were passaged when they reached 80-90% confluence.

HUTU-80: Complete medium was formulated using DMEM high-glucose medium, 10% FBS, and a penicillin-streptomycin solution. The cells were cultured in a constant temperature incubator at 37° C. with 5% CO₂. The culture medium was regularly replaced, and the cells were passaged. The culture medium was generally changed every 2 to 3 days. The growth of the tumor cells was observed daily under a microscope, including monitoring cell growth status, checking for adherence, examining cell morphology for any abnormalities, checking for contamination, and observing medium color for any abnormalities. Cells were passaged when they reached 80-90% confluence.

U-118 MG: Complete medium was formulated using DMEM high-glucose medium, 10% FBS, and a penicillin-streptomycin solution. The cells were cultured in a constant temperature incubator at 37° C. with 5% CO₂. The culture medium was regularly replaced, and the cells were passaged. The culture medium was generally changed every 2 to 3 days. The growth of the tumor cells was observed daily under a microscope, including monitoring cell growth status, checking for adherence, examining cell morphology for any abnormalities, checking for contamination, and observing medium color for any abnormalities. Cells were passaged when they reached 80-90% confluence.

### Cell Plating

When cell confluence in the culture flask reached 80%, the cells were digested and centrifuged, and the supernatant was removed. An appropriate amount of medium was added to dilute so that each well contained 500 μL of medium for plating. After thorough mixing by pipetting, the cell suspension was seeded into a 24-well plate at a density of 5×10⁵ cells per well. The plate was then placed into the incubator for continued growth. The cells were ready for cellular experiments when they reached 80% confluence.

### Test Example 1: Cell Uptake and Inhibition Assay

(1) The cell uptake assay established four incubation time points: 15, 30, 60, and 120 minutes, respectively. For each time point, four groups were set up: non-inhibition group, a first single inhibition group (FAP single inhibition), a second single inhibition group (CXCR4/GRPR/αᵥβ₃ single inhibition), and a dual inhibition group (FAP inhibitor+CXCR4/GRPR/αᵥβ₃ inhibitor). Each group at each time point was set up with 3 triplicate wells.

(2) Cells were plated into a 24-well plate according to the cell plating procedure described above.

(3) The original culture medium was removed from the 24-well plate. Then, 400 μL of 100 mg/mL skimmed milk solution was added to each well for blocking for one hour. After removing the skimmed milk solution, each of the wells was washed three times by adding 400 μL of PBS solution. Subsequently, 146 μL of FBS-free medium was added to each well. Next, the non-inhibition group received 4 μL of cold PBS per well, while the first single inhibition group, the second single inhibition group, and the dual inhibition group received the following additions per well according to Table 3: 2 μL of non-radiolabeled first single inhibitor solution and 2 μL of cold PBS: 2 μL of non-radiolabeled second single inhibitor solution and 2 μL of cold PBS; 2 μL of non-radiolabeled first single inhibitor solution (10 mM) and 2 μL of non-radiolabeled second single inhibitor solution (10 mM). The plate was incubated at room temperature for one hour.

(4) The ⁶⁸Ga- and ¹⁷⁷Lu-labeled radioactive ligands were formulated by adding FBS-free medium to achieve a radioligand system concentration of 20 nM. Then, 50 μL of this radioligand system was added to each well. Each group was incubated according to the designated time points: 15, 30, 60, and 120 minutes.

(5) After the incubation was complete, the supernatant was removed, and the wells were washed three times with 400 μL of PBS solution. Then, 200 μL of 1 M NaOH solution was added to each well for cell lysis. After waiting approximately 5 minutes until flocculent material was observed in the wells, the lysed cell solution was collected into radioimmunoassay tubes. Each well was then washed once with 400 μL of PBS solution, and this wash solution was also added to the corresponding radioimmunoassay tubes.

(6) The radioactivity in the cells was measured using a γ counter. A bar graph was plotted using GraphPad Prism software. The inhibition rate for each inhibition group was calculated as follows: Inhibition Rate (%)=(1−[Radioactive Count of the Inhibition Group]/[Radioactive Count of the Non-inhibition Group])×100%.

For example: FAP Single Inhibition Group Inhibition Rate (%)=(1−[Radioactive Count of the FAP Single Inhibition Group]/[Radioactive Count of the Non-inhibition Group])×100%.

TABLE 3
Experimental Grouping for Cell Uptake Assay of Each Compound

| | | First single | Second single | |
|---|---|---|---|---|
| Compound | Cell line | inhibitor solution | inhibitor solution | Dual inhibition group |
| SDYD01 | SUM149PT | FAPI-04 solution | T140 solution | T140 solution (10 mM) and |
| | | (FAP inhibitor, | (CXCR4 | FAPI-04 solution (10 mM) |
| | | 10 mM) | inhibitor, 10 mM) | |
| SDYD03 | HUTU-80 | FAPI-04 solution | BBN solution | BBN solution (20 mM) and |
| | | (FAP inhibitor, | (GRPR inhibitor, | FAPI-04 solution (20 mM) |
| | | 20 mM) | 20 mM) | |
| SDYD04 | HUTU-80 | FAPI-04 solution | BBN solution | BBN solution (2 mM) and |
| | | (FAP inhibitor, | (GRPR inhibitor, | FAPI-04 solution (2 mM) |
| | | 20 mM) | 20 mM) | |
| SDYD05 | SUM149PT | FAPI-04 solution | T140 solution | T140 solution (10 mM) and |
| | | (FAP inhibitor, | (CXCR4 | FAPI-04 solution (10 mM) |
| | | 10 mM) | inhibitor, 10 mM) | |
| SDYD06 | SUM149PT | FAPI-04 solution | T140 solution | T140 solution (10 mM) and |
| | | (FAP inhibitor, | (CXCR4 | FAPI-04 solution (10 mM) |
| | | 10 mM) | inhibitor, 10 mM) | |
| SDYD07 | U-118MG | FAPI-04 solution | RGD solution | RGD solution (10 mM) and |
| | | (FAP inhibitor, | (integrin αᵥβ₃ | FAPI-04 solution (10 mM) |
| | | 10 mM) | inhibitor, 10 mM) | |
| SDYD08 | U-118MG | FAPI-04 solution | RGD solution | RGD solution (10 mM) and |
| | | (FAP inhibitor, | (integrin αᵥβ₃ | FAPI-04 solution (10 mM) |
| | | 10 mM) | inhibitor, 10 mM) | |

Experimental Results Indicate that:

SDYD01: In the FAP single inhibition group and the non-inhibition group during the first 30 minutes, the uptake of ⁶⁸Ga-SDYD01 by SUM149PT cells increased with incubation time, and the uptake slowly increased and reached a plateau from 30 to 120 minutes. Starting from 30 minutes, the cell uptake in the non-inhibition group was higher than that in the other groups, especially at 120 minutes, where the cell uptake in the non-inhibition group was significantly higher than that in the FAP single inhibition group, CXCR4 single inhibition group, and dual inhibition group. Moreover, the dual inhibition group exhibited the lowest uptake (FIG. 44a). The inhibition rates of the FAP single inhibition group, CXCR4 single inhibition group, and dual inhibition group at different time points are shown in FIG. 44b. The results indicate that compound SDYD01 can rapidly and efficiently bind specifically to the CXCR4 protein, quickly target the objective, and also bind specifically to fibroblast activation protein to a certain extent.

SDYD03: Uptake in all groups increased slowly and reached a plateau from 15 to 120 minutes. Starting from 15 minutes, the cell uptake in the non-inhibition group was consistently higher than that in the other three groups. The dual inhibition group showed lower cell uptake counts compared to the FAP single inhibition group and the GRPR single inhibition group (FIG. 45a). The inhibition rates of the FAP single inhibition group, GRPR single inhibition group, and dual inhibition group at different time points are shown in FIG. 45b. It can be observed that the inhibition rate of the dual inhibition group was higher than that of the FAP single inhibition group and the GRPR single inhibition group, indicating that this compound can specifically bind to both FAP and GRPR.

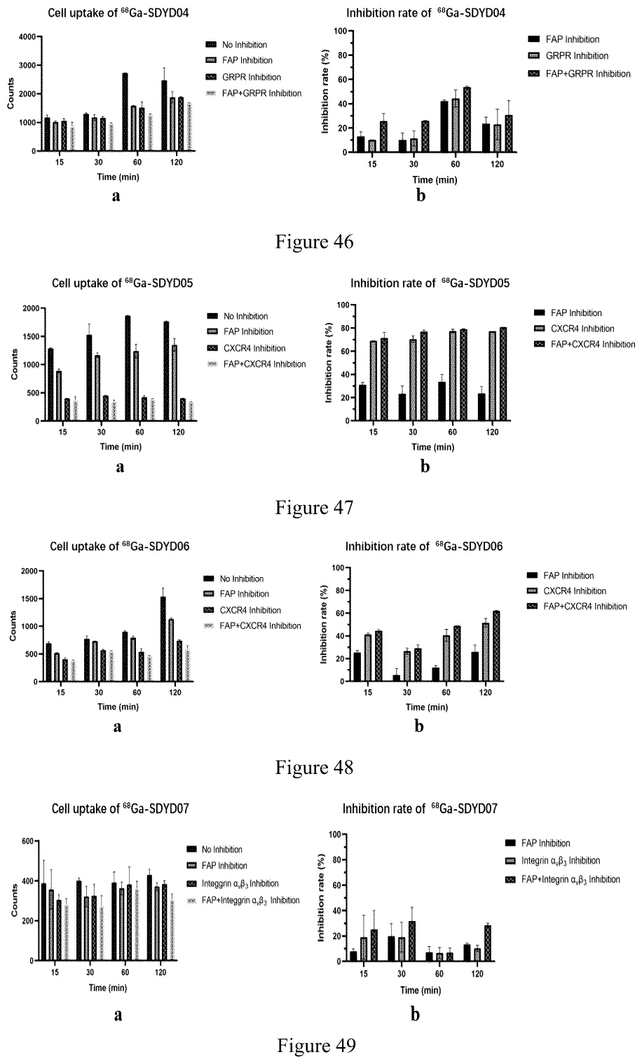

SDYD04: The trends of all groups over time were consistent, with a significant increase in cell uptake within the first 15 minutes. The non-inhibition group reached peak uptake at 60 minutes. The uptake levels of the FAP single inhibition group and the GRPR single inhibition group were similar at all time points, while the dual inhibition group exhibited the lowest cell uptake, and the non-inhibition group showed the highest cell uptake (FIG. 46a). The inhibition rates of the FAP single inhibition group, GRPR single inhibition group, and dual inhibition group at different time points are shown in FIG. 46b. The results demonstrate that SDYD04 can specifically bind to both FAP and GRPR, and the combination of dual ligands enhances the rate of cell uptake of the radioactive compound.

SDYD05: In the non-inhibition group and the FAP single inhibition group, cell uptake increased significantly within the first 15 minutes, and slowly increased and reached a plateau from 15 to 120 minutes. In the CXCR4 single inhibition group and the dual inhibition group, cell uptake increased within the first 15 minutes, with no significant change from 15 to 120 minutes, approaching a dynamic equilibrium. Starting from 15 minutes, the non-inhibition group exhibited the highest cell uptake, followed by the FAP single inhibition group, while the dual inhibition group showed the lowest uptake rate (FIG. 47a). The inhibition rates of the FAP single inhibition group. CXCR4 single inhibition group, and dual inhibition group at different time points are shown in FIG. 47b. The results indicate that compound SDYD05 can rapidly and efficiently bind specifically to the CXCR4 protein, quickly target the objective, and also bind specifically to fibroblast activation protein to a certain extent.

SDYD06: In the non-inhibition group and the FAP single inhibition group, cell uptake increased significantly within the first 15 minutes, and slowly increased and reached a plateau from 15 to 120 minutes. Within 60 minutes, cell uptake in the CXCR4 single inhibition group and the dual inhibition group slowly increased with incubation time, with no significant change from 60 to 120 minutes, reaching a dynamic equilibrium. Starting from 15 minutes, the cell uptake levels, from high to low, were as follows: non-inhibition group, FAP single inhibition group, CXCR4 single inhibition group, and dual inhibition group. The cell uptake results of the non-inhibition group and the FAP single inhibition group were similar, while the results of the CXCR4 single inhibition group and the dual inhibition group were comparable (FIG. 48a). The inhibition rates of the FAP single inhibition group, CXCR4 single inhibition group, and dual inhibition group at different time points are shown in FIG. 48b.

SDYD07: In all groups, cell uptake increased significantly within the first 15 minutes, and then slowly increased. At 30 minutes, the cell uptake levels, from high to low, were as follows: non-inhibition group, FAP single inhibition group, integrin αᵥβ₃ single inhibition group, and dual inhibition group (FIG. 49a). The inhibition rates of the FAP single inhibition group, integrin αᵥβ₃ single inhibition group, and dual inhibition group at different time points are shown in FIG. 49b.

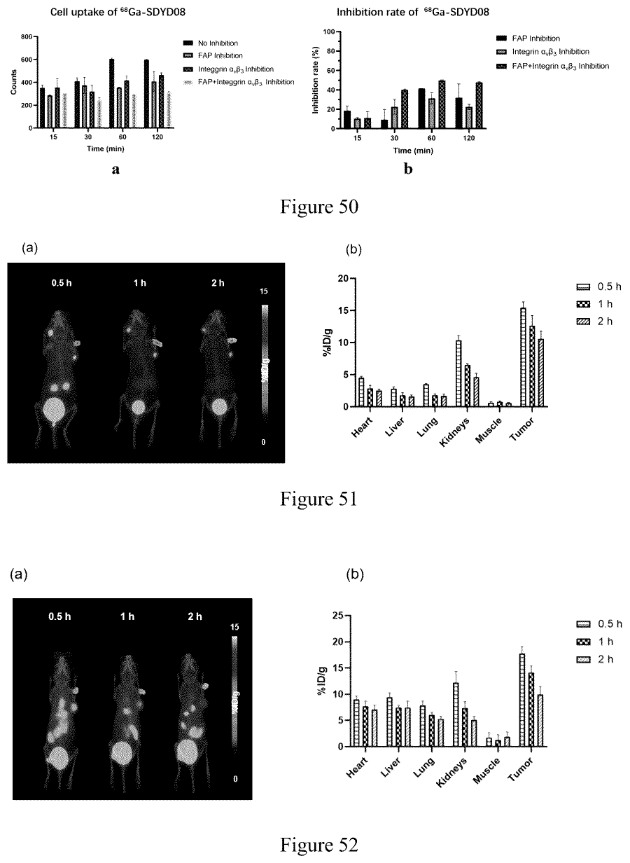

SDYD08: In all groups, cell uptake increased significantly within the first 15 minutes. In the non-inhibition group, cell uptake increased slowly from 15 to 30 minutes, significantly increased from 30 to 60 minutes, with no significant change from 60 to 120 minutes. In the FAP single inhibition group and the integrin αᵥβ₃ single inhibition group, cell uptake increased significantly within the first 15 minutes, with no significant change from 15 to 120 minutes, reaching a dynamic equilibrium. At 30 minutes, the cell uptake levels, from high to low, were as follows: non-inhibition group, FAP single inhibition group, integrin αᵥβ₃ single inhibition group, and dual inhibition group (FIG. 50a). The inhibition rates of the FAP single inhibition group, integrin αᵥβ₃ single inhibition group, and dual inhibition group at different time points are shown in FIG. 50b. The results indicate that compound SDYD08 can specifically bind to fibroblast activation protein and integrin αᵥβ₃, demonstrating good specific targeting effects for these two targets.

### Test Example 2: Cell Saturation Binding Assay

(1) For the cell saturation binding assay, a total of 8 radiolabeled ligand concentrations were set at intervals within the anticipated range. For each concentration, there were 3 groups: the non-inhibition group, the first single inhibition group (FAP single inhibition group), and the second single inhibition group (CXCR4/GRPR/αᵥβ₃ single inhibition group). For each concentration and each group, 3 replicate wells were established.

(2) Cells were plated into a 24-well plate according to the cell plating procedure described above.

(3) The original culture medium was removed from the 24-well plate. Then, 400 μL of 100 mg/mL skimmed milk solution was added to each well for blocking for one hour. After removing the skimmed milk solution, each of wells was washed three times by adding 400 μL of PBS solution. Subsequently, 146 μL of FBS-free medium was added to each well. Next, the non-inhibition group received 4 μL of cold PBS per well, while the first single inhibition group and the second single inhibition group received the following additions per well according to Table 4: 2 μL of non-radiolabeled first single inhibitor solution and 2 μL of cold PBS; 2 μL of non-radiolabeled second single inhibitor solution and 2 μL of cold PBS. The plate was then incubated at room temperature for one hour.

(4) The ligands were radiolabeled with 68Ga using the labeling method described previously. Different volumes of FBS-free medium were added to formulate the radioligand systems at corresponding concentrations. Then, 50 μL of the corresponding concentration of the radioligand system was added to each well.

(5) The supernatant was removed, and the wells were washed three times with 400 μL of PBS. 200 μL of IM NaOH solution was added to each well for cell lysis. After waiting approximately 5 minutes, until flocculent material was observed in the wells, the lysed cell solution was collected into radioimmunoassay tubes. Each well was washed once with 400 μL of PBS solution, and the washings were sequentially added to the corresponding radioimmunoassay tubes.

(6) Radioactivity in the cells was measured using a γ counter. The equilibrium dissociation constant (K_{D}) was obtained by fitting the curve using nonlinear regression analysis in GraphPad Prism software. K_{D} is the drug concentration that causes half of the maximum effect (50% receptor occupancy). A larger K_{D} value indicates that a higher drug concentration is required to achieve the maximum effect, corresponding to lower affinity. The Ko values for each compound are shown in Table 5 below.

The results indicate that the ⁶⁸Ga-labeled compounds SDYD01, SDYD02, SDYD03, SDYD04, SDYD05, SDYD06, SDYD07, and SDYD08 all have relatively low K_{D} values, demonstrating good binding affinity to the receptors.

TABLE 4
Experimental Grouping for Cell Saturation Binding Assay for Each Compound

| | | Final Concentrations Range | First single | Second single |
|---|---|---|---|---|
| Compound | Cell line | of Radiolabeled Compounds | inhibitor solution | inhibitor solution |
| SDYD01 | SUM149PT | 0.25 nM-16 nM | FAPI-04 solution | T140 solution |
| | | | (FAP inhibitor, | (CXCR4 inhibitor, |
| | | | 10 mM) | 10 mM) |
| SDYD02 | SUM149PT | 0.15625 nM-20 nM | FAPI-04 solution | T140 solution |
| | | | (FAP inhibitor, | (CXCR4 inhibitor, |
| | | | 10 mM) | 10 mM) |
| SDYD03 | HUTU-80 | 0.15625 nM-2.5 nM | FAPI-04 solution | BBN solution |
| | | | (FAP inhibitor, | (GRPR inhibitor, |
| | | | 20 mM) | 20 mM) |
| SDYD04 | HUTU-80 | 0.3125 nM-30 nM | FAPI-04 solution | BBN solution |
| | | | (FAP inhibitor, | (GRPR inhibitor, |
| | | | 20 mM) | 20 mM) |
| SDYD05 | SUM149PT | 0.039 nM-10 nM | FAPI-04 solution | T140 solution |
| | | | (FAP inhibitor, | (CXCR4 inhibitor, |
| | | | 10 mM) | 10 mM) |
| SDYD06 | SUM149PT | 0.15625 nM-10 nM | FAPI-04 solution | T140 solution |
| | | | (FAP inhibitor, | (CXCR4 inhibitor, |
| | | | 10 mM) | 10 mM) |
| SDYD07 | U-118MG | 1.25 nM-40 nM | FAPI-04 solution | RGD solution |
| | | | (FAP inhibitor, | (integrin αᵥβ₃ |
| | | | 10 mM) | inhibitor, 10 mM) |
| SDYD08 | U-118MG | 0.3125 nM-40 nM | FAPI-04 solution | RGD solution |
| | | | (FAP inhibitor, | (integrin αᵥβ₃ |
| | | | 10 mM) | inhibitor, 10 mM) |

TABLE 5
Dissociation Constants (K_{D}) from Cell Saturation
Binding Assays for Each Compound

| | K_{D} value (nM) | | |
|---|---|---|---|
| | | First single | Second single |
| | Non- | inhibition group | inhibition group |
| | inhibition | (FAP single | (CXCR4/GRPR/αᵥβ₃ |
| Compound | group (nM) | inhibition) | single inhibition) |
| SDYD01 | 12.27 ± 2.07 | 15.82 ± 1.22 | 11.35 ± 1.36 |
| SDYD02 | 7.105 ± 1.17 | 3.352 ± 0.92 | 6.303 ± 0.78 |
| SDYD03 | 1.226 ± 0.331 | 0.884 ± 0.152 | 1.756 ± 0.224 |
| SDYD04 | 1.919 ± 0.41 | 2.511 ± 0.96 | 1.246 ± 0.67 |
| SDYD05 | 0.217 ± 0.018 | 0.3735 ± 0.035 | 1.993 ± 0.312 |
| SDYD06 | 1.414 ± 0.213 | 0.455 ± 0.056 | 0.173 ± 0.012 |
| SDYD07 | 10.52 ± 1.874 | 7.098 ± 1.065 | 7.625 ± 0.923 |
| SDYD08 | 8.426 ± 0.475 | 7.125 ± 0.558 | 10.53 ± 1.062 |

### Test Example 3: Small Animal PET Imaging and Biodistribution Study

Eleven NSG mice bearing subcutaneous xenograft tumors (U87 MG cells), with tumor sizes approximately 150 mm³, were used. The mice were intravenously injected via the tail vein with approximately 7.4 MBq of ⁶⁸Ga-labeled SDYD01, SDYD02, SDYD03. SDYD04, SDYD05, SDYD06, SDYD07, SDYD08 or SDYD07-1 compounds, or with ⁶⁵Ga-labeled FAPI-04 or FAP2286 compounds as tracers. Micro PET/CT imaging was performed at 30 min, 60 min, and 120 min post-injection. Static images were acquired in 3D mode for 20 minutes. Reconstruction was performed using the OSEM3D/MAP method to obtain attenuation-corrected PET/CT fusion images. Tumor imaging was observed for each group.

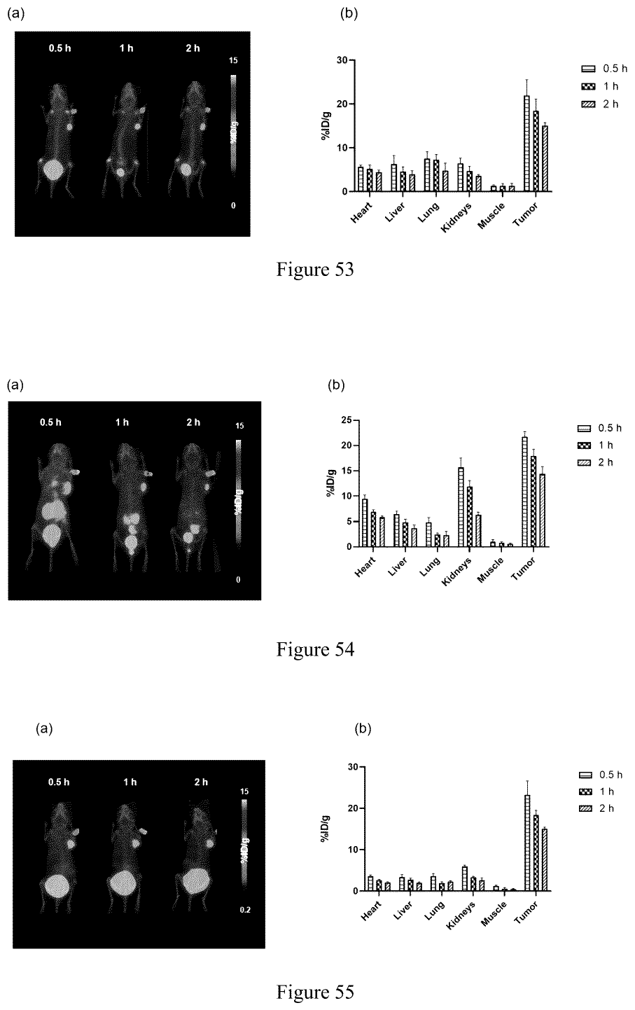

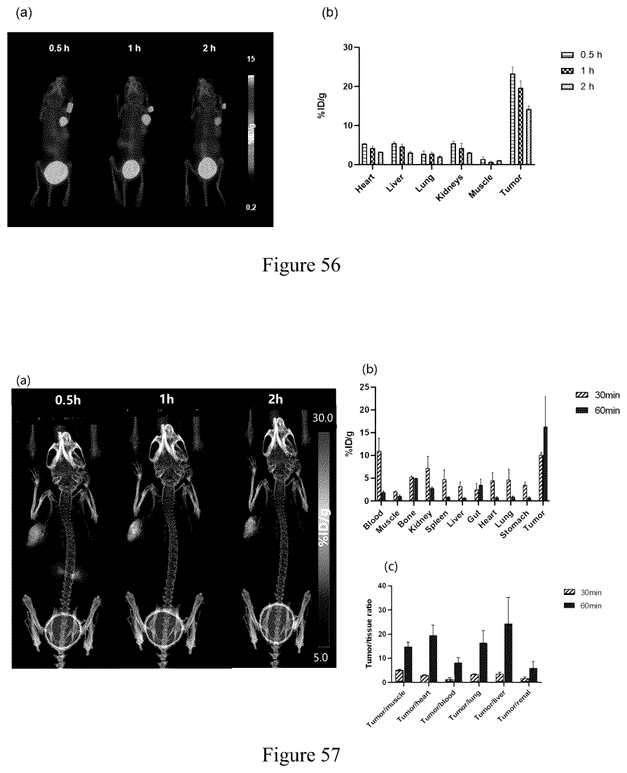

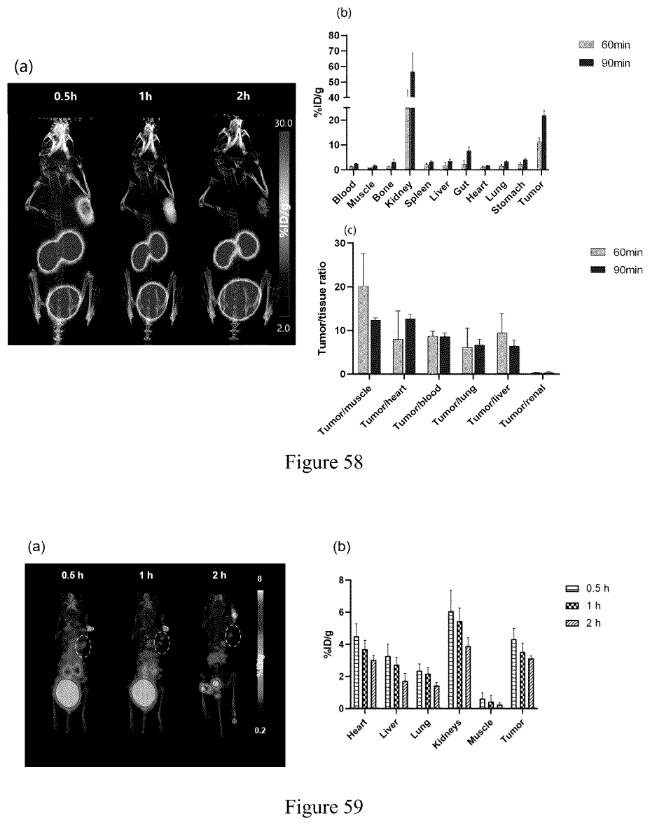

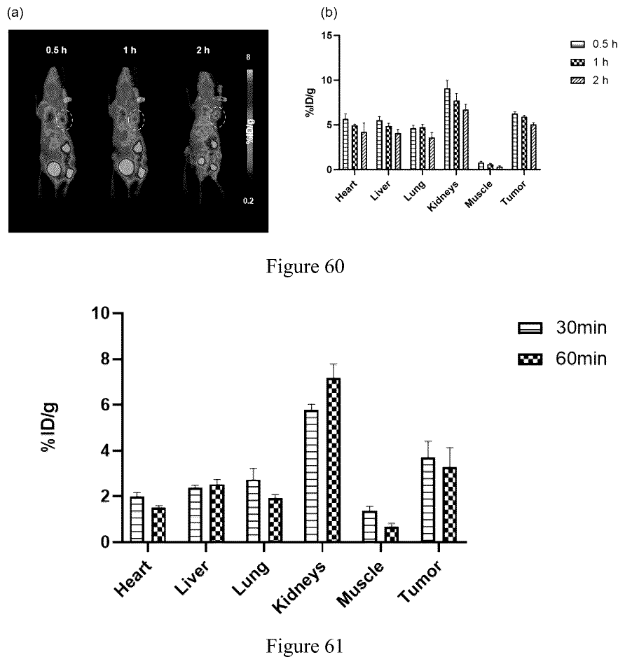

NSG mice bearing subcutaneous xenograft tumors (U87 MG cells) were divided into 3 groups, with 11 mice in each group. They were intravenously injected via the tail vein with ⁶⁸Ga-labeled SDYD01, SDYD02, SDYD03. SDYD04, SDYD05, SDYD06. SDYD07, SDYD08, or SDYD07-1 compounds, or with ⁶⁸Ga-labeled FAPI-04 or FAP2286 compounds as tracers. Tumor-bearing mice injected with ⁶⁸Ga-labeled compounds SDYD01, SDYD02, SDYD03, SDYD04, SDYD05, SDYD06, FAPI-04, as well as 68Ga-labeled compounds FAPI-04 and FAP2286, were euthanized at 30 min. 60 min, and 120 min, respectively, for each group. Tumor-bearing mice injected with ⁶⁸Ga-labeled compounds SDYD07, SDYD08, and SDYD07-1 were euthanized at 30 min, 60 min, and 90 min, respectively, for each group. Blood, tumors, and other major organs and tissues were collected, weighed, and measured for radioactivity. After radioactive decay correction, the standardized uptake value (SUV) and percentage injected dose per gram of tissue (% ID/g) were calculated. The imaging results and biodistribution data for the various ⁶⁸Ga-labeled compound used as tracers are shown in FIGS. 51-61. In these figures, (a) represents the PET/CT imaging results, (b) represents the biodistribution results, and (c) represents the relative ratios of radioactive uptake in tumor versus normal tissues.

The results showed that tumor-bearing mice injected with ⁶⁸Ga-SDYD01, ⁶⁸Ga-SDYD02, ⁶⁸Ga-SDYD03, ⁶⁸Ga-SDYD04, ⁶⁸Ga-SDYD05, ⁶⁸Ga-SDYD06, ⁶⁸Ga-SDYD07, and ⁶⁸Ga-SDYD08 all exhibited significant radioactive uptake in the tumor sites. The absolute uptake values of the radionuclide-labeled compounds in the tumors reached 10% ID/g or higher at 30 and 60 minutes. Furthermore, the radionuclide-labeled compounds were rapidly cleared from non-tumor sites. In contrast, tumor-bearing mice injected with ⁶⁸Ga-FAPI-04 and ⁶⁸Ga-FAPI-2286 showed significantly lower radioactive deposition in the tumor sites, indicating that the dual-targeting compounds provided in the examples of the present invention possess superior tumor-targeting specificity and uptake efficiency, and can be used for radiodiagnosis and radiotherapy.

For example, as showed in FIG. 57 (a), the absolute uptake value of 68Ga-SDYD07 in the tumor site reached 10% ID/g or higher at 30 minutes (0.5 h), reached 15% ID/g at 60 minutes (1 h), and remained at around 10% ID/g at 120 minutes (2 h). However, the absolute uptake value of ⁶⁸Ga-SDYD07-1 in the tumor site of mice was only about 3% ID/g at both the 30- and 60-min time points, and decreased significantly after 60 minutes. Thus, ⁶⁸Ga-SDYD07 exhibits higher tumor uptake and longer tumor retention time compared to ⁶⁸Ga-SDYD07-1. It is anticipated that SDYD07, when labeled with therapeutic radionuclides, will yield better tumor treatment efficacy. According to FIGS. 57 (b) and (c), the radioactive uptake levels in other non-target organs for ⁶⁸Ga-SDYD07 were significantly reduced at 60 minutes and were much lower than the tumor uptake levels in the tumor, resulting in significantly increased target/non-target ratios. This indicates that ⁶⁸Ga-SDYD07 has excellent tumor-specific uptake and low non-specific uptake levels. In comparison. ⁶⁸Ga-SDYD07-1 exhibited relatively high non-specific uptake in both the kidneys and liver, suggesting that ⁶⁸Ga-SDYD07 can provide better PET-CT image quality and has good diagnostic potential.

Although the present invention has been described in detail herein with general descriptions, specific embodiments, and experiments, it is obvious to those skilled in the art that some modifications or improvements can be made on this basis. Therefore, all modifications or improvements made without departing from the spirit of the present invention fall within the scope of protection claimed by the present invention.

1. A compound having a structure of formula (I), or a stereoisomer, tautomer, or pharmaceutically acceptable salt thereof: 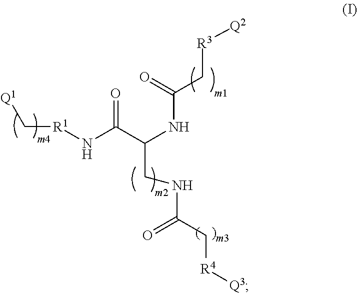
wherein,
R¹, R³ and R⁴ are each independently selected from the group consisting of a bond, —U¹-G¹-A¹-L-A²-G²-U²—, and a heteroalkylene, at least one of R¹, R³ and R⁴ is —U¹-G¹-A¹-L-A²-G²-U²—; or 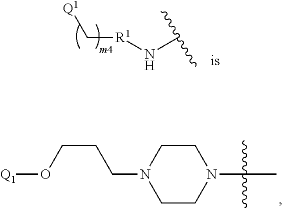
and at least one of R³ and R⁴ is —U¹-G¹-A¹-L-A²-G²-U²—;
L is 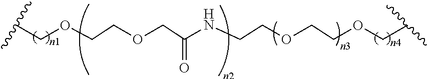
or a bond, n1, n2, n3 and n4 are each independently 0, 1, 2, or 3;
A¹ and A² are each independently selected from the group consisting of a bond, —C(═O) NH—, —NHC(═O)—, and —C(═O)—;
G¹ and G² are each independently selected from the group consisting of a bond, a heteroalkylene, and 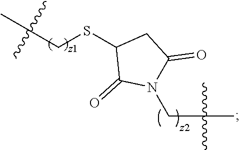
z1 and z2 are each independently selected from the group consisting of 0, 1, 2, and 3;
U¹ and U² are each independently selected from the group consisting of a bond, 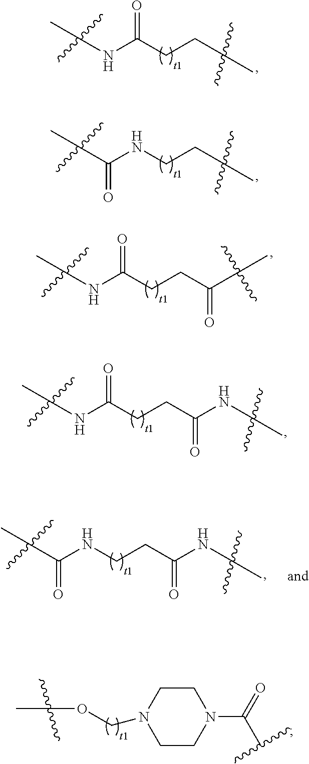
each t1 is independently selected from the group consisting of 1, 2, 3, and 4:
U¹, G¹, A¹, L, A², G² and U² are not a bond at the same time;
m1, m2, m3 and m4 are each independently selected from the group consisting of 0, 1, 2, 3, 4, and 5;
one of Q¹, Q² and Q³ is a chelating group, another is a FAP receptor-targeting moiety, and the third is an additional receptor-targeting moiety, wherein the additional receptor is selected from the group consisting of a CXCR4 receptor, a GRPR receptor, and an αᵥβ₃ receptor;
alternatively, the heteroalkylene is a alkylene comprising at least one sulfur atom; or
alternatively, the heteroalkylene is —(CH₂)_{q}—S—(CH₂)ₚ—, wherein p and q are each independently selected from the group consisting of 0, 1, 2, 3, 4, and 5, and, p and q are not both 0.

2. The compound according to claim 1, or a stereoisomer, tautomer, or pharmaceutically acceptable salt thereof, characterized in that:
(1) the compound has a structure of formula (I′-A): 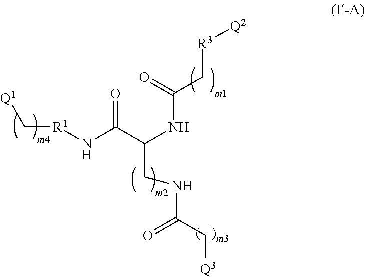
each symbol has the definition given in Formula (I),
preferably, Q¹ is a FAP receptor-targeting moiety, Q² is an additional receptor-targeting moiety such as a CXCR4 receptor, an αᵥβ₃ receptor or a GRPR receptor, Q³ is a chelating group; or Q² is a FAP receptor-targeting moiety, Q¹ is an additional receptor-targeting moiety such as a CXCR4 receptor, an αᵥβ₃ receptor or a GRPR receptor, Q³ is a chelating group;
preferably, one of R¹ and R³ is —U¹-G¹-A¹-L-A²-G²-U²—, and the other is selected from the group consisting of a bond, U¹, and G¹;
L is 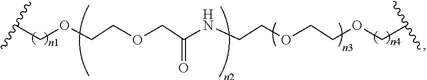
n1, n2, n3 and n4 are each independently 0, 1, 2, or 3;
A¹ and A² are each independently selected from the group consisting of a bond, —C(═O) NH—, —NHC(═O)—, and —C(═O)—;
G¹ and G² are each independently selected from the group consisting of a bond, —(CH₂)_{q}—S—(CH₂)ₚ—, and 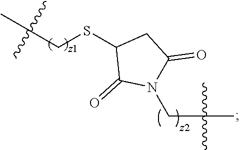
z1 and z2 are each independently selected from the group consisting of 0, 1, 2, and 3; p and q are each independently selected from the group consisting of 0, 1, 2, 3, 4, and 5, and, p and q are not both 0;
U¹ and U² are each independently selected from the group consisting of a bond, 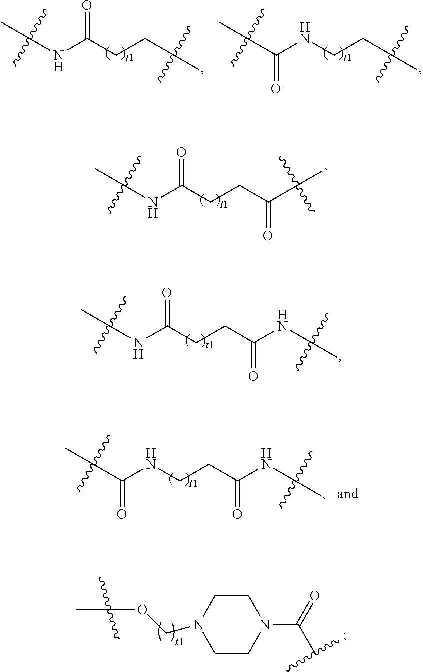
each t1 is independently selected from the group consisting of 1, 2, 3, and 4;
m1, m2, m3 and m4 are each independently selected from the group consisting of 0, 1, 2, 3, 4, and 5; or
(2) the compound has a structure of formula I′—B: 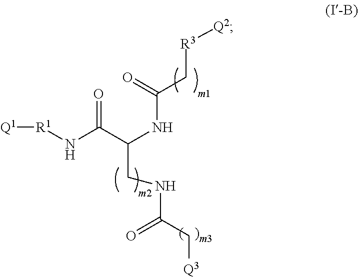
wherein, Q¹ is a FAP receptor-targeting moiety, Q² is an additional receptor-targeting moiety such as a GRPR receptor, Q³ is a chelating group; or Q² is a FAP receptor-targeting moiety, Q¹ is an additional receptor-targeting moiety such as a GRPR receptor, Q³ is a chelating group;
one of R¹ and R³ is —U¹-G¹-A¹-L-A²-G²-U²—, and the other is selected from the group consisting of a bond, —(CH₂)_{q}—S—(CH₂)ₚ—, U¹, and G¹; p and q are each independently selected from the group consisting of 0, 1, 2, 3, 4, and 5, and p and q are not both 0;
L is 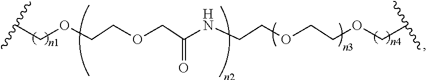
n1, n2, n3 and n4 are each independently 0, 1, 2, or 3;
A¹ and A² are each independently selected from the group consisting of a bond, —C(═O) NH—, —NHC(═O)—, and —C(═O)—;
G¹ and G² are each independently selected from the group consisting of a bond and 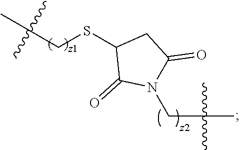
z1 and z2 are each independently selected from the group consisting of 0, 1, 2, and 3;
U¹ and U² are each independently selected from the group consisting of a bond, 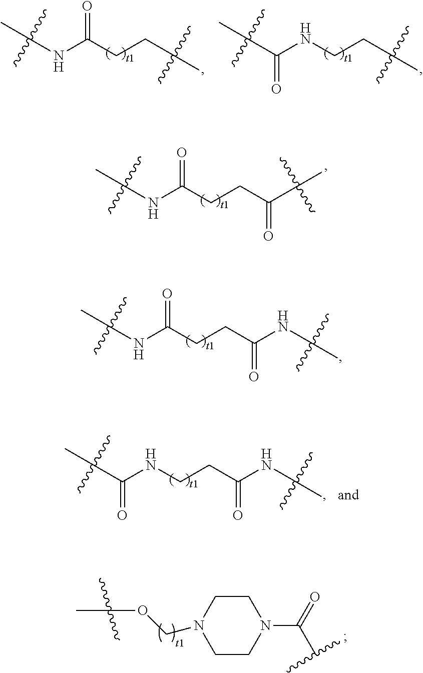
each t1 is independently selected from the group consisting of 1, 2, 3, and 4;
m1, m2 and m3 are each independently selected from the group consisting of 0, 1, 2, 3, 4, and 5;
or
(3) the compound has a structure of formula I′—C: 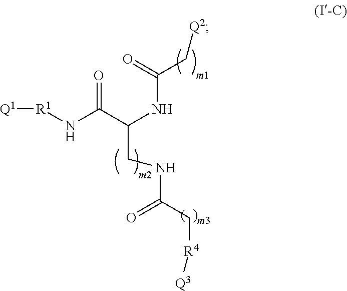
each symbol is as defined in Formula (I), preferably, Q¹ is a FAP receptor-targeting moiety, Q³ is an additional receptor-targeting moiety such as an αᵥβ₃ receptor, Q² is a chelating group; or Q³ is a FAP receptor-targeting moiety, Q¹ is an additional receptor-targeting moiety such as an αᵥβ₃ receptor, Q² is a chelating group;
preferably, one of R¹ and R⁴ is —U¹-G¹-A¹-L-A²-G²-U²—, and the other is selected from the group consisting of a bond, —(CH₂)_{q}—S—(CH₂)ₚ—, U¹, and G¹; p and q are each independently selected from the group consisting of 0, 1, 2, 3, 4, and 5, and p and q are not both 0;
or 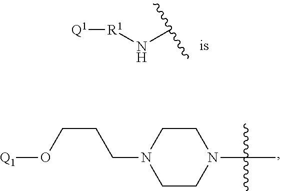
R⁴ is —U¹-G¹-A¹-L-A²-G²-U²—;
L is 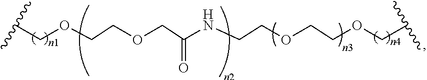
n1, n2, n3 and n4 are each independently 0, 1, 2, or 3;
A¹ and A² are each independently selected from the group consisting of a bond, —C(═O) NH—, —NHC(═O)—, and —C(═O)—;
G¹ and G² are each independently selected from the group consisting of a bond and 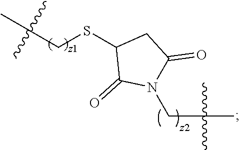
z1 and z2 are each independently selected from the group consisting of 0, 1, 2, and 3;
U¹ and U² are each independently selected from the group consisting of a bond, 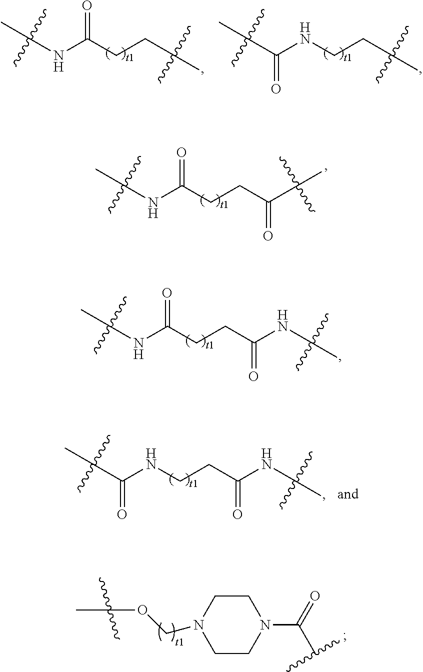
each t1 is independently selected from the group consisting of 1, 2, 3, and 4;
m1, m2 and m3 are each independently selected from the group consisting of 0, 1, 2, 3, 4, and 5.

3. The compound according to claim 1, or a stereoisomer, tautomer, or pharmaceutically acceptable salt thereof, characterized in that the compound has a structure of formula I-2, I-1, I-3, I-4, I-5, I-6, I-7, I-8, II-A, II-B, II-C1, II-C2, II-D, III-1, or III-2: 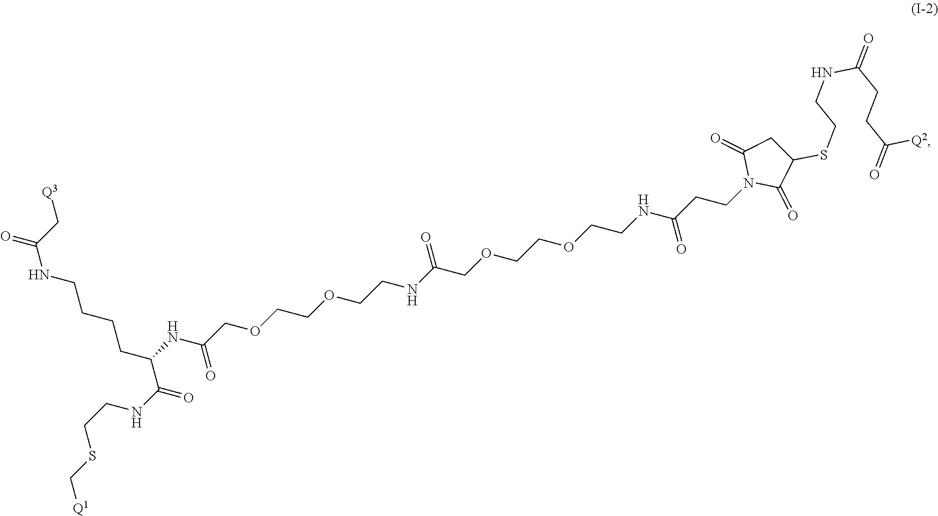
in formula I-2, Q¹, Q², Q³ are as defined in Formula (I); preferably, Q¹ is a FAP receptor-targeting moiety; Q² is an additional receptor-targeting moiety such as a CXCR4 receptor; Q³ is a chelating group; or Q¹ is an additional receptor-targeting moiety such as a CXCR4 receptor; Q² is a FAP receptor-targeting moiety; Q³ is a chelating group; 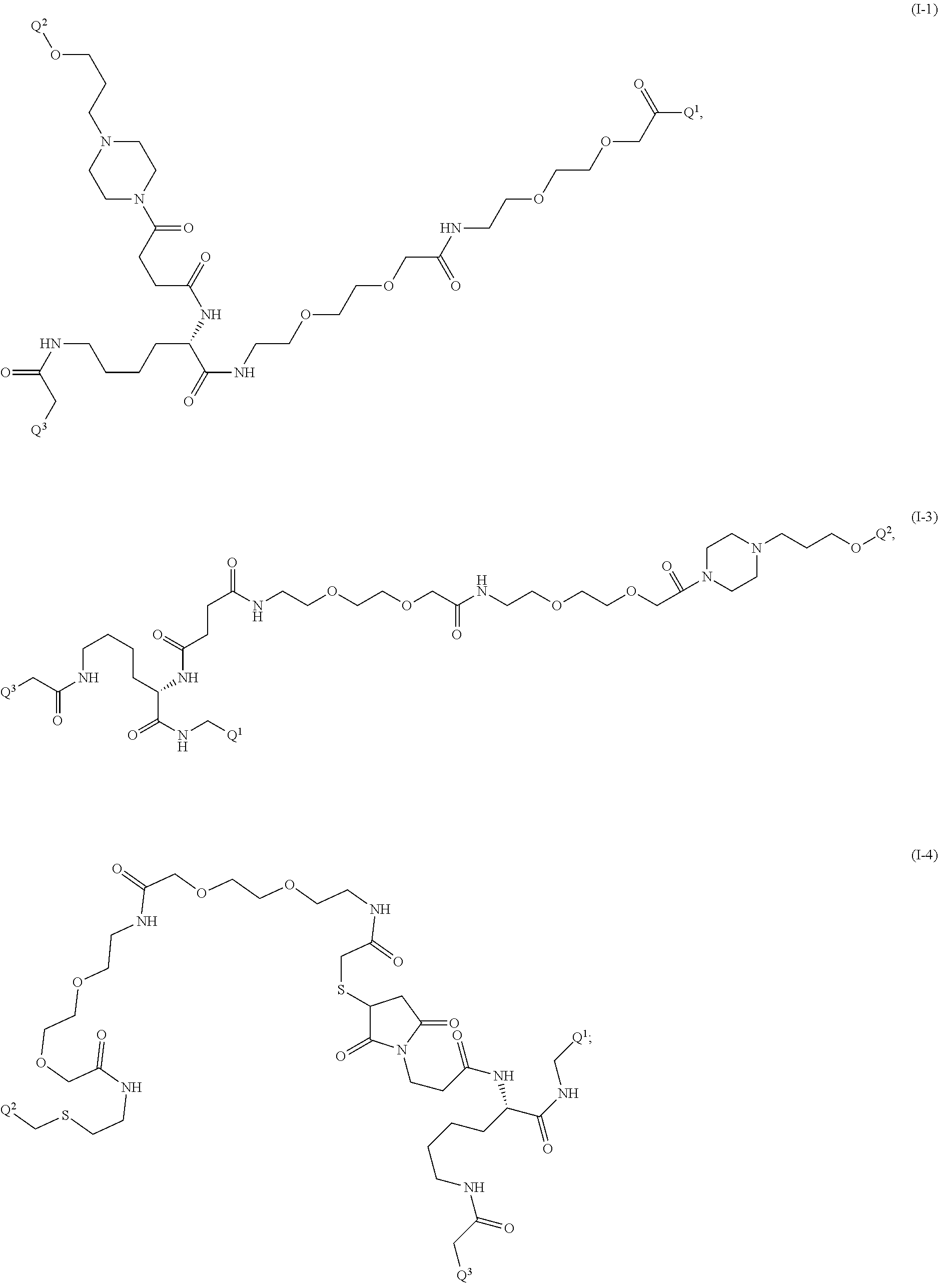
in formulas I-1, I-3 and I-4, Q¹, Q², Q³ are as defined in Formula (I); preferably, Q¹ is an additional receptor-targeting moiety such as a CXCR4 receptor, Q² is a FAP receptor-targeting moiety, Q³ is a chelating group; or Q² is a FAP receptor-targeting moiety, Q¹ is an additional receptor-targeting moiety such as a CXCR4 receptor, Q³ is a chelating group; 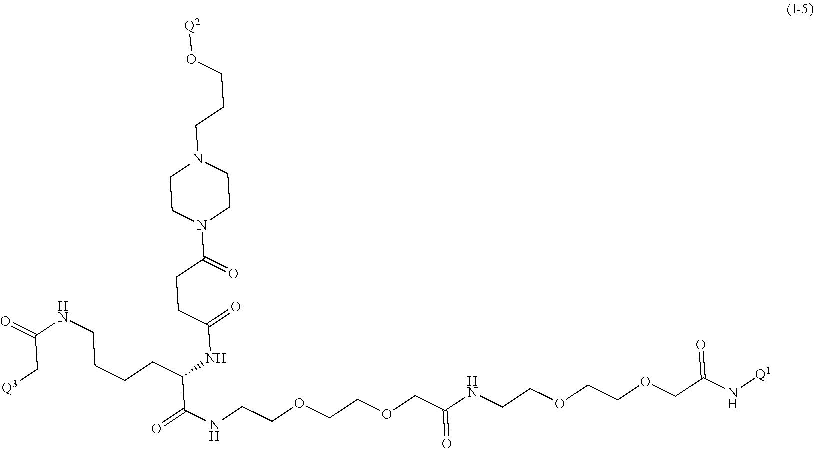
in formula I-5, Q¹, Q², Q³ are as defined in Formula (I); preferably, Q¹ is an additional receptor-targeting moiety such as a GRPR receptor; Q² is a FAP receptor-targeting moiety; Q³ is a chelating group; or Q² is an additional receptor-targeting moiety such as a GRPR receptor; Q¹ is a FAP receptor-targeting moiety; Q³ is a chelating group; 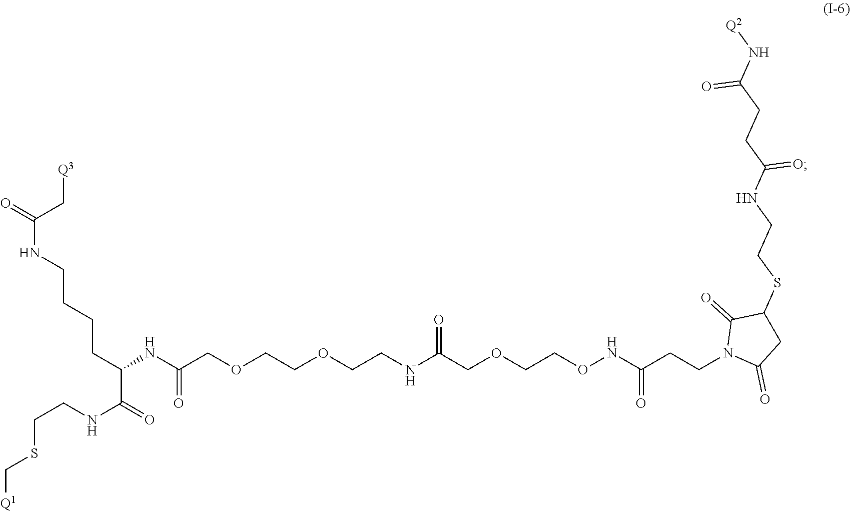
in formula I-6, Q¹, Q², Q³ are as defined in Formula (I); preferably, Q¹ is a FAP receptor-targeting moiety; Q² is an additional receptor-targeting moiety such as a GRPR receptor, Q³ is a chelating group; or Q² is a FAP receptor-targeting moiety; Q¹ is an additional receptor-targeting moiety such as a GRPR receptor; Q³ is a chelating group; 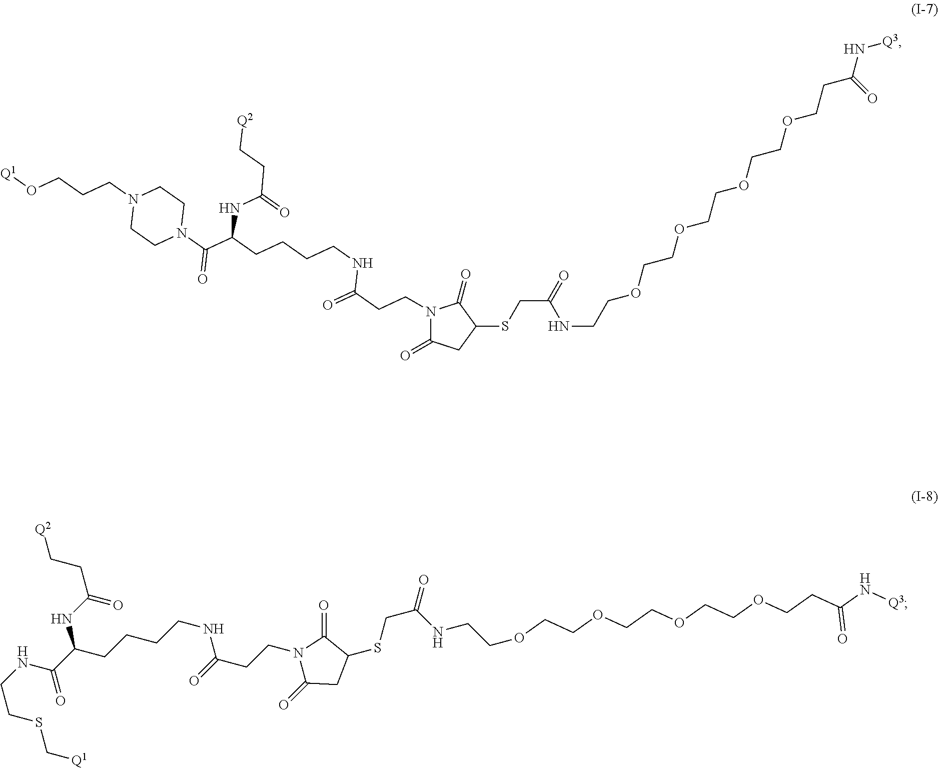
in formulas I-7 and I-8, Q¹, Q², Q¹ are as defined in Formula (I); preferably, Q¹ is a FAP receptor-targeting moiety; Q¹ is a chelating group; Q¹ is an additional receptor-targeting moiety such as an αᵥβ₃ receptor; or Q³ is a FAP receptor-targeting moiety; Q¹ is a chelating group; Q¹ is an additional receptor-targeting moiety such as an αᵥβ₃ receptor; 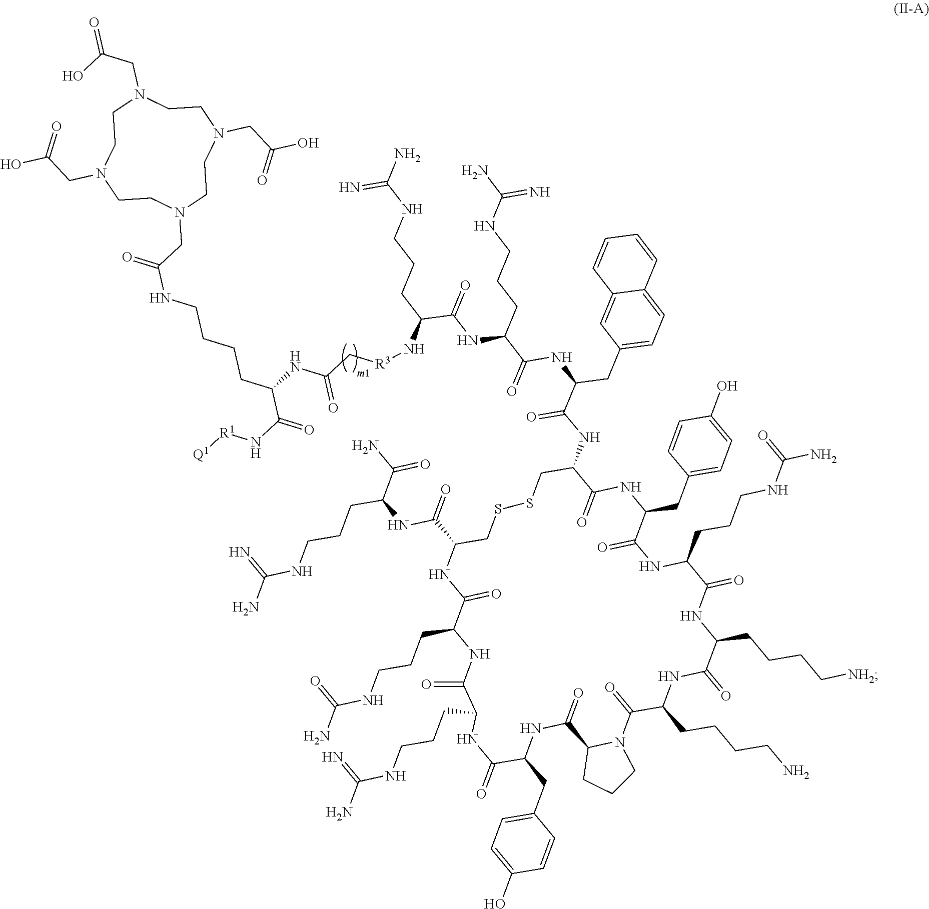
in formula II-A, each symbol is as defined in Formula (I), preferably, Q¹ is a FAP receptor-targeting moiety; 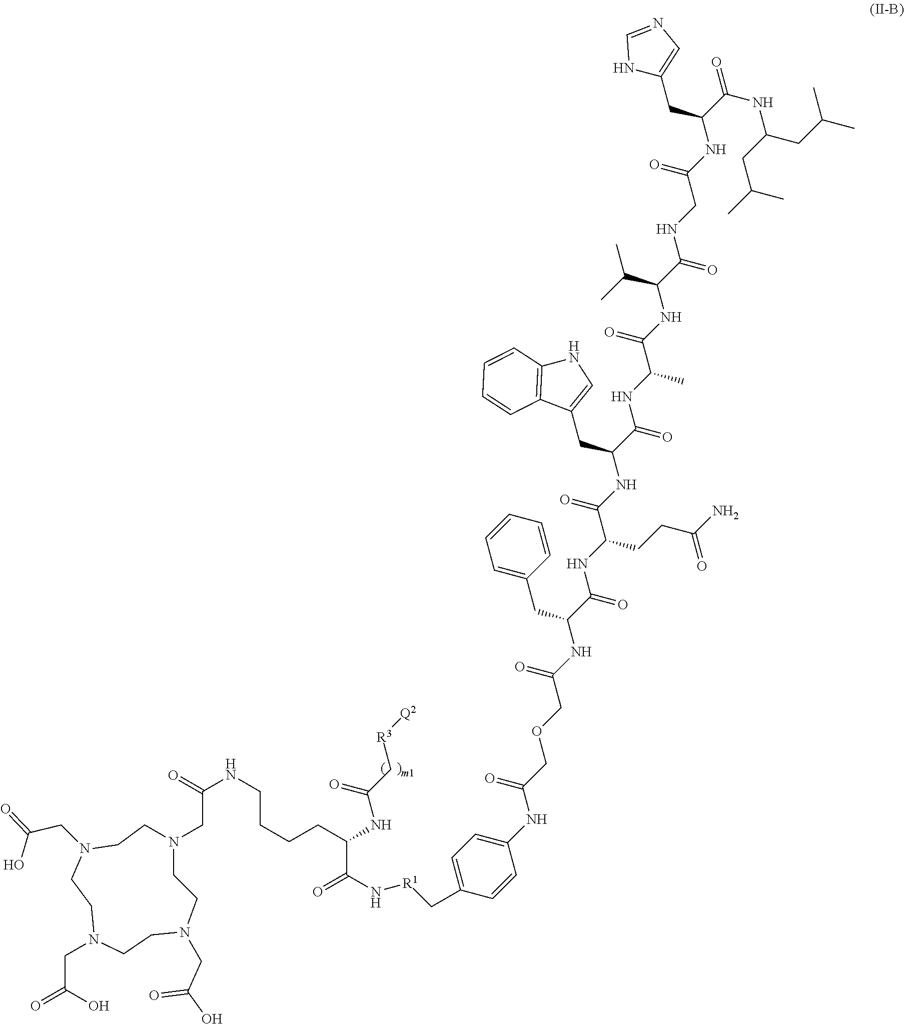
in formula II-B, each symbol is as defined in Formula (I), preferably, Q¹ is a FAP receptor-targeting moiety; 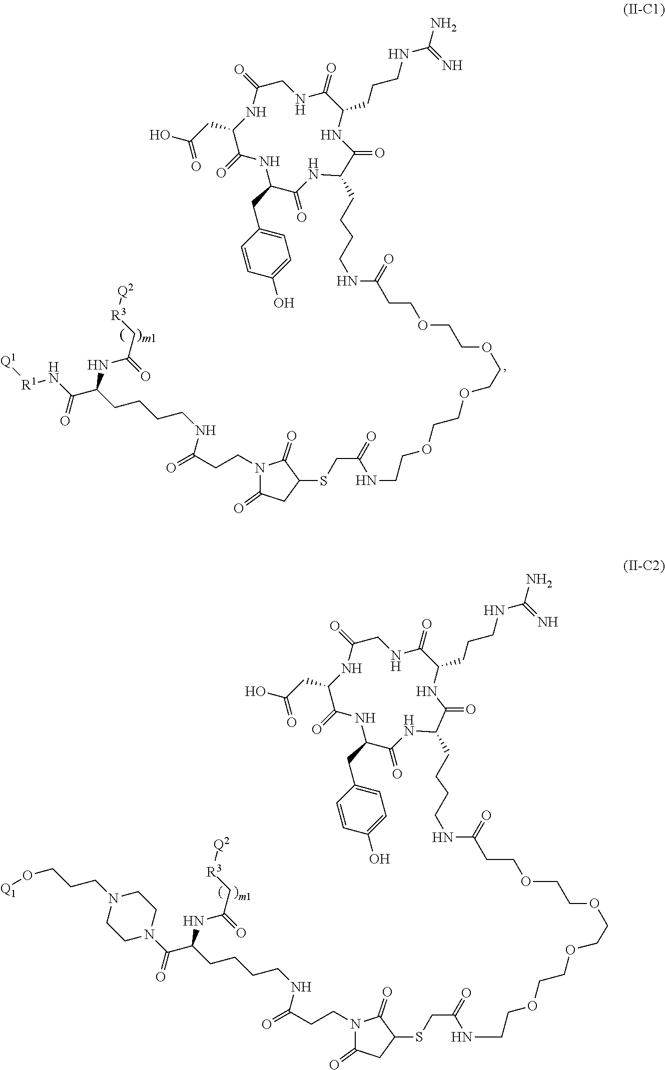
in formulas II-C1 and II-C2, each symbol is as defined in Formula (I), preferably, Q¹ is a FAP receptor-targeting moiety; Q¹ is a chelating group; 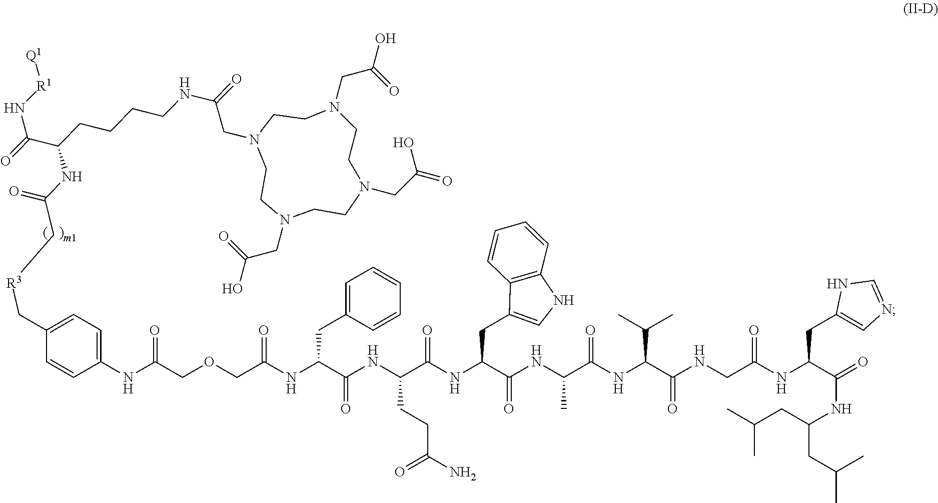
in formula II-D, each symbol is as defined in Formula (I), preferably, Q¹ is a FAP receptor-targeting moiety; 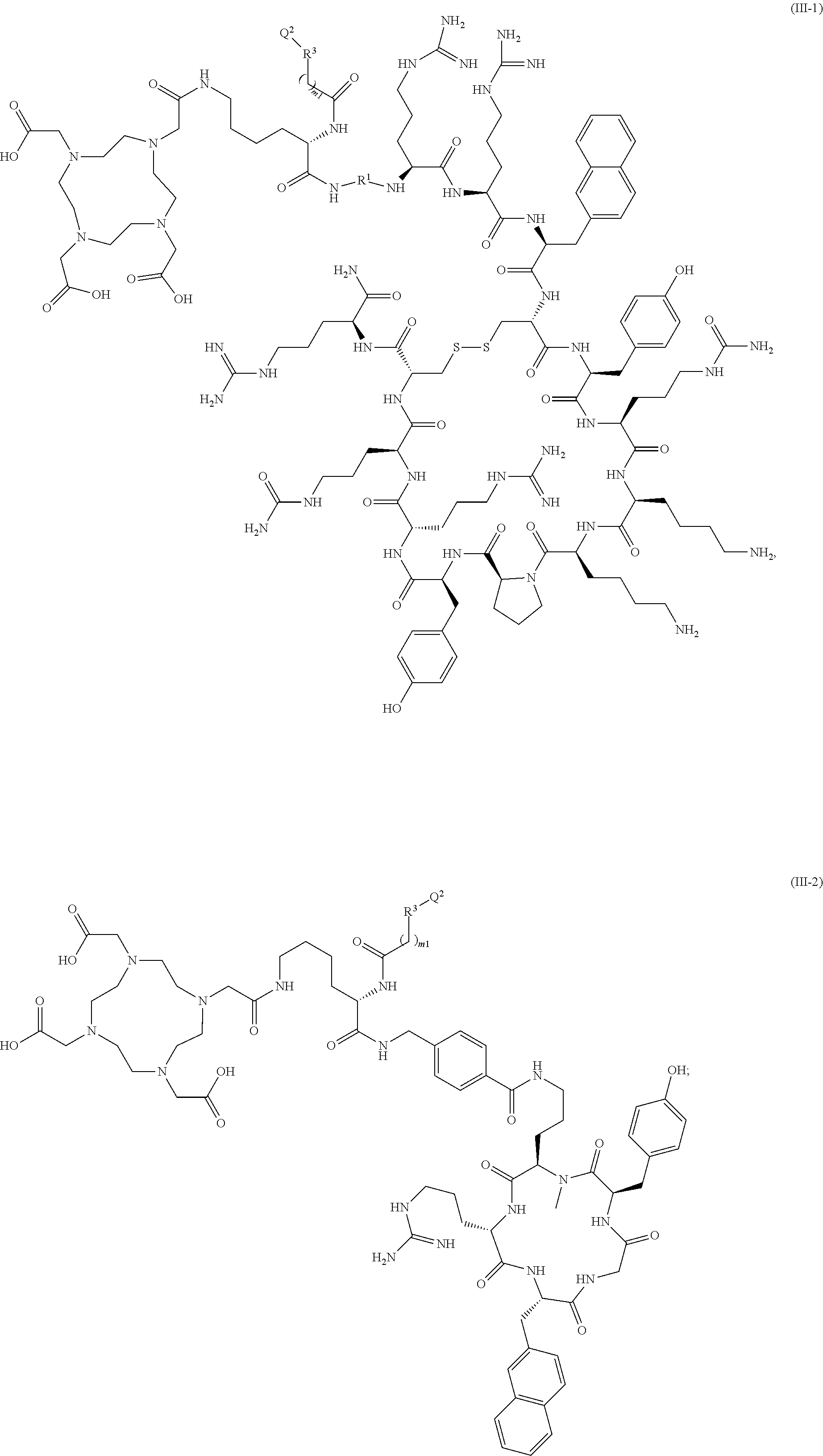
wherein, in formula III-1 or III-2, each symbol is as defined in Formula (I); preferably, Q¹ is a FAP receptor-targeting moiety.

4. The compound according to claim 1, or a stereoisomer, tautomer, or pharmaceutically acceptable salt thereof, characterized in that the compound has a structure of formula V: 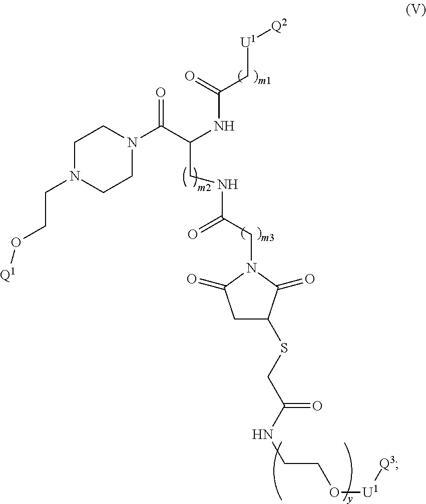
wherein, each U¹ is independently selected from the group consisting of a bond, 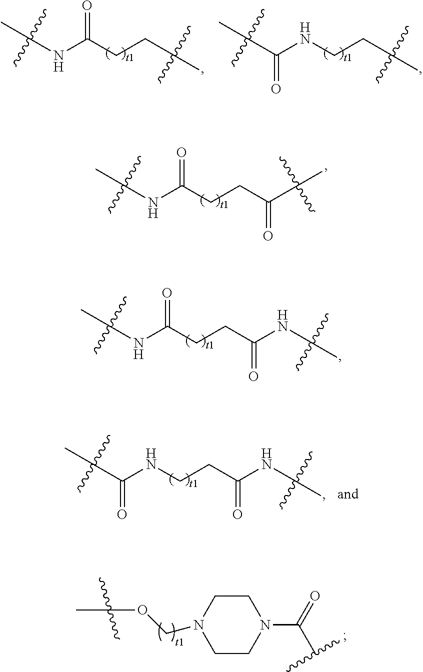
each t1 is independently selected from the group consisting of 1, 2, 3, and 4;
m1, m2, m3 and m4 are each independently selected from the group consisting of 0, 1, 2, 3, 4, and 5;
y is an integer selected from the group consisting of 3 to 10;
Q¹ is a FAP receptor-targeting moiety,
Q² is a chelating group,
Q³ is an αᵥβ₃ receptor-targeting moiety,
preferably, Q¹ is selected from the group consisting of:

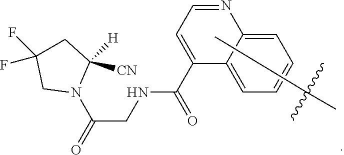

5. The compound according to claim 1, characterized in that the FAP receptor-targeting moiety has a structure of formula II-1: 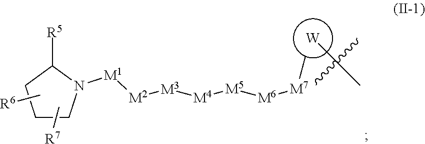
M¹, M², M³, M⁴, M⁵, M⁶, M² are each independently selected from the group consisting of a bond, —O—, —CH₂—, —NR⁸—, —C(═O)—, —C(═S)—, —C(═NH)R⁸—, —CHR⁸—, and —C(R⁸)₂—, with the proviso that: two O atoms in the structure are not directly adjacent to each other; at most four of M¹, M², M³, M⁴, M⁵, M⁶, M⁷ are bonds at the same time;
R⁶ and R⁷ are each independently selected from the group consisting of —H, —OH, halogen atom, C₁₋₆alkyl, —O—C₁₋₆alkyl, and —S—C₁₋₆alkyl;
R⁵ is selected from the group consisting of —H, —CN, —B(OH)₂, —C(═O)-alkyl, —C(═O)-aryl-, —C═C—C(═O)-aryl, —C═C—S(═O)₂—Ar—, —C(═O)OH, —S(═O)₂OH, —S(═O)₂NH₂, —P(═O) (OH)₂, and 5-tetrazolyl;
R⁸ is selected from the group consisting of —H, C₁₋₆alkyl, —O—C₁₋₆alkyl, —S—C₁₋₆alkyl, C₂₋₆alkenyl, C₂₋₆heteroalkenyl, C₅₋₆cycloalkenyl, C₄₋₆heterocycloalkenyl, C₂₋₆alkynyl, C₆₋₁₀aryl, and C₆₋₁₀arylC₁₋₆alkyl, wherein the C₁₋₆alkyl is optionally substituted by 1-3 substituents selected from the group consisting of —OH, oxygen, and halogen atom;
the ring W is selected from the group consisting of naphthyl, and 5-10-membered nitrogen-containing heteroaryl.

6. The compound according to claim 5, characterized in that:
-M¹-M²-M³-M⁴-M⁵-M⁶-M⁷- is —C(═O)—CH₂—NR⁸—C(═O)—; and/or
R⁶ and R⁷ are each independently selected from the group consisting of H and F; and/or
the ring W is quinolinyl; preferably, the ring W is 4-quinolinyl; and/or

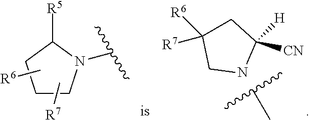

7. The compound according to claim 1, characterized in that the FAP receptor-targeting moiety has a structure of formula II-2: 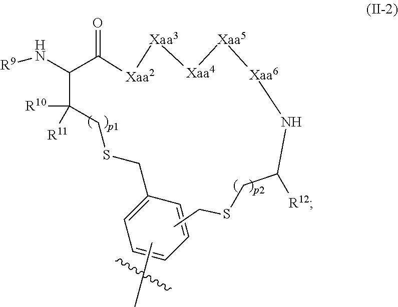
R⁹ is alkylacyl;
R¹⁰, and R¹¹ are each independently selected from the group consisting of H and CH₃;
p1 is selected from the group consisting of 0 and 1; p2 is selected from the group consisting of 1 and 2;
Xaa², Xaa³, Xaa⁴, Xaa⁵ and Xaa⁶ are each independently selected from the group consisting of common amino acid residues and non-common amino acid residues;
preferably,
Xaa² is 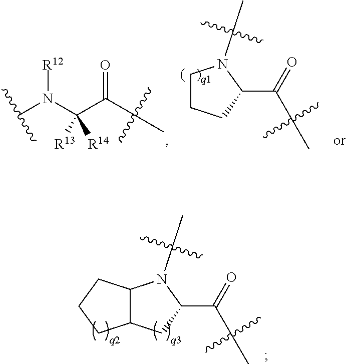
R¹², R¹³, and R¹⁴ are each independently selected from the group consisting of C₁₋₂alkyl and carboxyl, and H, wherein the C₁₋₂alkyl is optionally substituted by 1 or 2 substituents selected from the group consisting of —OH, NH₂, halogen atom, and C₅₋₇cycloalkyl; 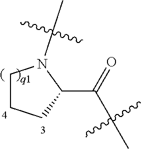
is optionally substituted at positions 3 and 4 by 1 or 2 substituents selected from the group consisting of methyl, OH, NH₂, and F;
q1 is selected from the group consisting of 0, 1, and 2;
q2 is selected from the group consisting of 1, 2, and 3;
q3 is selected from the group consisting of 1 and 2;
Xaa³ is 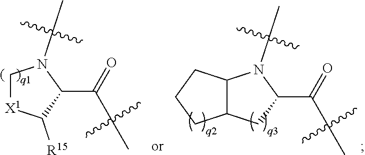
X¹ is selected from the group consisting of CH₂, CF₂, CHR¹⁶, S, O, and NH;
R¹⁵ is H, methyl, OH, NH₂, or F,
R¹⁶ is methyl, OH, NH₂, or F;
Xaa⁴ is 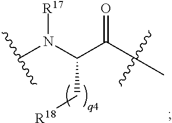
R¹⁷ is methyl or H;
R¹⁸ is selected from the group consisting of H, —OH, —C(═O)OH, —(C═O)NH₂, X², and —NH—C(═O)—X², wherein X² is selected from the group consisting of C₁₋₆alkyl, phenyl, and C₅₋₆ heteroaryl, and X² is optionally substituted by 1 or 2 substituents selected from the group consisting of methyl, C(═O)NH₂, halogen atom, NH₂ and OH;
q4 is selected from the group consisting of 1, 2, and 3; wherein one or two hydrogen atoms in the 1, 2 or 3 CH₂ moieties, independently from each other, are optionally replaced by methyl, ethyl, phenyl, and C₅₋₆heteroaryl,
Xaa⁵ is 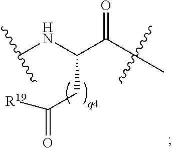
R¹⁹ is selected from the group consisting of OH and NH₂;
q5 is selected from the group consisting of 1, 2, and 3;
Xaa⁶ is selected from the group consisting of amino acid residues of aromatic L-α-amino acids and heteroaromatic L-α-amino acids.

8. The compound according to claim 1, characterized in that the FAP receptor-targeting moiety is: 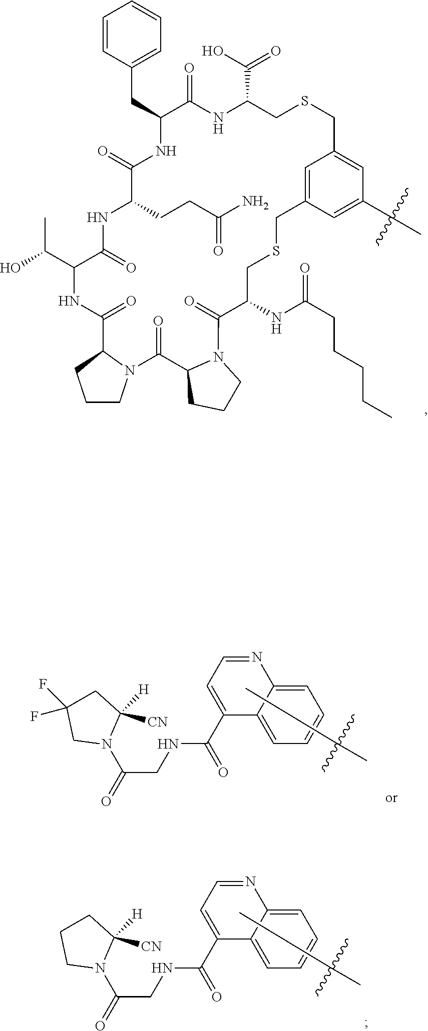
the CXCR4 receptor-targeting moiety is selected from the group consisting of: 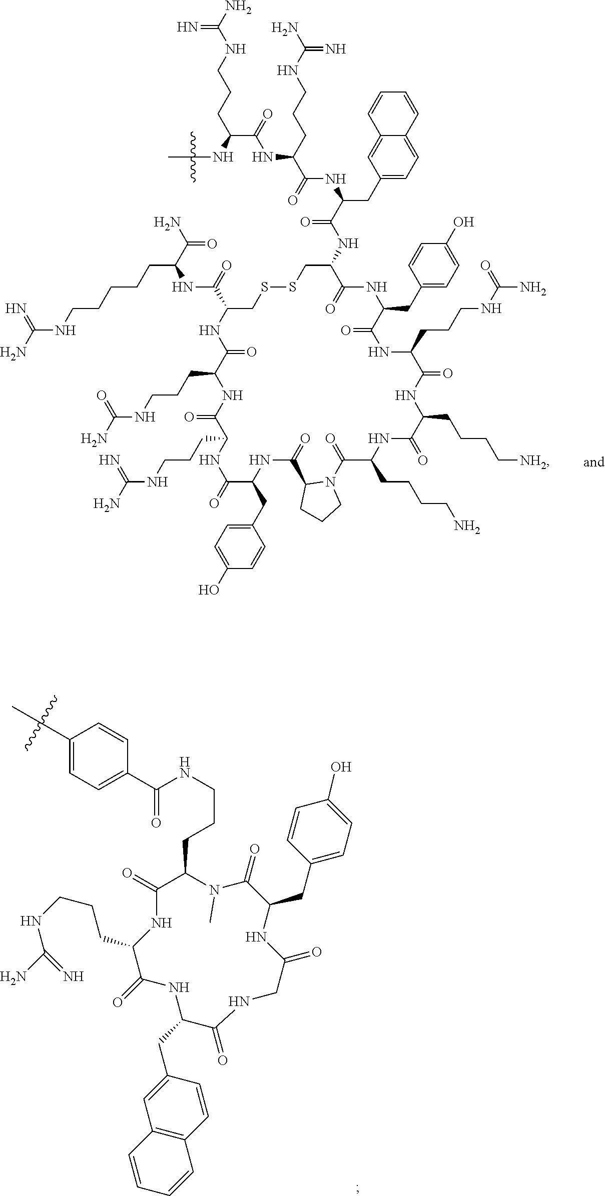 [SMILES: *N[C@@H](CCCNC(=N)N)C(=O)N[C@@H](CCCNC(=N)N)C(=O)N[C@@H](Cc1ccc2ccccc2c1)C(=O)N[C@H]1CSSC[C@@H](C(=O)N[C@@H](CCCCCNC(=N)N)C(N)=O)NC(=O)[C@H](CCCNC(N)=O)NC(=O)[C@H](CCCNC(=N)N)NC(=O)[C@H](Cc2ccc(O)cc2)NC(=O)[C@@H]2CCCN2C(=O)[C@H](CCCCN)NC(=O)[C@H](CCCCN)NC(=O)[C@H](CCCNC(N)=O)NC(=O)[C@H](Cc2ccc(O)cc2)NC1=O ; *c1ccc(C(=O)NCCC[C@@H]2C(=O)N[C@@H](CCCNC(=N)N)C(=O)N[C@@H](Cc3ccc4ccccc4c3)C(=O)NCC(=O)N[C@H](Cc3ccc(O)cc3)C(=O)N2C)cc1]
the αᵥβ₃ receptor-targeting moiety is: 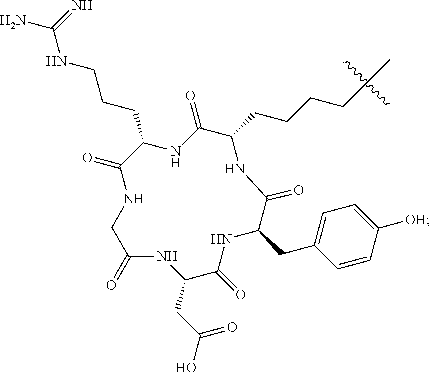 [SMILES: *CCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]
the GRPR receptor-targeting moiety is:

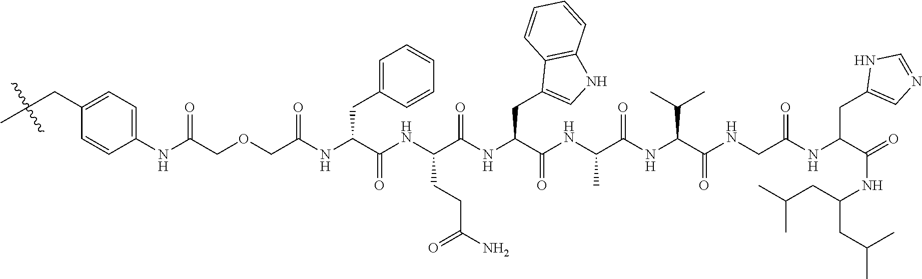 [SMILES: *Cc1ccc(NC(=O)COCC(=O)N[C@H](Cc2ccccc2)C(=O)N[C@@H](CCC(N)=O)C(=O)N[C@@H](Cc2c[nH]c3ccccc23)C(=O)N[C@@H](C)C(=O)N[C@H](C(=O)NCC(=O)NC(Cc2cnc[nH]2)C(=O)NC(CC(C)C)CC(C)C)C(C)C)cc1]

9. The compound according to claim 1, characterized in that: the chelating group is selected from the group consisting of 1,4,7,10-tetraazacyclododecane-1,4,7,10-tetraacetic acid (DOTA), 1,4,7,10-tetraazacyclododecane-1-(glutaric acid)-4,7,10-triacetic acid (DOTAGA), 1,4,7-triazacyclononane triacetic acid (NOTA), 1,4,7-triazacyclononane-N-glutaric acid-N′,N″-diacetic acid (NODAGA), 1,4,7-triazacyclononane-1,4-diacetic acid-methylphenylacetic acid (NODA-MPAA), bis-(2-hydroxybenzyl)ethylenediamine diacetic acid (HBED), 4,11-bis-(carboxymethyl)-1,4,8,11-tetraazabicyclo[6.6.2]-hexadecane (CB-TE2A), desferrioxamine (DFO), and a hexadentate tris(3,4-hydroxypyridinone) (THP)-derived group;
alternatively, the chelating group is one of the following: 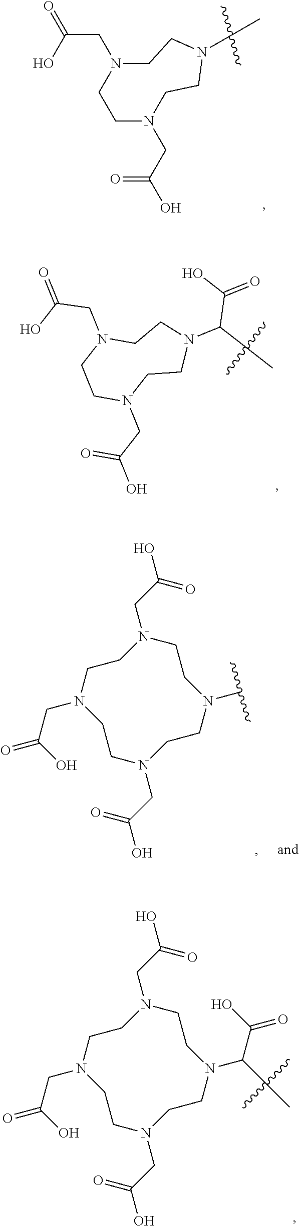 [SMILES: *N1CCN(CC(=O)O)CCN(CC(=O)O)CC1 ; *C(C(=O)O)N1CCN(CC(=O)O)CCN(CC(=O)O)CC1 ; *N1CCN(CC(=O)O)CCN(CC(=O)O)CCN(CC(=O)O)CC1 ; *C(C(=O)O)N1CCN(CC(=O)O)CCN(CC(=O)O)CCN(CC(=O)O)CC1]
preferably, the chelating group is

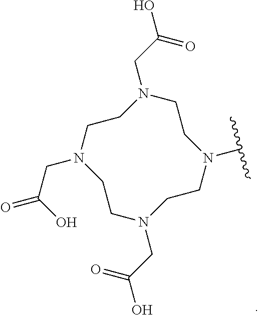 [SMILES: *N1CCN(CC(=O)O)CCN(CC(=O)O)CCN(CC(=O)O)CC1]

10. The compound according to claim 1, or a stereoisomer, tautomer, or pharmaceutically acceptable salt thereof, characterized in that the compound is selected from the group consisting of the following structures:

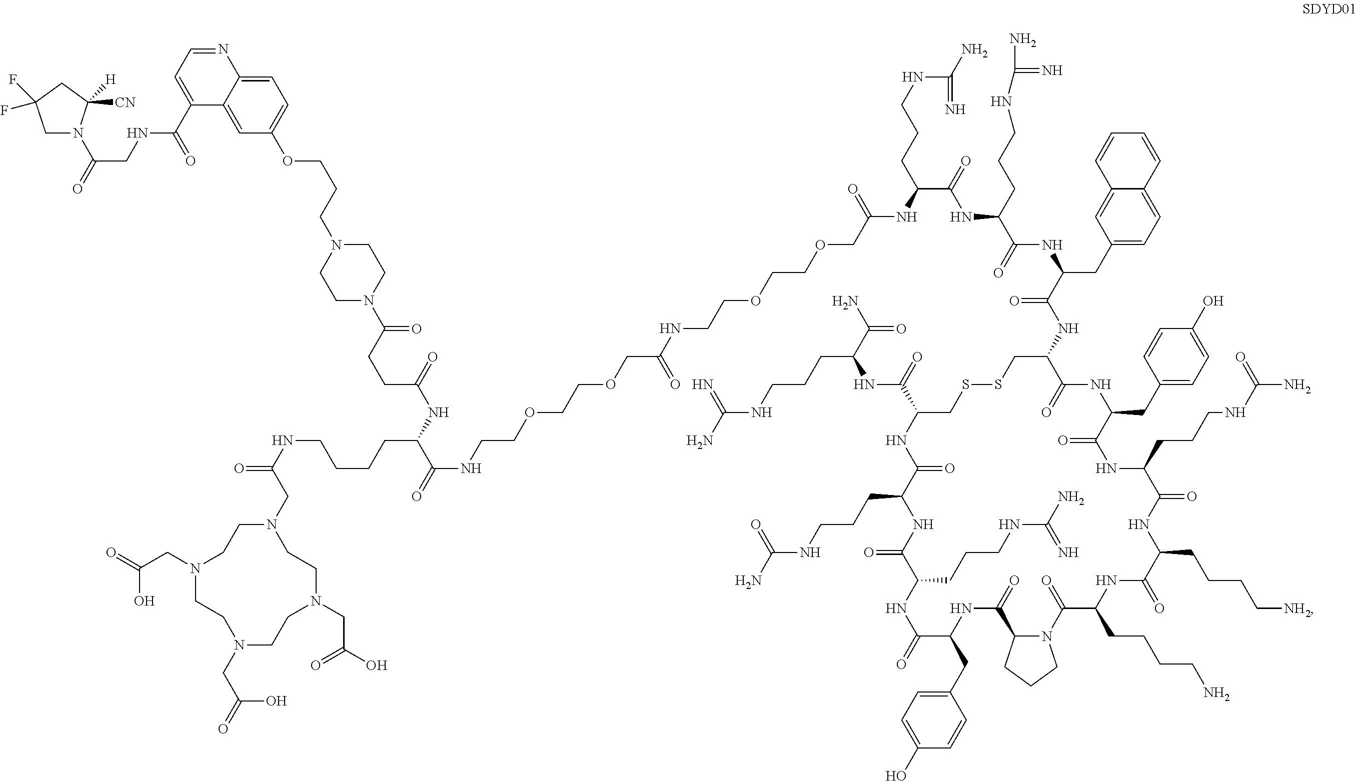 [SMILES: N#C[C@@H]1CC(F)(F)CN1C(=O)CNC(=O)c1ccnc2ccc(OCCCN3CCN(C(=O)CCC(=O)N[C@@H](CCCCNC(=O)CN4CCN(CC(=O)O)CCN(CC(=O)O)CCN(CC(=O)O)CC4)C(=O)NCCOCCOCC(=O)NCCOCCOCC(=O)N[C@@H](CCCNC(=N)N)C(=O)N[C@@H](CCCNC(=N)N)C(=O)N[C@@H](Cc4ccc5ccccc5c4)C(=O)N[C@H]4CSSC[C@@H](C(=O)N[C@@H](CCCNC(=N)N)C(N)=O)NC(=O)[C@H](CCCNC(N)=O)NC(=O)[C@H](CCCNC(=N)N)NC(=O)[C@H](Cc5ccc(O)cc5)NC(=O)[C@@H]5CCCN5C(=O)[C@H](CCCCN)NC(=O)[C@H](CCCCN)NC(=O)[C@H](CCCNC(N)=O)NC(=O)[C@H](Cc5ccc(O)cc5)NC4=O)CC3)cc12]

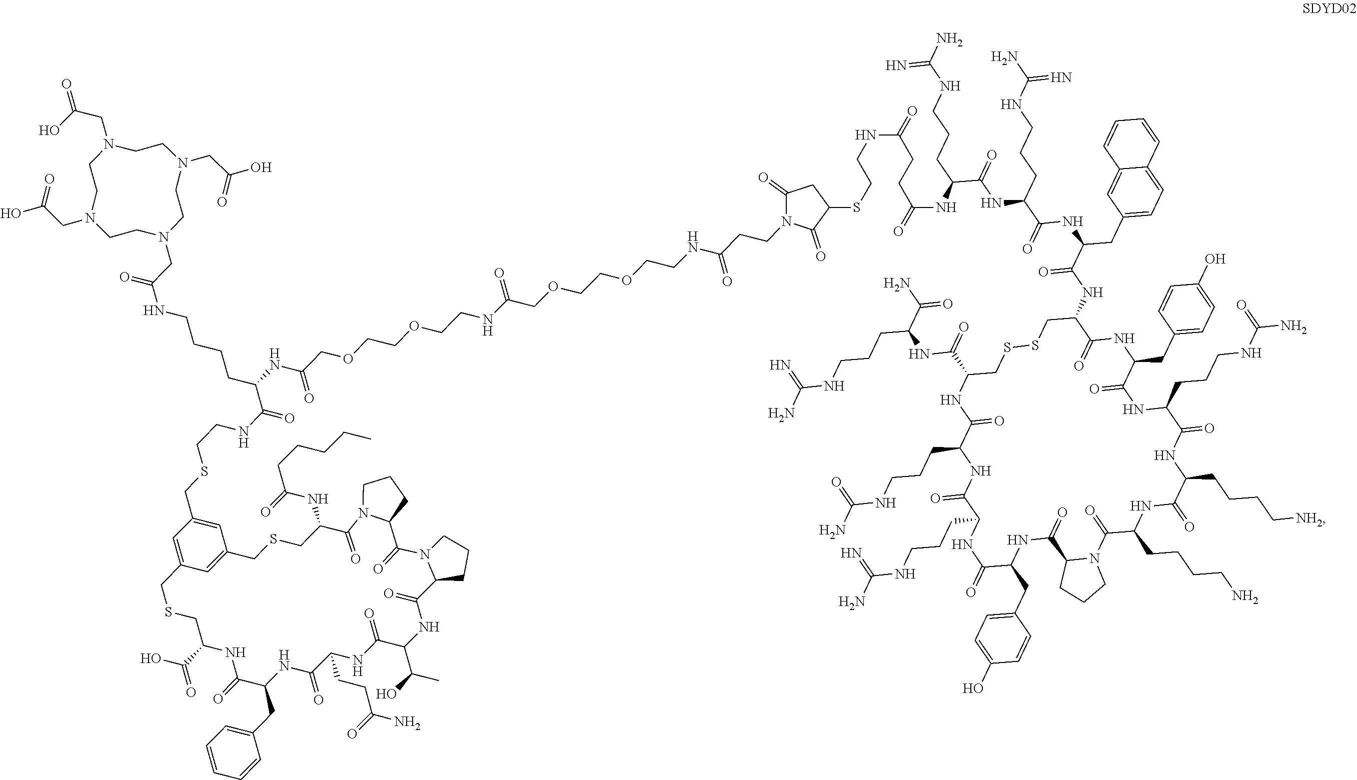

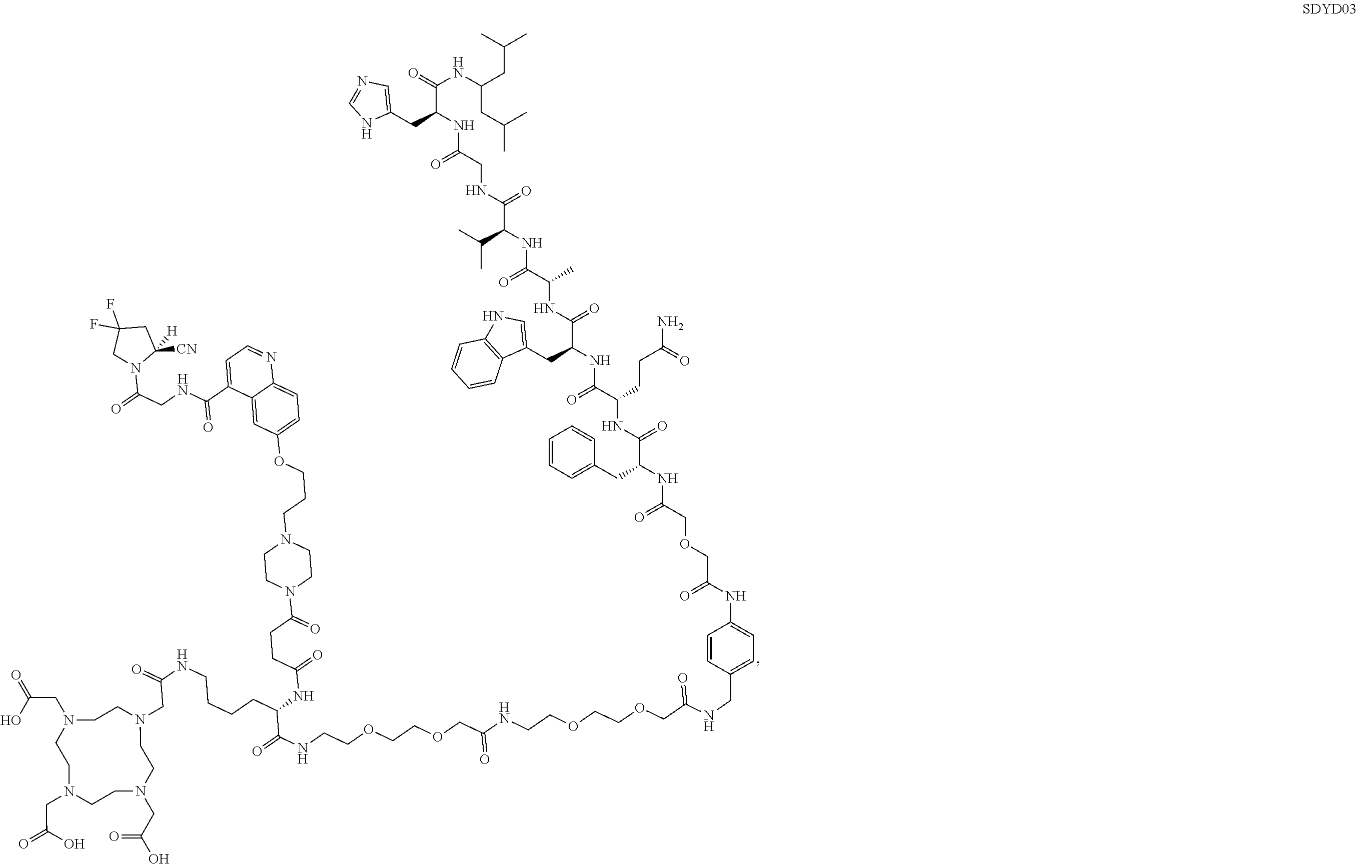 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)COCCOCCNC(=O)COCCOCCNC(=O)[C@H](CCCCNC(=O)CN2CCN(CC(=O)O)CCN(CC(=O)O)CCN(CC(=O)O)CC2)NC(=O)CCC(=O)N2CCN(CCCOc3ccc4nccc(C(=O)NCC(=O)N5CC(F)(F)C[C@H]5C#N)c4c3)CC2)cc1)C(C)C]

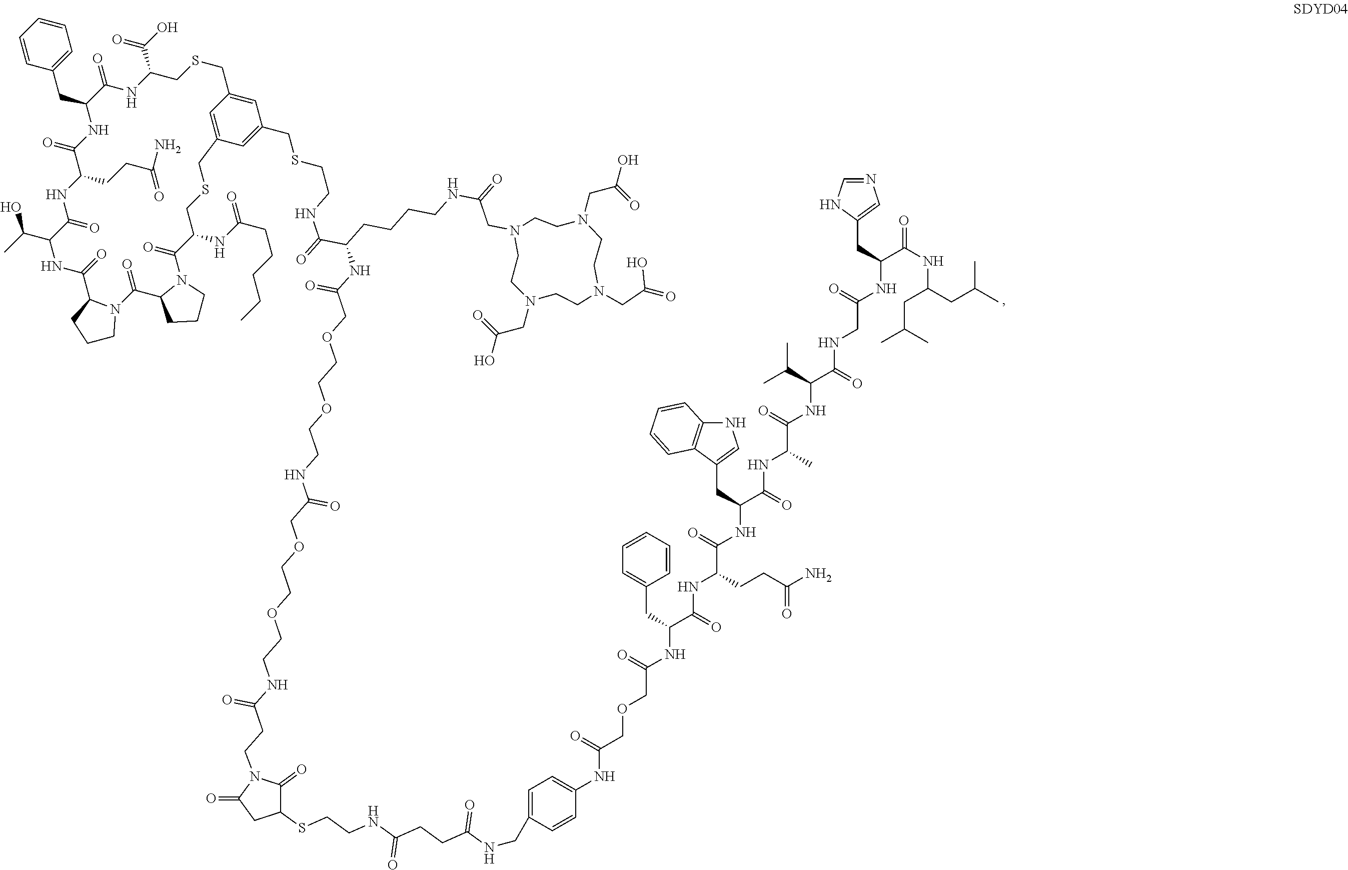 [SMILES: CCCCCC(=O)N[C@H]1CSCc2cc(CSCCNC(=O)[C@H](CCCCNC(=O)CN3CCN(CC(=O)O)CCN(CC(=O)O)CCN(CC(=O)O)CC3)NC(=O)COCCOCCNC(=O)COCCOCCNC(=O)CCN3C(=O)CC(SCCNC(=O)CCC(=O)NCc4ccc(NC(=O)COCC(=O)N[C@H](Cc5ccccc5)C(=O)N[C@@H](CCC(N)=O)C(=O)N[C@@H](Cc5c[nH]c6ccccc56)C(=O)N[C@@H](C)C(=O)N[C@H](C(=O)NCC(=O)N[C@@H](Cc5cnc[nH]5)C(=O)NC(CC(C)C)CC(C)C)C(C)C)cc4)C3=O)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O]

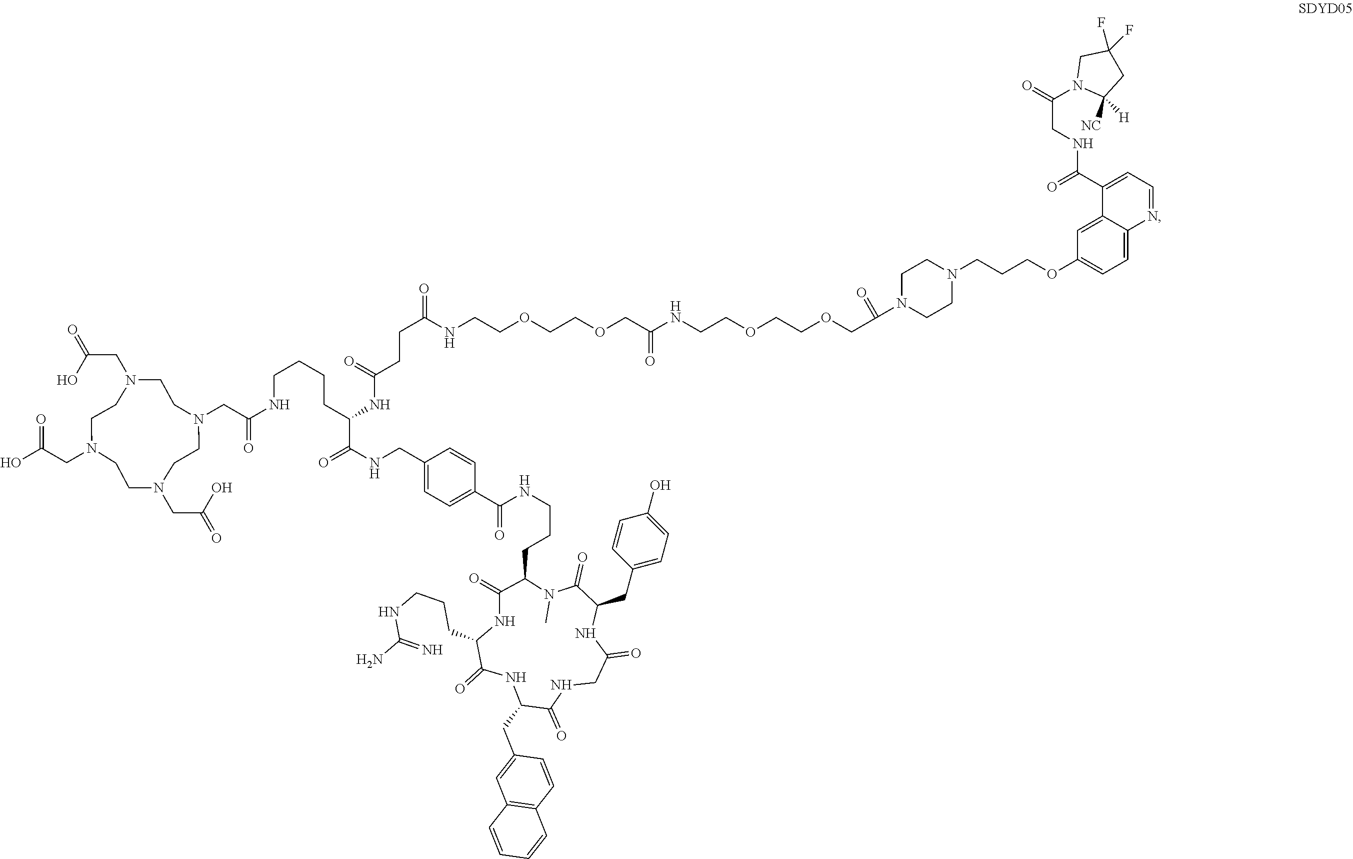 [SMILES: CN1C(=O)[C@@H](Cc2ccc(O)cc2)NC(=O)CNC(=O)[C@H](Cc2ccc3ccccc3c2)NC(=O)[C@H](CCCNC(=N)N)NC(=O)[C@H]1CCCNC(=O)c1ccc(CNC(=O)[C@H](CCCCNC(=O)CN2CCN(CC(=O)O)CCN(CC(=O)O)CCN(CC(=O)O)CC2)NC(=O)CCC(=O)NCCOCCOCC(=O)NCCOCCOCC(=O)N2CCN(CCCOc3ccc4nccc(C(=O)NCC(=O)N5CC(F)(F)C[C@H]5C#N)c4c3)CC2)cc1]

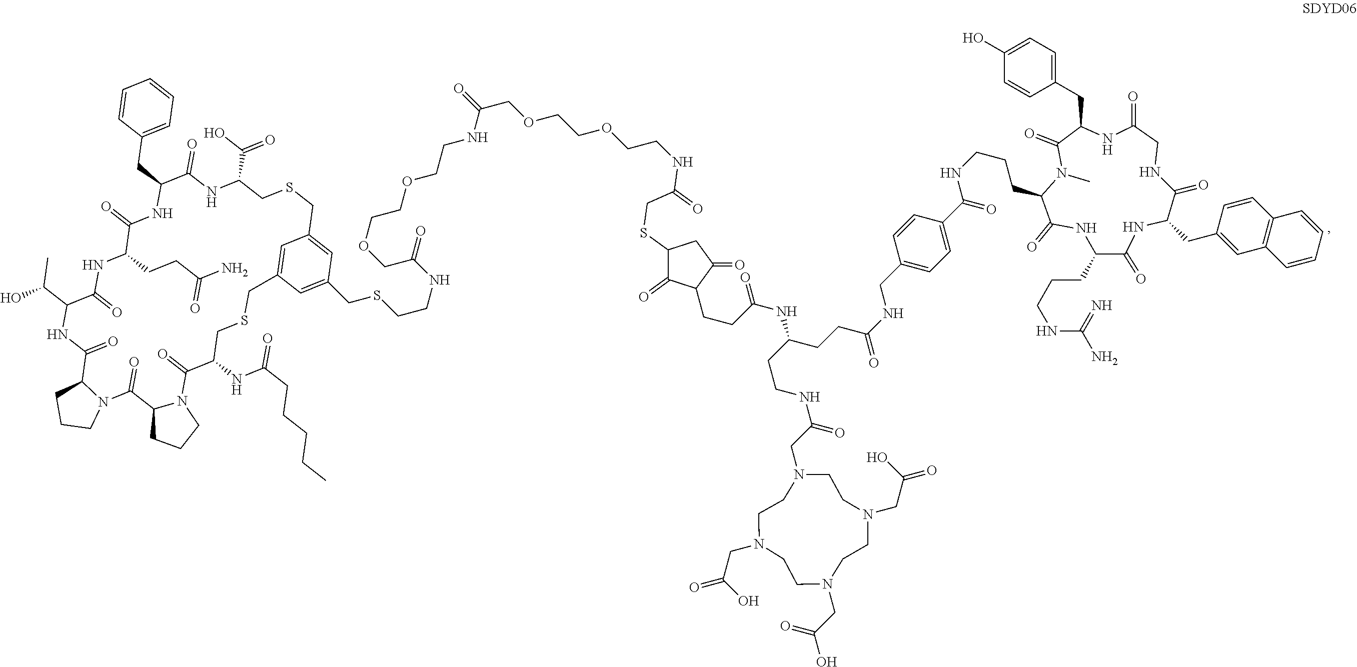 [SMILES: CCCCCC(=O)N[C@H]1CSCc2cc(CSCCNC(=O)COCCOCCNC(=O)COCCOCCNC(=O)CSC3CC(=O)C(CCC(=O)N[C@@H](CCNC(=O)CN4CCN(CC(=O)O)CCN(CC(=O)O)CCN(CC(=O)O)CC4)CCC(=O)NCc4ccc(C(=O)NCCC[C@@H]5C(=O)N[C@@H](CCCNC(=N)N)C(=O)N[C@@H](Cc6ccc7ccccc7c6)C(=O)NCC(=O)N[C@H](Cc6ccc(O)cc6)C(=O)N5C)cc4)C3=O)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O]

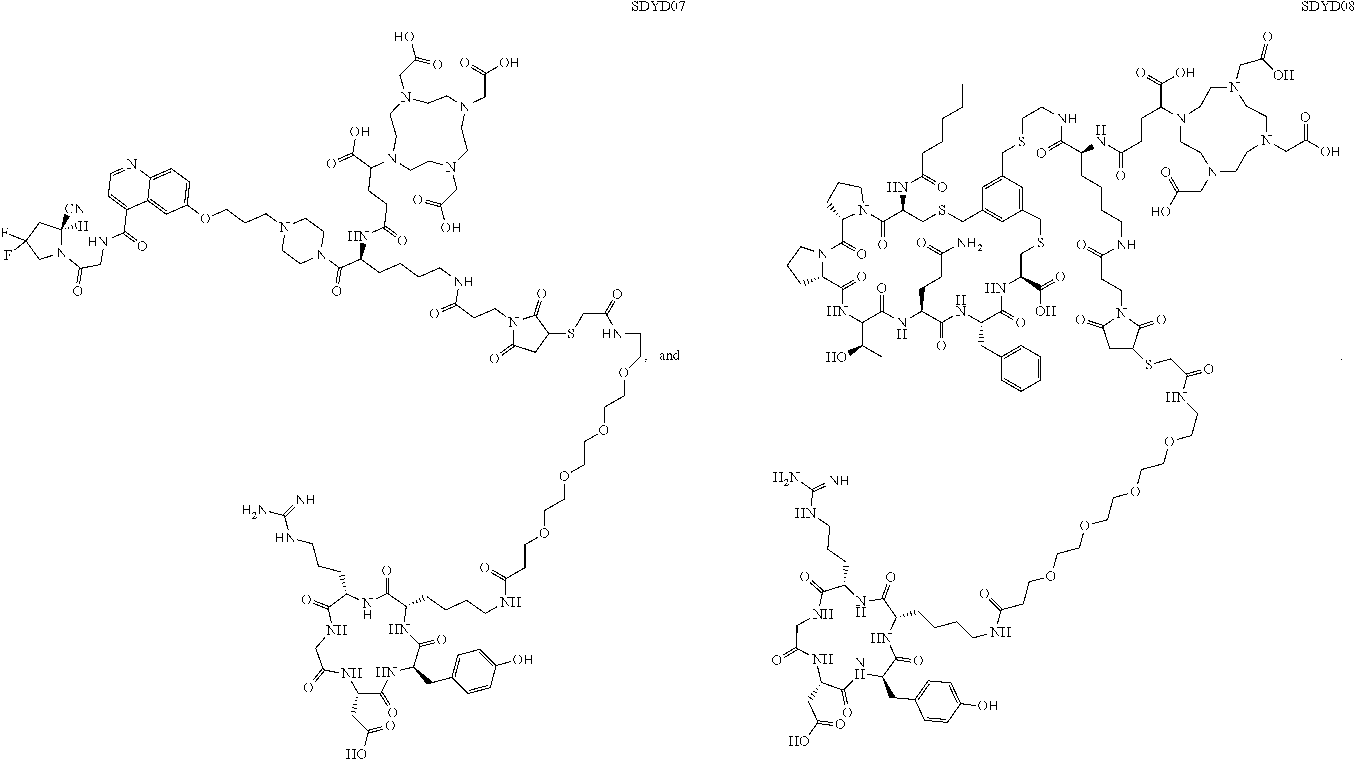

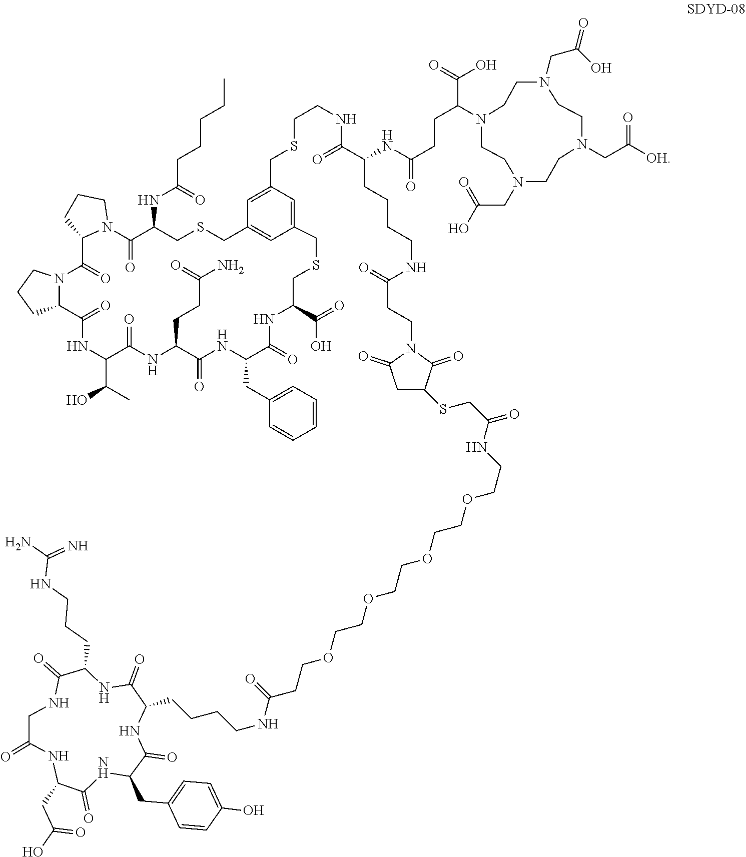 [SMILES: CCCCCC(=O)N[C@H]1CSCc2cc(CSCCNC(=O)[C@@H](CCCCNC(=O)CCN3C(=O)CC(SCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C3=O)NC(=O)CCC(C(=O)O)N3CCN(CC(=O)O)CCN(CC(=O)O)CCN(CC(=O)O)CC3)cc(c2)CSC[C@@H](C(=O)O)NC(=O)[C@H](Cc2ccccc2)NC(=O)[C@H](CCC(N)=O)NC(=O)C([C@@H](C)O)NC(=O)[C@@H]2CCCN2C(=O)[C@@H]2CCCN2C1=O]

11. A radionuclide-labeled compound obtained by labeling the compound according to claim 1 with a radionuclide.

12. The radionuclide-labeled compound according to claim 11, characterized in that the radionuclide is selected from the group consisting of ¹⁸F, ⁵¹Cr, ⁶⁷Ga, ⁶⁸Ga, ¹¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁸Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ⁶⁴Cu, ⁶⁷Cu, ¹⁸⁸Re, ¹⁸⁶Re, ¹⁹⁸Au, ²²⁵Ac, ²²⁷Th, and ¹⁹⁹Ag.

13. The radionuclide-labeled compound according to claim 11, characterized in that the radionuclide-labeled compound is selected from the group consisting of:

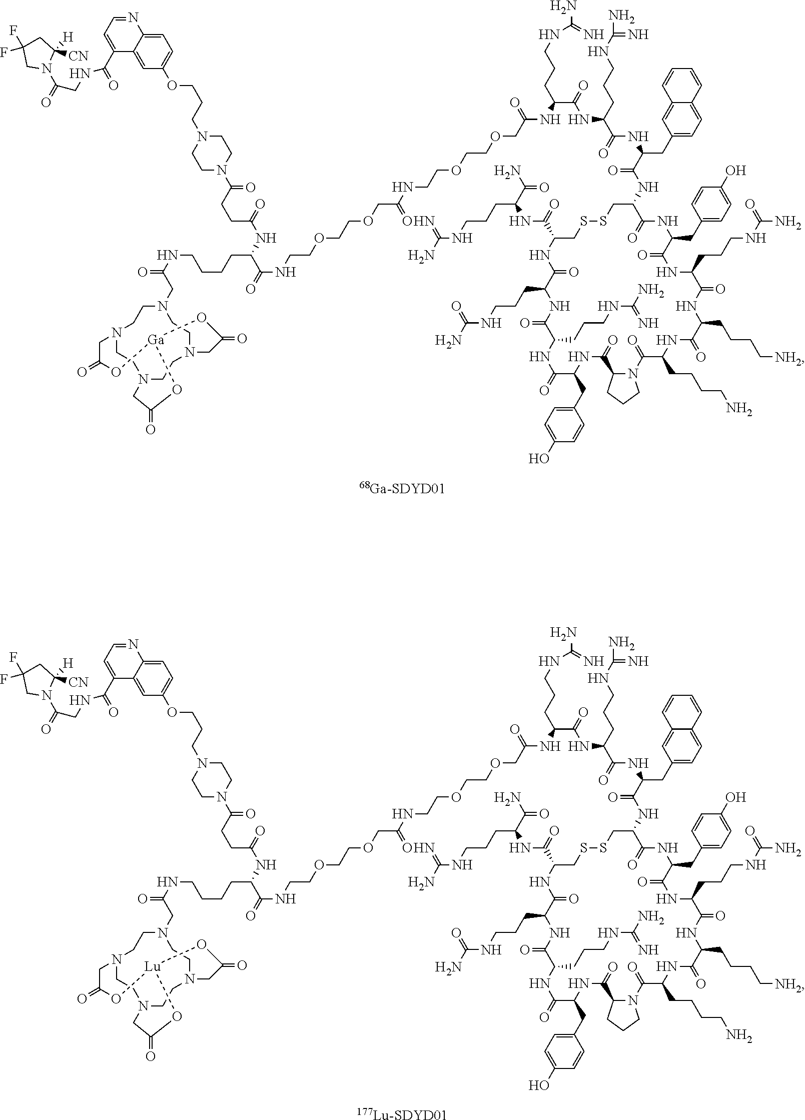

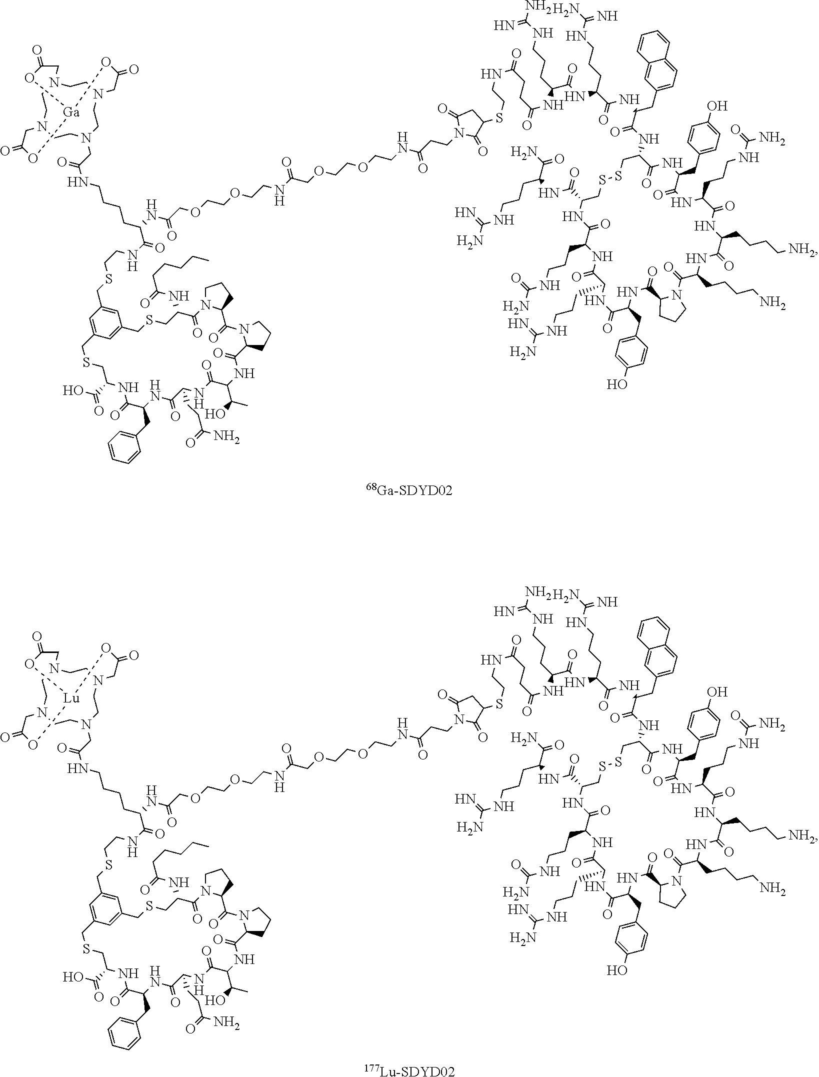

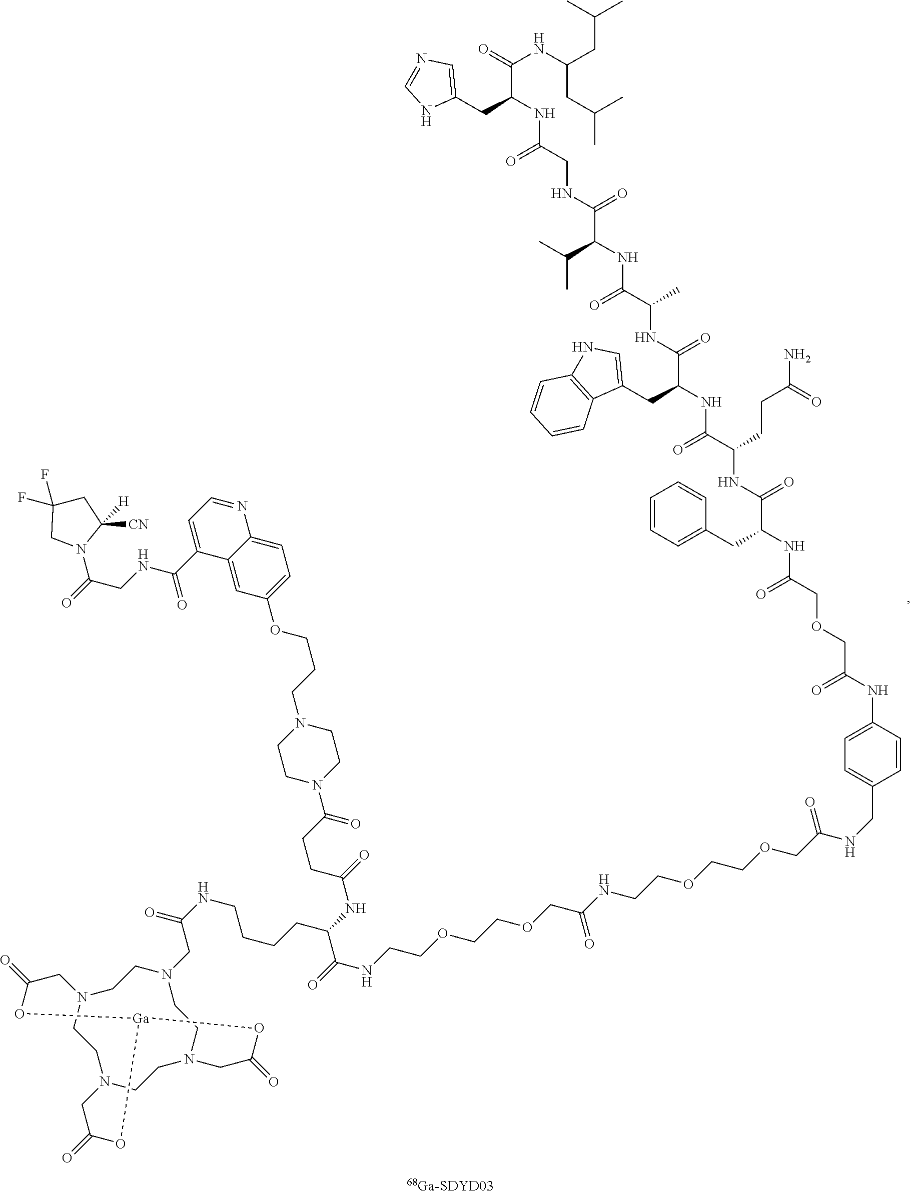

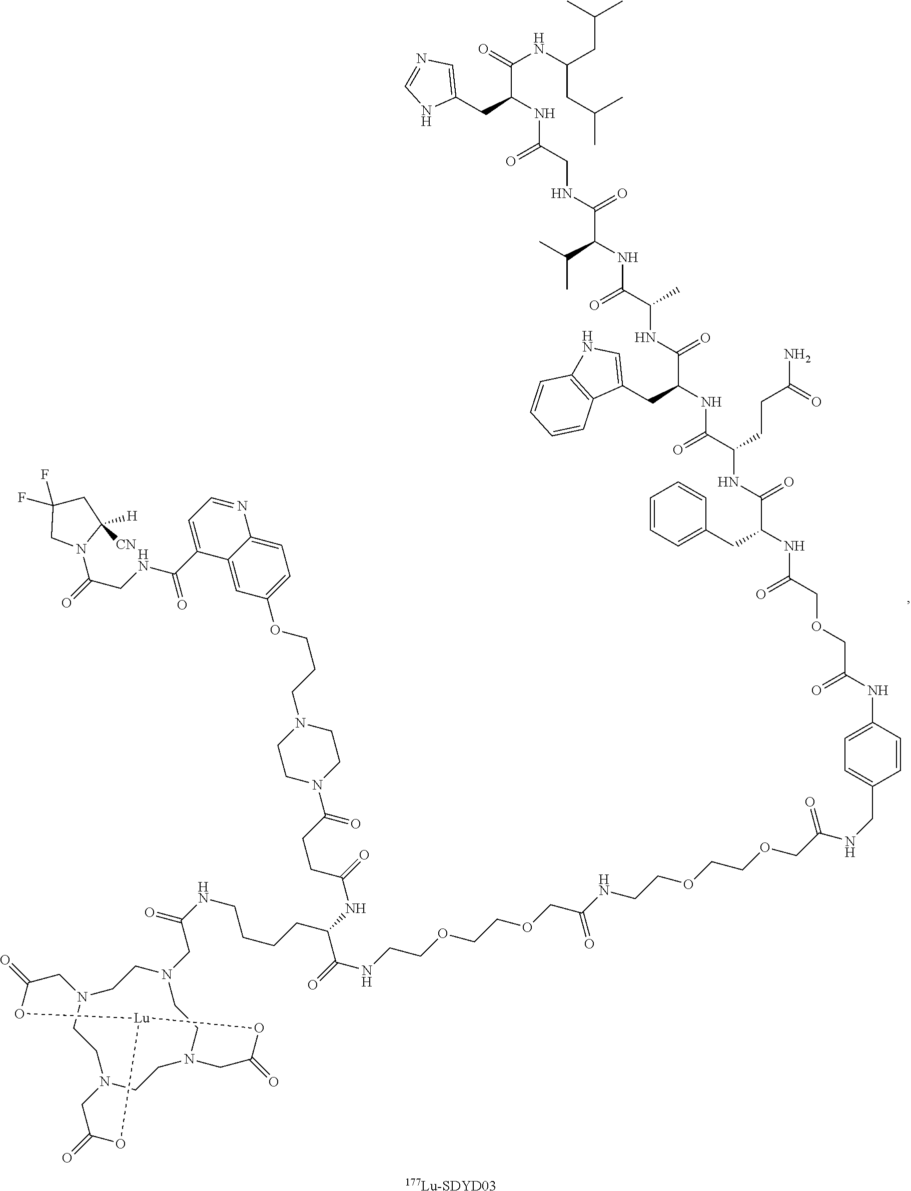

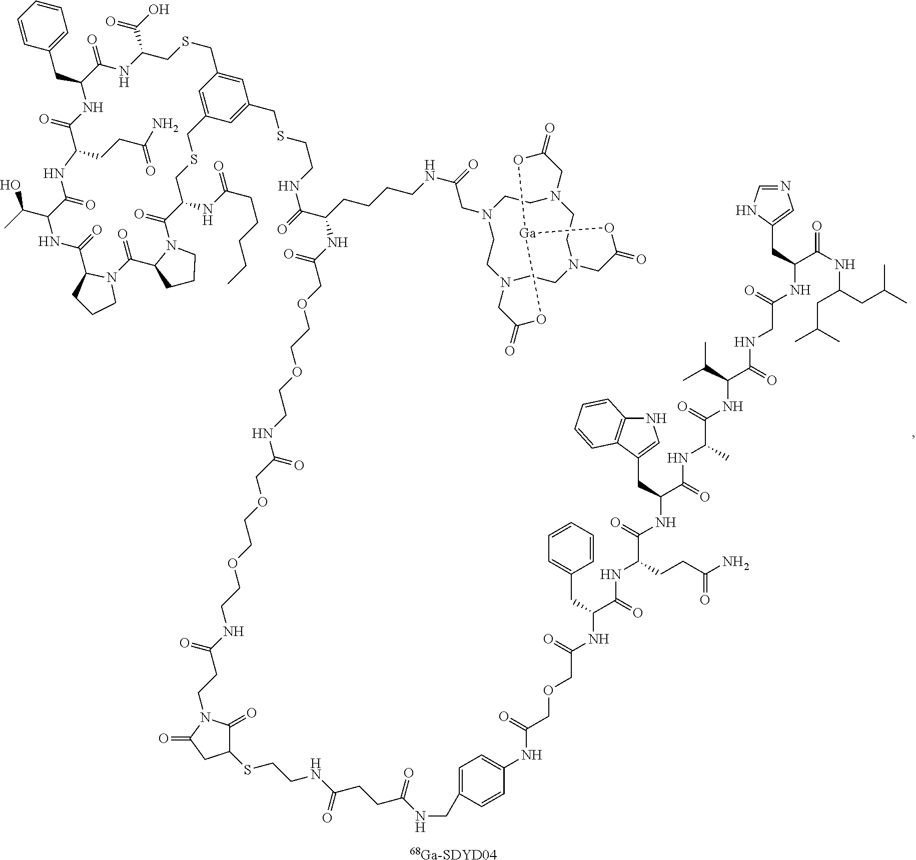

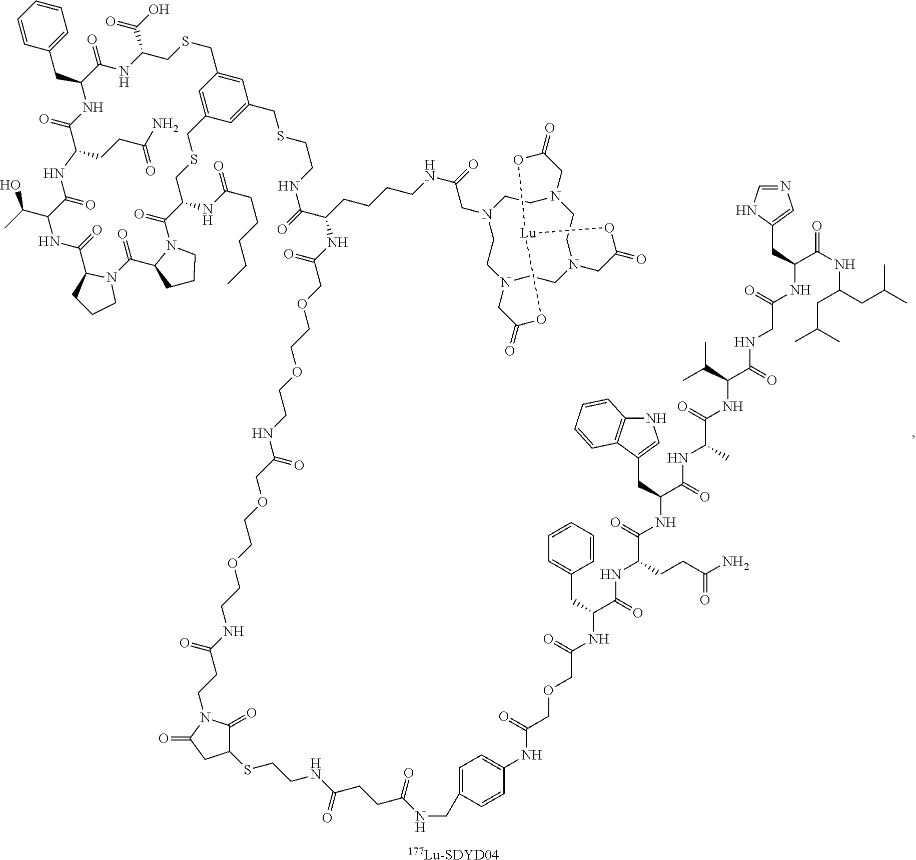

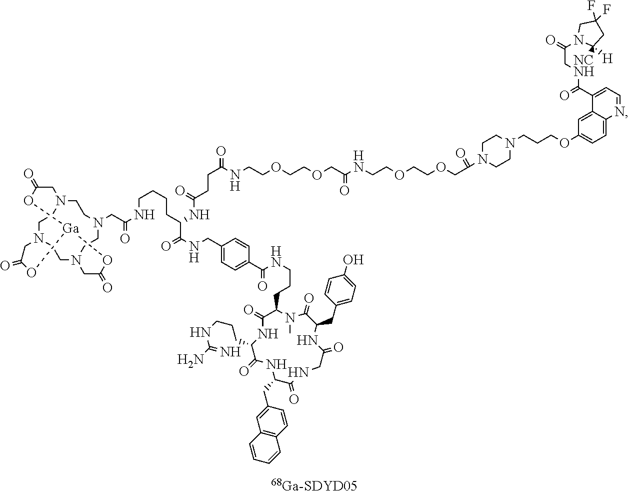

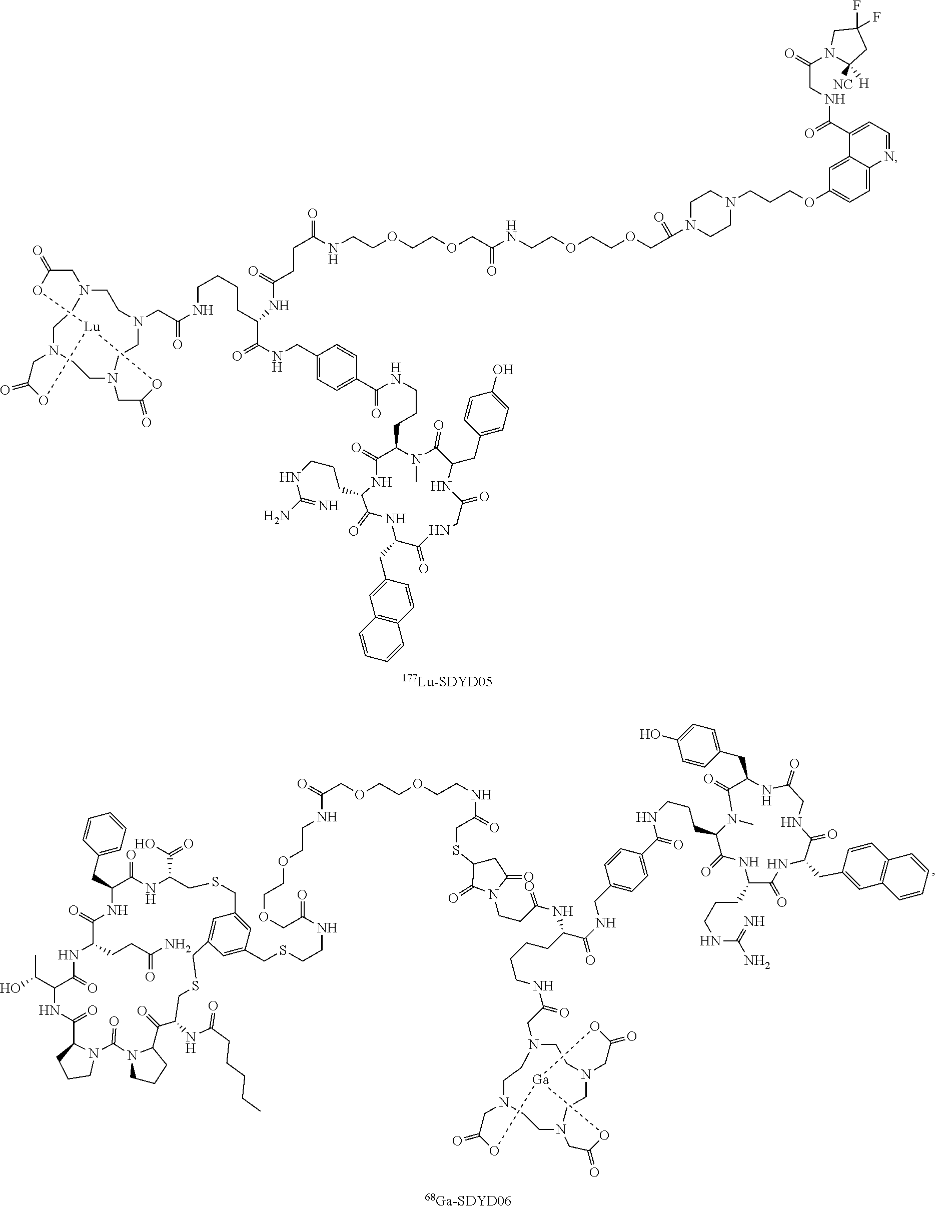

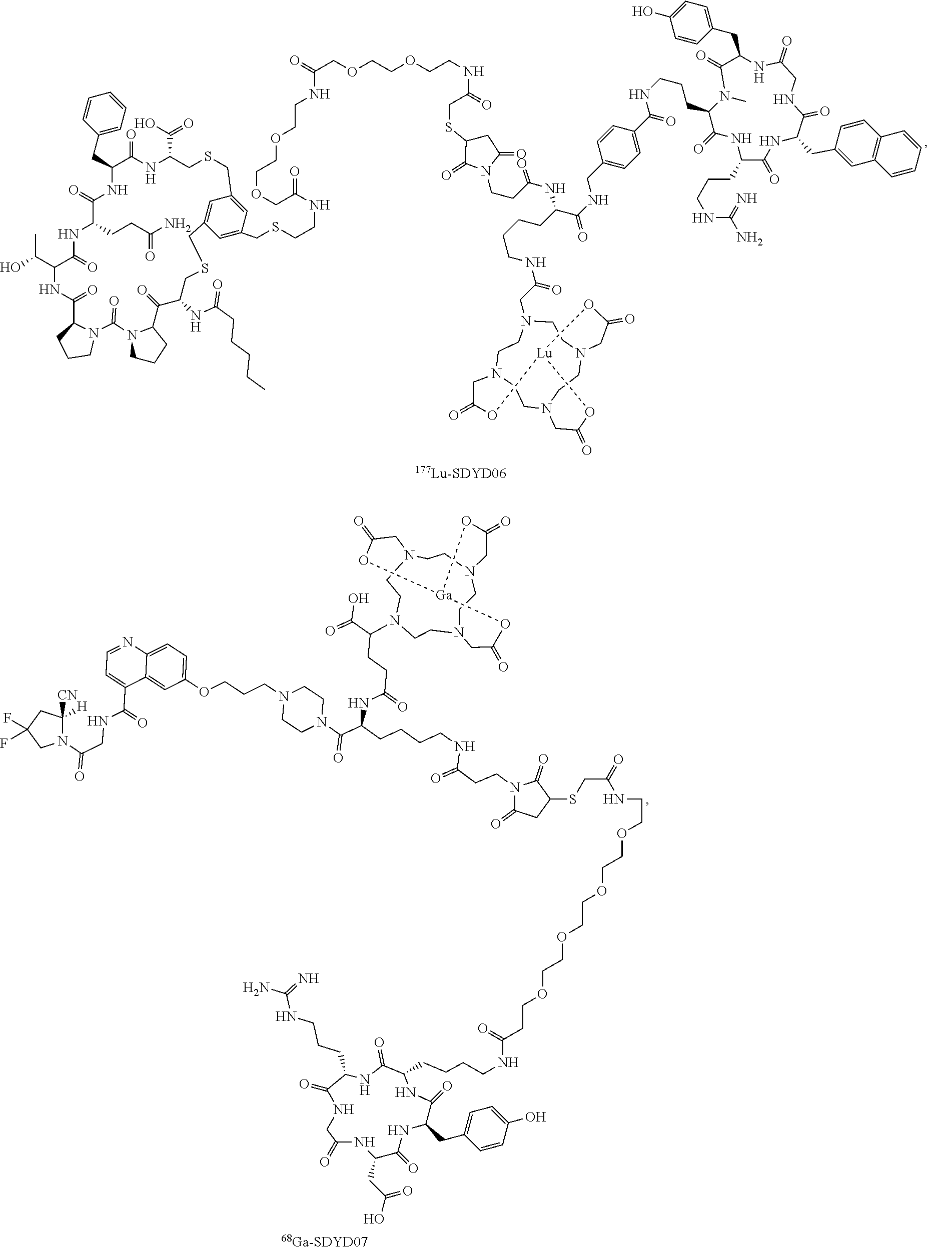

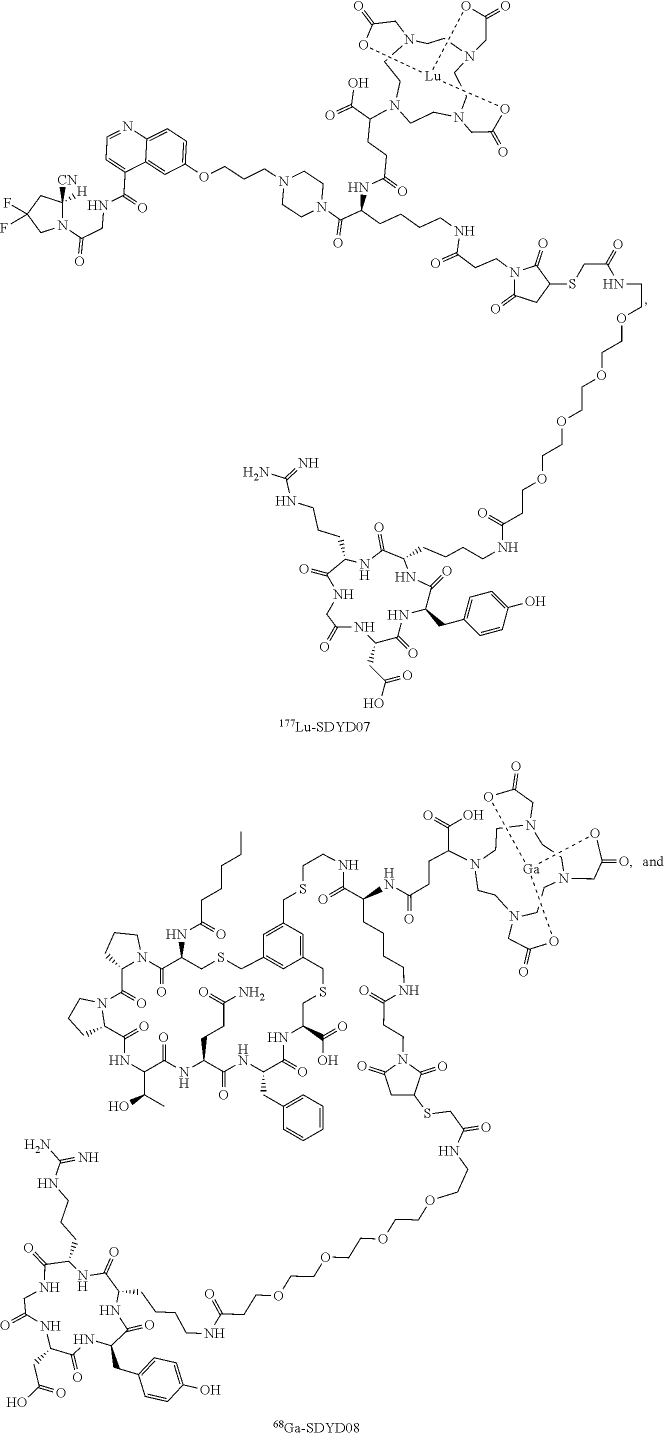

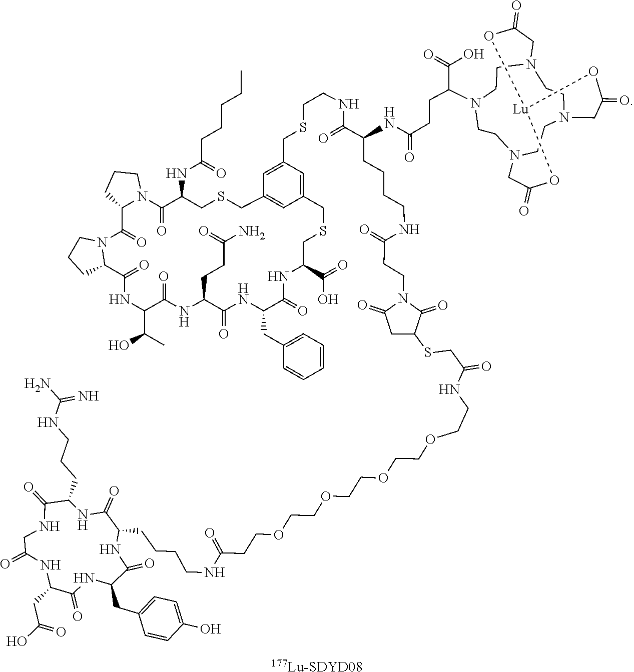

14. A pharmaceutical composition comprising or consisting of at least one compound according to claim 1, and optionally, a pharmaceutically acceptable carrier and/or excipient.

15. A pharmaceutical composition comprising or consisting of at least one radionuclide-labeled compound according to claim 11, and optionally, a pharmaceutically acceptable carrier and or excipient.

16. A method of diagnosis or treatment of a disease in a subject in need thereof, wherein the disease is characterized by abnormal expression of one or two of FAP, CXCR4, GRPR, and αᵥβ₃ in the subject, wherein the method comprising administering the compound according to claim 1 to the subject;
optionally, the disease is characterized by abnormal expression of one or both FAP and CXCR4;
optionally, the disease is characterized by abnormal expression of one or both FAP and GRPR;
optionally, the disease is characterized by abnormal expression of one or both of FAP and αᵥβ₃;
preferably, wherein the disease is selected from the group consisting of cancer, chronic inflammation, atherosclerosis, fibrosis, tissue remodeling and scarring disorder; preferably, wherein the cancer is selected from the group consisting of breast cancer, pancreatic cancer, small bowel cancer, colon cancer, rectal cancer, lung cancer, head and neck cancer, ovarian cancer, hepatocellular carcinoma, esophageal cancer, hypopharyngeal cancer, nasopharyngeal cancer, laryngeal cancer, myeloma cells, bladder cancer, cholangiocarcinoma, clear cell renal cancer, neuroendocrine tumor, oncogenic osteomalacia, sarcoma, CUP (carcinoma of unknown primary), thymic carcinoma, glioma, neuroglioma, astrocytoma, cervical cancer, and prostate cancer.

17. A method of diagnosis or treatment of a disease in a subject in need thereof, wherein the disease is characterized by abnormal expression of one or two of FAP, CXCR4, GRPR, and αᵥβ₃ in the subject, wherein the method comprising administering the radionuclide-labeled compound according to claim 11 to the subject;
optionally, the disease is characterized by abnormal expression of one or both FAP and CXCR4;
optionally, the disease is characterized by abnormal expression of one or both FAP and GRPR;
optionally, the disease is characterized by abnormal expression of one or both of FAP and αᵥβ₃;
preferably, wherein the disease is selected from the group consisting of cancer, chronic inflammation, atherosclerosis, fibrosis, tissue remodeling and scarring disorder; preferably, wherein the cancer is selected from the group consisting of breast cancer, pancreatic cancer, small bowel cancer, colon cancer, rectal cancer, lung cancer, head and neck cancer, ovarian cancer, hepatocellular carcinoma, esophageal cancer, hypopharyngeal cancer, nasopharyngeal cancer, laryngeal cancer, myeloma cells, bladder cancer, cholangiocarcinoma, clear cell renal cancer, neuroendocrine tumor, oncogenic osteomalacia, sarcoma, CUP (carcinoma of unknown primary), thymic carcinoma, glioma, neuroglioma, astrocytoma, cervical cancer, and prostate cancer.

18. A kit comprising or consisting of the compound according to claim 1.

19. A kit comprising or consisting of the radionuclide-labeled compound according to claim 11.
